(19) United States
(12) Patent Application Publication
(10) Pub. No.: US 2024/0277852 A1
(43) Pub. Date: Aug. 22, 2024

(54) INHIBITORS AND DEGRADERS OF PIP4K PROTEIN

(71) Applicants: DANA-FARBER CANCER INSTITUTE, INC., Boston, MA (US); CORNELL UNIVERSITY, Ithaca, NY (US)

(72) Inventors: Nathanael S. Gray, Stanford, CA (US); Mingxing Teng, Boston, MA (US); Eric Wang, Brookline, MA (US); Jie Jiang, Brookline, MA (US); Tinghu Zhang, Brookline, MA (US); Lewis C. Cantley, Cambridge, MA (US); Eric S. Fischer, Chestnut Hill, MA (US); Katherine A. Donovan, Brookline, MA (US)

(73) Assignees: DANA-FARBER CANCER INSTITUTE, INC., Boston, MA (US); CORNELL UNIVERSITY, Ithaca, NY (US)

(21) Appl. No.: 18/559,655

(22) PCT Filed: May 19, 2022
(86) PCT No.: PCT/US2022/029966

§ 371 Date: Nov. 8, 2023

Related U.S. Application Data
(60) Provisional application No. 63/318,114, filed on Mar. 9, 2022.
(60) Provisional application No. 63/190,931, filed on May 20, 2021.

(51) Int. Cl. A61K 47/55; A61K 31/505; A61K 31/506; A61K 31/5383; A61K 47/54; C07D 239/42; C07D 401/04; C07D 401/10; C07D 401/12; C07D 401/14; C07D 403/04; C07D 409/04; C07D 409/10; C07D 417/04; C07D 498/14

(52) U.S. Cl. CPC A61K 47/55; A61K 31/505; A61K 31/506; A61K 31/5383; A61K 47/545; C07D 239/42; C07D 401/04; C07D 401/10; C07D 401/12; C07D 401/14; C07D 403/04; C07D 409/04; C07D 409/10; C07D 417/04; C07D 498/14

(57) ABSTRACT

The present invention relates to compounds, compositions, and methods for treating diseases or conditions by modulating (e.g., reducing) the level or activity of at least one of PIP4K2A, PIP4K2B, and PIP4K2C.

### RELATED APPLICATIONS

This application claims the benefit of priority under 35 U.S.C. § 119(e) to U.S. Provisional Application No. 63/190,931, filed May 20, 2021, and U.S. Provisional Application No. 63/318,114, filed Mar. 9, 2022, each of which is incorporated herein by reference in its entirety.

### GOVERNMENT LICENSE RIGHTS

This invention was made with government support under grant number 5R01CA197329-05 awarded by the National Institutes of Health. The government has certain rights in the invention.

### BACKGROUND OF THE INVENTION

The phosphatidylinositol 5-phosphate 4-kinases (PI5P4Ks), consisting of the three isoforms, PI5P4Kα, β, and γ, are lipid kinases that catalyze phosphorylation of phosphatidylinositol 5-phosphate (PI5P) on its 4-position to form phosphatidylinositol-4,5-bisphosphate (PI-4,5-P2) (Rameh et al., Nature 390(6656):192-196 (1997). In the cellular membrane, PI-4,5-P2 is also produced by another signaling pathway in which phosphatidylinositol 4-phosphate (PI4P) is phosphorylated by phosphatidylinositol 4-phosphate 5-kinases (PI4P5Ks).

Although the majority of PI-4,5-P2 is produced via the PI4P5K pathway, PI5P4Ks have been recognized as key regulators of many cell functions including metabolism, stress response, autophagy, and immunological processes (Hu et al., J. Lipid Res. 59:507-514 (2018); Lamia et al., Mol. Cell. Biol. 24(11):5080-5087 (2004); Shim et al., Proc. Natl. Acad. Sci. U.S.A 113(27):7596-7601 (2016); Al-Ramahi et al., eLife 6:e29123 (2017); Lundquist et al., Mol. Cell 70(3):531-543 (2018); Bulley et al., Proc. Natl. Acad. Sci. U.S.A 113(38):10571-10576 (2016), Keune et al., Adv. Biol. Regul. 53(2):179-189 (2013)). PIP4Ks have distinct catalytic and non-catalytic functions in controlling cellular metabolism and suppress PIP5K activity and insulin-stimulated production of PI(3,4,5)P3 (Wang et al., Cell Rep. 27:1991-2001 (2019)).

Dysregulation of the PI5P4K signaling pathway has been further linked to diseases such as diabetes, neurodegenerative disorders, and cancers (Lamia et al., Mol. Cell. Biol. 24(11): 5080-5087 (2004); Al-Ramahi et al., eLife 6:e29123 (2017); Jude et al., Oncogene 34(10):1253-1262 (2015); Luoh el al., Oncogene 23:1354-1363 (2004); Emerling el al., Cell 155(4):844-857 (2013). Analysis of PI(4,5)P2 levels in cells with single or double knockdown of PIP4K isoforms revealed an additive effect among all three isoforms that does not correlate with their relative catalytic activities. Double knockdown of the most active isoforms, PIP4KA/B, failed to phenocopy the triple knockdown, suggesting that catalytic activity is not the most important factor in regulating PI(4,5)P2 levels (Wang el al., Cell Rep. 27:1991-2001 (2019).

These findings indicate that the inhibition of PI5P4K kinase activity might have therapeutic potential across various diseases.

### SUMMARY OF THE INVENTION

A first aspect of the present invention is directed to a compound having a structure represented by formula (I):

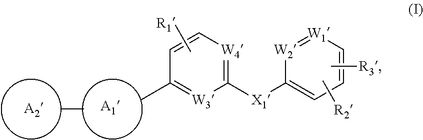

wherein R₁′, R₂′, R₃′, X₁′, W₁′, W₂′, W₃′, W₄′, A₁′ and A₂′ are as defined herein, or a pharmaceutically acceptable salt or stereoisomer thereof.

A second aspect of the present invention is directed to a bifunctional compound, comprising a targeting ligand that binds at least one of phosphatidylinositol-5-phosphate 4-kinase type 2 alpha (PIP4K2A), PIP4K2B, and PIP4K2C and a degron covalently attached to the targeting ligand by a linker, wherein the bifunctional compound has a structure represented by formula (II):

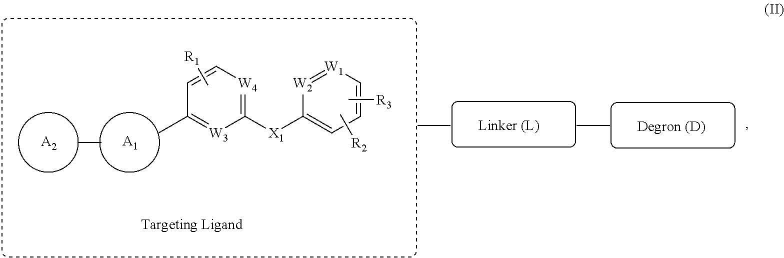

wherein R₁, R₂, R₃, X₁, W₁, W₂, W₃, W₄, A₁ and A₂ are as defined herein; and
the degron is a moiety that binds an E3 ubiquitin ligase, or a pharmaceutically acceptable salt or stereoisomer thereof.

Another aspect of the present invention is directed to a pharmaceutical composition containing a therapeutically effective amount of a compound of formula (I) or a bifunctional compound of formula (II), or a pharmaceutically acceptable salt or stereoisomer thereof, and a pharmaceutically acceptable carrier.

In another aspect of the present invention, methods of making the compounds are provided.

A further aspect of the present invention is directed to a method of treating a disease or disorder by modulating (e.g., reducing) the level or activity of at least one of PIP4K2A, PIP4K2B, and PIP4K2C, that includes administering a therapeutically effective amount of a compound of formula (I), or a pharmaceutically acceptable salt or stereoisomer thereof, to a subject in need thereof.

Another aspect of the present invention is directed to a method of treating a disease or disorder by modulating (e.g., reducing) the level or activity of at least one of PIP4K2A, PIP4K2B, and PIP4K2C, that includes administering a therapeutically effective amount of a bifunctional compound of formula (II), or a pharmaceutically acceptable salt or stereoisomer thereof, to a subject in need thereof.

In some aspects, the present invention is directed to methods of enhancing immune functions in a subject in need thereof, comprising administering to a subject a therapeutically effective amount of a compound of formula (I) or a bifunctional compound of formula (II), or pharmaceutically acceptable salt or stereoisomer thereof.

In some aspects, the present invention is directed to methods of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of a compound of formula (I) or a bifunctional compound of formula (II), or pharmaceutically acceptable salt or stereoisomer thereof.

As shown in working examples, compounds embraced by formula (I) inhibit the activity of at least one of PIP4K2A, PIP4K2B, and PIP4K2C.

As shown in working examples, bifunctional compounds embraced by formula (II) (also referred to herein as degraders) promote the degradation of at least one of PIP4K2A, PIP4K2B, and PIP4K2C.

Without intending to be bound by any particular theory of operation, the bifunctional compounds of formula (II) are believed to cause degradation of at least one of PIP4K2A, PIP4K2B, and PIP4K2C by recruitment of cells' Ubiquitin/Proteasome System, whose function is to routinely identify and remove damaged proteins, into close proximity with PIP4K2A, PIP4K2B, or PIP4K2C as a result of binding between PIP4K2A, PIP4K2B, or PIP4K2C, and the targeting ligand. After destruction of a PIP4K2A, PIP4K2B, or PIP4K2C protein, the degrader is released and continues to be active. Thus, by engaging and exploiting the body's own natural protein disposal system, the compounds of the present invention may represent a potential improvement over current small molecule inhibitors of PIP4K2A, PIP4K2B, and PIP4K2C and may overcome one or more limitations regarding their use. Therefore, effective intracellular concentrations of the degraders may be significantly lower than for small molecule PIP4K2A, PIP4K2B, or PIP4K2C inhibitors. Also, although PIP4K2A, PIP4K2B, and/or PIP4K2C genetic knockdown or knockout can be used to reduce the cellular concentration or amount of these proteins, it may be preferable to post-translationally disrupt, degrade, or destabilize PIP4K2A, PIP4K2B, and/or PIP4K2C proteins. Targeting proteins directly, rather than via the DNA or mRNA molecules that encode them, is a more direct and rapid method for reducing the scaffolding function of PIP4K proteins. Hence, degradation can allow some PIP4K2A, PIP4K2B, and/or PIP4K2C catalytic function to proceed while reducing the non-catalytic functions such as scaffolding between PIP4K2A, PIP4K2B, and/or PIP4K2C proteins and other cellular proteins and structures. Collectively, the present compounds may represent a set of new chemical tools for at least one of PIP4K2A, PIP4K2B, and PIP4K2C knockdown and may provide a potential treatment modality for PIP4K2A, PIP4K2B, and/or PIP4K2C-associated cancers and autophagy-dependent diseases (e.g., neurodegenerative disorders, insulin). The compounds may be used to enhance immune functions as described in WO2020210686 and Wang et al., Cell Rep. 27:1991-2001 (2019), each of which is incorporated herein by reference in its entirety.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Therefore, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2%, or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-Cis group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O— alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocyclyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, I-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., substituted C₁-C₆, C₁-C₅, C₁-C₄, C₁-C₃, C₁-C₂, C₁), haloalkyl (e.g., CF₃), haloalkoxy (e.g., OCF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3-12 membered, 5-6 membered), substituted heterocyclic (e.g., substituted 3-12 membered, 5-6 membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand present in the compounds of formula (II) and the targeted protein, which in this invention is at least one of phosphatidylinositol-5-phosphate 4-kinase type 2 alpha (PIP4K2A), PIP4K2B, and PIP4K2C, typically refers to an inter-molecular interaction that may be preferential (also referred to herein as “selective”) in that binding of other proteins present in the cell is less and in some cases substantially less or even functionally insignificant.

The term “binding” as it relates to interaction between the degron present in the compounds of formula (II) and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted protein.

### Compounds of Formula (I)

In one aspect, compounds of the invention are represented by formula (I):

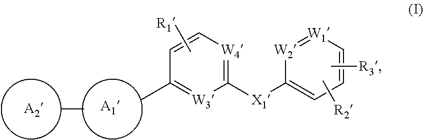

or a pharmaceutically acceptable salt or stereoisomer thereof,
wherein:

X₁′ is a bond, CH₂, CHMe, NH, NMe, or O;

W₁′ is CR₄′ or N;

W₂′ is CR₄′ or N;

each R₄′ is independently hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, C(O)NH₂, C(O)NH(C₁-C₆) alkyl, NHC(O)(C₁-C₆) alkyl, optionally substituted C₅-C₆ carbocyclyl or optionally substituted 5- or 6-membered heterocyclyl;

W₃′ is CH or N;

W₄′ is CH or N;

R₁′ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;

R₂′ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl or optionally substituted 5- or 6-membered heterocyclyl, or

R₂′ and R₃′, together with the carbon atoms to which they are attached, form an optionally substituted 5- to 10-membered heterocyclyl:

R₃′ is hydrogen, (C₁-C₆) alkoxy, NH(C₁-C₆) alkyl, SO₂NH₂, SO₂NH(C₁-C₆) alkyl, SO₂N((C₁-C₆) alkyl)₂, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, NHC(O)aryl, or NHC(O)NH(C₁-C₆) alkyl, wherein said (C₁-C₆) alkyl group of R₃′ is optionally substituted; and

A₁′ and A₂′ are each independently optionally substituted aryl or optionally substituted 5- to 6-membered heteroaryl.

In some embodiments, compounds are represented by Formula I′:

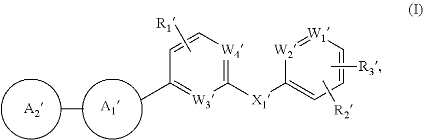

or a pharmaceutically acceptable salt or stereoisomer thereof, wherein:

X₁′ is a bond, CH₂, CHMe, NH, NMe, or O;

W₁′ is CR₄′ or N;

W₂′ is CR₄′ or N;

each R₄′ is independently hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, C(O)NH₂, C(O)NH(C₁-C₆) alkyl, NHC(O)(C₁-C₆) alkyl, optionally substituted C₅-C₆ carbocyclyl or optionally substituted 5- or 6-membered heterocyclyl;

W₃′ is CH or N;

W₄′ is CH or N;

R₁′ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;

R₂′ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl or optionally substituted 5- or 6-membered heterocyclyl, or

R₂′ and R₃′, together with the carbon atoms to which they are attached, form an optionally substituted heterocyclyl;

R₃′ is (C₁-C₆) alkoxy, NH(C₁-C₆) alkyl, SO₂NH₂, SO₂NH(C₁-C₆) alkyl, SO₂N((C₁-C₆) alkyl)₂, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, NHC(O)aryl, or NHC(O)NH(C₁-C₆) alkyl, wherein said alkyl group is optionally substituted; and

A₁′ and A₂′ are each independently optionally substituted aryl or optionally substituted 5- to 6-membered heteroaryl.

In some embodiments, X₁′ is NH.

In some embodiments, R₁′ is halogen.

In some embodiments, R₁′ is chloro.

In some embodiments, R₁′ is CN.

In some embodiments, W₁′ is CR₄′.

In some embodiments, R₄′ is hydrogen or methyl.

In some embodiments, W₁′ is N.

In some embodiments, W₂′ is CR₄′.

In some embodiments, R₄′ is —C(O)NH₂, or —NHC(O)Me.

In some embodiments, W₂′ is N.

In some embodiments, R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S. In some embodiments, R₂′ is hydrogen. In some embodiments, R₂′ is methyl. In some embodiments, R₂′ is

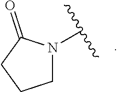 [SMILES: *N1CCCC1=O]

In some embodiments, R₃′ is SO₂NH₂, SO₂NH(C₁-C₆) alkyl, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, or NHC(O)aryl, wherein said alkyl group is optionally substituted. In some embodiments, R₃′ is SO₂NH₂, C(O)NH₂,

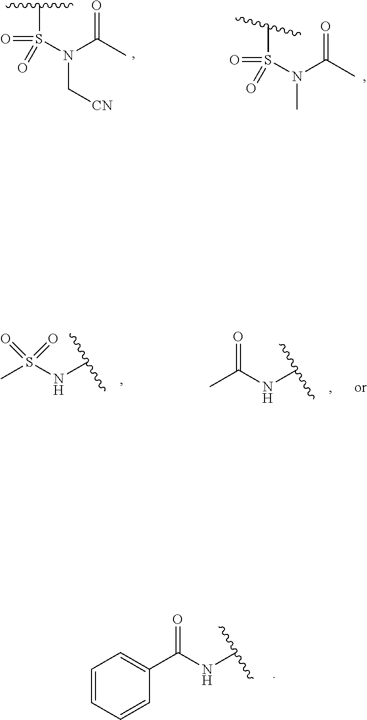

In some embodiments, R₃′ is SO₂NH₂.

In some embodiments, R₂′ and R₃′, together with the carbon atoms to which they are attached, form an optionally substituted 5- to 10-membered heterocyclyl, containing 1 to 3 heteroatoms selected from N, O, and S. In some embodiments, R₂′ and R₃′, together with the carbon atoms to which they are attached form

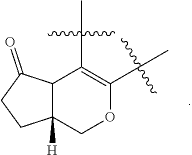 [SMILES: *C1=C(*)C2C(=O)CC[C@H]2CO1]

In some embodiments, W₃′ is N.

In some embodiments, W₄′ is N.

In some embodiments, A₁′ is optionally substituted phenyl. In some embodiments, A₁′ is phenyl.

In some embodiments, A₁′ is optionally substituted 5- to 6-membered heteroaryl, containing 1 to 3 heteroatoms selected from N, S, and O. In some embodiments, A₁′ is 5- to 6-membered heteroaryl, containing 1 to 3 heteroatoms selected from N, O, and S. In some embodiments, A₁′ is optionally substituted pyrrole, imidazole, pyrazole, thiazole, triazole, or pyridine.

In some embodiments, A₂′ is optionally substituted phenyl. In some embodiments, A₂′ is phenyl.

In some embodiments, A₂′ is optionally substituted 5- to 6-membered heteroaryl, containing 1 to 3 heteroatoms selected from N, O, and S. In some embodiments, A₂′ is 5- to 6-membered heteroaryl, containing 1 to 3 heteroatoms selected from N, O, and S. In some embodiments, A₂′ is optionally substituted thiophene, pyrrole, pyrimidine, or pyridazine.

In some embodiments, X₁′ is NH, R₁′ is chloro or CN, W₁′ is N or CH, and W₂′ is N or CH.

In some embodiments, X₁′ is NH, R₁′ is chloro or CN, W₁′ is N or CH, W₂′ is N or CH and R₄′ is —C(O)NH₂, or —NHC(O)Me.

In some embodiments, X₁′ is NH, R₁′ is chloro or CN, W₁′ is N or CH, W₂′ is N or CH, R₄′ is —C(O)NH₂, or —NHC(O)Me, and R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S.

In some embodiments, X₁′ is NH, R₁′ is chloro or CN, W₁′ is N or CH, W₂′ is N or CH, R₄′ is —C(O)NH₂, or —NHC(O)Me, R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S, and R₃′ is SO₂NH₂, SO₂NH(C₁-C₆) alkyl, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, or NHC(O)aryl, wherein said alkyl group is optionally substituted.

In some embodiments, X₁′ is NH, R₁′ is chloro or CN, W₁′ is N or CH, W₂′ is N or CH, R₄′ is —C(O)NH₂, or —NHC(O)Me, R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S, and R₃′ is SO₂NH₂, C(O)NH₂,

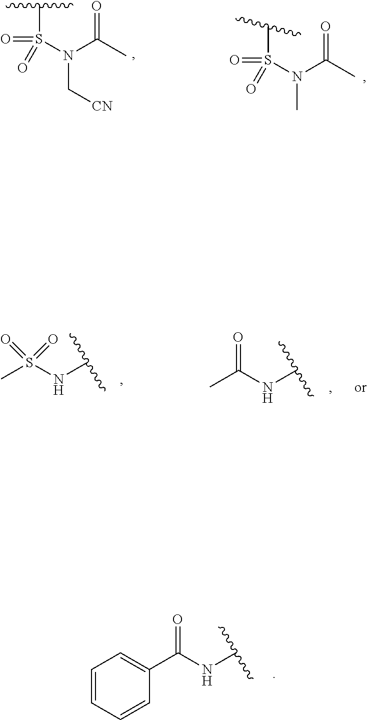

In some embodiments, X₁′ is NH, R₁′ is chloro or CN, W₁′ is N or CH, W₂′ is N or CH, R₄′ is —C(O)NH₂, or —NHC(O)Me, and R₂′ and R₃′, together with the carbon atoms to which they are attached, form an optionally substituted 5- to 10-membered heterocyclyl, containing 1 to 3 heteroatoms selected from N, O, and S.

In some embodiments, X₁′ is NH, R₁′ is chloro or CN, W₁′ is N or CH, W₂′ is N or CH, R₄′ is —C(O)NH₂, or —NHC(O)Me, R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S, R₃′ is SO₂NH₂, SO₂NH(C₁-C₆) alkyl, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, or NHC(O)aryl, wherein said alkyl group is optionally substituted, W₃′ is N, and W₄′ is N.

In some embodiments, X₁′ is NH, R₁′ is chloro or CN, W₁′ is N or CH, W₂′ is N or CH, R₄′ is —C(O)NH₂, or —NHC(O)Me, R₂′ and R₃′, together with the carbon atoms to which they are attached, form an optionally substituted 5- to 10-membered heterocyclyl, containing 1 to 3 heteroatoms selected from N, O, and S, W₃′ is N, and W₄′ is N.

In some embodiments, X₁′ is NH, R₁′ is chloro or CN, W₁′ is N or CH, W₂′ is N or CH, R₄′ is —C(O)NH₂, or —NHC(O)Me, R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S, R₃′ is SO₂NH₂, SO₂NH(C₁-C₆) alkyl, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, or NHC(O)aryl, wherein said alkyl group is optionally substituted, W₃′ is N, W₄′ is N, A₁′ is optionally substituted phenyl, and A₂′ is optionally substituted phenyl or optionally substituted 5- to 6-membered heteroaryl, containing 1 to 3 heteroatoms selected from N, O, and S.

In some embodiments, X₁′ is NH, R₁′ is chloro or CN, W₁′ is N or CH, W₂′ is N or CH, R₄′ is —C(O)NH₂, or —NHC(O)Me, R₂′ and R₃′, together with the carbon atoms to which they are attached, form an optionally substituted 5- to 10-membered heterocyclyl, containing 1 to 3 heteroatoms selected from N, O, and S, W₃′ is N, W₄′ is N, and A₁′ is optionally substituted phenyl, and A₂′ is optionally substituted phenyl or optionally substituted 5- to 6-membered heteroaryl, containing 1 to 3 heteroatoms selected from N, O, and S.

In some embodiments, the optional substituent for a compound of Formula (I or I′) is (C₁-C₆) alkyl, (C₂-C₆) alkenyl, (C₂-C₆) alkynyl, halo, (C₁-C₆) haloalkyl, (C₁-C₆) cycloalkyl, 5- to 10-membered heterocycloalkyl, hydroxy, (C₁-C₆) alkoxy, (C₆-C₁₀) cycloalkoxy, 5- to 10-membered heterocycloalkoxy, (C₁-C₆) haloalkoxy, (C₆-C₁₀) aryloxy, 5- to 10-membered heteroaryloxy, (C₆-C₁₀) aralkyloxy, (C₂-C₆) alkyenyloxy, (C₂-C₆) alkynyloxy, amino, (C₁-C₆) alkylamino, (C₆-C₁₀) cycloalkylamino, 5- to 10-membered heterocycloalkylamino, (C₆-C₁₀) arylamino, 5- to 10-membered heteroarylamino, (C₆-C₁₀) aralkylamino, N—(C₁-C₆) alkyl-N—(C₆-C₁₀) arylamino, N—(C₁-C₆) alkyl-N-5- to 10-membered heteroarylamino, N—(C₁-C₆) alkyl-N—(C₆-C₁₀) aralkylamino, (C₁-C₆) hydroxyalkyl, (C₁-C₆) aminoalkyl, (C₁-C₆) alkylthio, (C₁-C₆) haloalkylthio, (C₁-C₆) alkylsulfonyl, (C₁-C₆) haloalkylsulfonyl, (C₆-C₁₀) cycloalkylsulfonyl, 5- to 10-membered heterocycloalkylsulfonyl, (C₆-C₁₀) arylsulfonyl, 5- to 10-membered heteroarylsulfonyl, aminosulfonyl, (C₁-C₆) alkylaminosulfonyl, (C₆-C₁₀) cycloalkylaminosulfonyl, 5- to 10-membered heterocycloalkylaminosulfonyl, (C₆-C₁₀) arylaminosulfonyl, 5- to 10-membered heteroarylaminosulfonyl, N—(C₁-C₆) alkyl-N—(C₆-C₁₀) arylaminosulfonyl, N—(C₁-C₆) alkyl-N-5- to 10-membered heteroarylaminosulfonyl, formyl, (C₁-C₆) alkylcarbonyl, (C₁-C₆) haloalkylcarbonyl, (C₂-C₆) alkenylcarbonyl, (C₂-C₆) alkynylcarbonyl, carboxy, (C₁-C₆) alkoxycarbonyl, (C₁-C₆) alkylcarbonyloxy, amino, (C₁-C₆) alkylsulfonylamino, (C₁-C₆) haloalkylsulfonylamino, (C₆-C₁₀) cycloalkylsulfonylamino, 5- to 10-membered heterocycloalkylsulfonylamino, (C₆-C₁₀) arylsulfonylamino, 5- to 10-membered heteroarylsulfonylamino, (C₆-C₁₀) aralkylsulfonylamino, (C₁-C₆) alkylcarbonylamino, (C₁-C₆) haloalkylcarbonylamino, (C₆-C₁₀) cycloalkylcarbonylamino, 5- to 10-membered heterocycloalkylcarbonylamino, (C₆-C₁₀) arylcarbonylamino, 5- to 10-membered heteroarylcarbonylamino, (C₆-C₁₀) aralkylsulfonylamino, aminocarbonyl, (C₁-C₆) alkylaminocarbonyl, (C₆-C₁₀) cycloalkylaminocarbonyl, 5- to 10-membered heterocycloalkylaminocarbonyl, (C₆-C₁₀) arylaminocarbonyl, 5- to 10-membered heteroarylaminocarbonyl, N—(C₁-C₆) alkyl-N—(C₆-C₁₀) arylaminocarbonyl, N—(C₁-C₆) alkyl-N-5- to 10-membered heteroarylaminocarbonyl, cyano, nitro, oxo, azido, 3- to 10-membered carbocyclyl, or 4- to 10-membered heterocyclyl. In some embodiments, the optional substituent is not further substituted. In some embodiments, the optional substituent is further substituted.

In some embodiments, the optional substituent for a compound of Formula (I or I′) is (C₁-C₆) alkyl, (C₁-C₆) haloalkyl, (C₁-C₆) alkoxy, amino, (C₁-C₆) aminoalkyl, hydroxy, halo, cyano, oxo, (C₆-C₁₂) aryl, 5- to 10-membered heterocyclyl, sulfonylamino, or (C₁-C₆) alkylsulfonylamino.

In some embodiments, the optional substituent for a compound of Formula (I or I′) is methyl, fluoro, cholo, cyano, —OMe, —OCF₃, —NH₂, —NHMe, —NMe₂, —SO₂NH₂, —C(O)NH₂,

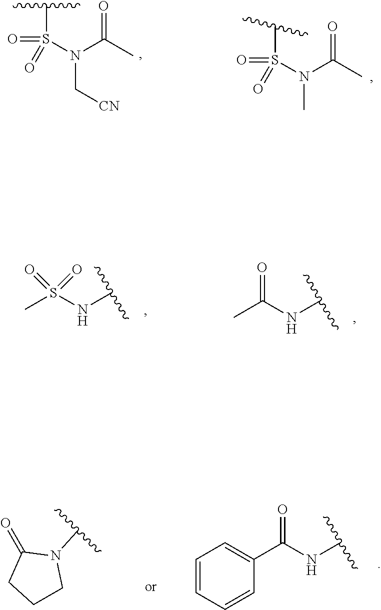

In some embodiments, the compound of formula I is one of formulas Ia-Ii:

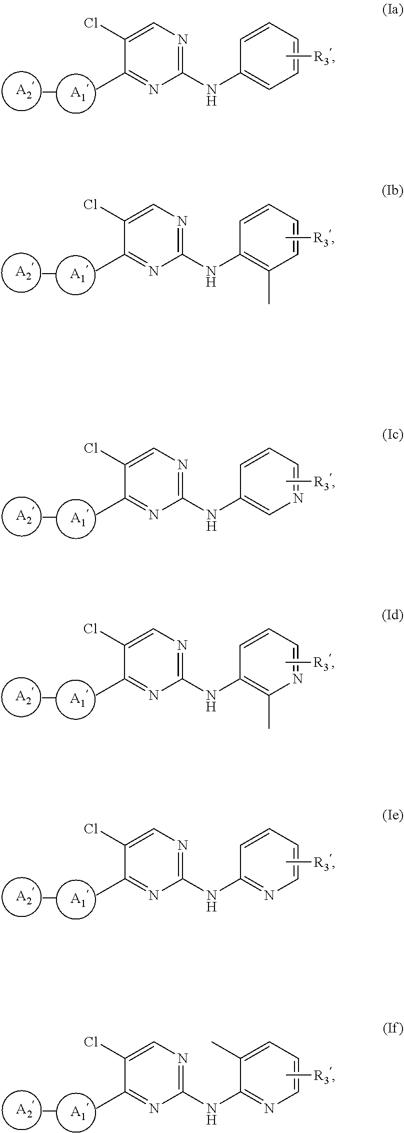

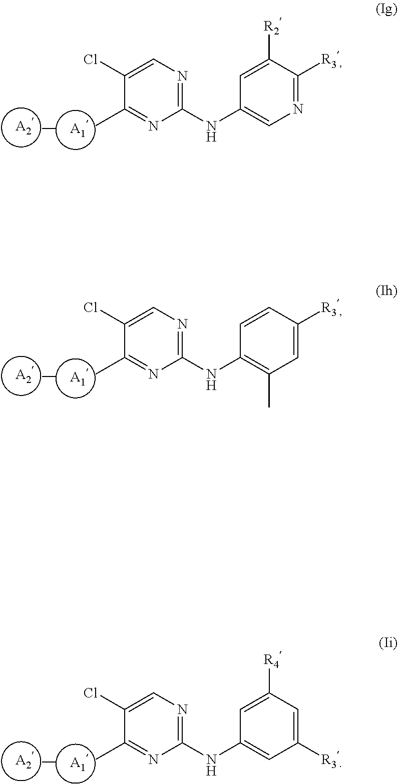

In some embodiments, the compound of formula I is represented by one of the following structures:

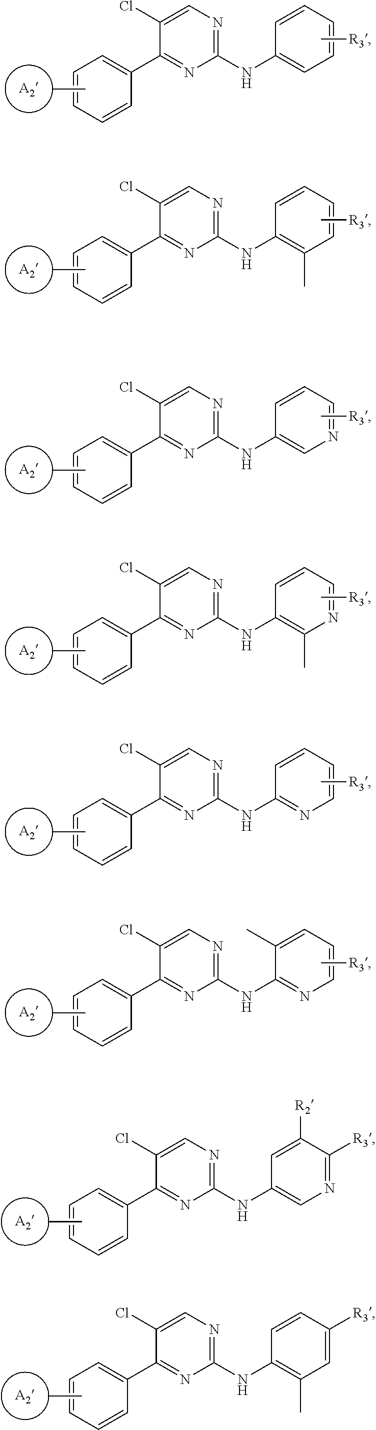

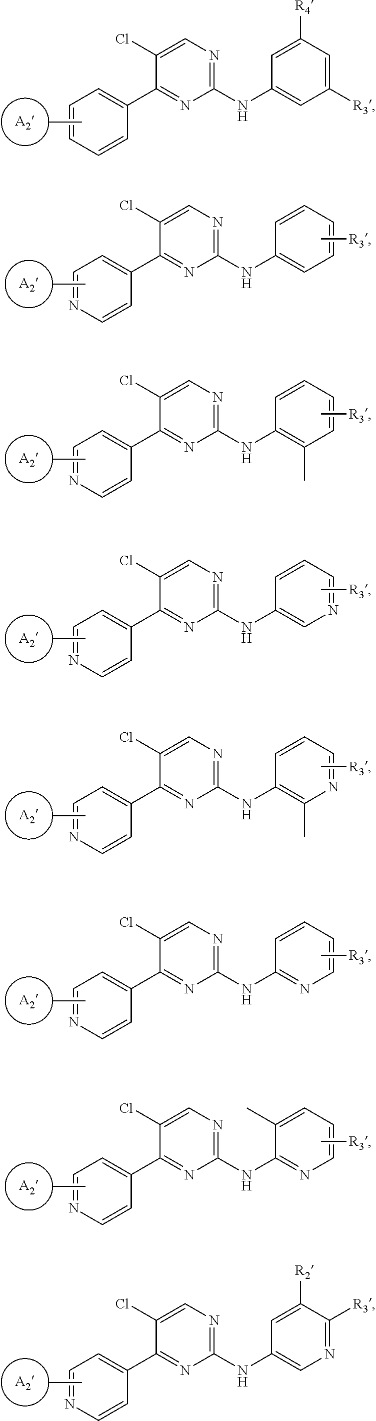

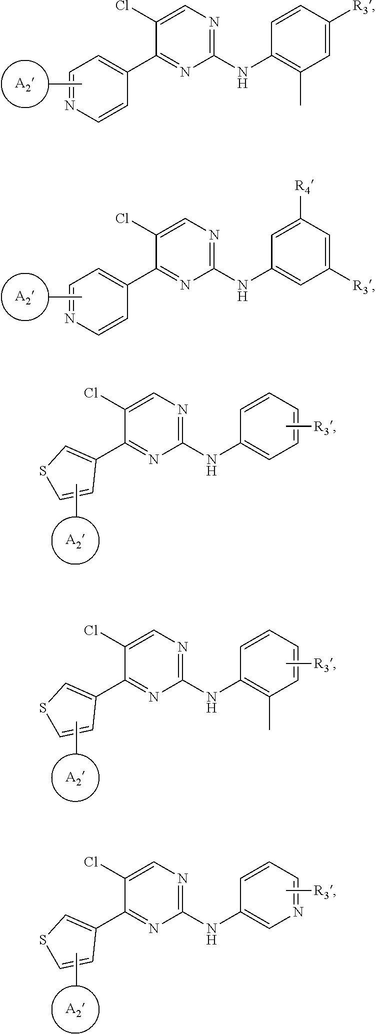

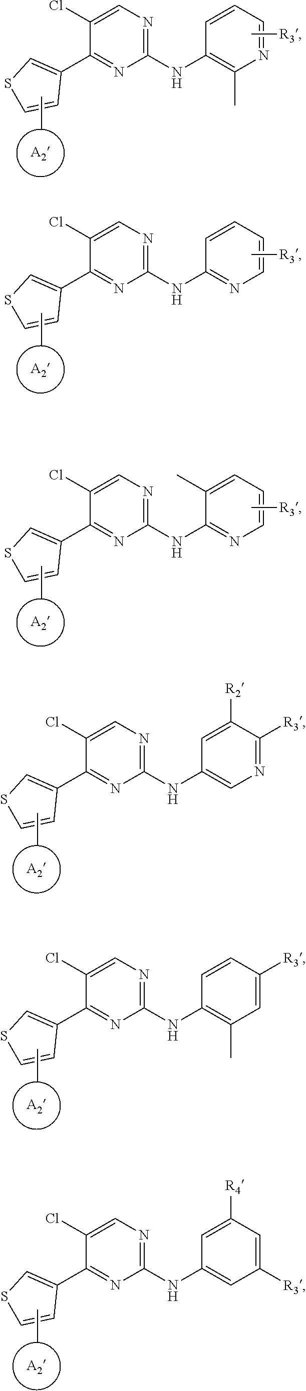

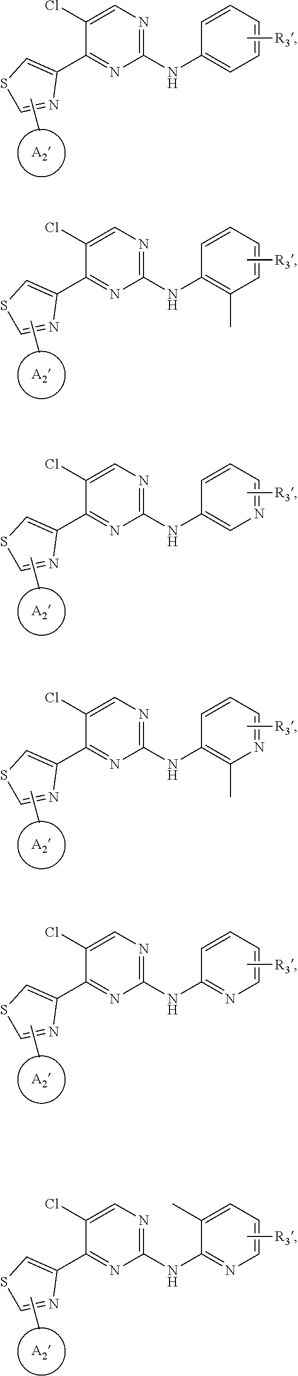

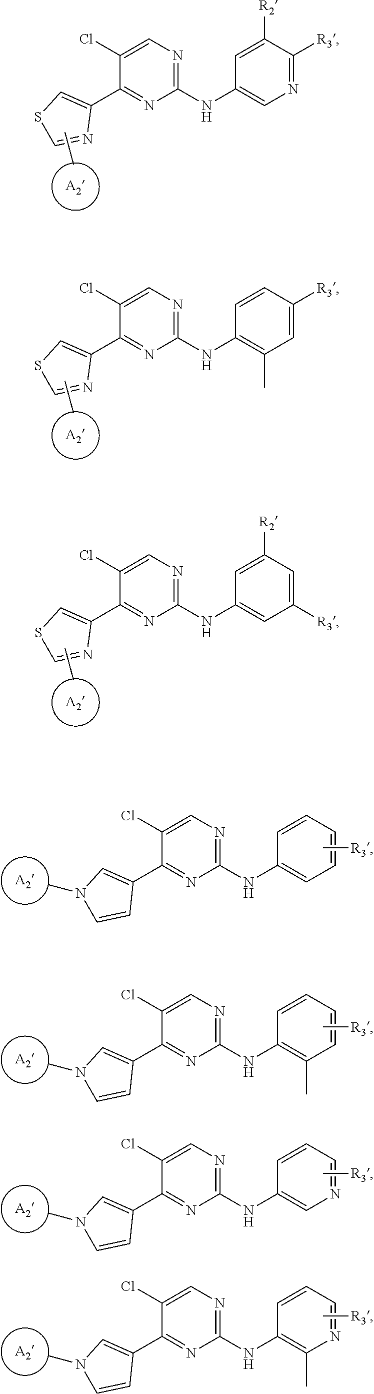

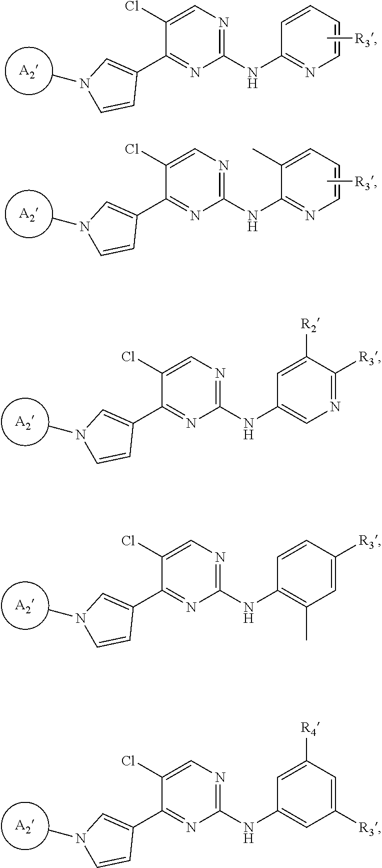

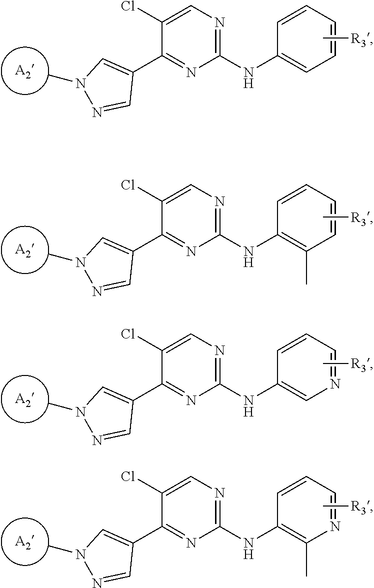

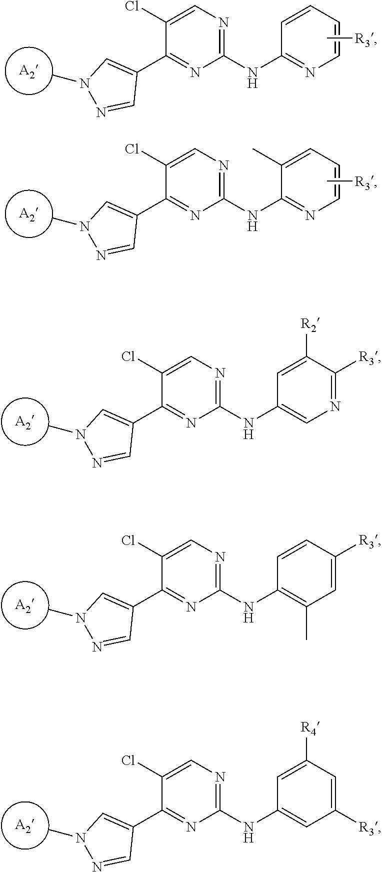

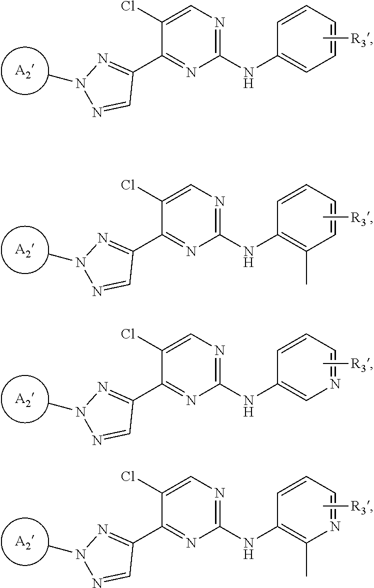

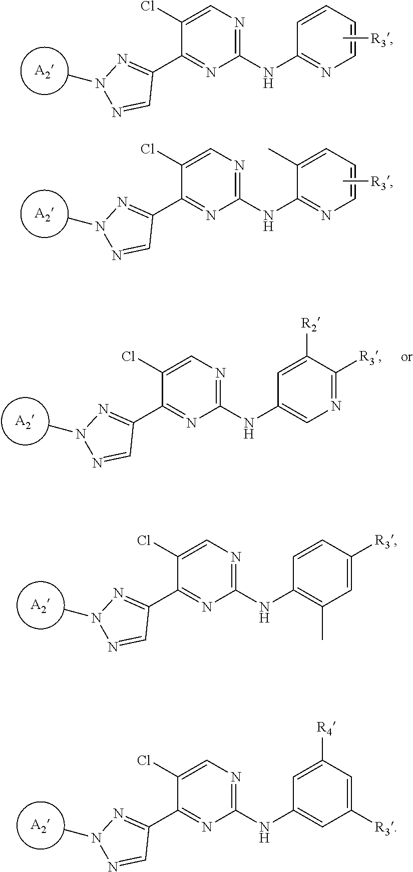

In some embodiments, A₂′ is optionally substituted phenyl. In some embodiments, A₂′ is phenyl. In some embodiments, A₂′ is optionally substituted 5- to 6-membered heteroaryl, containing 1 to 3 heteroatoms selected from N, O, and S. In some embodiments, A₂′ is 5- to 6-membered heteroaryl, containing 1 to 3 heteroatoms selected from N, O, and S. In some embodiments, A₂′ is optionally substituted thiophene, pyrrole, pyrimidine, or pyridazine. In some embodiments, R₃′ is SO₂NH₂, SO₂NH(C₁-C₆) alkyl, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, or NHC(O)aryl, wherein said alkyl group is optionally substituted. In some embodiments, R₃′ is SO₂NH₂, C(O)NH₂,

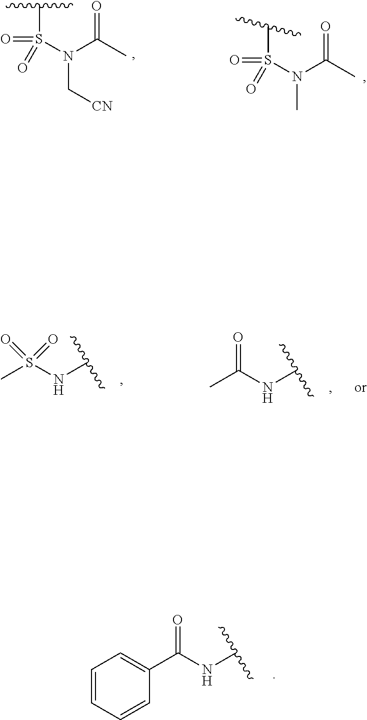

In some embodiments, R₄′ is C(O)NH₂. In some embodiments, R₄′ is NHC(O)Me.

In some embodiments, compounds of formula (I) are represented by any one of the following structures:

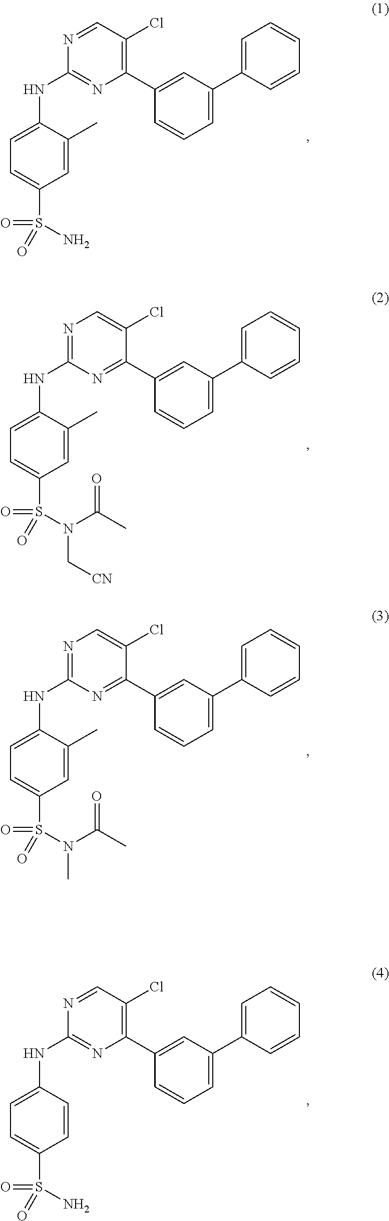 [SMILES: Cc1cc(S(N)(=O)=O)ccc1Nc1ncc(Cl)c(-c2cccc(-c3ccccc3)c2)n1 ; CC(=O)N(CC#N)S(=O)(=O)c1ccc(Nc2ncc(Cl)c(-c3cccc(-c4ccccc4)c3)n2)c(C)c1 ; CC(=O)N(C)S(=O)(=O)c1ccc(Nc2ncc(Cl)c(-c3cccc(-c4ccccc4)c3)n2)c(C)c1 ; NS(=O)(=O)c1ccc(Nc2ncc(Cl)c(-c3cccc(-c4ccccc4)c3)n2)cc1]

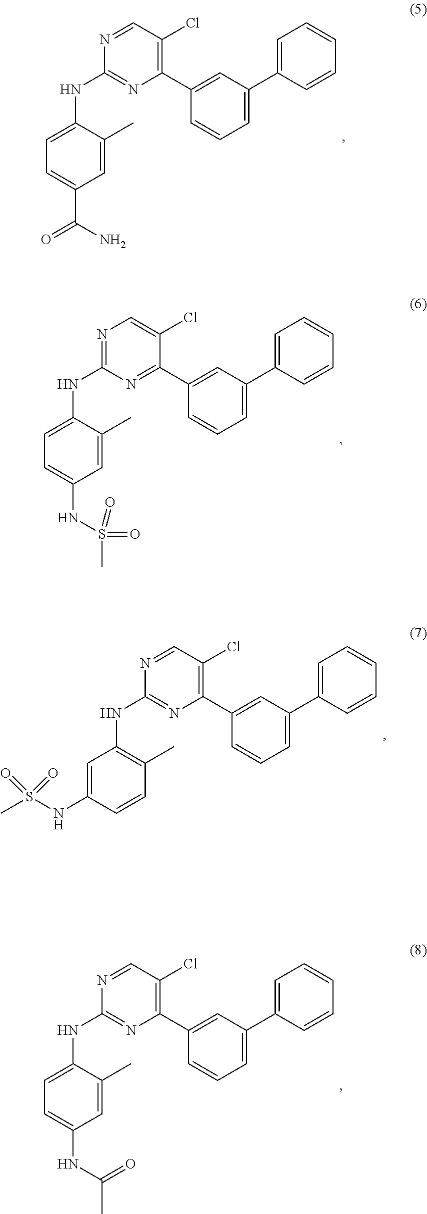 [SMILES: Cc1cc(C(N)=O)ccc1Nc1ncc(Cl)c(-c2cccc(-c3ccccc3)c2)n1 ; Cc1cc(NS(C)(=O)=O)ccc1Nc1ncc(Cl)c(-c2cccc(-c3ccccc3)c2)n1 ; Cc1ccc(NS(C)(=O)=O)cc1Nc1ncc(Cl)c(-c2cccc(-c3ccccc3)c2)n1 ; CC(=O)Nc1ccc(Nc2ncc(Cl)c(-c3cccc(-c4ccccc4)c3)n2)c(C)c1]

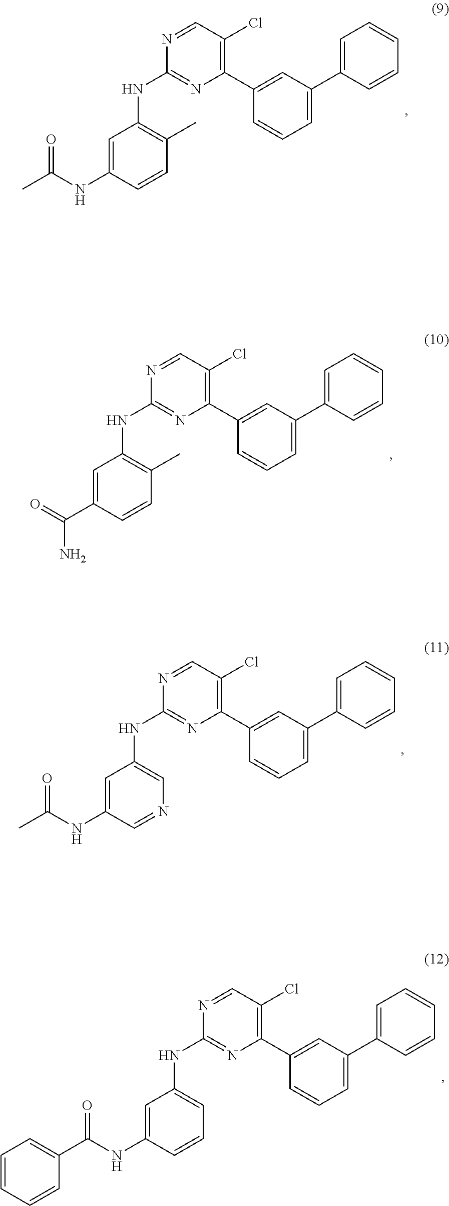 [SMILES: CC(=O)Nc1ccc(C)c(Nc2ncc(Cl)c(-c3cccc(-c4ccccc4)c3)n2)c1 ; Cc1ccc(C(N)=O)cc1Nc1ncc(Cl)c(-c2cccc(-c3ccccc3)c2)n1 ; CC(=O)Nc1cncc(Nc2ncc(Cl)c(-c3cccc(-c4ccccc4)c3)n2)c1 ; O=C(Nc1cccc(Nc2ncc(Cl)c(-c3cccc(-c4ccccc4)c3)n2)c1)c1ccccc1]

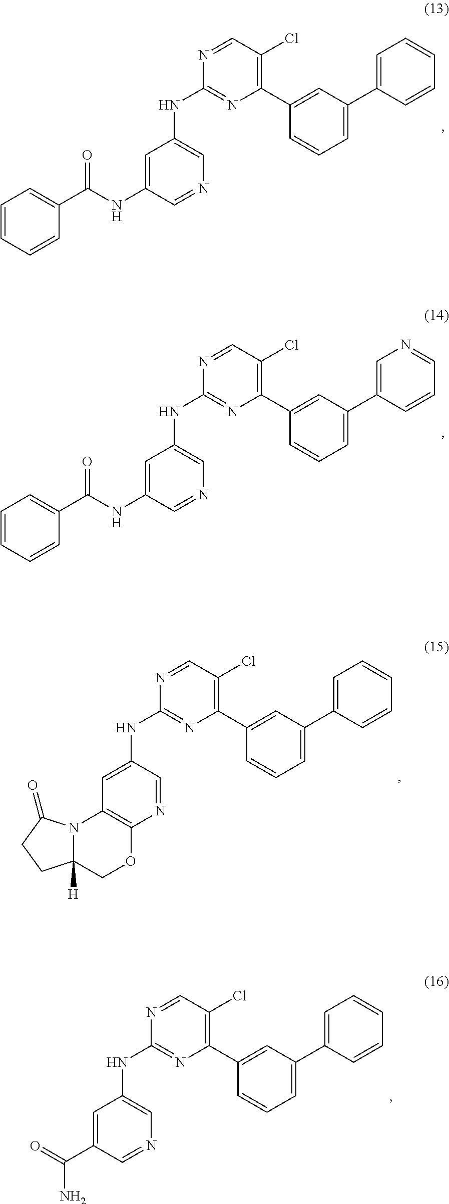 [SMILES: O=C(Nc1cncc(Nc2ncc(Cl)c(-c3cccc(-c4ccccc4)c3)n2)c1)c1ccccc1 ; O=C(Nc1cncc(Nc2ncc(Cl)c(-c3cccc(-c4cccnc4)c3)n2)c1)c1ccccc1 ; O=C1CC[C@H]2COc3ncc(Nc4ncc(Cl)c(-c5cccc(-c6ccccc6)c5)n4)cc3N12 ; NC(=O)c1cncc(Nc2ncc(Cl)c(-c3cccc(-c4ccccc4)c3)n2)c1]

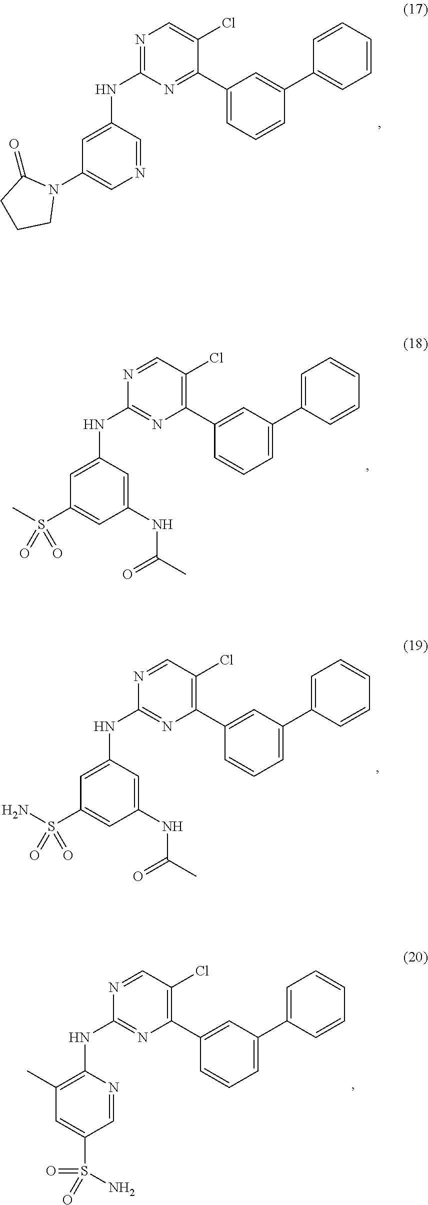 [SMILES: O=C1CCCN1c1cncc(Nc2ncc(Cl)c(-c3cccc(-c4ccccc4)c3)n2)c1 ; CC(=O)Nc1cc(Nc2ncc(Cl)c(-c3cccc(-c4ccccc4)c3)n2)cc(S(C)(=O)=O)c1 ; CC(=O)Nc1cc(Nc2ncc(Cl)c(-c3cccc(-c4ccccc4)c3)n2)cc(S(N)(=O)=O)c1 ; Cc1cc(S(N)(=O)=O)cnc1Nc1ncc(Cl)c(-c2cccc(-c3ccccc3)c2)n1]

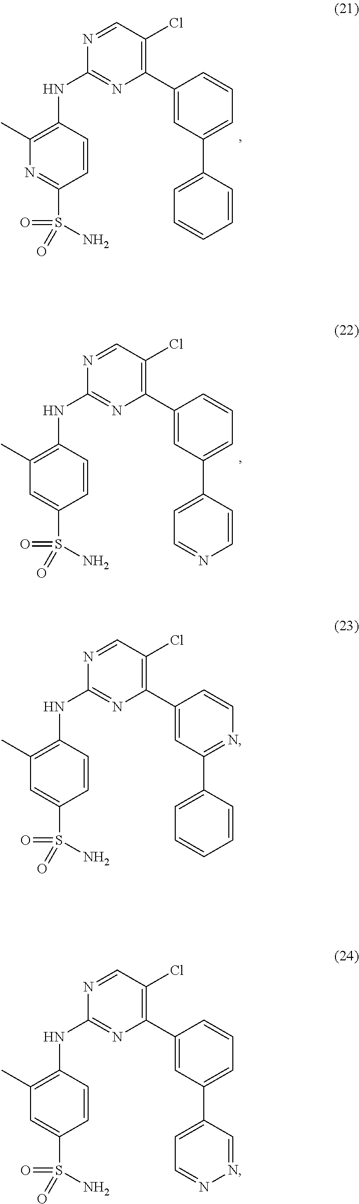 [SMILES: Cc1nc(S(N)(=O)=O)ccc1Nc1ncc(Cl)c(-c2cccc(-c3ccccc3)c2)n1 ; Cc1cc(S(N)(=O)=O)ccc1Nc1ncc(Cl)c(-c2cccc(-c3ccncc3)c2)n1 ; Cc1cc(S(N)(=O)=O)ccc1Nc1ncc(Cl)c(-c2ccnc(-c3ccccc3)c2)n1 ; Cc1cc(S(N)(=O)=O)ccc1Nc1ncc(Cl)c(-c2cccc(-c3ccnnc3)c2)n1]

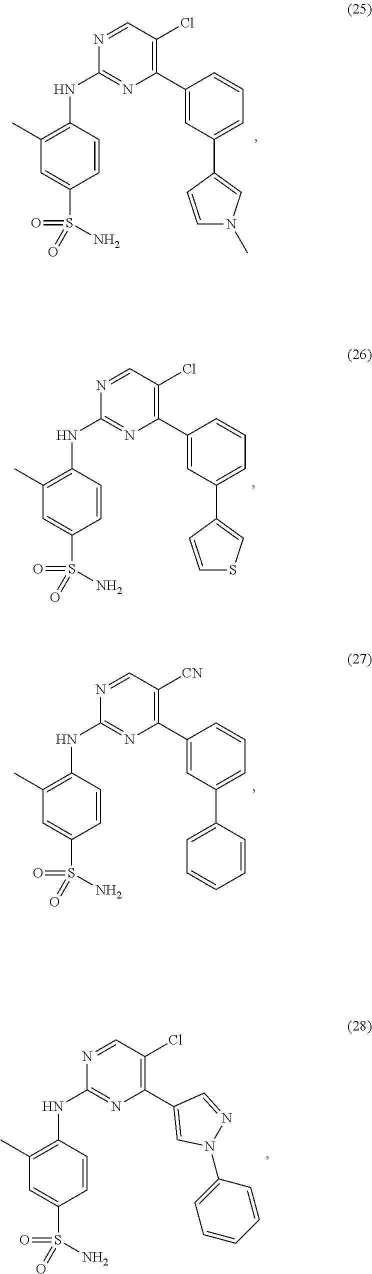

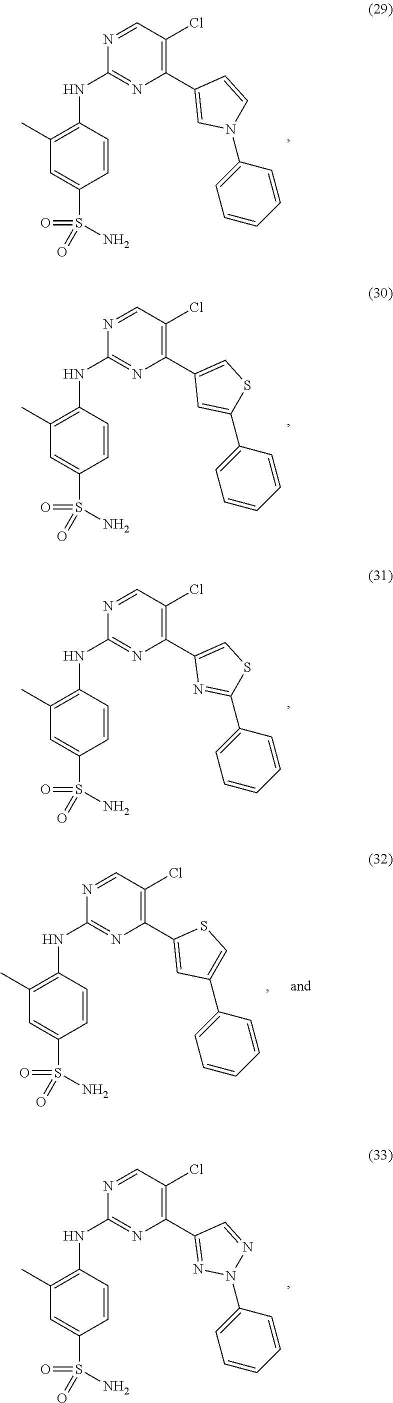 [SMILES: Cc1cc(S(N)(=O)=O)ccc1Nc1ncc(Cl)c(-c2ccn(-c3ccccc3)c2)n1 ; Cc1cc(S(N)(=O)=O)ccc1Nc1ncc(Cl)c(-c2csc(-c3ccccc3)c2)n1 ; Cc1cc(S(N)(=O)=O)ccc1Nc1ncc(Cl)c(-c2csc(-c3ccccc3)n2)n1 ; Cc1cc(S(N)(=O)=O)ccc1Nc1ncc(Cl)c(-c2cc(-c3ccccc3)cs2)n1 ; Cc1cc(S(N)(=O)=O)ccc1Nc1ncc(Cl)c(-c2cnn(-c3ccccc3)n2)n1]

or a pharmaceutically acceptable salt or stereoisomer thereof.

### Compounds of Formula (II)

Broadly, the bifunctional compounds of formula (II) comprise a targeting ligand that binds at least one of phosphatidylinositol-5-phosphate 4-kinase type 2 alpha (PIP4K2A), PIP4K2B, and PIP4K2C and a degron covalently attached to the targeting ligand by a linker, as follows:

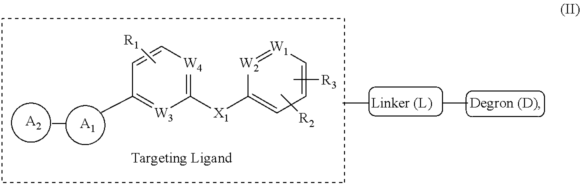

or a pharmaceutically acceptable salt or stereoisomer thereof, wherein:
X₁ is a bond, CH₂, CHMe, NH, NMe, or O;
W₁ is CR₄ or N;
W₂ is CR₄ or N;
each R₄ is independently hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl or optionally substituted 5- or 6-membered heterocyclyl;
W₃ is CH or N;
W₄ is CH or N;
R₁ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;
R₂ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl, optionally substituted aryl, optionally substituted 5- or 6-membered heterocyclyl, or optionally substituted heteroaryl;
R₃ is (C₁-C₆) alkoxy, NH(C₁-C₆) alkyl, NHaryl, NH-5- or 6-membered heteroaryl, NHC(O)aryl, NHC(O)-5- or 6-membered heteroaryl, NHC(O)(C₁-C₆) alkyl, NHSO₂(C₁-C₆) alkyl, NHSO₂aryl, SO₂NH₂, SO₂NH(C₁-C₆) alkyl, SO₂NHaryl, C(O)NH₂, C(O)NH(C₁-C₆) alkyl, C(O)NHaryl, C(O)NH-5- or 6-membered heteroaryl, or optionally substituted 5- to 10-membered heterocyclyl, or
R₃ is O, NH, N(C₁-C₆) alkyl, SO₂NH, SO₂N(C₁-C₆) alkyl, C(O)NH, or optionally substituted 5- to 10-membered heterocyclyl, when R₃ is bound to the Linker; and
A₁ and A₂ are each independently optionally substituted aryl or optionally substituted 5- to 6-membered heteroaryl;
the linker is a moiety that covalently connects the degron and the targeting ligand; and
the degron is a moiety that binds an E3 ubiquitin ligase.

In some embodiments, bifunctional compounds are represented by Formula II′:

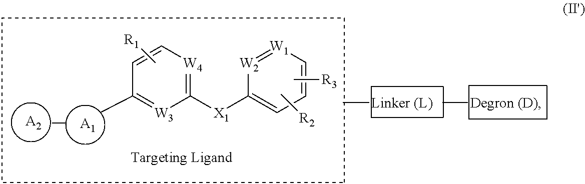

or a pharmaceutically acceptable salt or stereoisomer thereof, wherein:

X₁ is a bond, CH₂, CHMe, NH, NMe, or O;

W₁ is CR₄ or N;

W₂ is CR₄ or N;

each R₄ is independently hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl or optionally substituted 5- or 6-membered heterocyclyl;

### W₃ is CH or N;

W₄ is CH or N;

R₁ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;

R₂ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl, optionally substituted aryl, optionally substituted 5- or 6-membered heterocyclyl, or optionally substituted heteroaryl;

R₃ is O, NH, (C₁-C₆) alkoxy, N(C₁-C₆) alkyl, NH(C₁-C₆) alkyl, NHaryl, NH-5- or 6-membered heteroaryl, NHC(O)aryl, NHC(O)-5- or 6-membered heteroaryl, NHC(O)(C₁-C₆) alkyl, NHSO₂(C₁-C₆) alkyl, NHSO₂aryl, SO₂NH, SO₂NH₂, SO₂N(C₁-C₆) alkyl, SO₂NH(C₁-C₆) alkyl, SO₂NHaryl, C(O)NH, C(O)NH₂, C(O)N(C₁-C₆) alkyl, C(O)NH(C₁-C₆) alkyl, C(O)NHaryl, or C(O)NH-5- or 6-membered heteroaryl; and

A₁ and A₂ are each independently optionally substituted aryl or optionally substituted 5- to 6-membered heteroaryl; and

the linker is a moiety that covalently connects the degron and the targeting ligand; and

the degron is a moiety that binds an E3 ubiquitin ligase.

In some embodiments, bifunctional compounds of formula (II) may be represented by formula IIa:

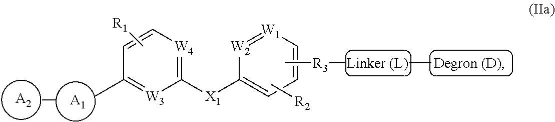

or a pharmaceutically acceptable salt or stereoisomer thereof.

In some embodiments, bifunctional compounds of formula (II) may be represented by formula IIb.

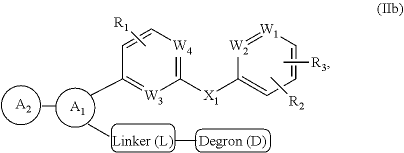

or a pharmaceutically acceptable salt or stereoisomer thereof.

In some embodiments, bifunctional compounds of formula (IT) may be represented by formula IIc:

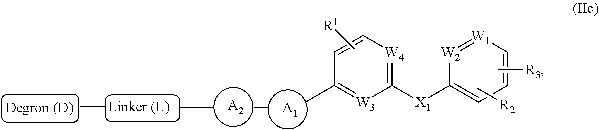

or a pharmaceutically acceptable salt or stereoisomer thereof.

In some embodiments, W₁ is CR₄.

In some embodiments, W₁ is N.

In some embodiments, W₂ is CR₄.

In some embodiments, W₂ is N.

In some embodiments, R₂ is hydrogen.

In some embodiments, R₂ is methyl.

In some embodiments, R₃ is SO₂NH, SO₂N(C₁-C₆) alkyl, or C(O)NH.

In some embodiments, R₃ is optionally substituted 5- to 10-membered heterocyclyl, containing 1 to 3 heteroatoms selected from N, O, and S. In some embodiments, R₃ is

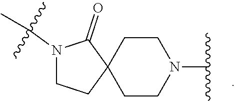 [SMILES: *N1CCC2(CC1)CCN(*)C2=O]

In some embodiments, A₁ is optionally substituted phenyl. In some embodiments, A₁ is phenyl.

In some embodiments, A₁ is optionally substituted 5- to 6-membered heteroaryl, containing 1 or 2 heteroatoms selected from N, O, and S. In some embodiments, A₁ is 5- to 6-membered heteroaryl, containing 1 or 2 heteroatoms selected from N, O, and S. In some embodiments, A₁ is optionally substituted pyrrole, imidazole, pyrazole, thiazole, triazole, or pyridine.

In some embodiments, the targeting ligand is of Formula TL-1a, TL-1b, TL-1c, or TL-1d:

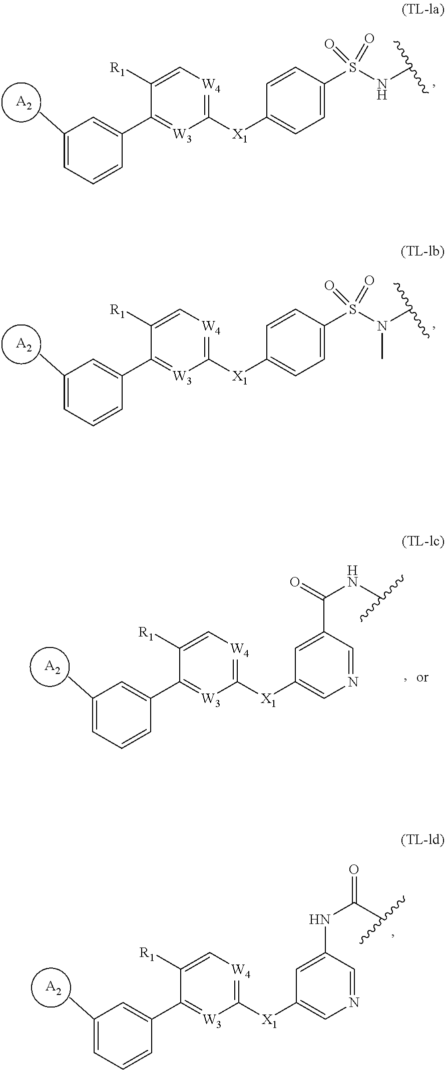

In some embodiments, the targeting ligand is of Formula TL-1e or TL-1f:

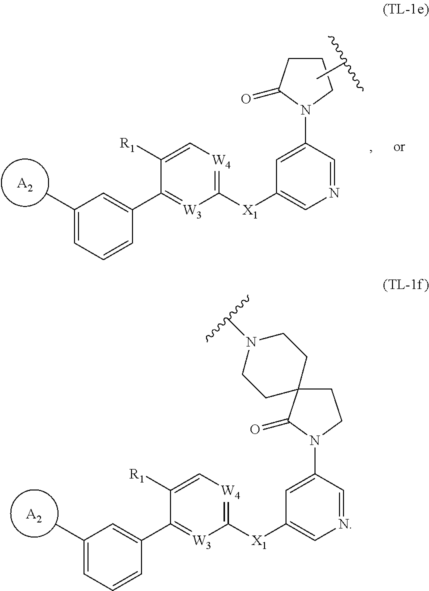

In some embodiments, X₁ is NH.

In some embodiments, W₄ is N.

In some embodiments, W₃ is N.

In some embodiments, R₁ is halogen.

In some embodiments, R₁ is chloro.

In some embodiments, R₁ is CN.

In some embodiments, A₂ is optionally substituted phenyl. In some embodiments, A₂ is phenyl.

In some embodiments, A₂ is optionally substituted 5- to 6-membered heteroaryl, containing 1 or 2 heteroatoms selected from N, O, and S. In some embodiments, A₂ is 5- to 6-membered heteroaryl, containing 1 or 2 heteroatoms selected from N, O, and S. In some embodiments, A₂ is optionally substituted thiophene, pyrrole, pyrimidine, or pyridazine.

In some embodiments, X₁ is NH, R₁ is chloro or CN, W₁ is N or CH, and W₂ is N or CH.

In some embodiments, X₁ is NH, R₁ is chloro or CN, W₁ is N or CH, W₂ is N or CH, and R₂ is hydrogen or methyl.

In some embodiments, X₁ is NH, R₁ is chloro or CN, W₁ is N or CH, W₂ is N or CH, R₂ is hydrogen or methyl, and R₃ is SO₂NH, SO₂N(C₁-C₆) alkyl, or C(O)NH, and wherein R₃ is bound to the Linker.

In some embodiments, X₁ is NH, R₁ is chloro or CN, W₁ is N or CH, W₂ is N or CH, R₂ is hydrogen or methyl, and R₃ is optionally substituted 5- to 10-membered heterocyclyl, containing 1 to 3 heteroatoms selected from N, O, and S, and wherein R₃ is bound to the Linker.

In some embodiments, X₁ is NH, R₁ is chloro or CN, W₁ is N or CH, W₂ is N or CH, R₂ is hydrogen or methyl, R₃ is SO₂NH, SO₂N(C₁-C₆) alkyl, or C(O)NH and wherein R₃ is bound to the Linker, W₃ is N, and W₄ is N.

In some embodiments, X₁ is NH, R₁ is chloro or CN, W, is N or CH, W₂ is N or CH, R₂ is hydrogen or methyl, R₃ is optionally substituted 5- to 10-membered heterocyclyl, containing 1 to 3 heteroatoms selected from N, O, and S, and wherein R₃ is bound to the Linker, W₃ is N, and W₄ is N.

In some embodiments, X₁ is NH, R₁ is chloro or CN, W₁ is N or CH, W₂ is N or CH, R₂ is hydrogen or methyl, R₃ is SO₂NH, SO₂N(C₁-C₆) alkyl, or C(O)NH and wherein R₃ is bound to the Linker, W₃ is N, W₄ is N, A₁ is optionally substituted phenyl, and A₂ is optionally substituted phenyl or optionally substituted 5- to 6-membered heteroaryl, containing 1 to 3 heteroatoms selected from N, O, and S.

In some embodiments, X₁ is NH, R₁ is chloro or CN, W, is N or CH, W₂ is N or CH, R₂ is hydrogen or methyl, R₃ is optionally substituted 5- to 10-membered heterocyclyl, containing 1 to 3 heteroatoms selected from N, O, and S, and wherein R₃ is bound to the Linker, W₃ is N, W₄ is N, A₁ is optionally substituted phenyl, and A₂ is optionally substituted phenyl or optionally substituted 5- to 6-membered heteroaryl, containing 1 to 3 heteroatoms selected from N, O, and S.

In some embodiments, the optional substituent for a bifunctional compound of Formula (II or II′) is (C₁-C₆) alkyl, (C₂-C₆) alkenyl, (C₂-C₆) alkynyl, halo, (C₁-C₆) haloalkyl, (C₁-C₆) cycloalkyl, 5- to 10-membered heterocycloalkyl, hydroxy, (C₁-C₆) alkoxy, (C₆-C₁₀) cycloalkoxy, 5- to 10-membered heterocycloalkoxy, (C₁-C₆) haloalkoxy, (C₆-C₁₀) aryloxy, 5- to 10-membered heteroaryloxy, (C₆-C₁₀) aralkyloxy, (C₂-C₆) alkyenyloxy, (C₂-C₆) alkynyloxy, amino, (C₁-C₆) alkylamino, (C₆-C₁₀) cycloalkylamino, 5- to 10-membered heterocycloalkylamino, (C₆-C₁₀) arylamino, 5- to 10-membered heteroarylamino, (C₆-C₁₀) aralkylamino, N—(C₁-C₆) alkyl-N—(C₆-C₁₀) arylamino, N—(C₁-C₆) alkyl-N-5- to 10-membered heteroarylamino, N—(C₁-C₆) alkyl-N—(C₆-C₁₀) aralkylamino, (C₁-C₆) hydroxyalkyl, (C₁-C₆) aminoalkyl, (C₁-C₆) alkylthio, (C₁-C₆) haloalkylthio, (C₁-C₆) alkylsulfonyl, (C₁-C₆) haloalkylsulfonyl, (C₆-C₁₀) cycloalkylsulfonyl, 5- to 10-membered heterocycloalkylsulfonyl, (C₆-C₁₀) arylsulfonyl, 5- to 10-membered heteroarylsulfonyl, aminosulfonyl, (C₁-C₆) alkylaminosulfonyl, (C₆-C₁₀) cycloalkylaminosulfonyl, 5- to 10-membered heterocycloalkylaminosulfonyl, (C₆-C₁₀) arylaminosulfonyl, 5- to 10-membered heteroarylaminosulfonyl, N—(C₁-C₆) alkyl-N—(C₆-C₁₀) arylaminosulfonyl, N—(C₁-C₆) alkyl-N-5- to 10-membered heteroarylaminosulfonyl, formyl, (C₁-C₆) alkylcarbonyl, (C₁-C₆) haloalkylcarbonyl, (C₂-C₆) alkenylcarbonyl, (C₂-C₆) alkynylcarbonyl, carboxy, (C₁-C₆) alkoxycarbonyl, (C₁-C₆) alkylcarbonyloxy, amino, (C₁-C₆) alkylsulfonylamino, (C₁-C₆) haloalkylsulfonylamino, (C₆-C₁₀) cycloalkylsulfonylamino, 5- to 10-membered heterocycloalkylsulfonylamino, (C₆-C₁₀) arylsulfonylamino, 5- to 10-membered heteroarylsulfonylamino, (C₆-C₁₀) aralkylsulfonylamino, (C₁-C₆) alkylcarbonylamino, (C₁-C₆) haloalkylcarbonylamino, (C₆-C₁₀) cycloalkylcarbonylamino, 5- to 10-membered heterocycloalkylcarbonylamino, (C₆-C₁₀) arylcarbonylamino, 5- to 10-membered heteroarylcarbonylamino, (C₆-C₁₀) aralkylsulfonylamino, aminocarbonyl, (C₁-C₆) alkylaminocarbonyl, (C₆-C₁₀) cycloalkylaminocarbonyl, 5- to 10-membered heterocycloalkylaminocarbonyl, (C₆-C₁₀) arylaminocarbonyl, 5- to 10-membered heteroarylaminocarbonyl, N—(C₁-C₆) alkyl-N—(C₆-C₁₀) arylaminocarbonyl, N—(C₁-C₆) alkyl-N-5- to 10-membered heteroarylaminocarbonyl, cyano, nitro, oxo, azido, 3- to 10-membered carbocyclyl, or 4- to 10-membered heterocyclyl. In some embodiments, the optional substituent is not further substituted. In some embodiments, the optional substituent is further substituted.

In some embodiments, the optional substituent for a bifunctional compound of Formula (II or II′) is (C₁-C₆) alkyl, (C₁-C₆) haloalkyl, (C₁-C₆) alkoxy, amino, (C₁-C₆) aminoalkyl, hydroxy, halo, cyano, oxo, (C₆-C₁₂) aryl, 5- to 10-membered heterocyclyl, sulfonylamino, (C₁-C₆) alkylsulfonylamino.

In some embodiments, the optional substituent for a bifunctional compound of Formula (II or II′) is methyl, fluoro, cholo, cyano, —OMe, —OCF₃, —NH₂, —NHMe, —NMe₂, —SO₂NH₂, —C(O)NH₂,

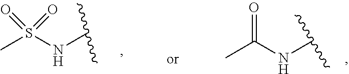

In some embodiments, the targeting ligand is of Formula TL-1a1, TL-1b1, TL-1c1, or TL-1d1:

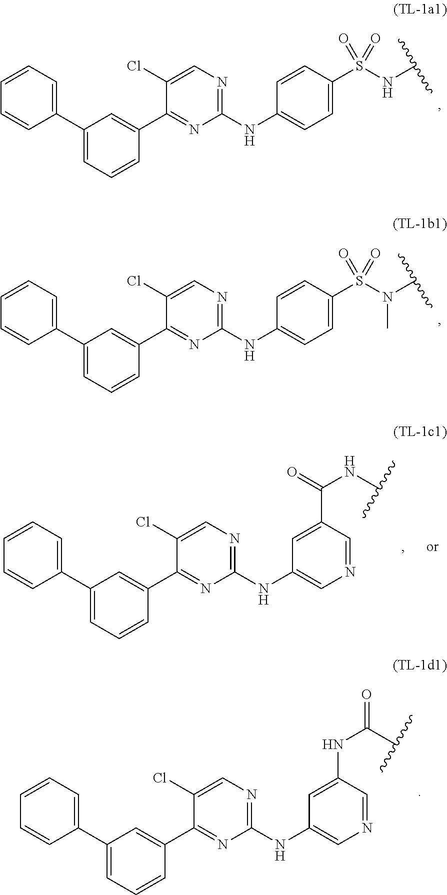 [SMILES: *NS(=O)(=O)c1ccc(Nc2ncc(Cl)c(-c3cccc(-c4ccccc4)c3)n2)cc1 ; *N(C)S(=O)(=O)c1ccc(Nc2ncc(Cl)c(-c3cccc(-c4ccccc4)c3)n2)cc1 ; *NC(=O)c1cncc(Nc2ncc(Cl)c(-c3cccc(-c4ccccc4)c3)n2)c1 ; *C(=O)Nc1cncc(Nc2ncc(Cl)c(-c3cccc(-c4ccccc4)c3)n2)c1]

In some embodiments, the targeting ligand is of Formula TL-1e1 or TL-1f1:

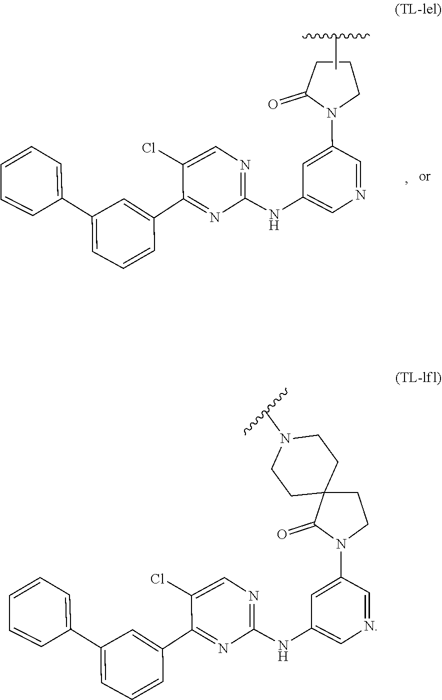

### Linkers

The linker (“L”) provides a covalent attachment between the targeting ligand and the degron. The structure of linker may not be critical, provided it is substantially non-interfering with the activity of the targeting ligand or the degron.

In some embodiments, the linker may include a single alkylene group or an alkylene chain or a bivalent alkylene chain. These chains may be interrupted by, and/or terminate (at either or both termini) in a —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, or a 5- to 12-membered heteroarylene, or any combination thereof, wherein R′ is H or C₁-C₆ alkyl, wherein the interrupting and the one or both terminating groups may be the same or different.

In some embodiments, the linker includes an alkylene chain having 2-20 alkylene units. In some embodiments, the linker includes an alkylene chain having 3-12 alkylene units. In some embodiments, the linker may include a C₃-C₁₂ alkylene chain terminating in NH-group wherein the nitrogen is also bound to the degron.

In some embodiments, the linker includes an alkylene chain having 1-10 alkylene units that is interrupted by and/or terminated (at either or both termini) with —O—,

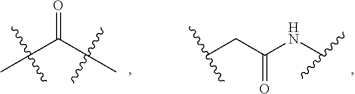

or a combination thereof.

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of linkers that may be suitable for use in the present invention include alkylene chains:

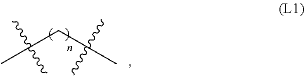

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

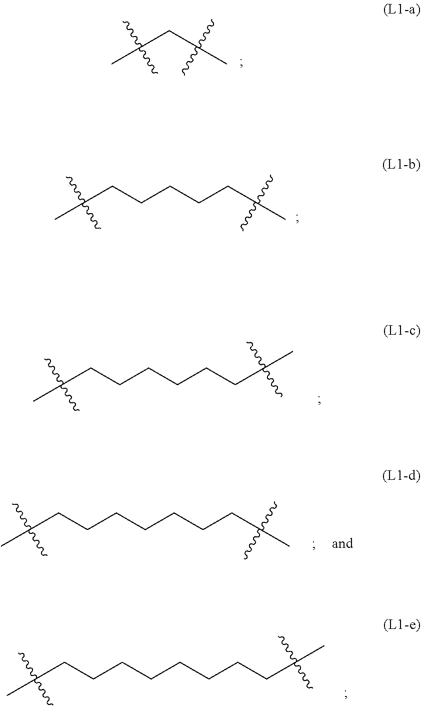

alkylene chains terminating in various functional groups (as described above), examples of which are as follows:

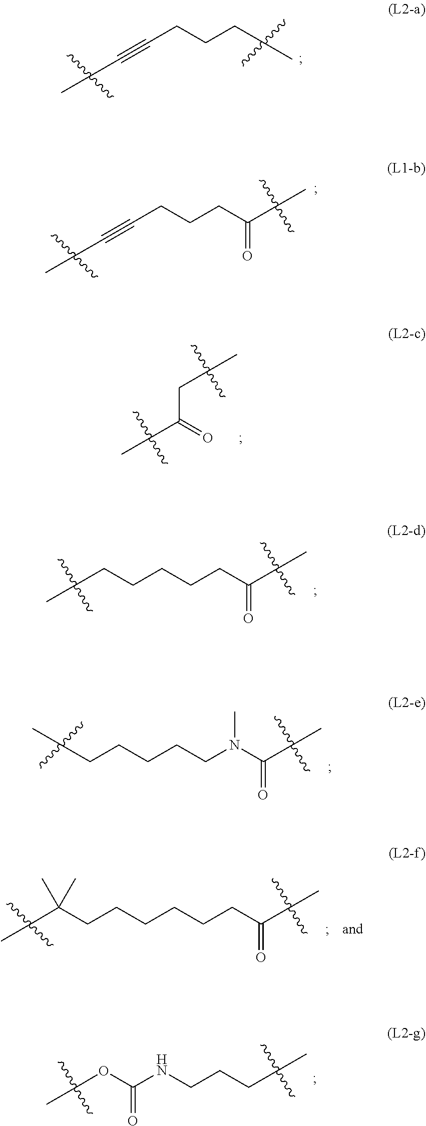

alkylene chains interrupted by various functional groups (as described above), examples of which are as follows:

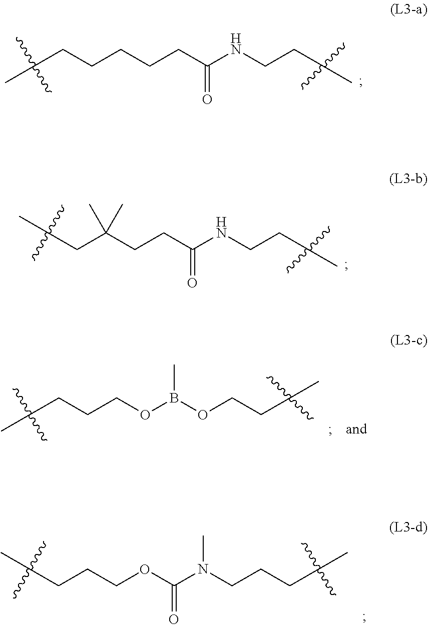 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted by or terminating with heterocyclene groups, e.g.,

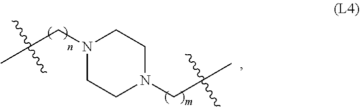

wherein m and n are independently integers of 0-10, examples of which include:

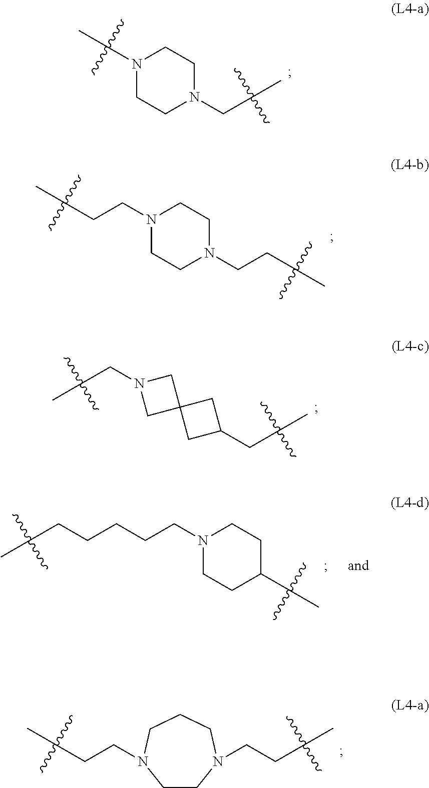 [SMILES: *CN1CCN(*)CC1 ; *CCN1CCN(CC*)CC1 ; *CC1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

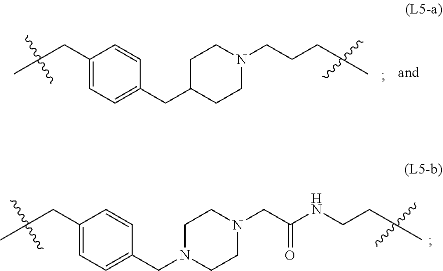 [SMILES: *CCCN1CCC(Cc2ccc(C*)cc2)CC1 ; *CCNC(=O)CN1CCN(Cc2ccc(C*)cc2)CC1]

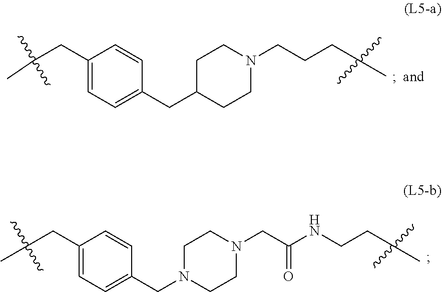 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

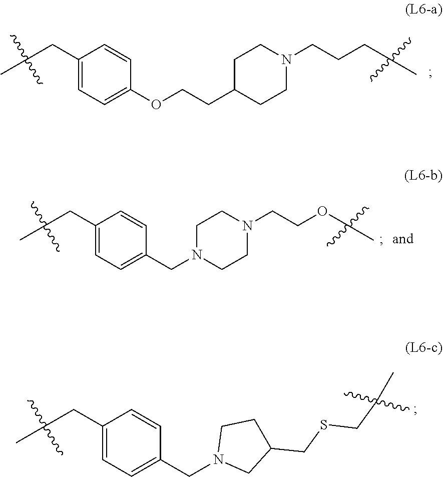 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by and/or terminating in a heteroatom such as N, O or B, e.g.,

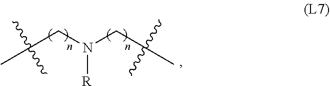

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4- 9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

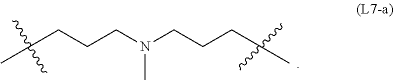 [SMILES: *CCCN(C)CCC*]

In some embodiments, the linker may include a single polyethylene glycol chain. These chains may terminate at either or both termini in a —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —R′C(O)N(R′)R′—, —C(O)N(R′)C(O)N(R′)—, —N(R′)C(O)—, —N(R′)C(O)N(R′)—, —N(R′)C(O)O—, —OC(O)N(R′)—, —C(NR)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R)—, —N(R′)S(O)N(R′)—, C₃₋₁₂ carbocyclene, 3- to 12-membered heterocyclene, or a 5- to 12-membered heteroarylene, or any combination thereof, wherein R′ is H or C₁-C₆ alkyl, wherein the one or both terminating groups may be the same or different.

In some embodiments, the linker includes a polyethylene glycol chain having 1-10 PEG units. In some embodiments, the linker includes a polyethylene glycol chain having 1-6 PEG units.

In some embodiments, the linker includes a polyethylene glycol chain having 2-8 PEG units and terminating (at either or both termini) with

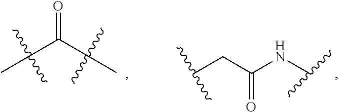

or a combination thereof.

Examples of linkers that include a polyethylene glycol chain include:

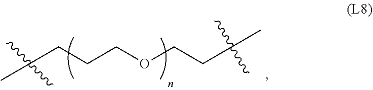

wherein n is an integer of 2-10, examples of which include:

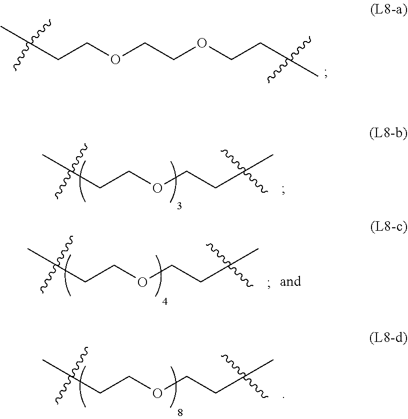

In some embodiments, the polyethylene glycol chain may terminate in a functional group, examples of which are as follows:

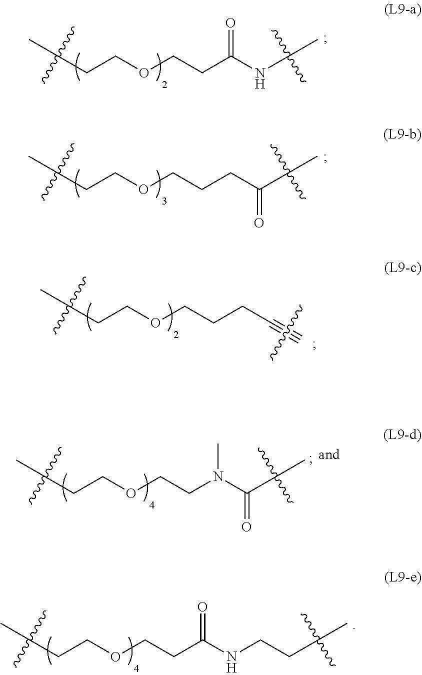

In some embodiments, bifunctional compound of formula (II) includes a linker that is represented by any one of the following structures:

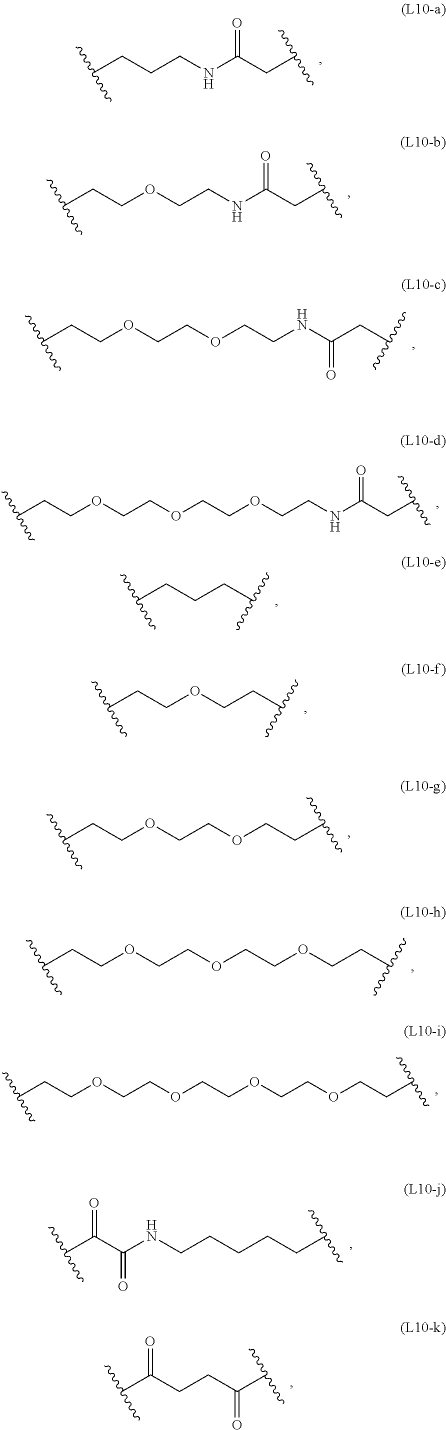

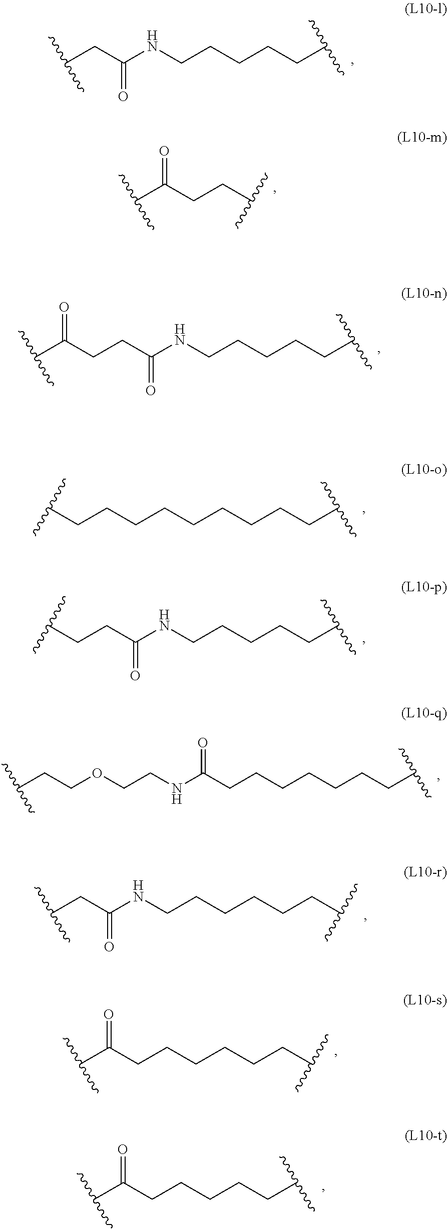

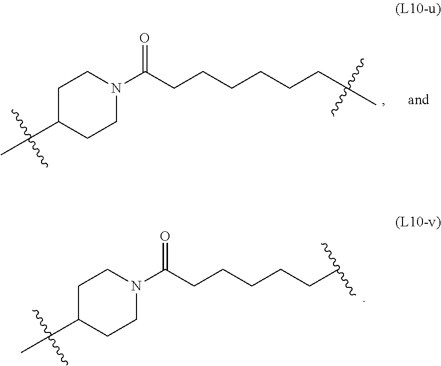 [SMILES: *CCCCCCC(=O)N1CCC(*)CC1 ; *CCCCCC(=O)N1CCC(*)CC1]

In some embodiments, bifunctional compounds of formula (II) are represented by any one of the following structures:

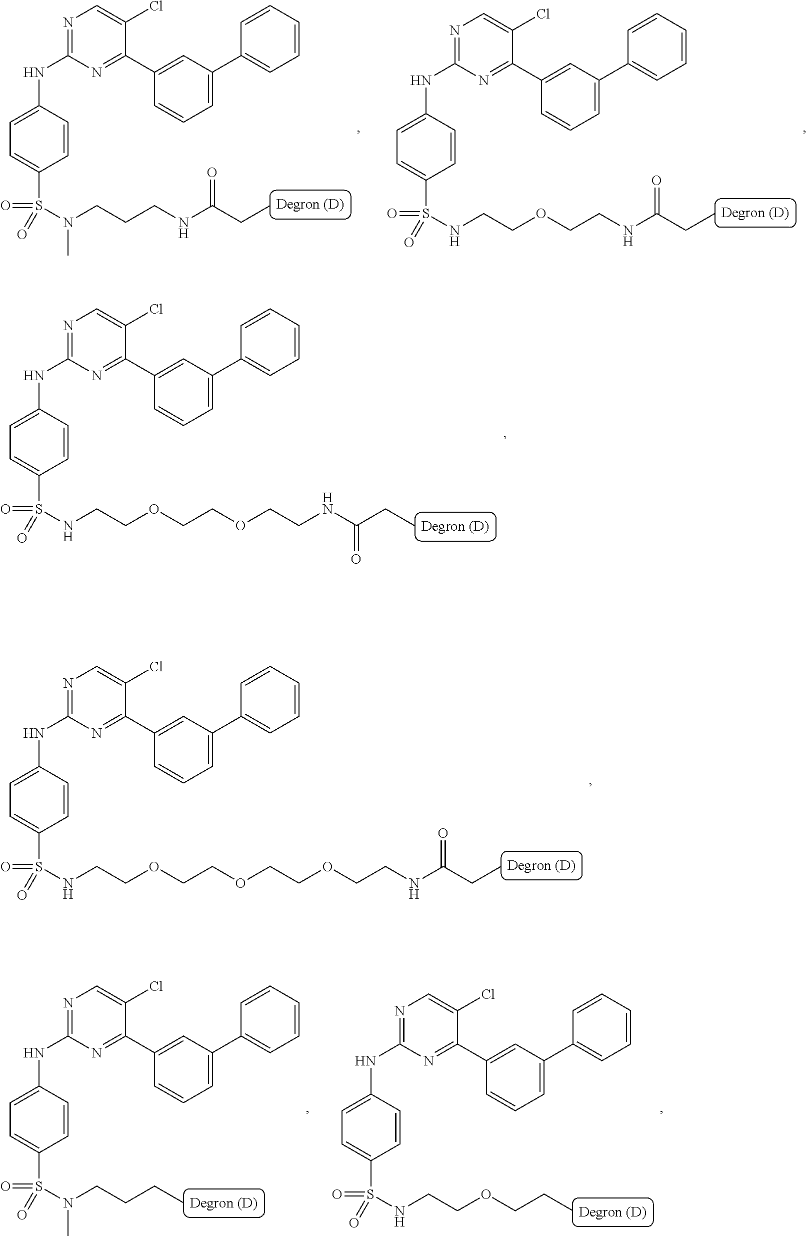 [SMILES: CCC(=O)NCCCN(C)S(=O)(=O)c1ccc(Nc2ncc(Cl)c(-c3cccc(-c4ccccc4)c3)n2)cc1 ; CCC(=O)NCCOCCNS(=O)(=O)c1ccc(Nc2ncc(Cl)c(-c3cccc(-c4ccccc4)c3)n2)cc1 ; CCC(=O)NCCOCCOCCNS(=O)(=O)c1ccc(Nc2ncc(Cl)c(-c3cccc(-c4ccccc4)c3)n2)cc1 ; CCC(=O)NCCOCCOCCOCCNS(=O)(=O)c1ccc(Nc2ncc(Cl)c(-c3cccc(-c4ccccc4)c3)n2)cc1 ; CCCCN(C)S(=O)(=O)c1ccc(Nc2ncc(Cl)c(-c3cccc(-c4ccccc4)c3)n2)cc1 ; CCCOCCNS(=O)(=O)c1ccc(Nc2ncc(Cl)c(-c3cccc(-c4ccccc4)c3)n2)cc1]

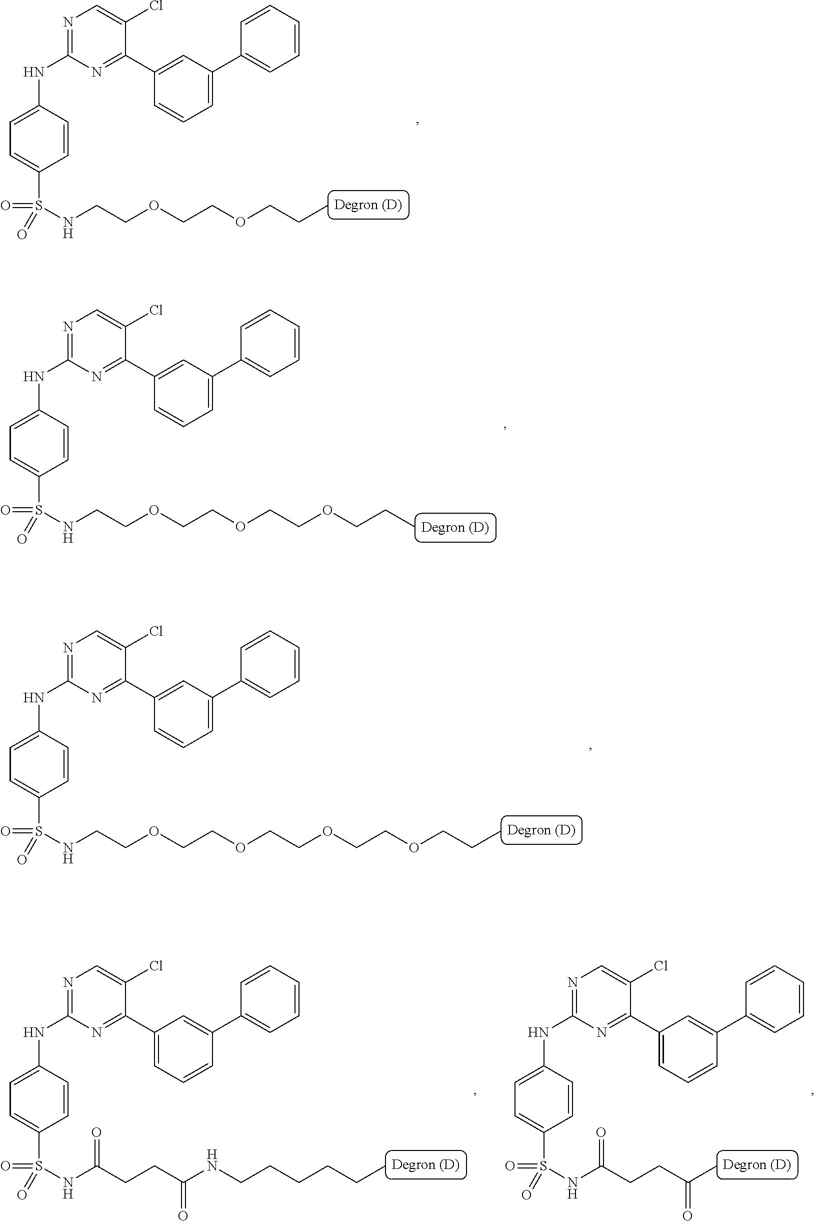 [SMILES: CCCOCCOCCNS(=O)(=O)c1ccc(Nc2ncc(Cl)c(-c3cccc(-c4ccccc4)c3)n2)cc1 ; CCCOCCOCCOCCNS(=O)(=O)c1ccc(Nc2ncc(Cl)c(-c3cccc(-c4ccccc4)c3)n2)cc1 ; CCCOCCOCCOCCOCCNS(=O)(=O)c1ccc(Nc2ncc(Cl)c(-c3cccc(-c4ccccc4)c3)n2)cc1 ; CCCCCCNC(=O)CCC(=O)NS(=O)(=O)c1ccc(Nc2ncc(Cl)c(-c3cccc(-c4ccccc4)c3)n2)cc1 ; CC(=O)CCC(=O)NS(=O)(=O)c1ccc(Nc2ncc(Cl)c(-c3cccc(-c4ccccc4)c3)n2)cc1]

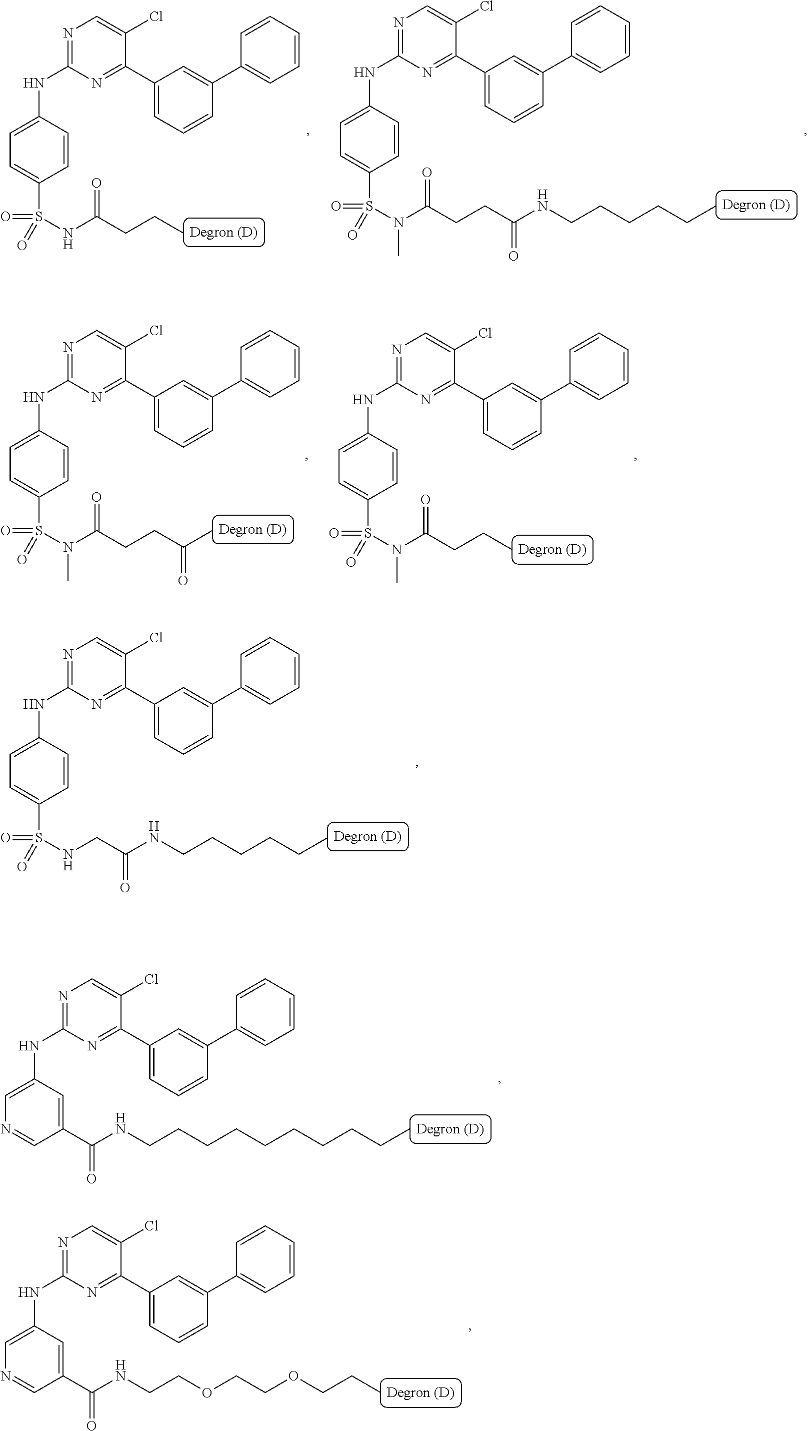

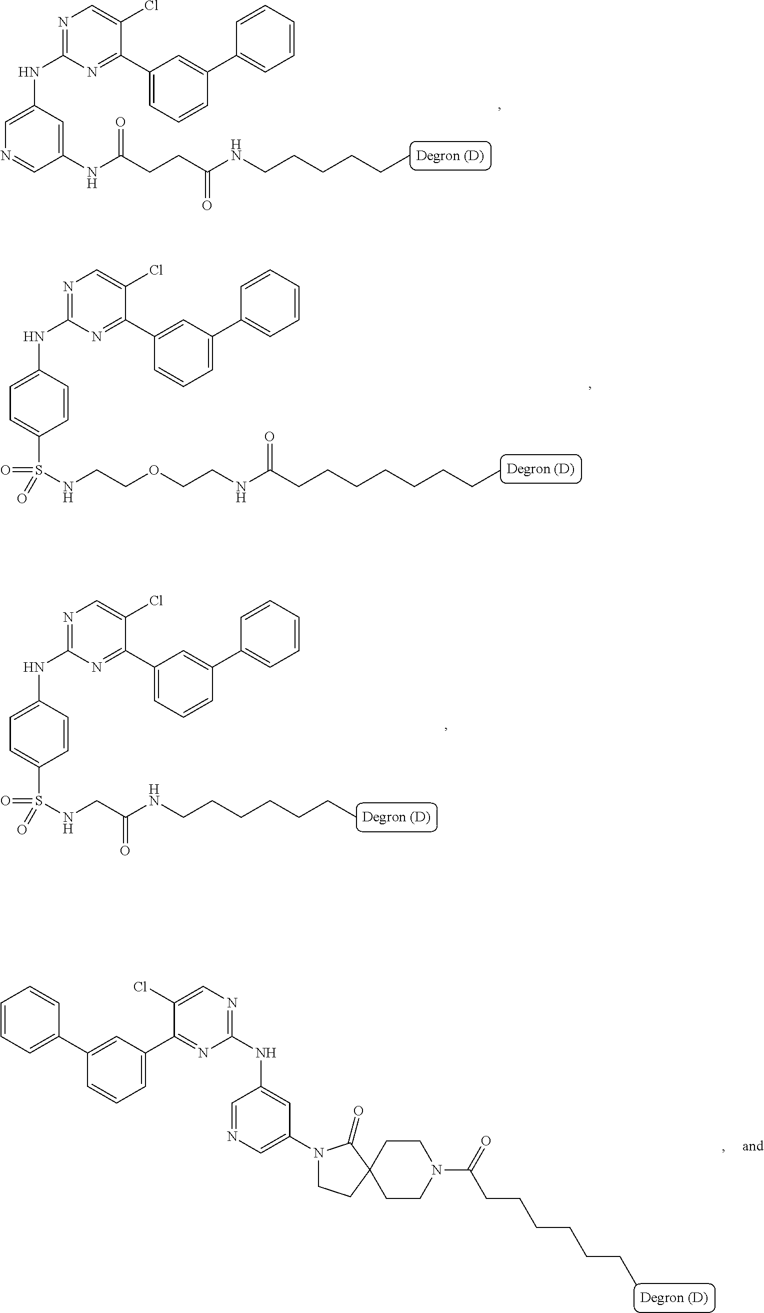 [SMILES: CCCCCCNC(=O)CCC(=O)Nc1cncc(Nc2ncc(Cl)c(-c3cccc(-c4ccccc4)c3)n2)c1 ; CCCCCCCCC(=O)NCCOCCNS(=O)(=O)c1ccc(Nc2ncc(Cl)c(-c3cccc(-c4ccccc4)c3)n2)cc1 ; CCCCCCCNC(=O)CNS(=O)(=O)c1ccc(Nc2ncc(Cl)c(-c3cccc(-c4ccccc4)c3)n2)cc1 ; CCCCCCCC(=O)N1CCC2(CC1)CCN(c1cncc(Nc3ncc(Cl)c(-c4cccc(-c5ccccc5)c4)n3)c1)C2=O]

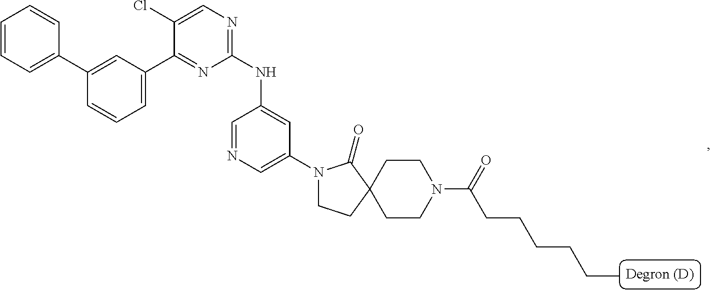 [SMILES: CCCCCCC(=O)N1CCC2(CC1)CCN(c1cncc(Nc3ncc(Cl)c(-c4cccc(-c5ccccc5)c4)n3)c1)C2=O]

or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron binds the E3 ligase which is cereblon (CRBN).

Representative examples of degrons that bind CRBN are represented by any one of structures (D1-a) to (D1-d):

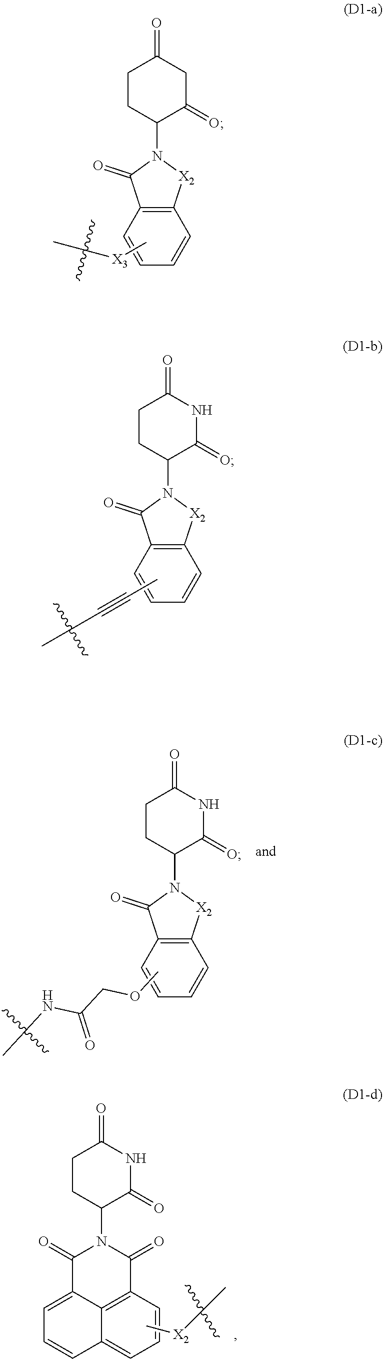

wherein X₂ is CH₂ or C(O) and X₃ is CR″₁R″₂, NR″₁, O, or S, wherein R″₁ and R″₂ are independently hydrogen, halogen, OH, NH₂, C₁-C₃ alkyl, C₁-C₃ alkoxyl, or C₁-C₃ alkylamine, or R″₁ and R″₂, together with the atoms to which they are bound, form a C₃-C₇ carbocyclic or 3- to 7-membered heterocyclic ring (e.g., azetidine, piperidine, pyrrolidine, cyclobutane, cyclohexane).

In some embodiments, bifunctional compounds of the present invention are represented by any one of the following structures:

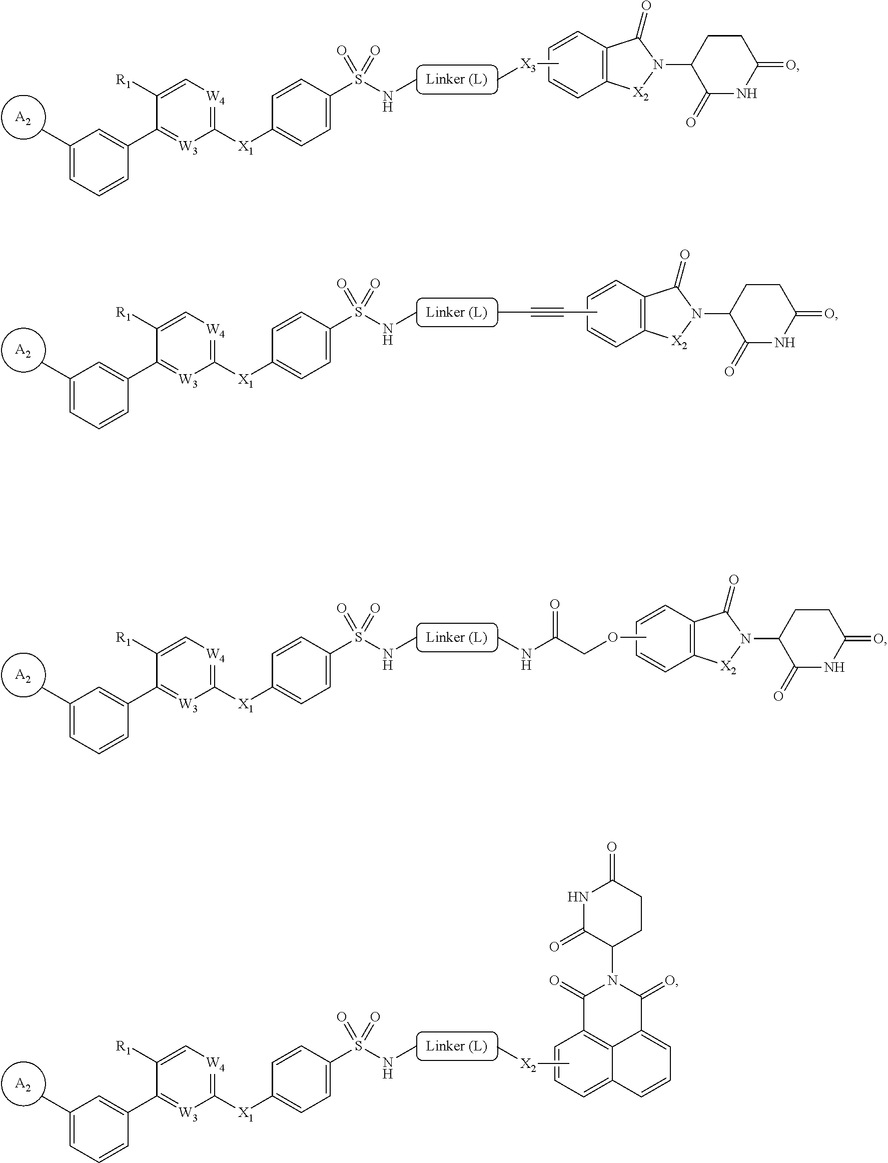

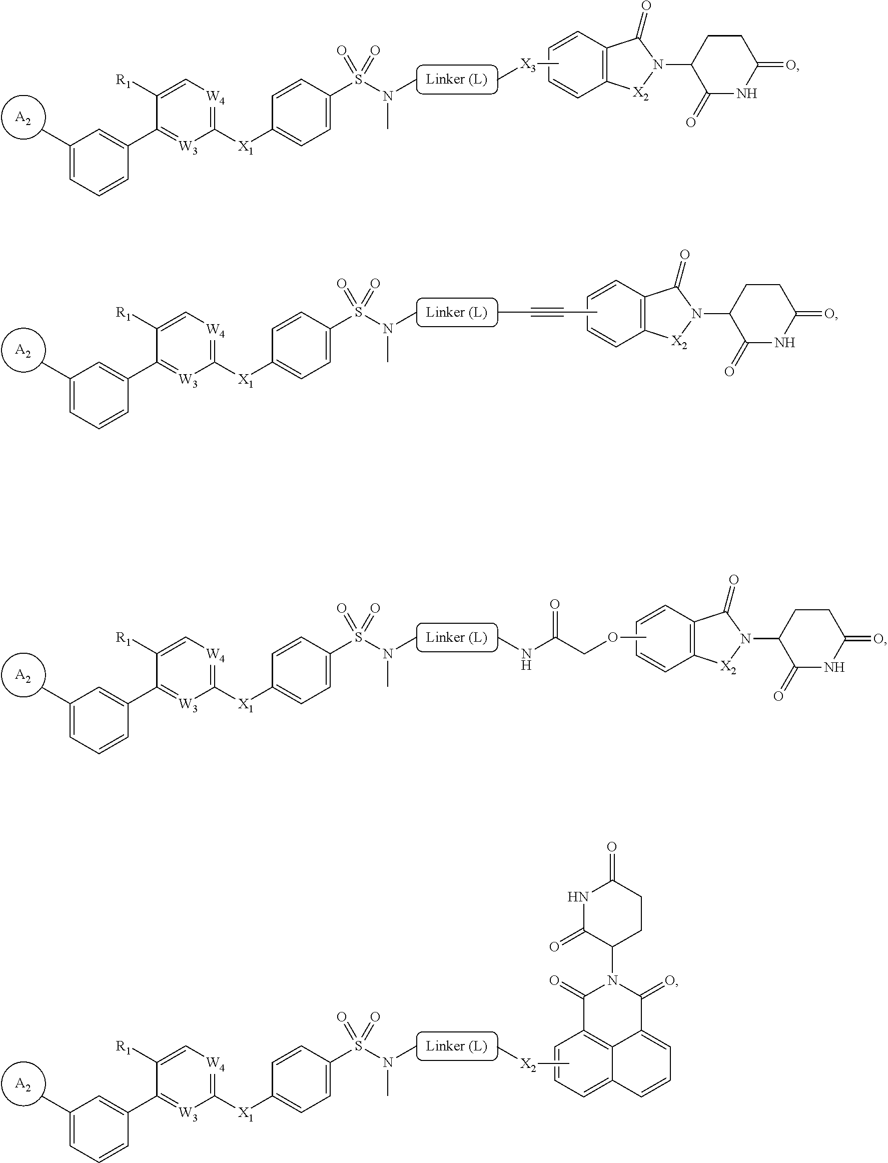

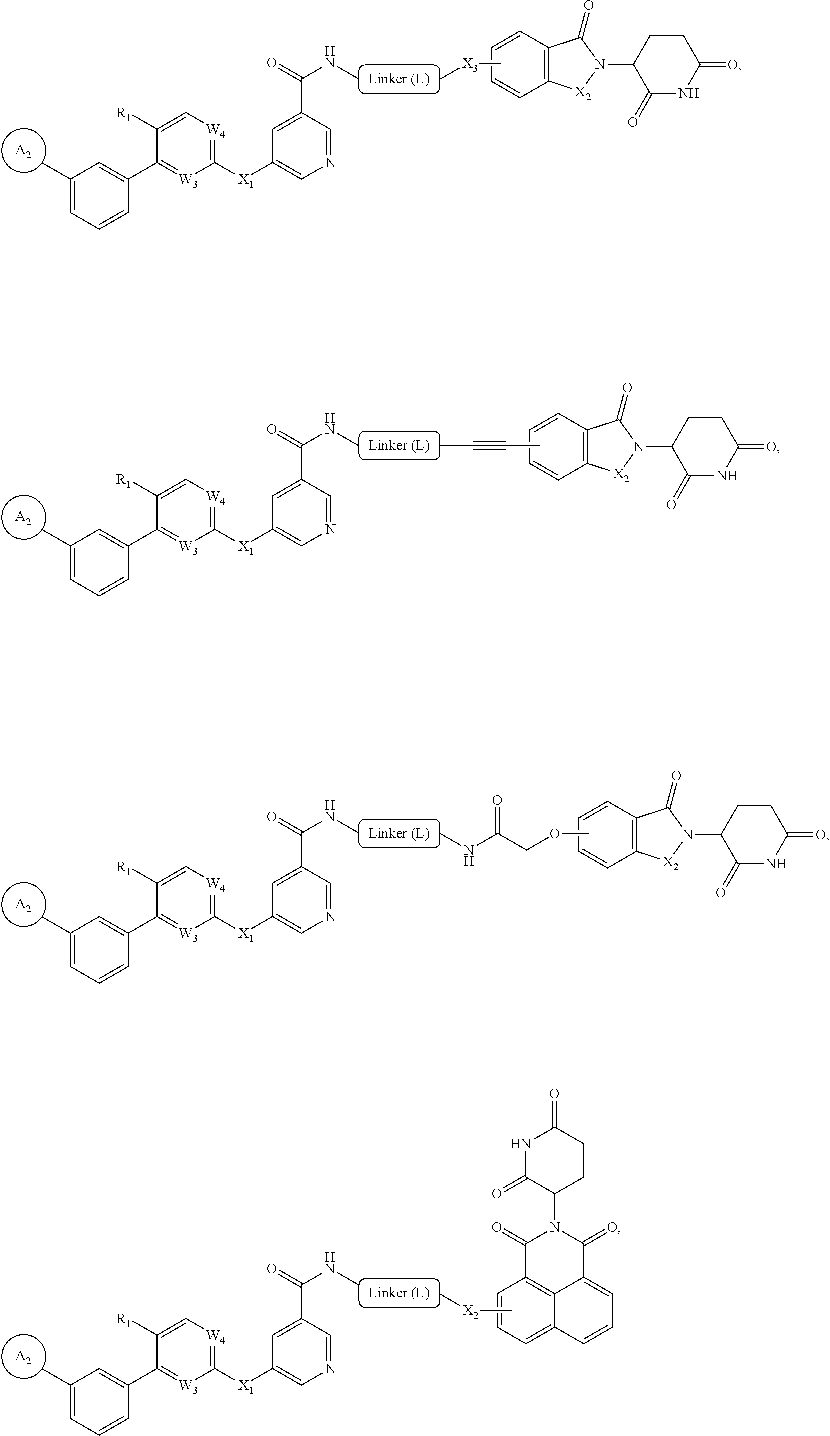

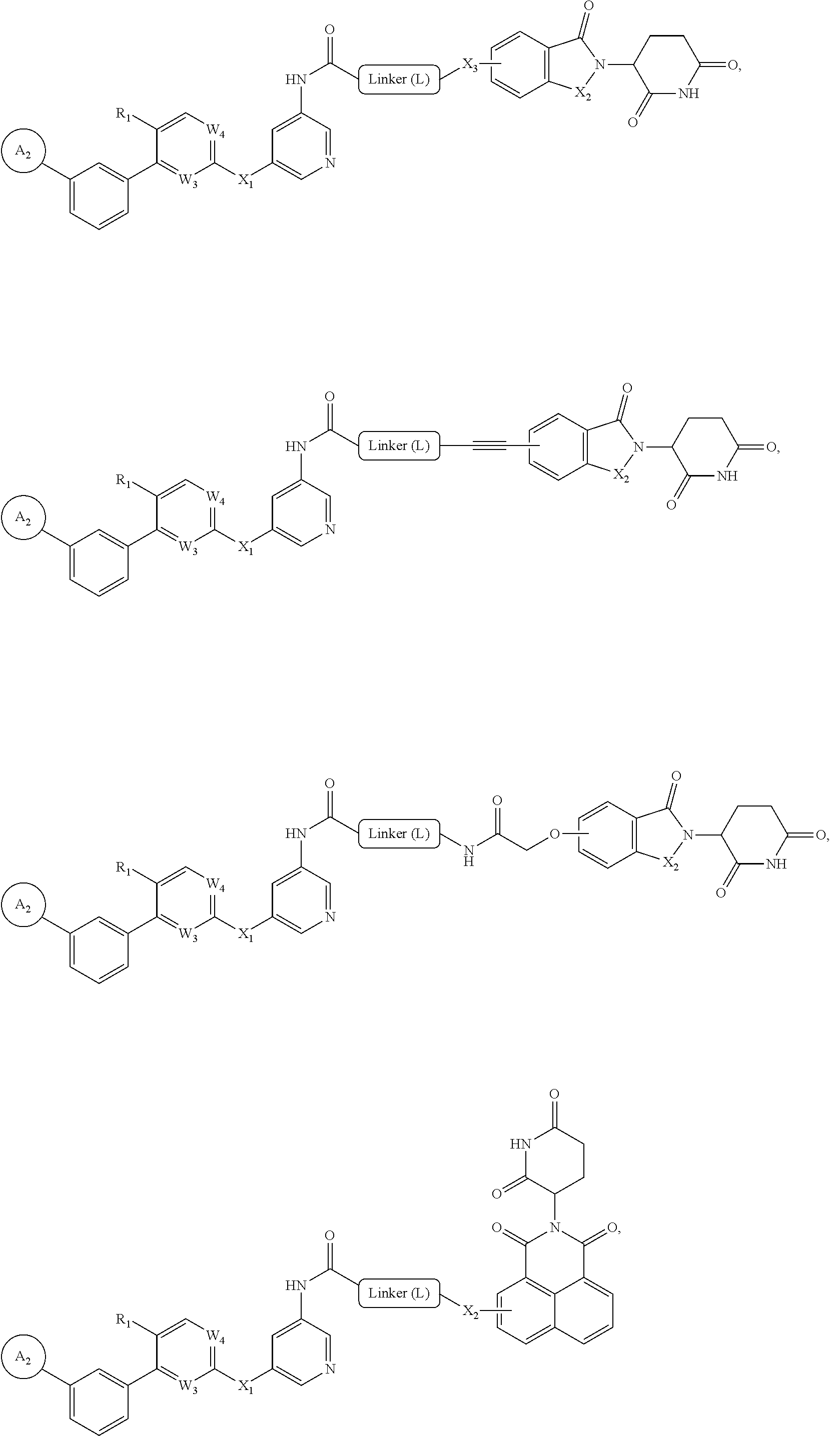

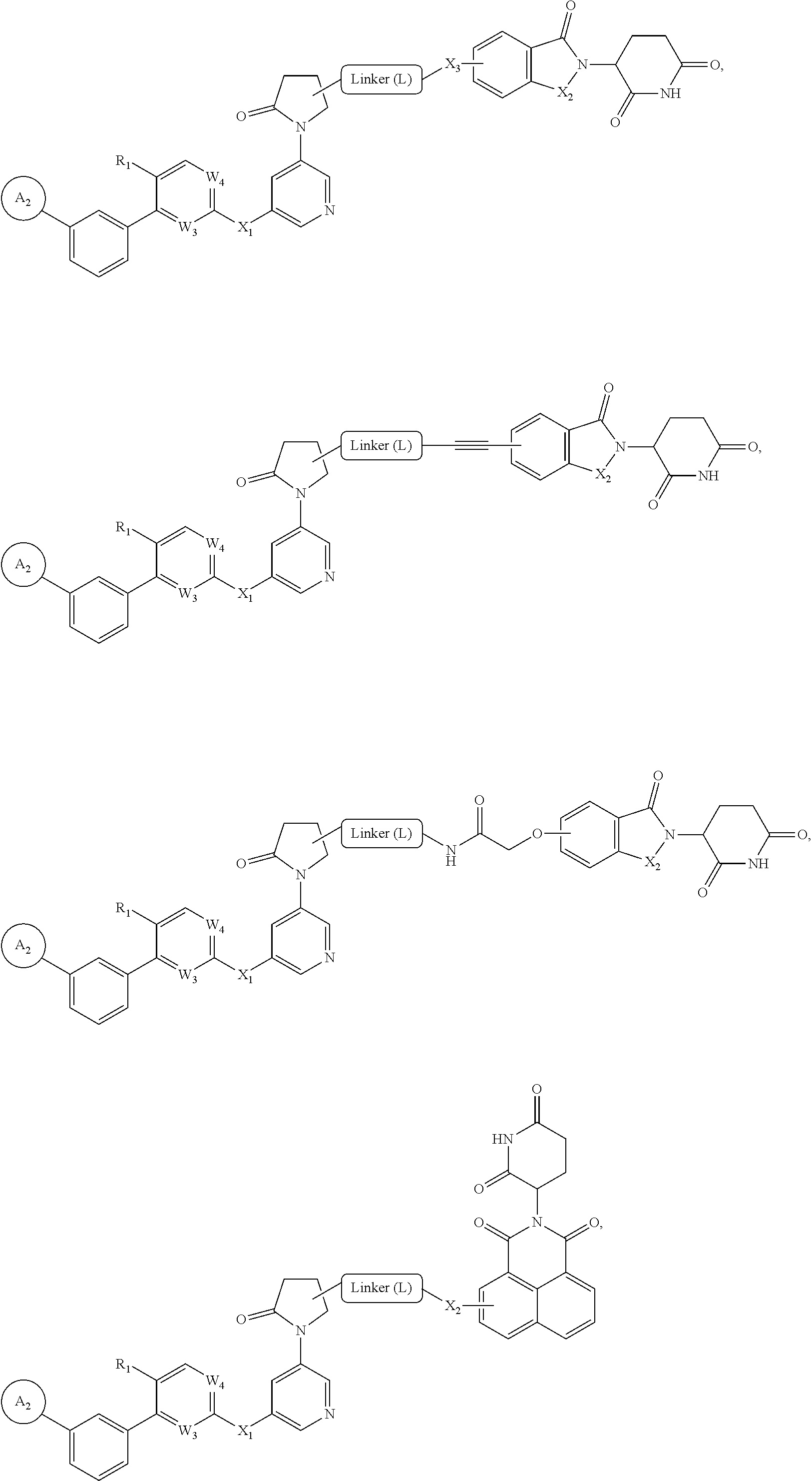

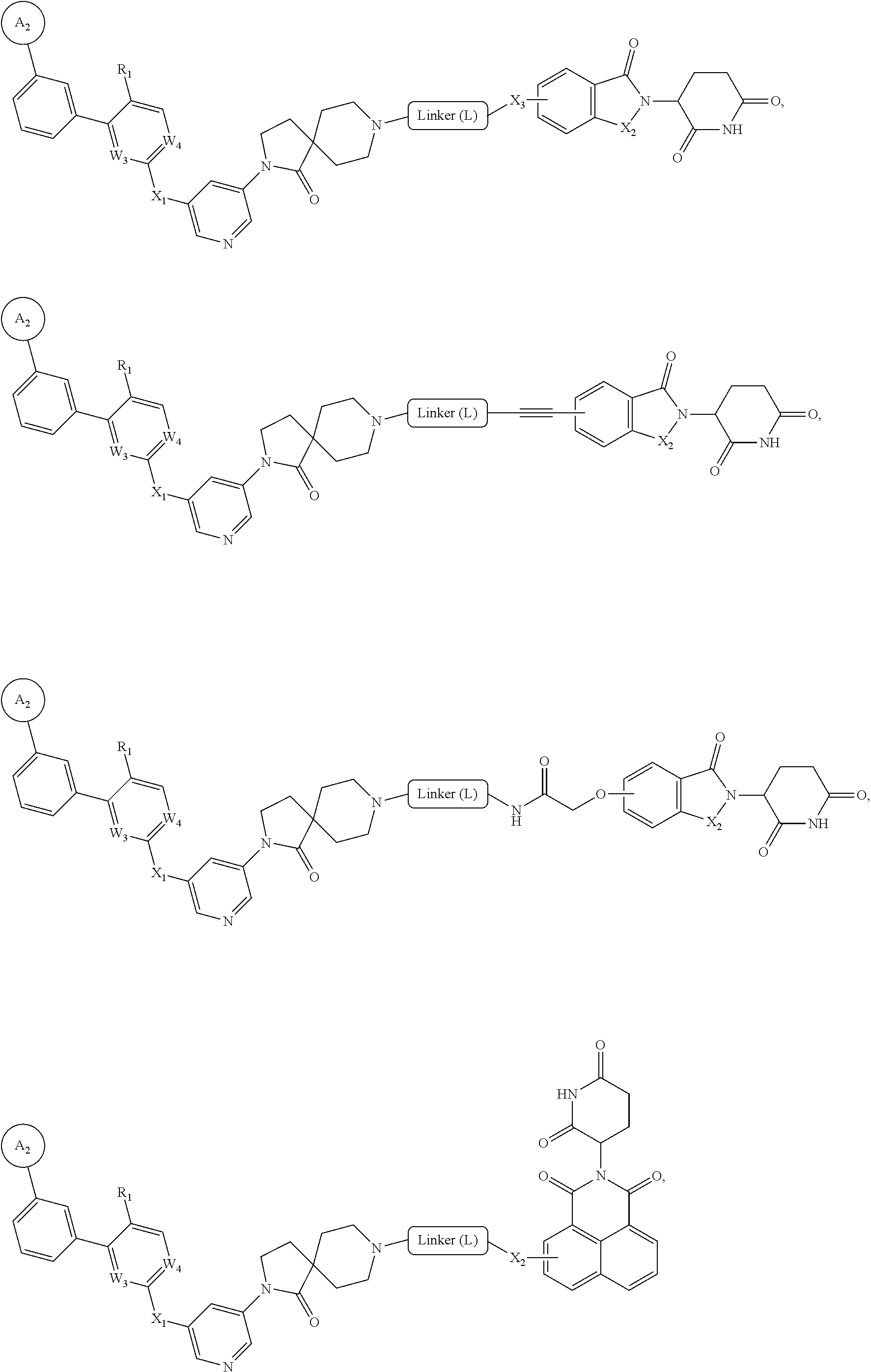

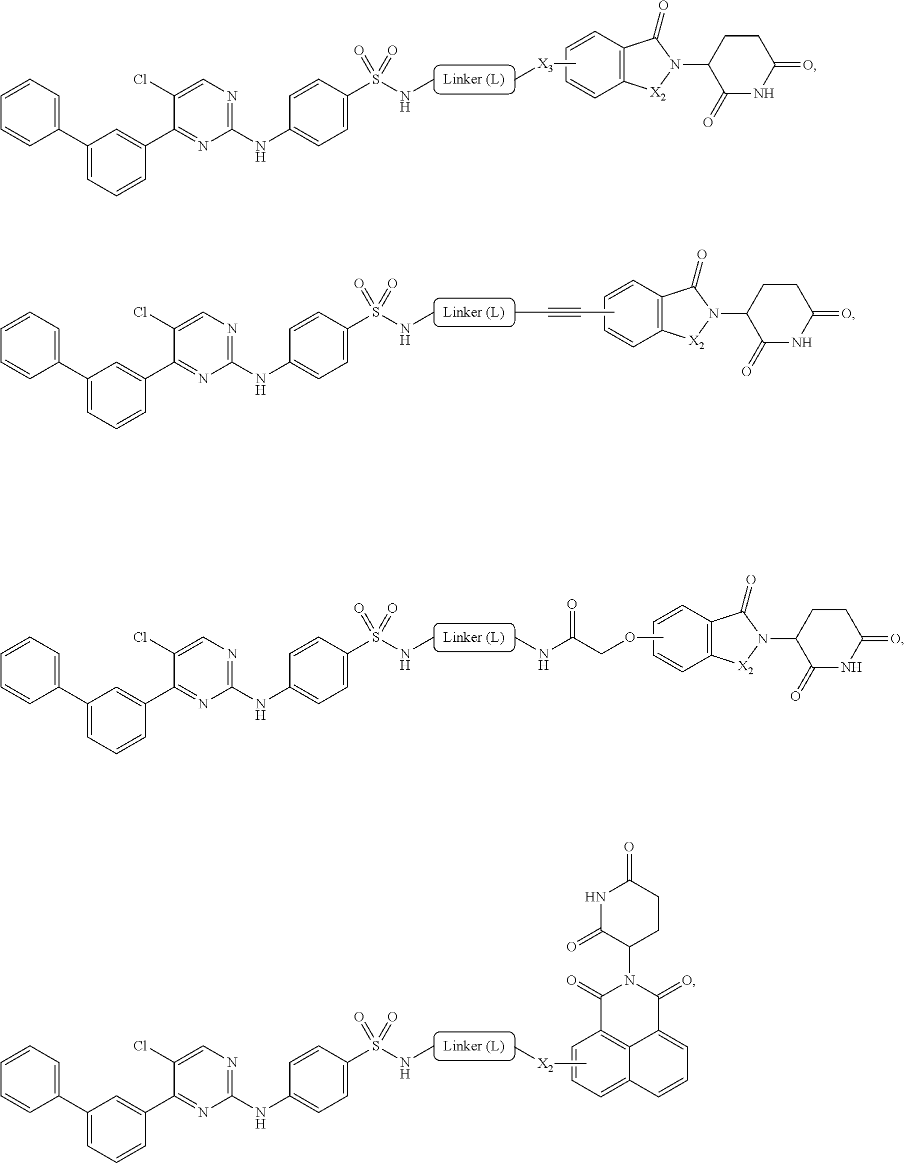

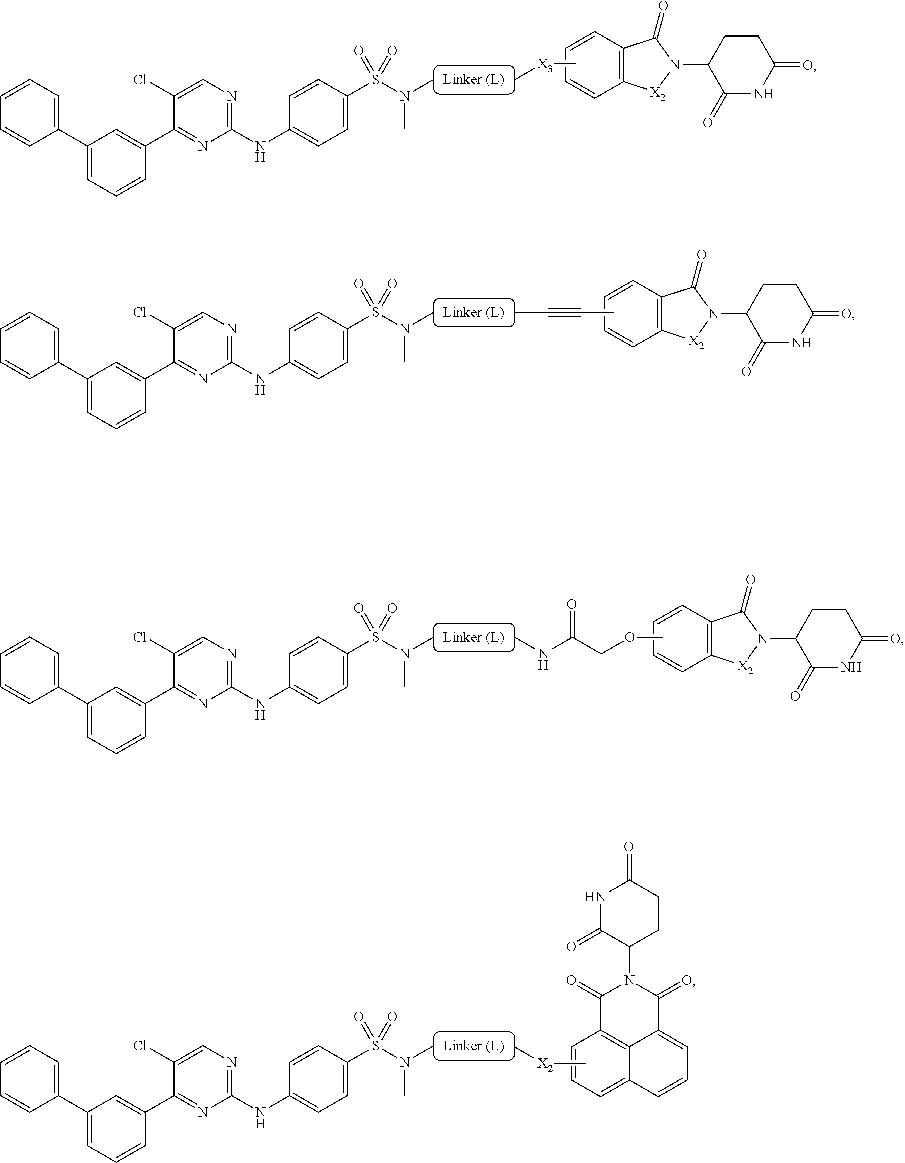

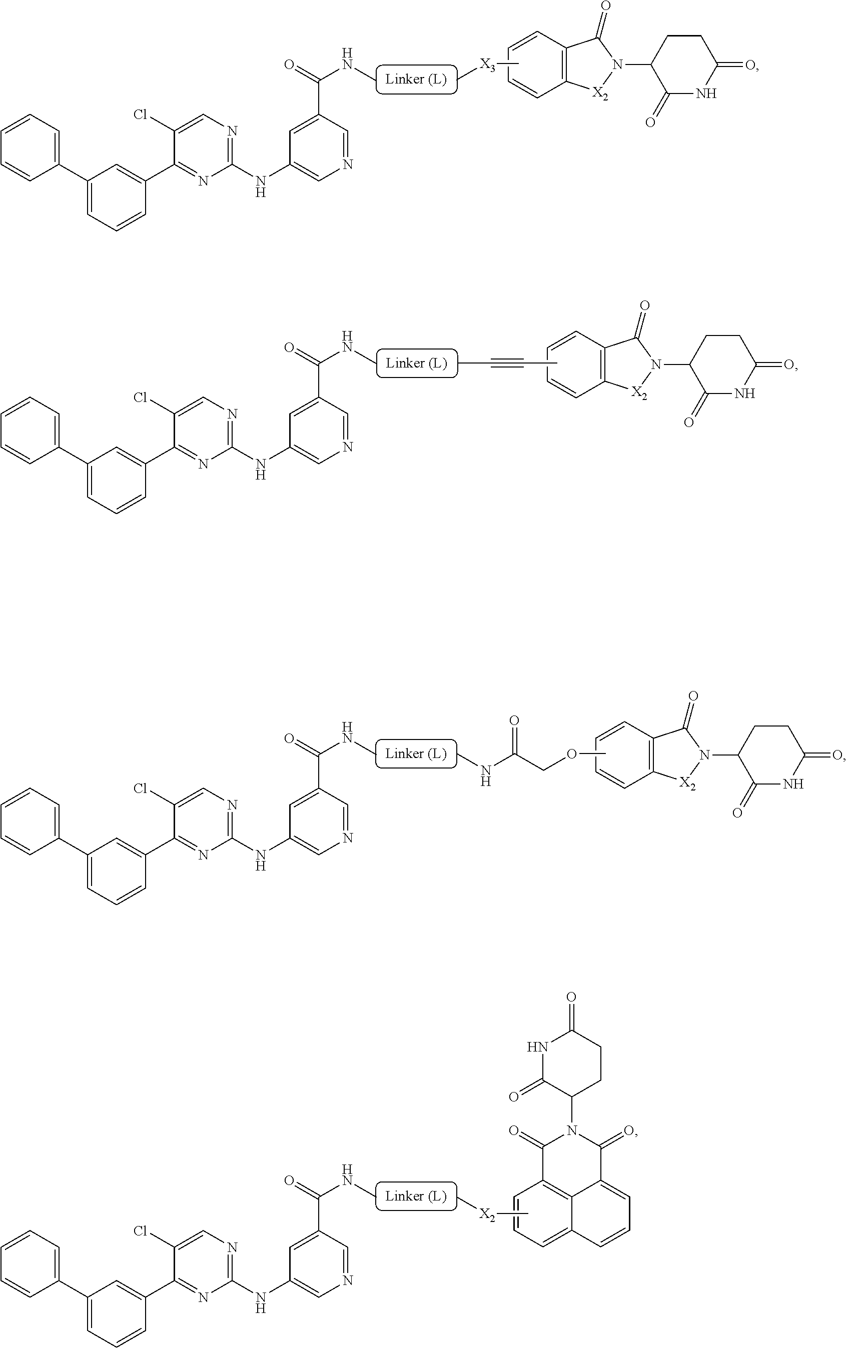

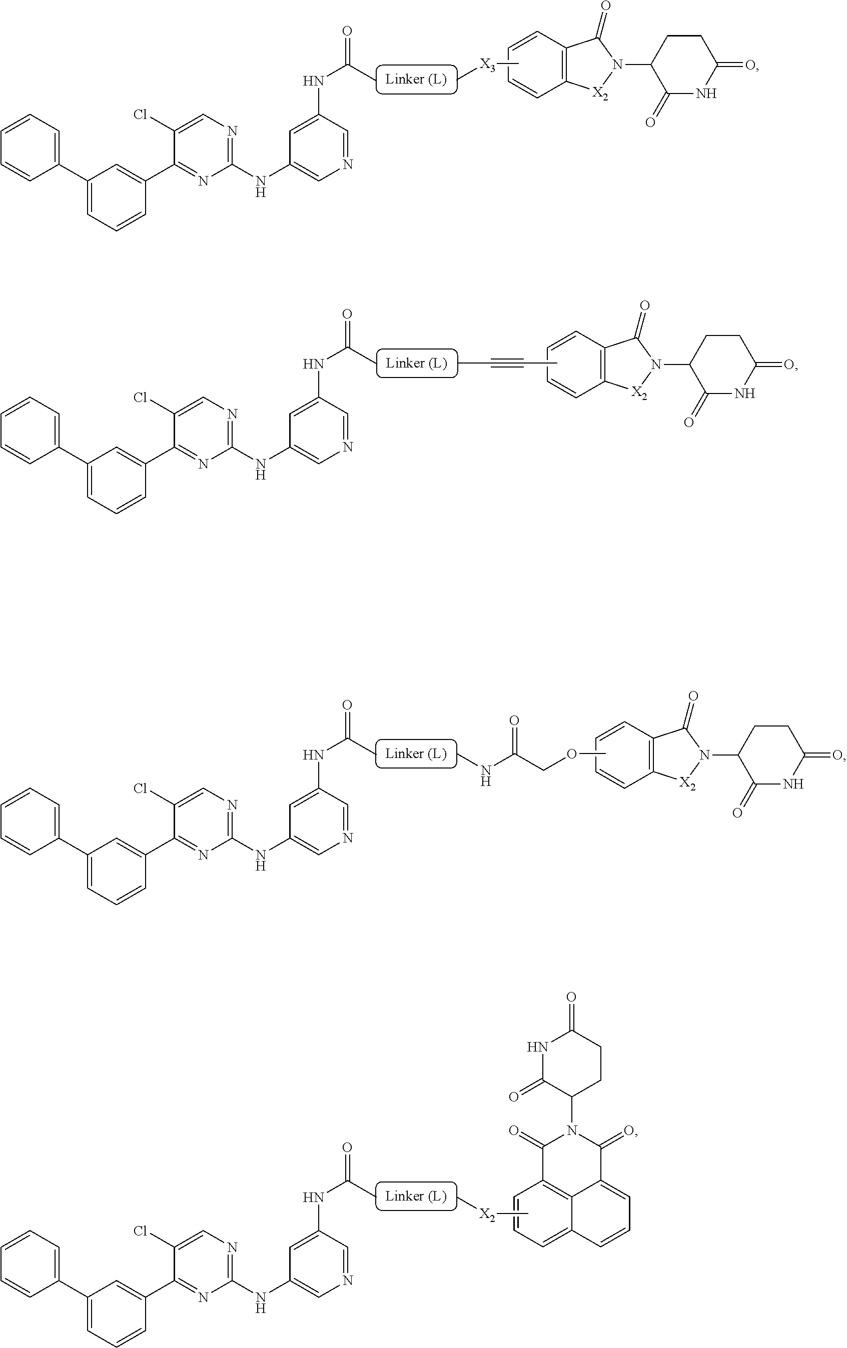

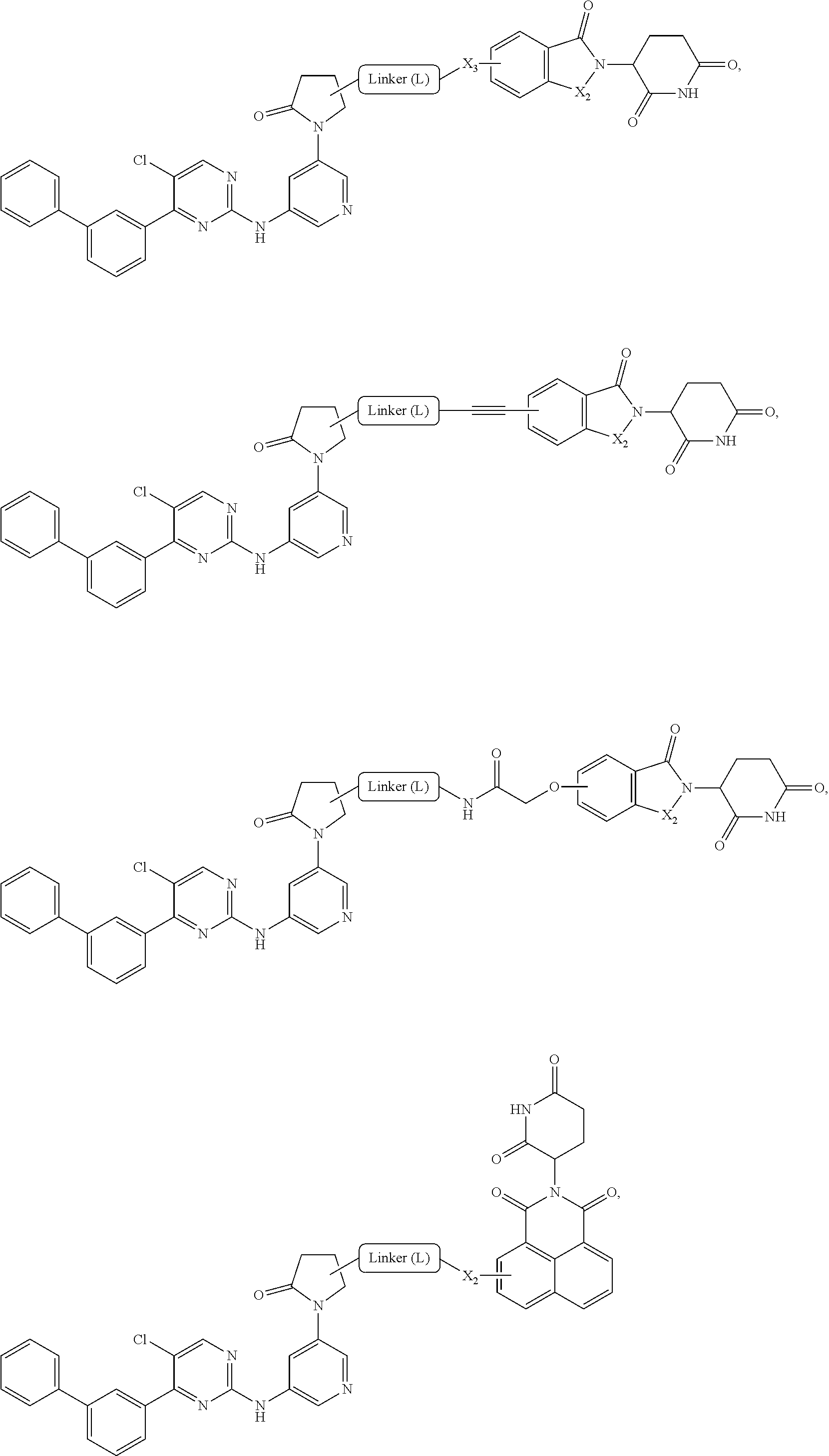

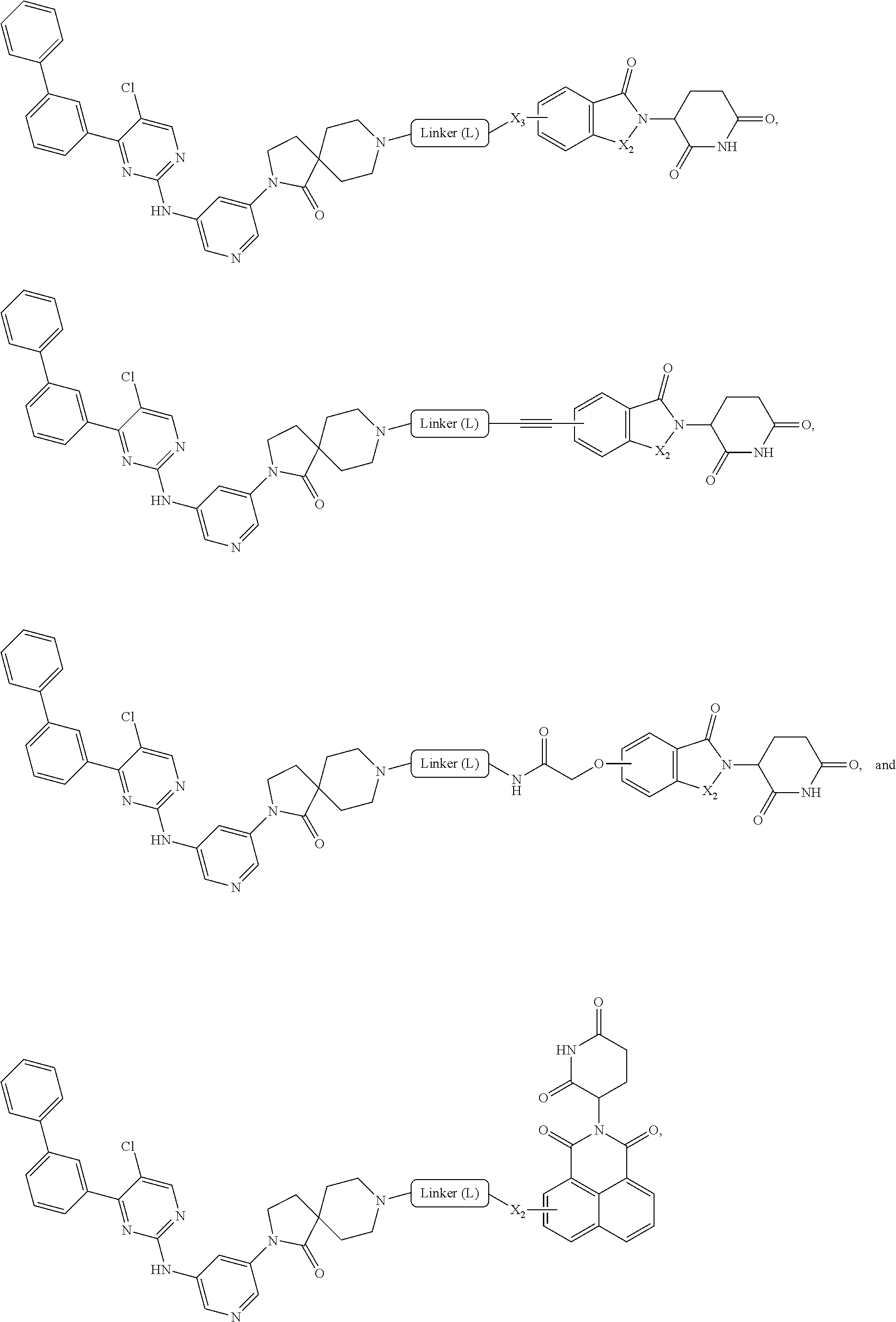

or a pharmaceutically acceptable salt or stereoisomer thereof.

Yet other degrons that bind cereblon and which may be suitable for use in the present invention are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046, each of which is incorporated herein by reference in its entirety.

In some embodiments, the E3 ubiquitin ligase that is bound by the degron is the von Hippel-Lindau (VHL) tumor suppressor.

Representative examples of degrons that bind VHL are represented by any one of structures (D2-a) to (D2-j):

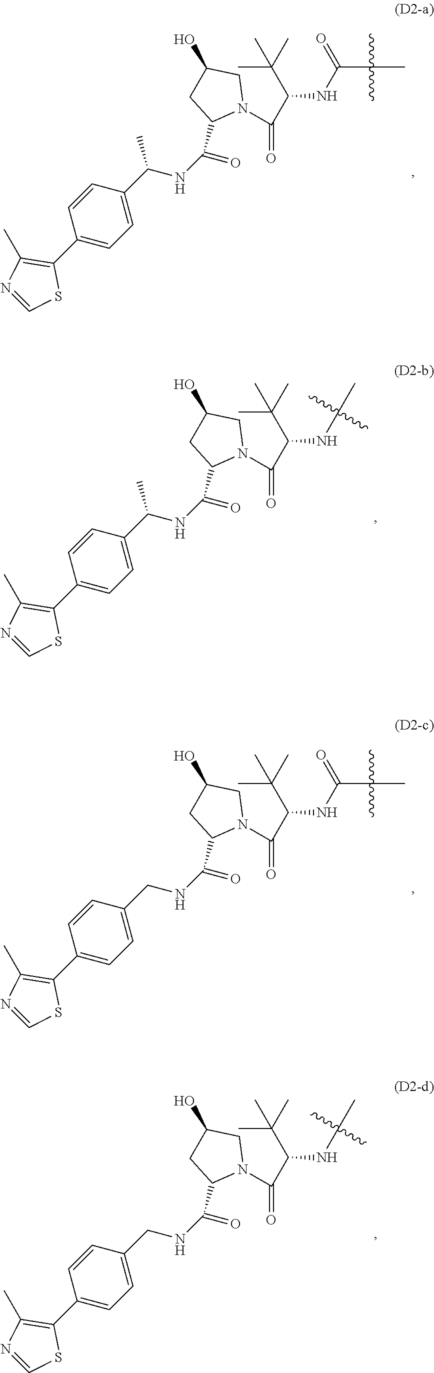

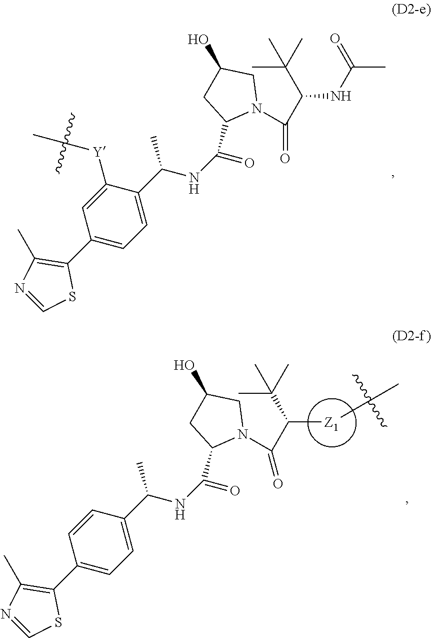

wherein Z₁ is a C₅-C₆ carbocyclic or 5- or 6-membered heterocyclic group, containing 1 to 3 heteroatoms selected from N, O, and S,

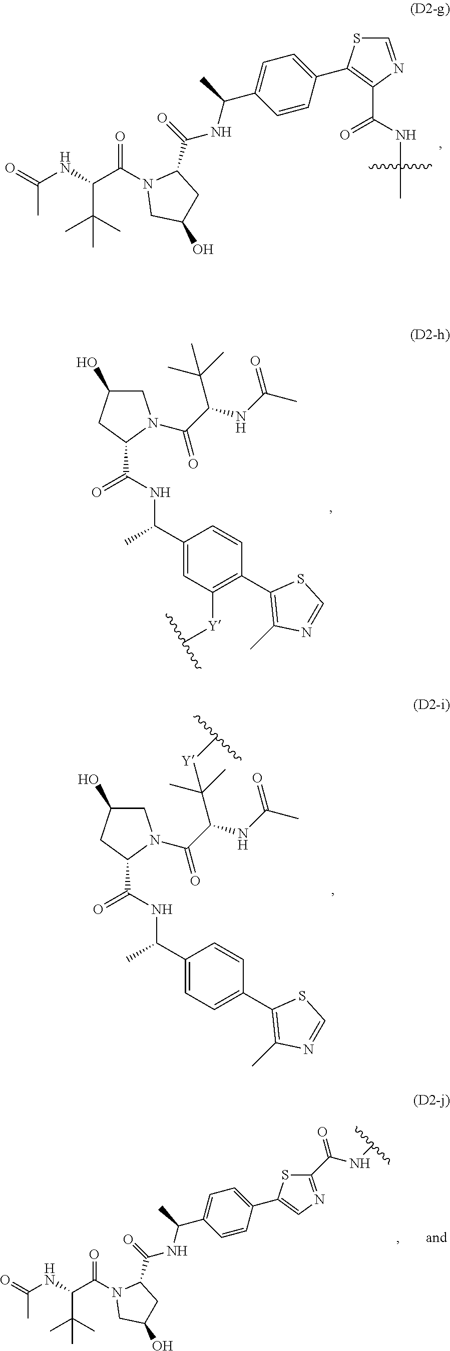

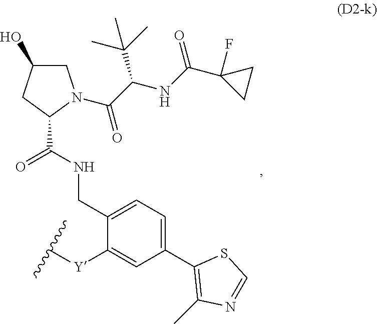

wherein Y′ is a bond, CH₂, NH, NMe, O, or S, or a stereoisomer thereof. In some embodiments, Y′ is O, In some embodiments, Y′ is NH.

In some embodiments, Z₁ is phenyl, pyrrolyl, furanyl, thiophenyl, pyrazolyl, imidazolyl, oxazolyl, thiazolyl, pyridinyl, pyridazinyl, or pyrimidinyl. In certain embodiments, Z is

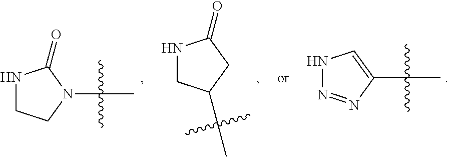 [SMILES: *N1CCNC1=O ; *C1CNC(=O)C1 ; *c1c[nH]nn1]

In some embodiments, bifunctional compounds of the present invention are represented by an one of the following structures:

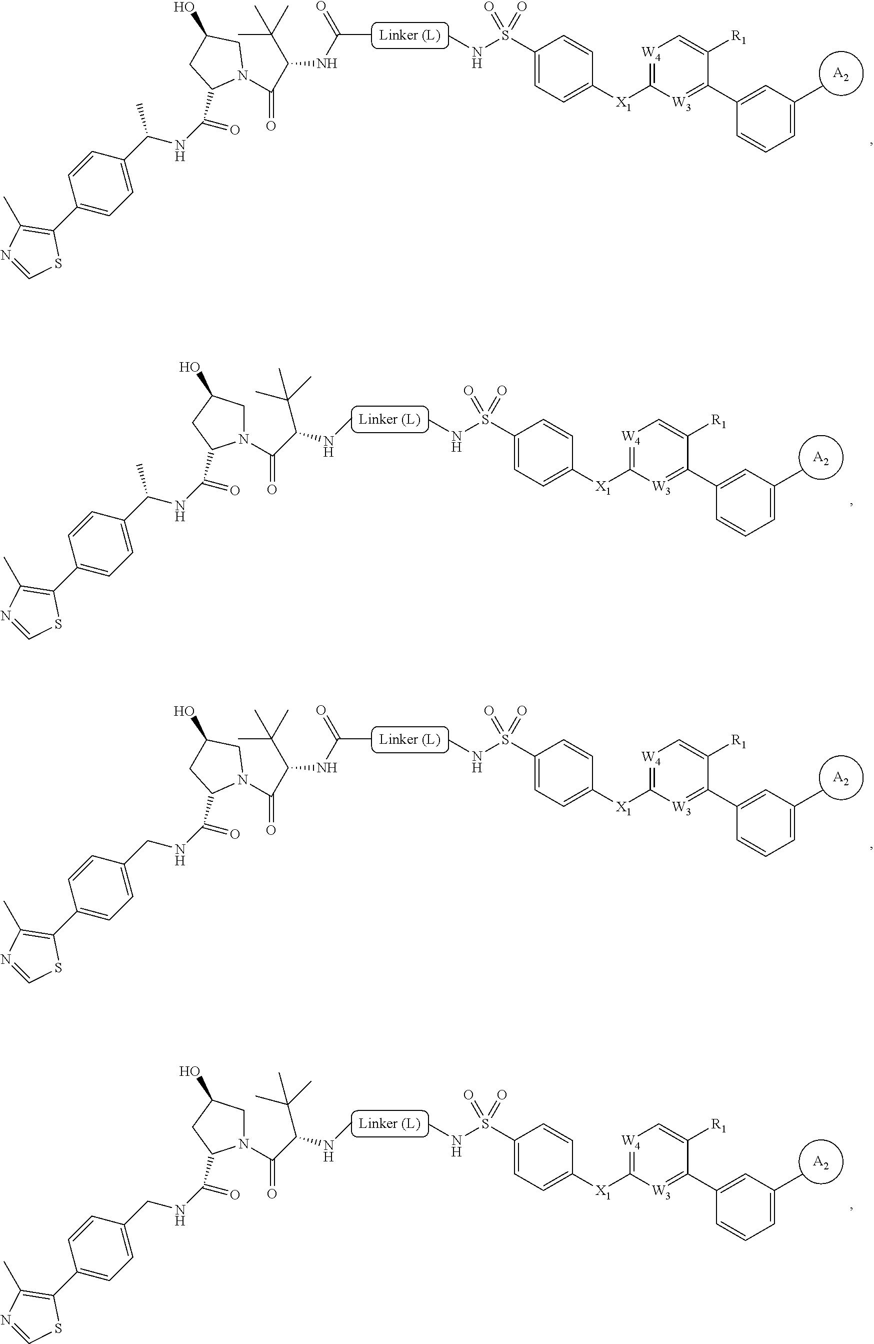

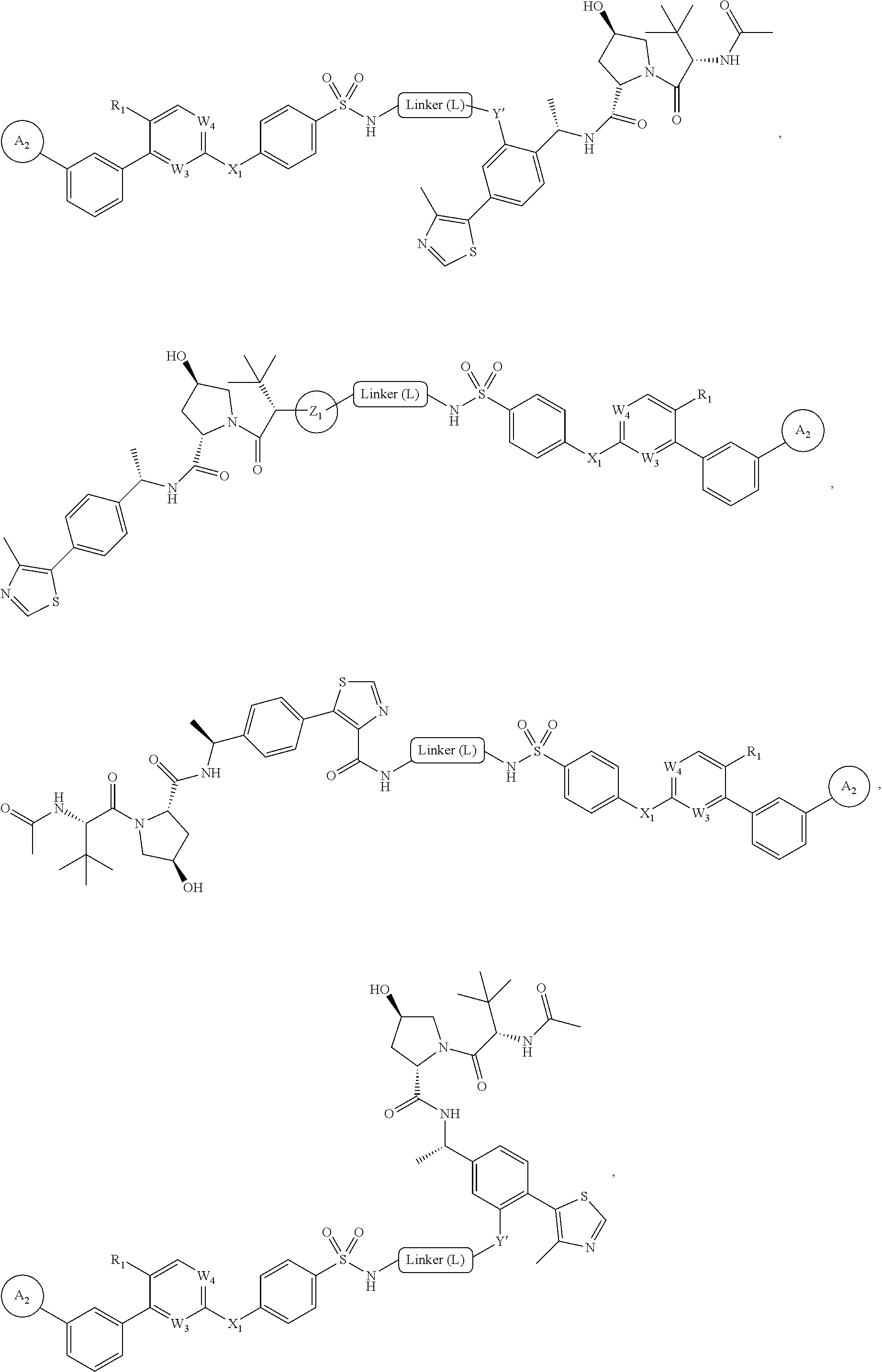

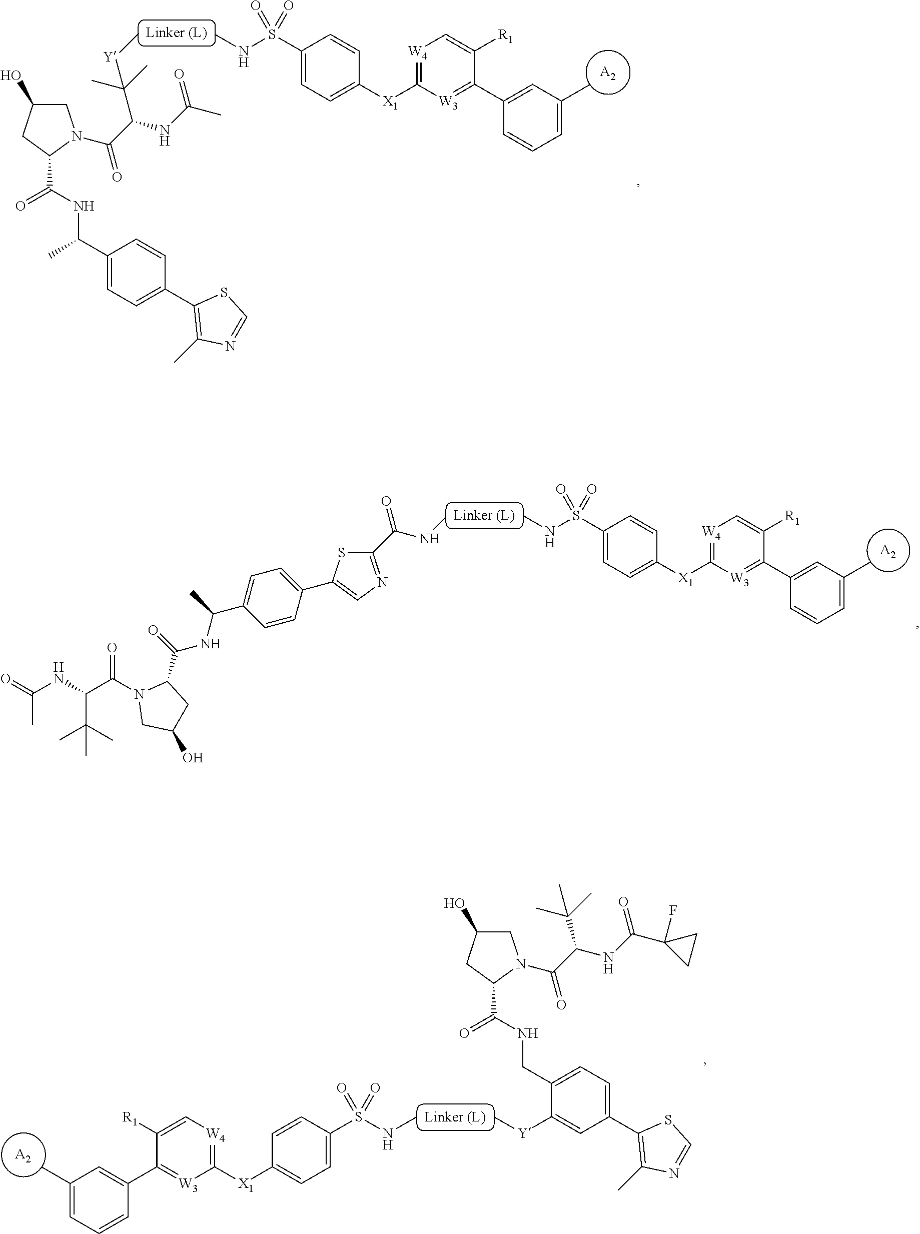

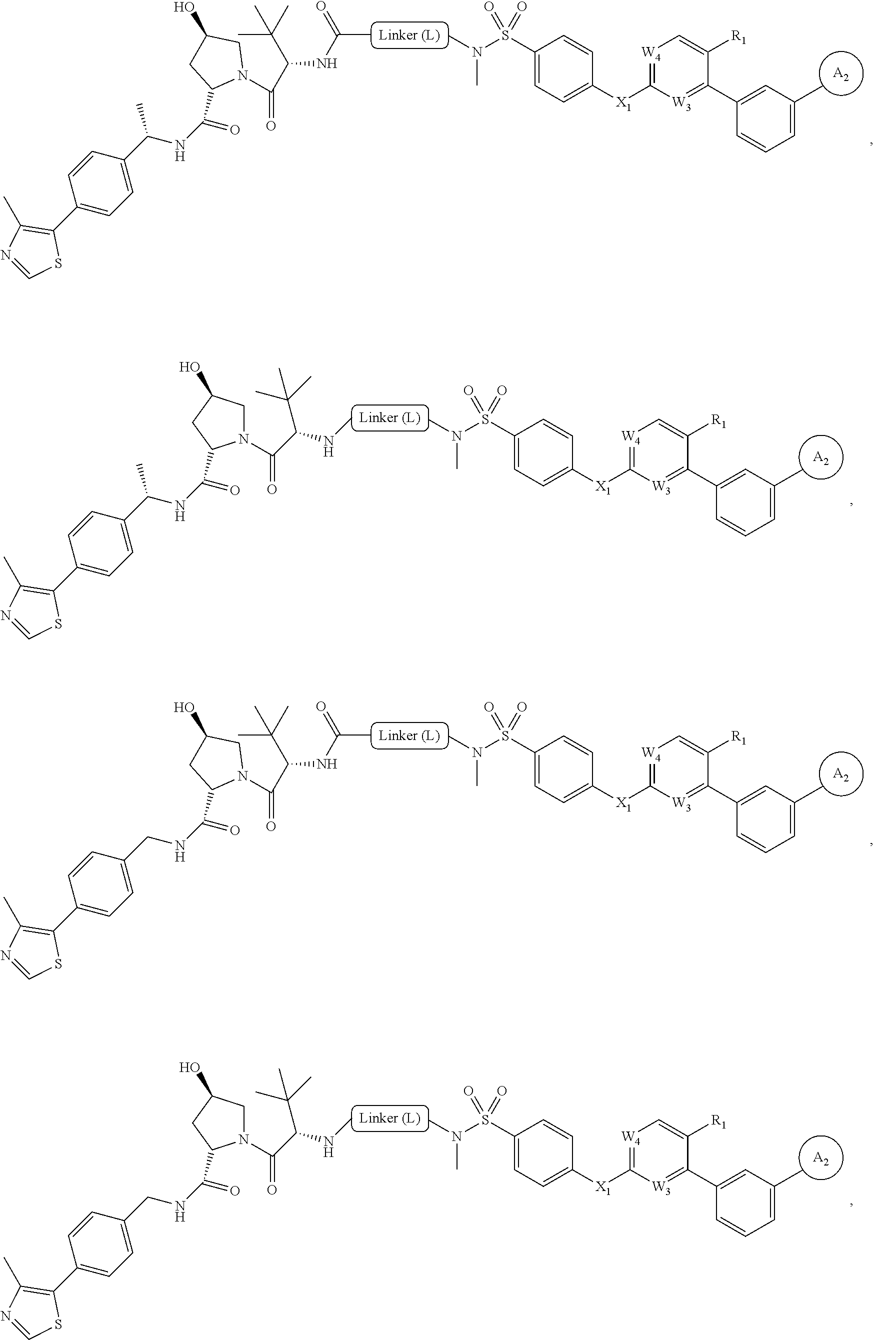

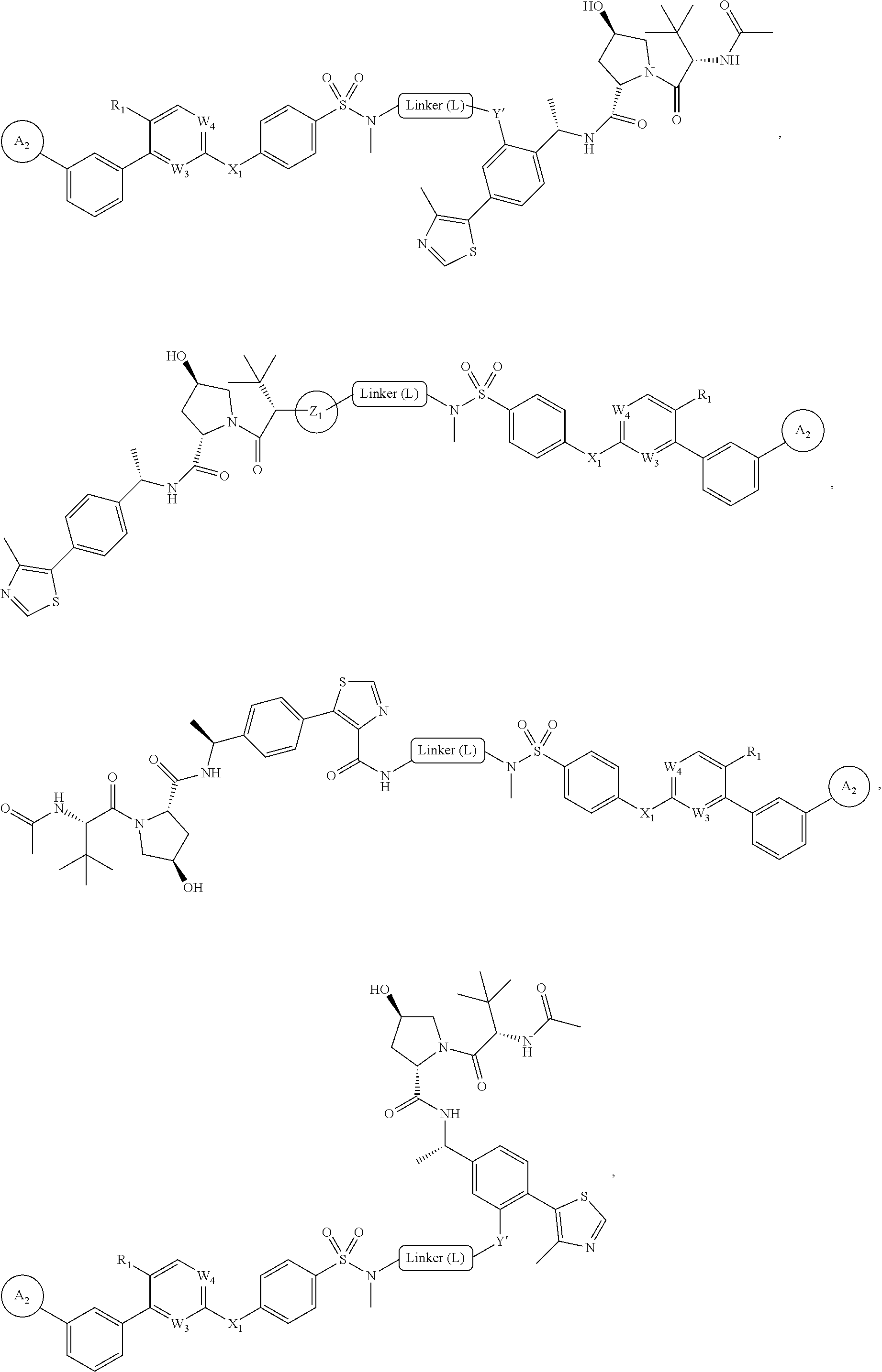

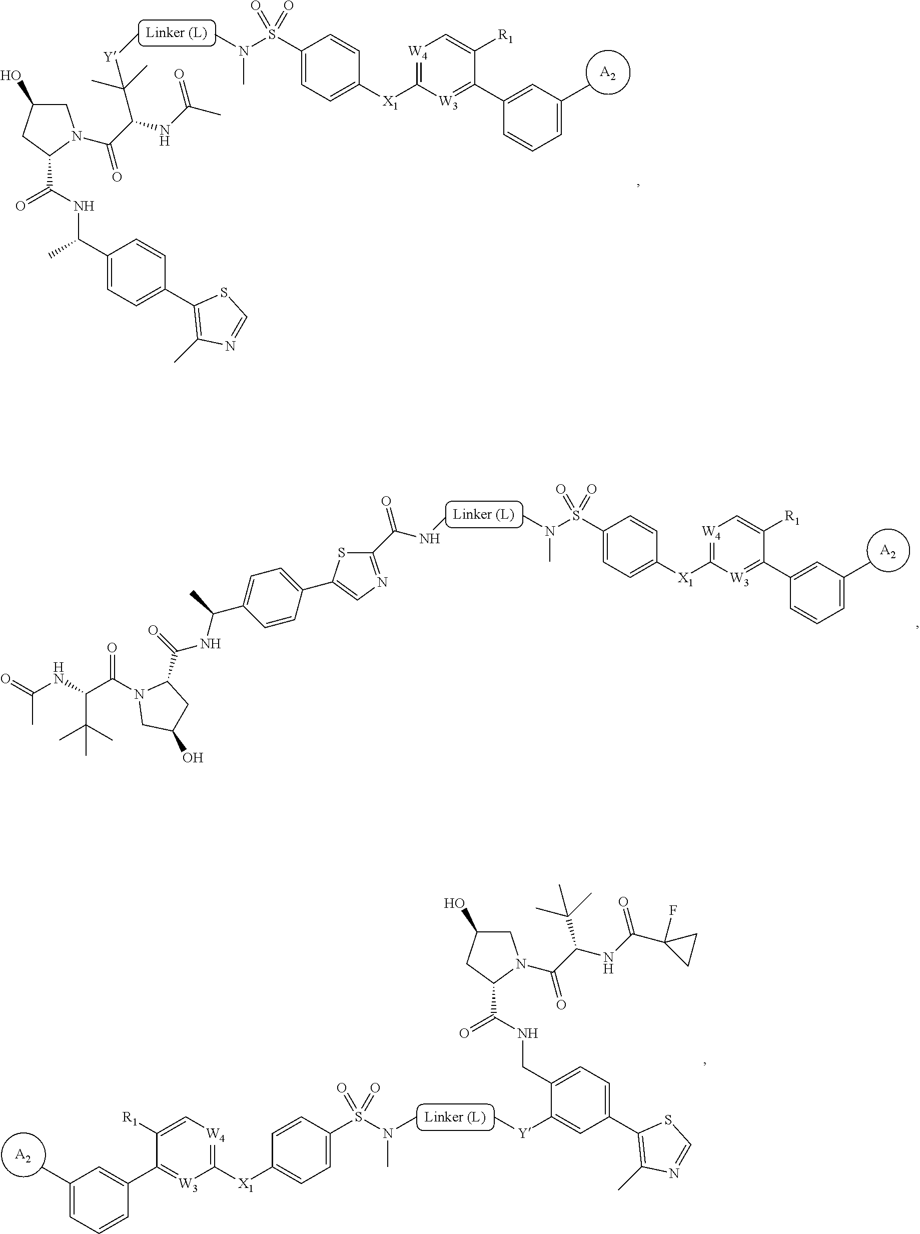

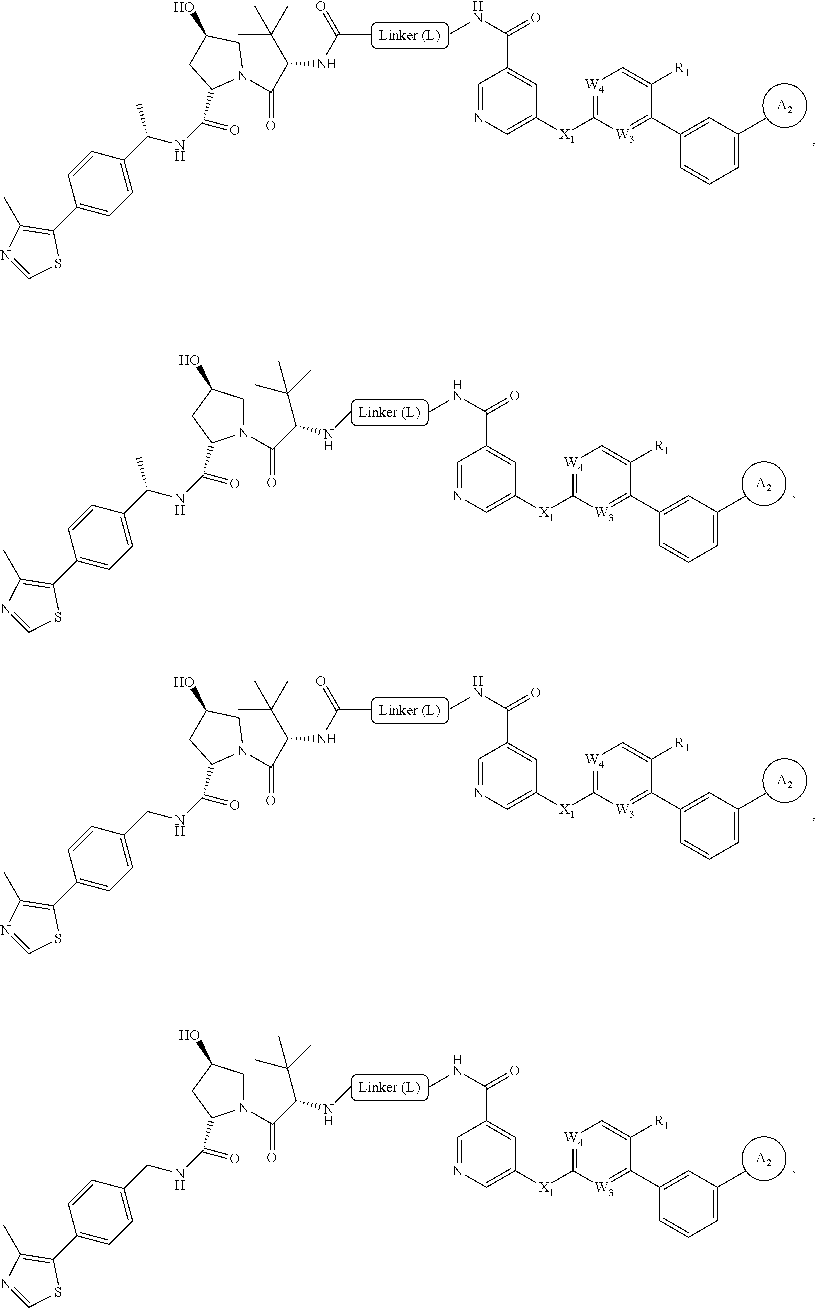

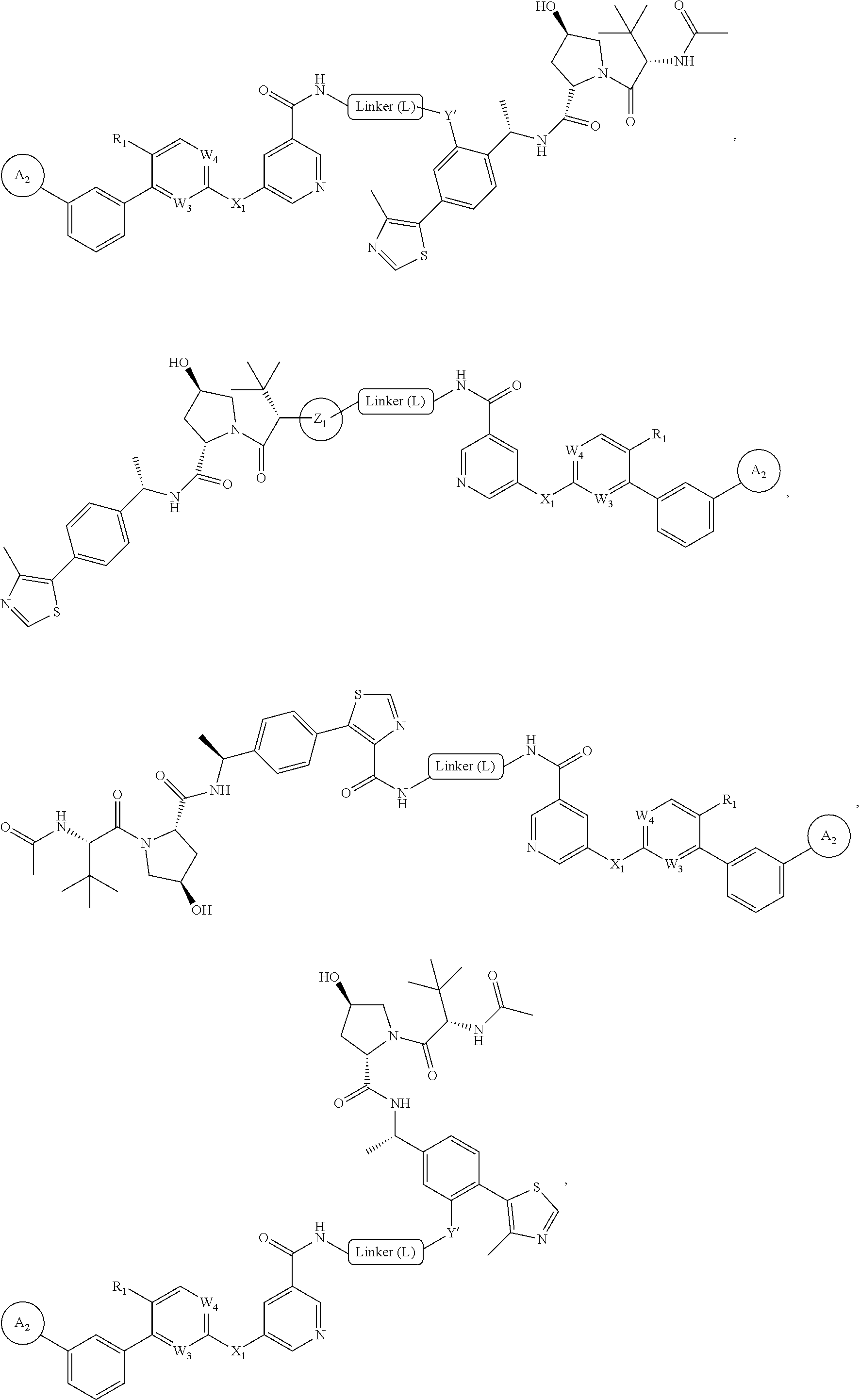

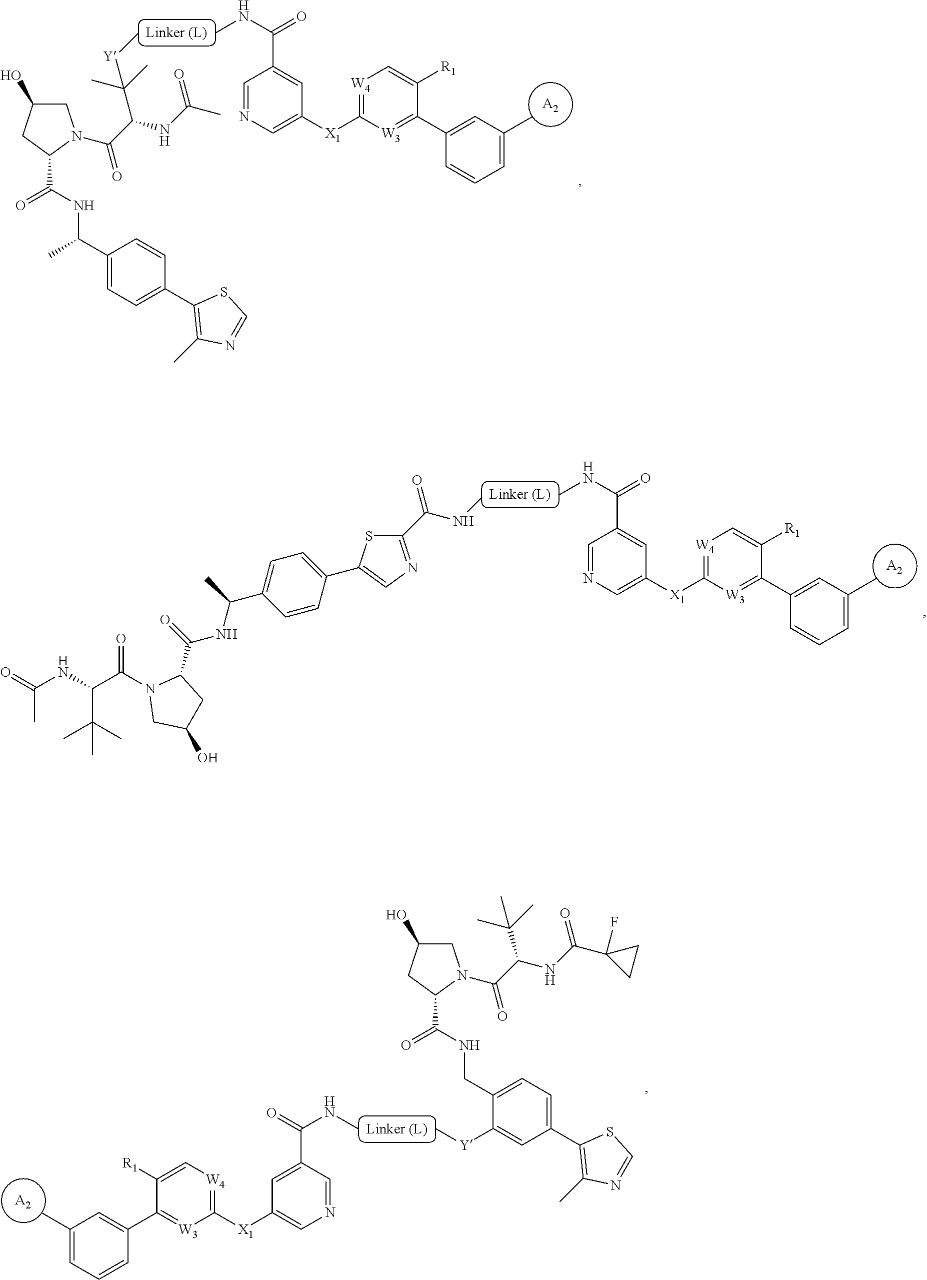

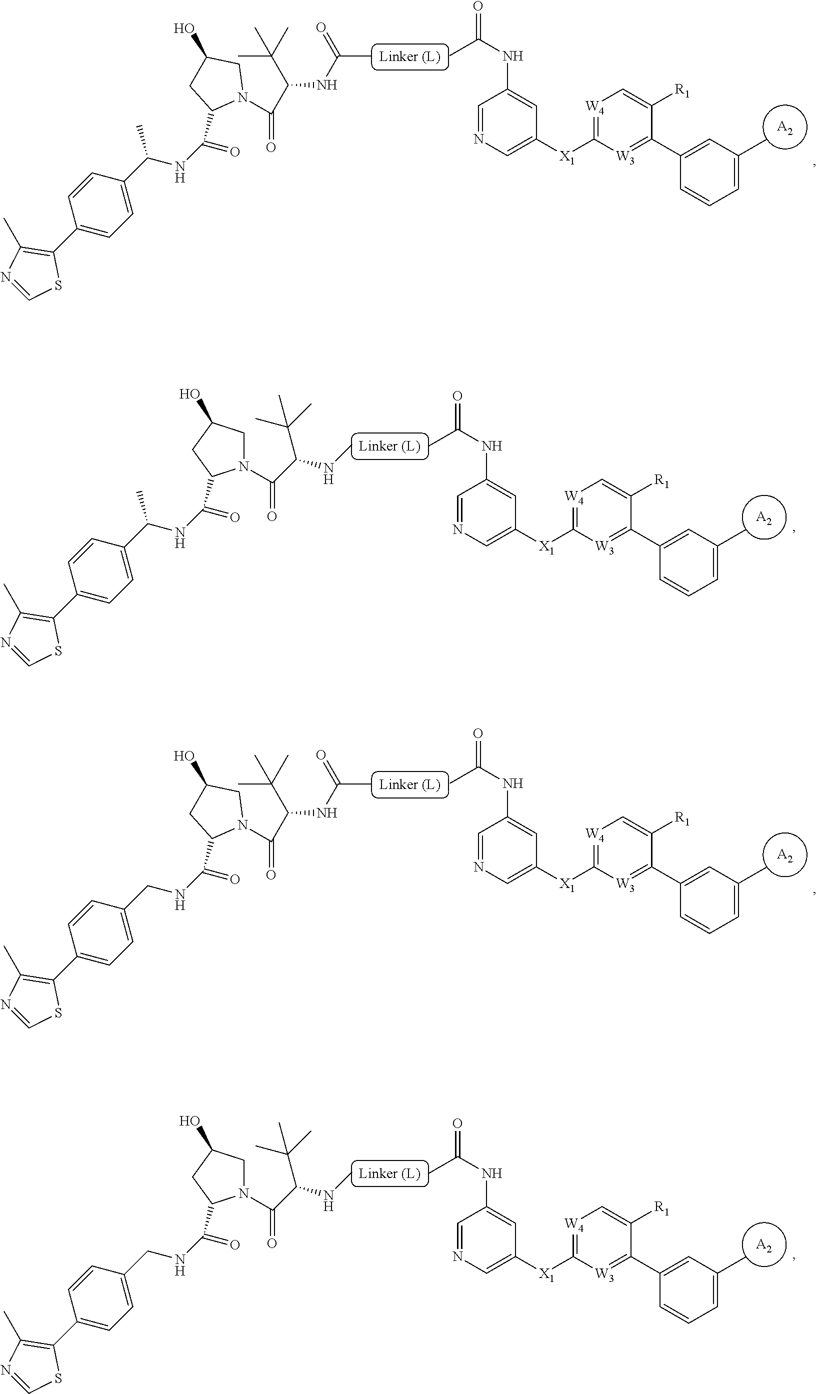

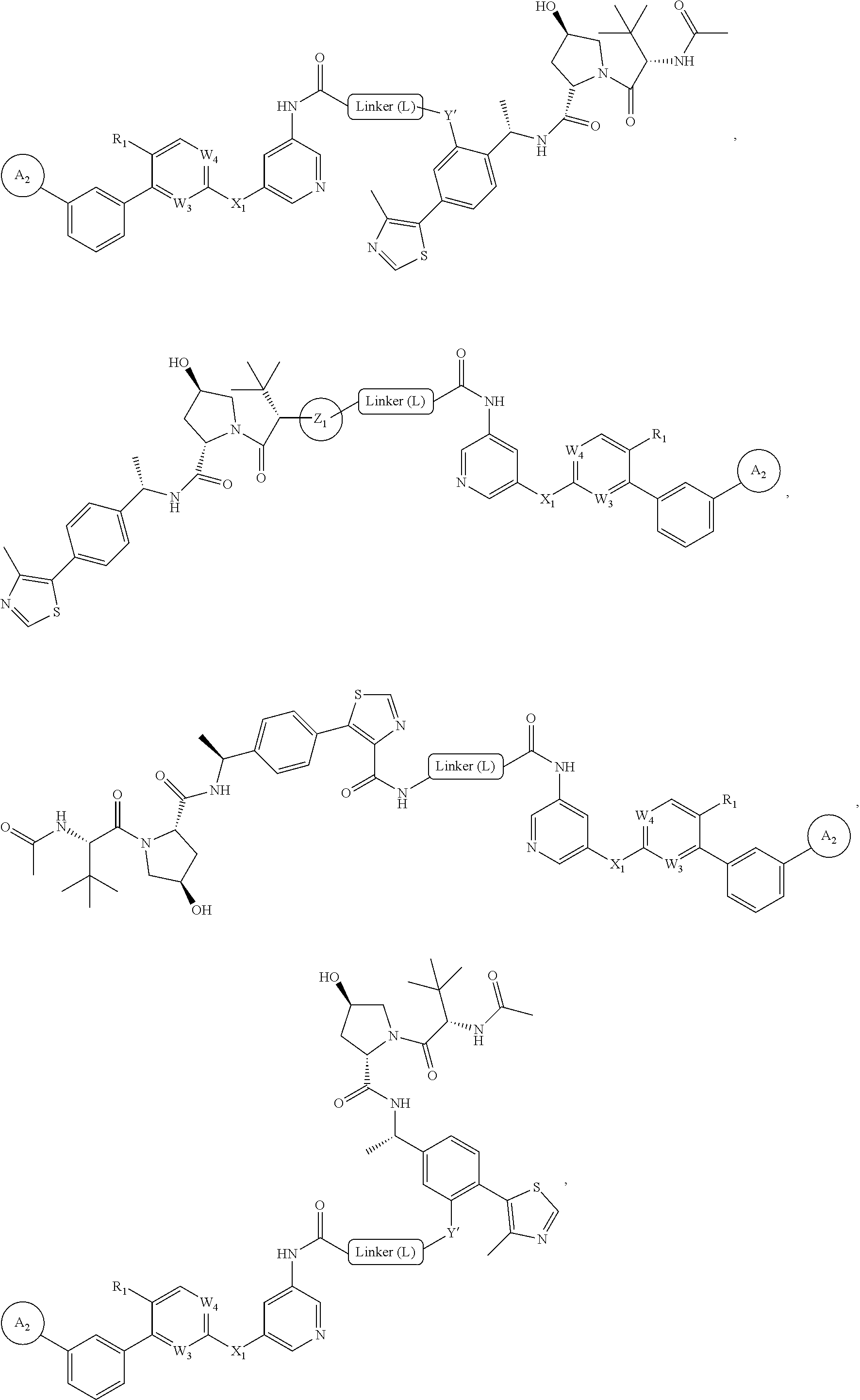

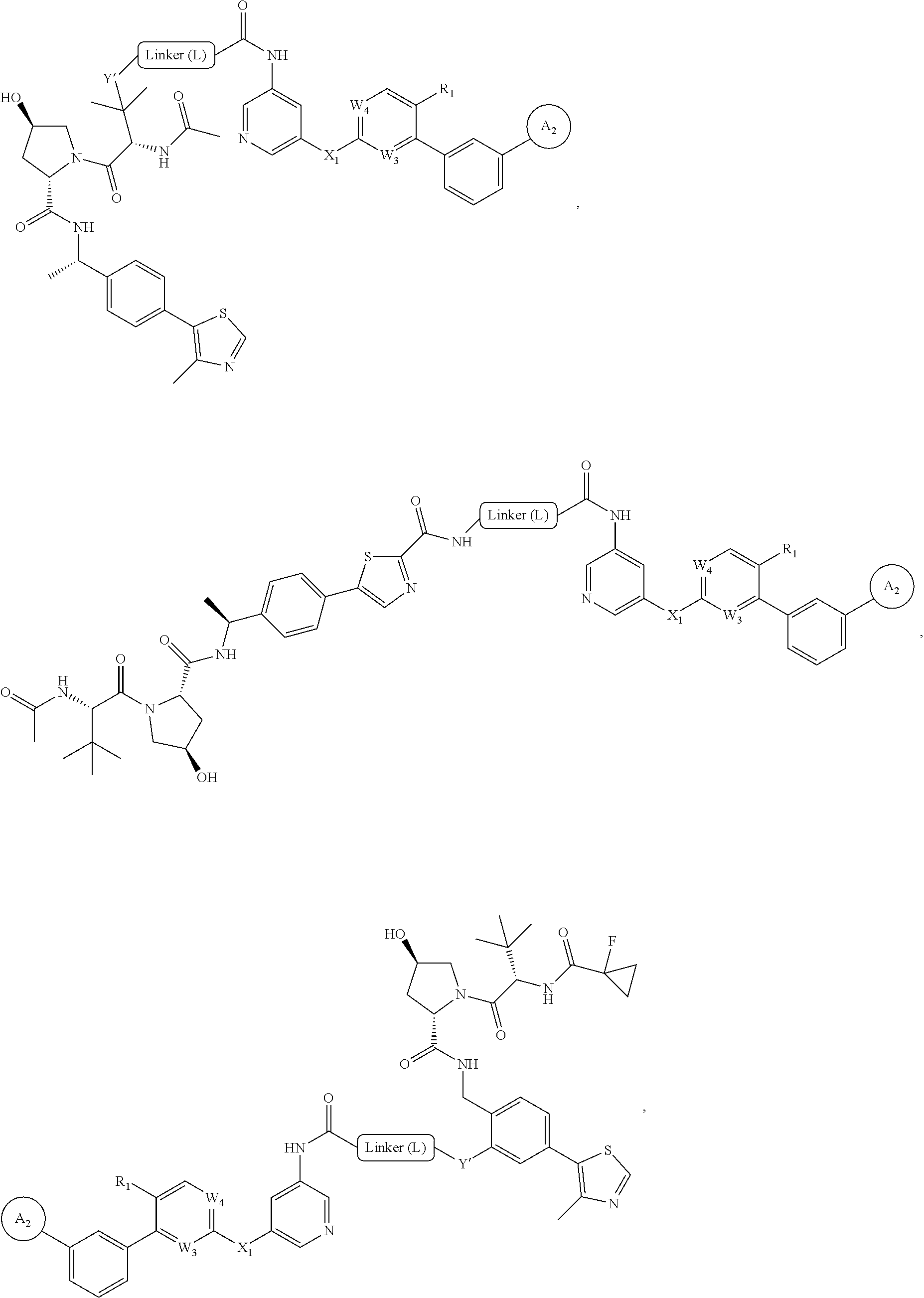

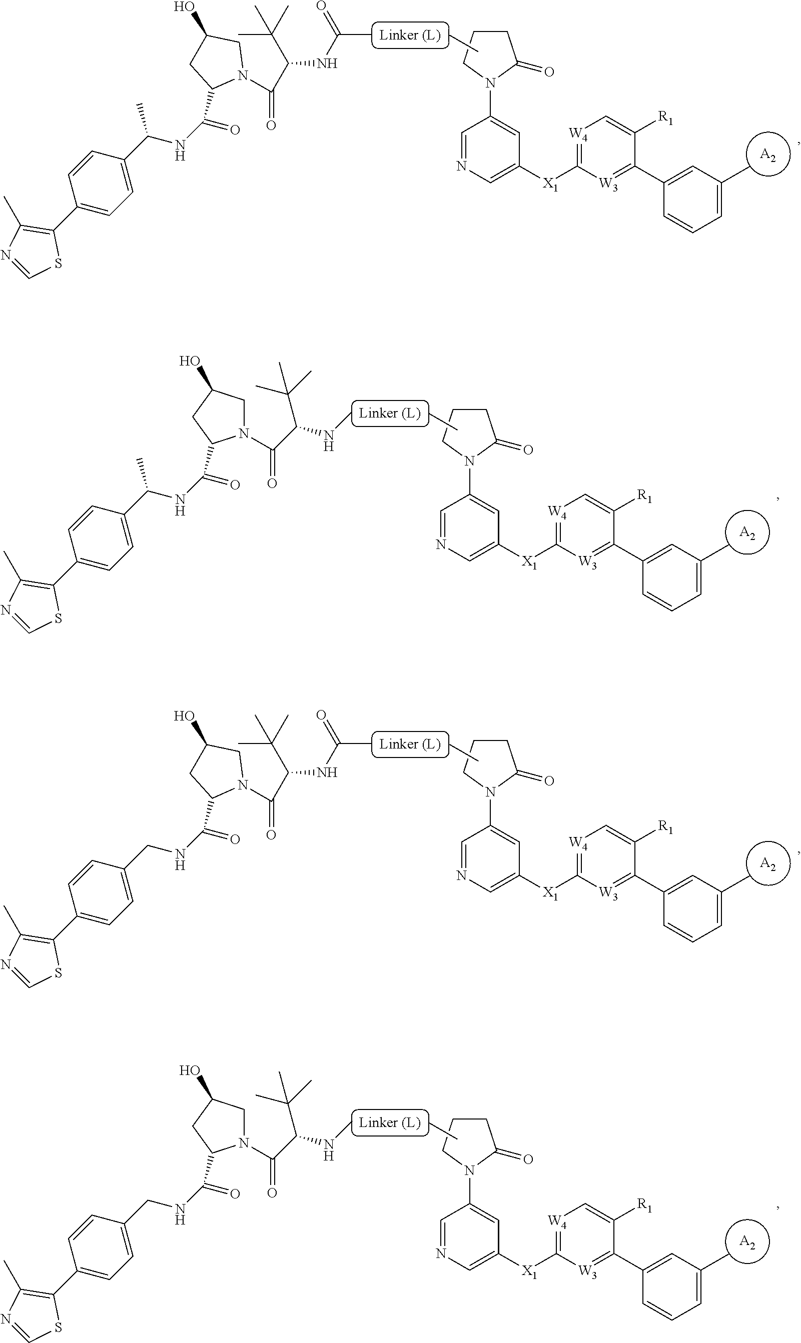

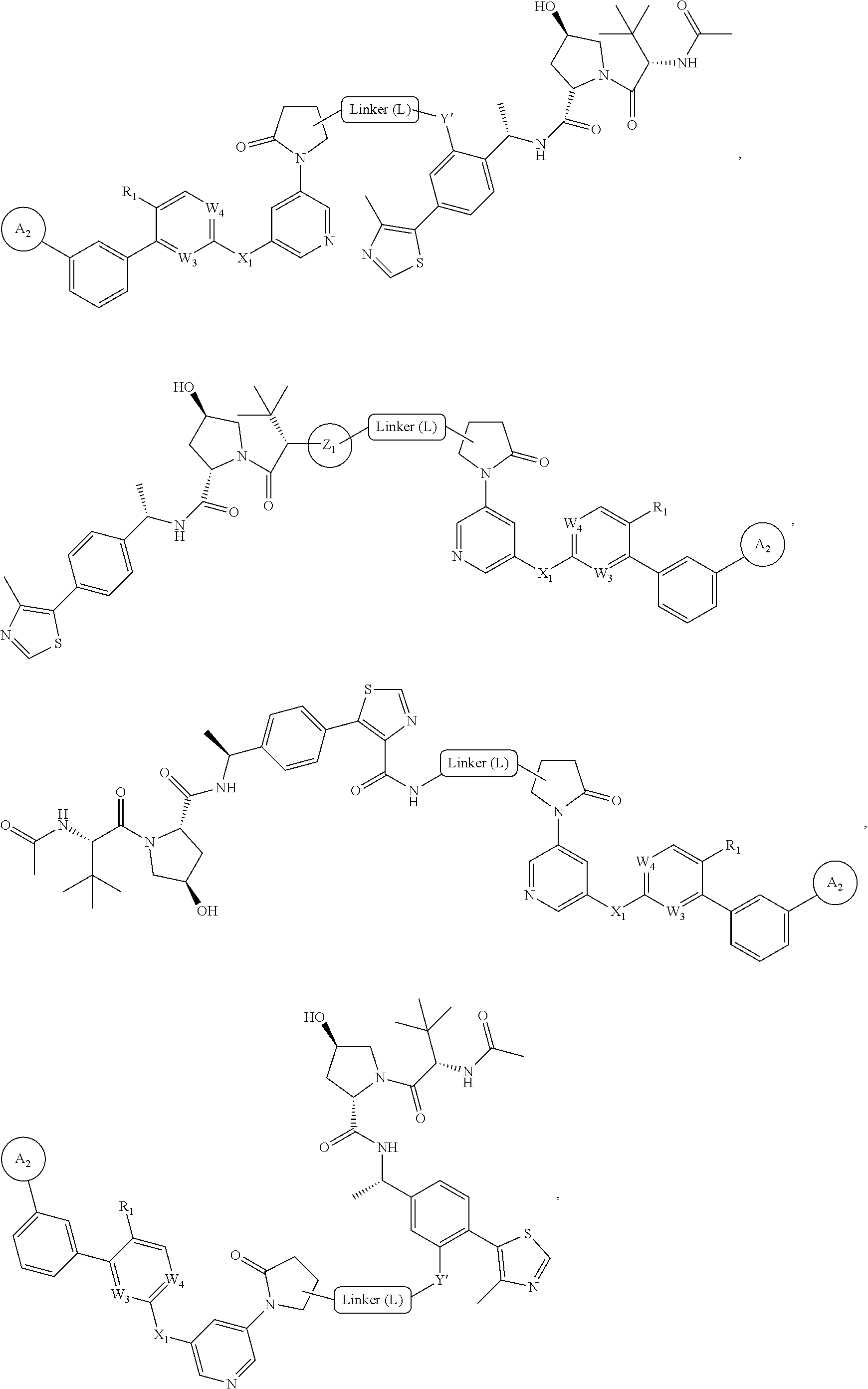

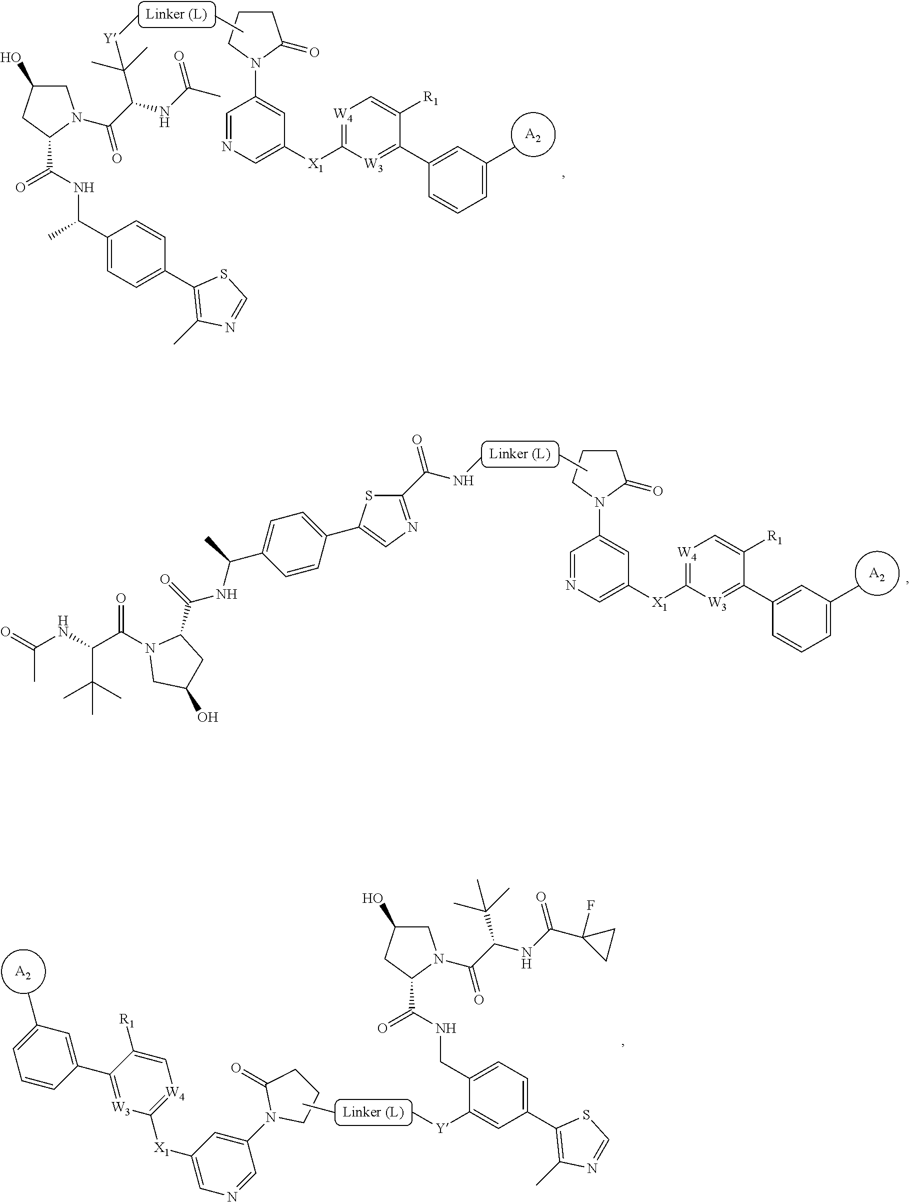

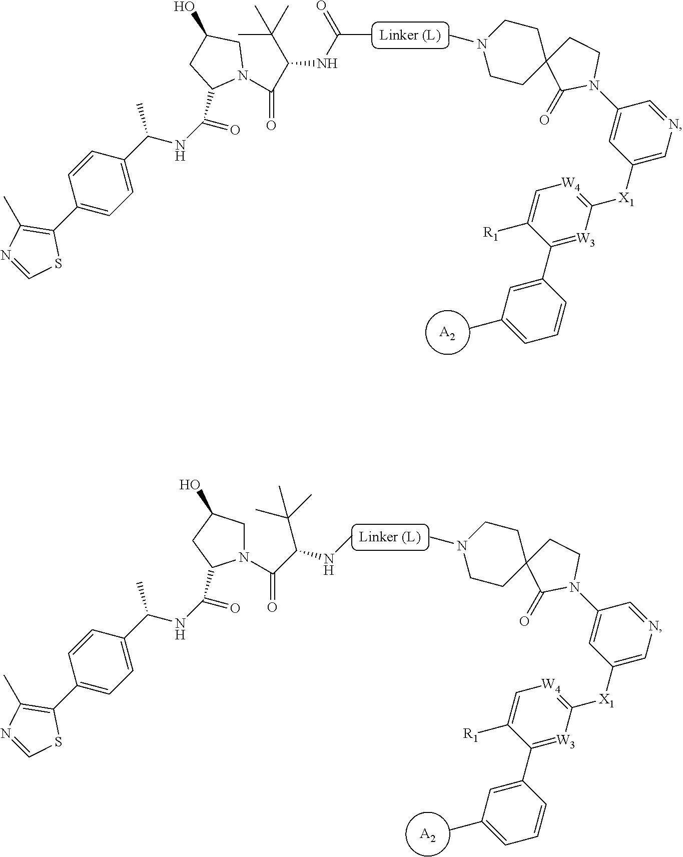

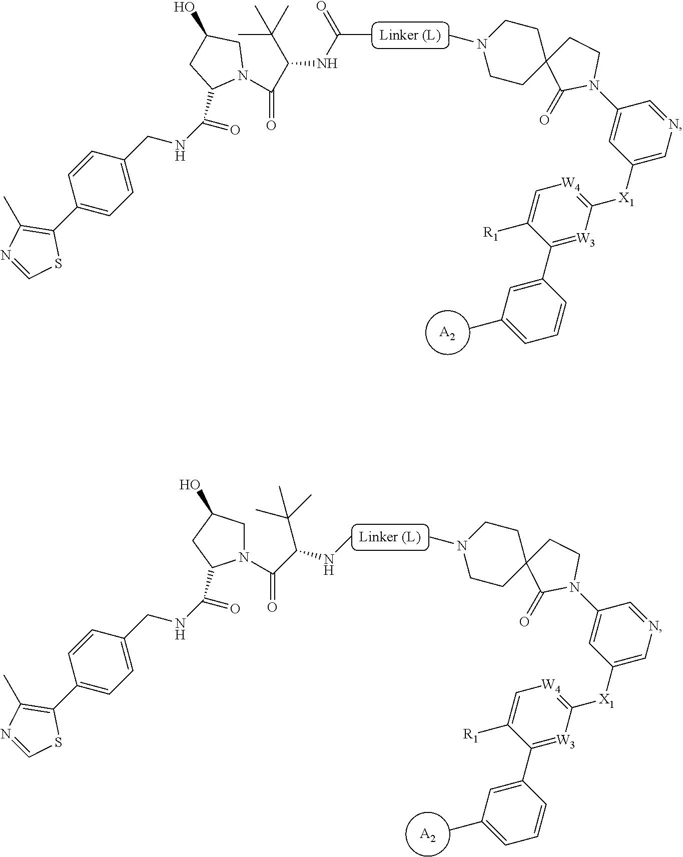

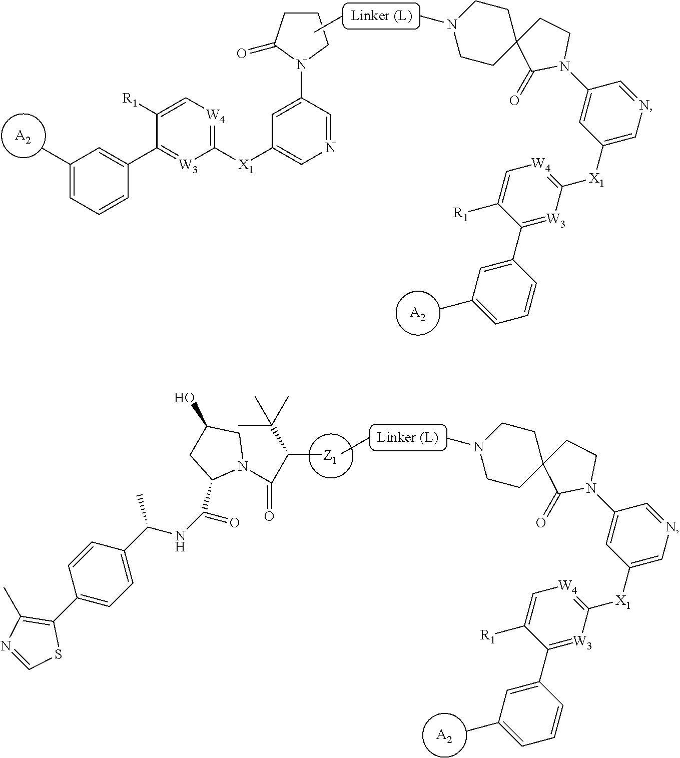

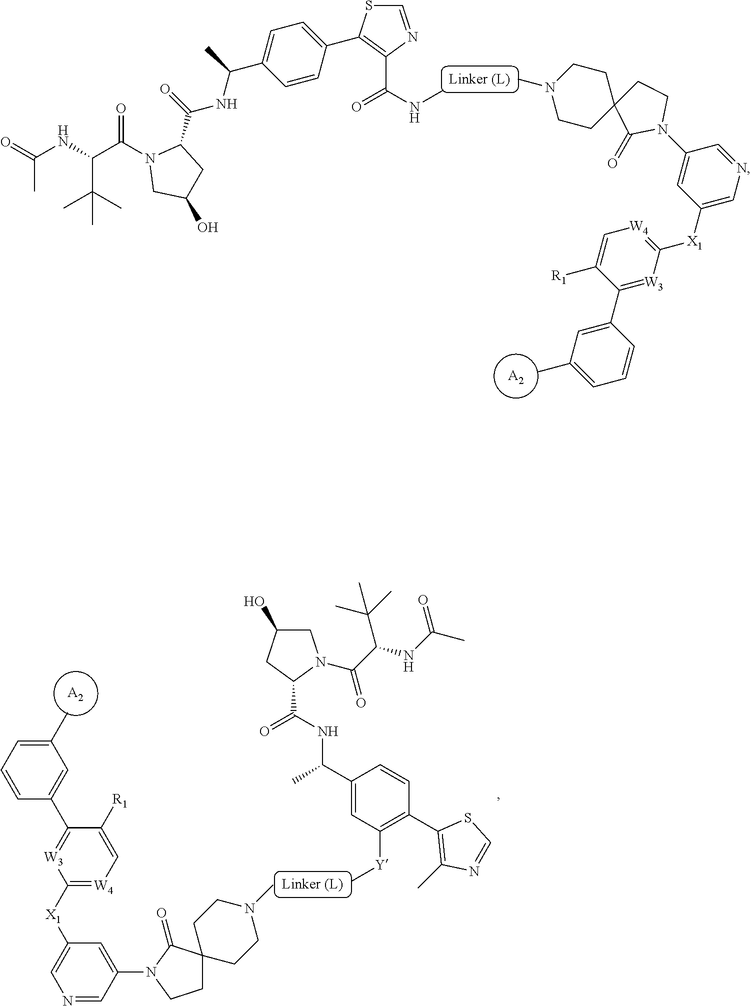

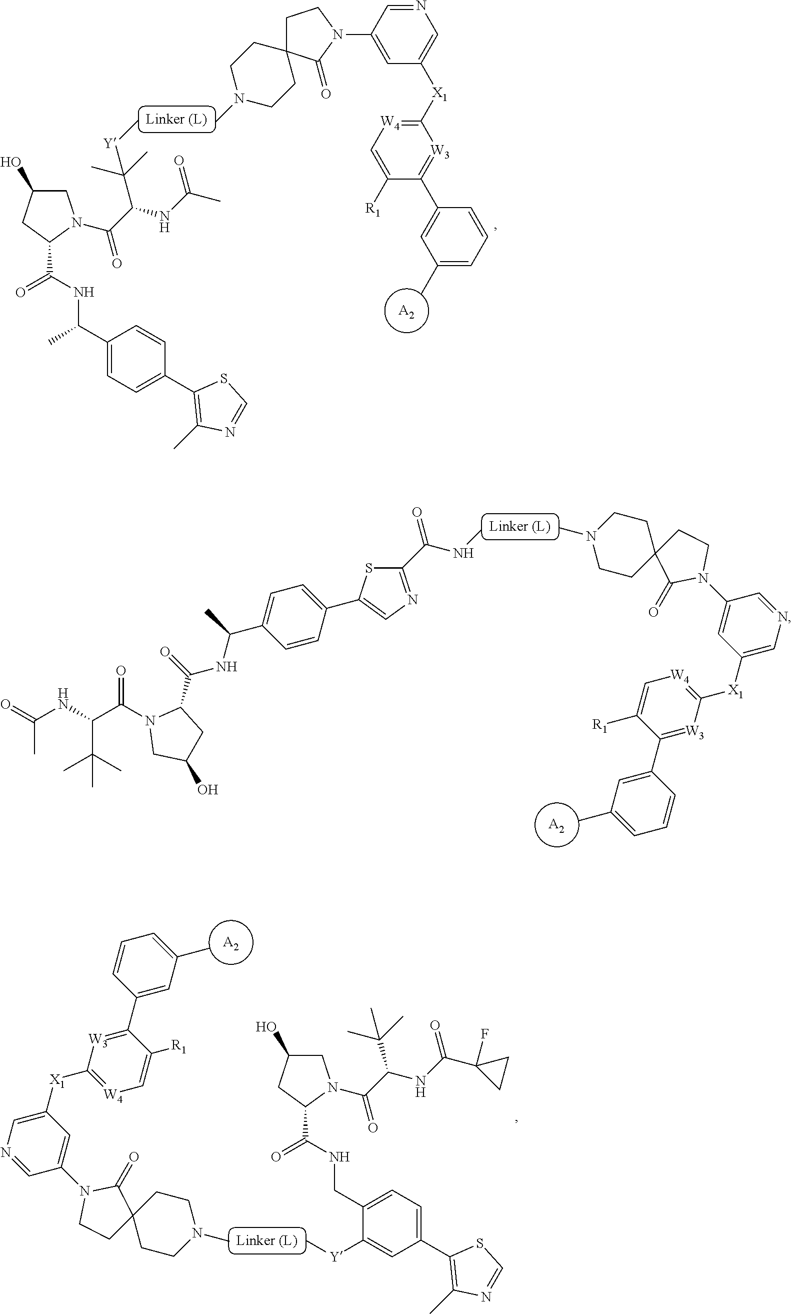

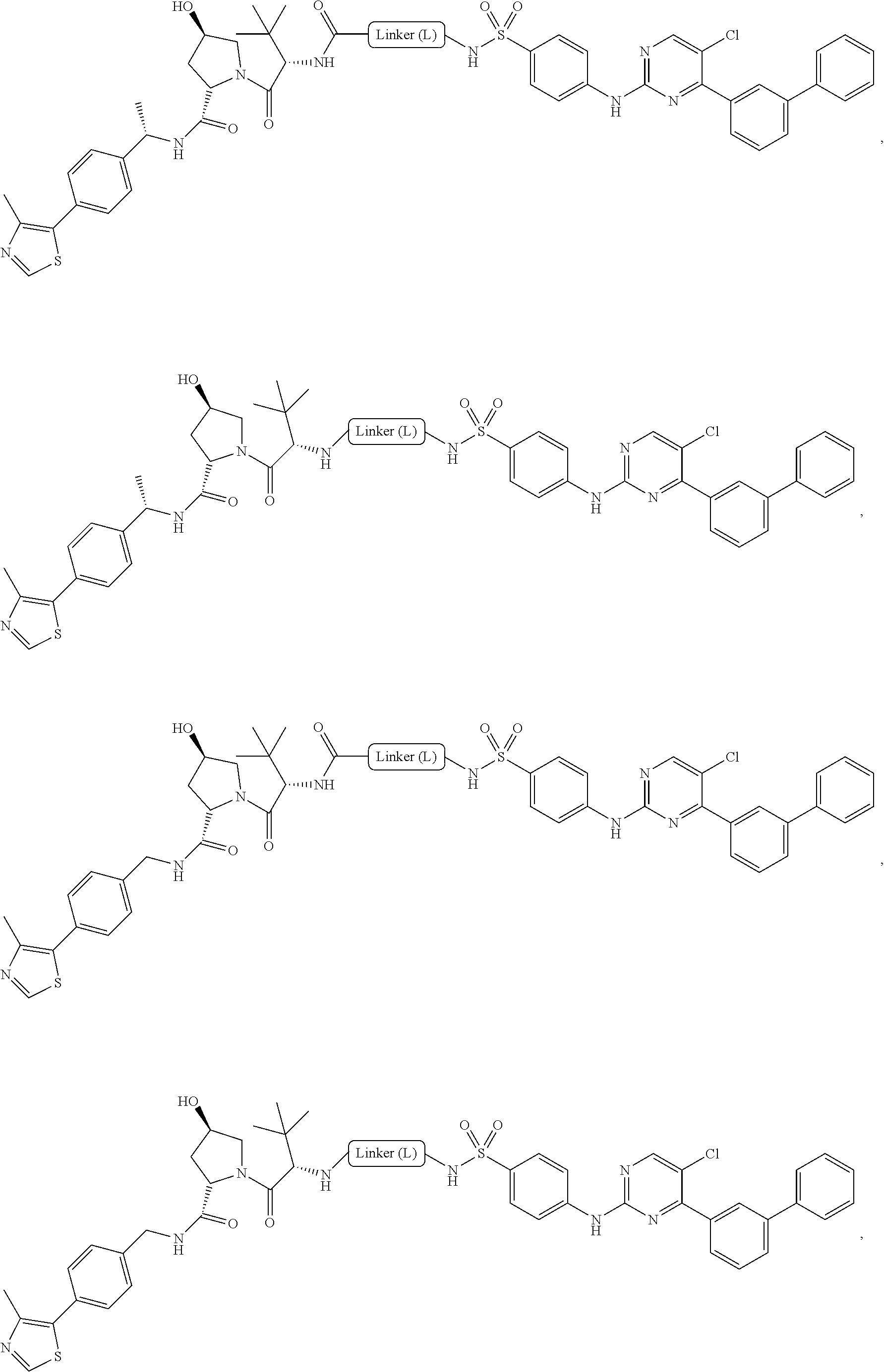

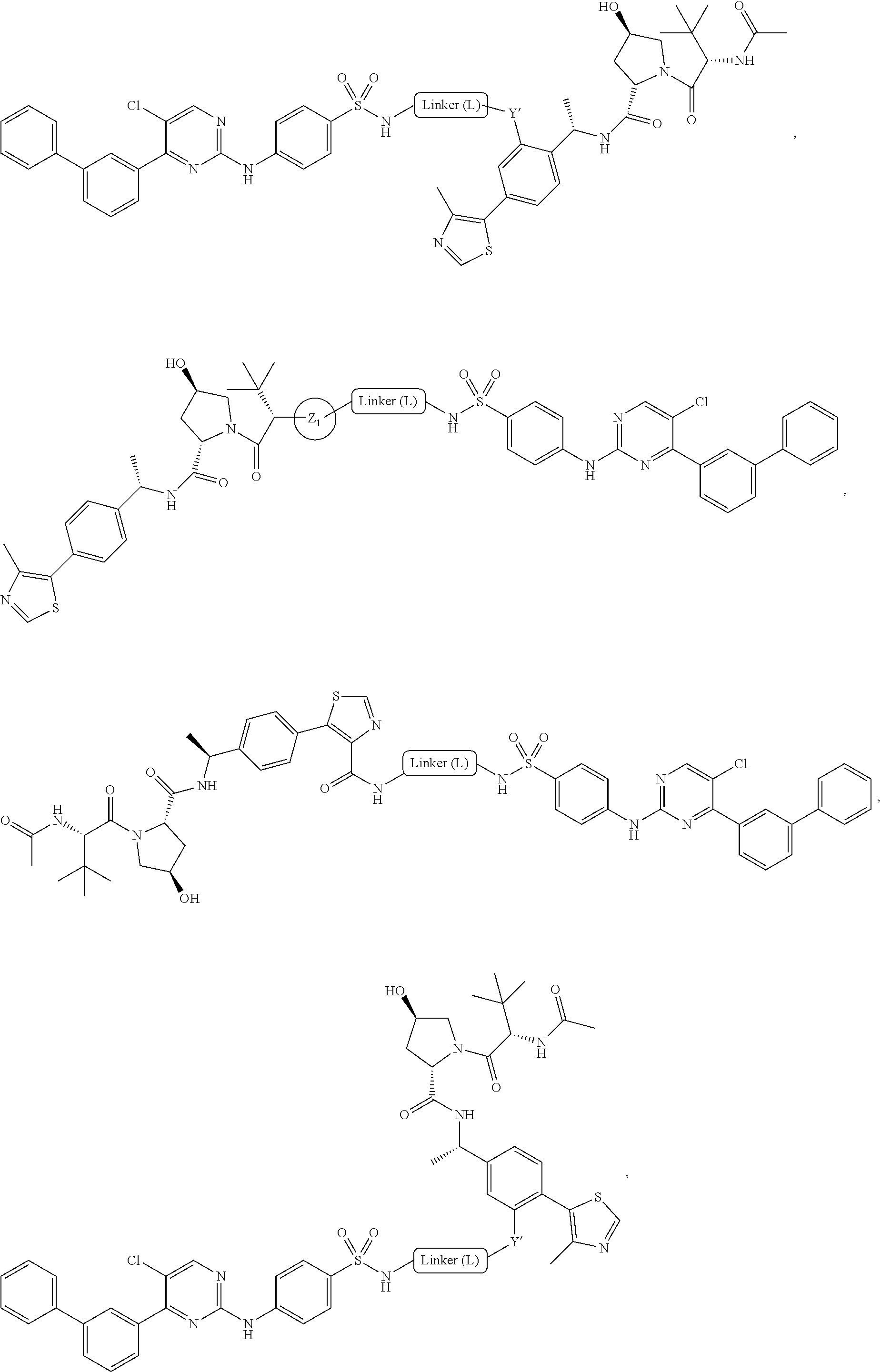

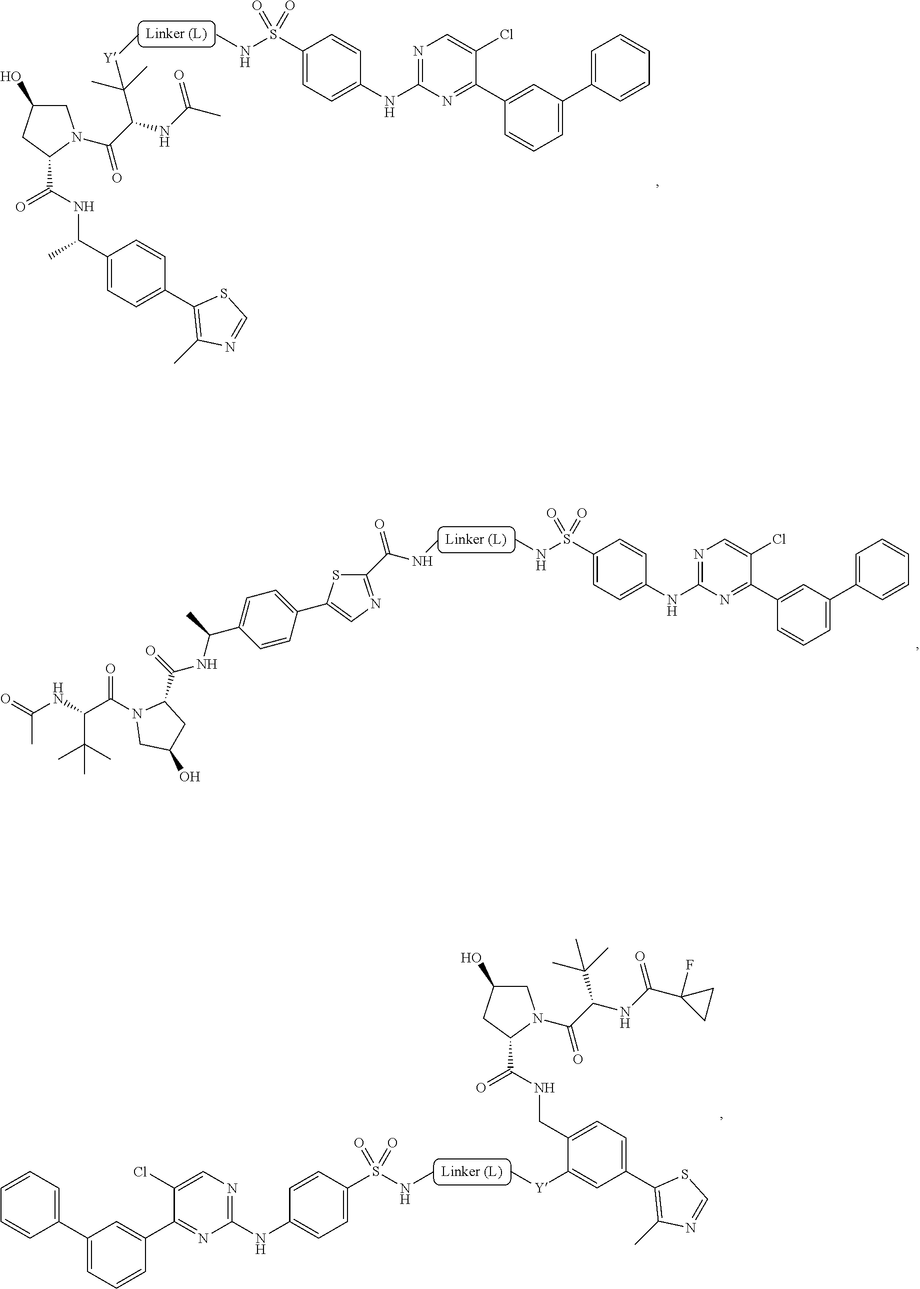

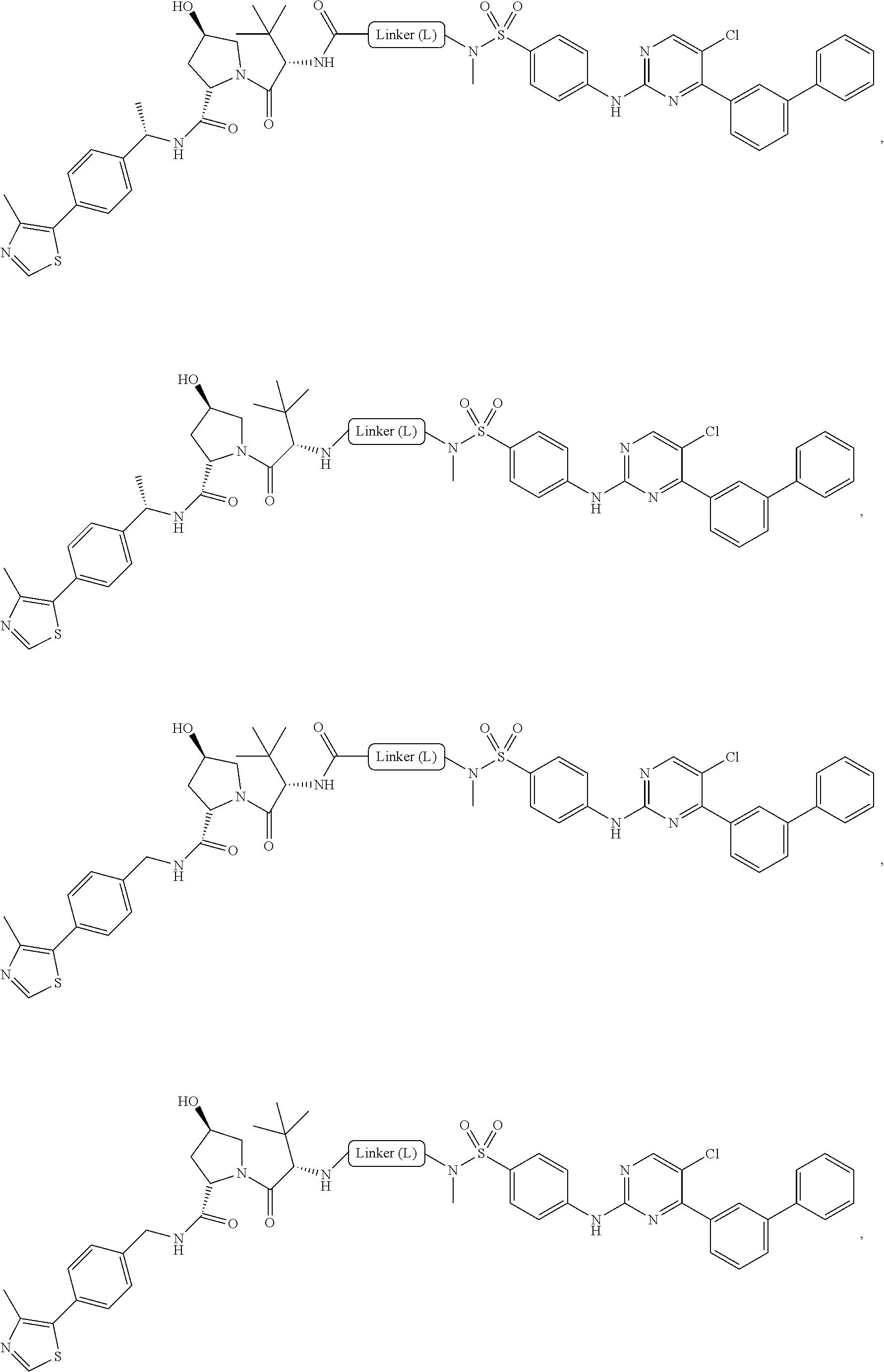

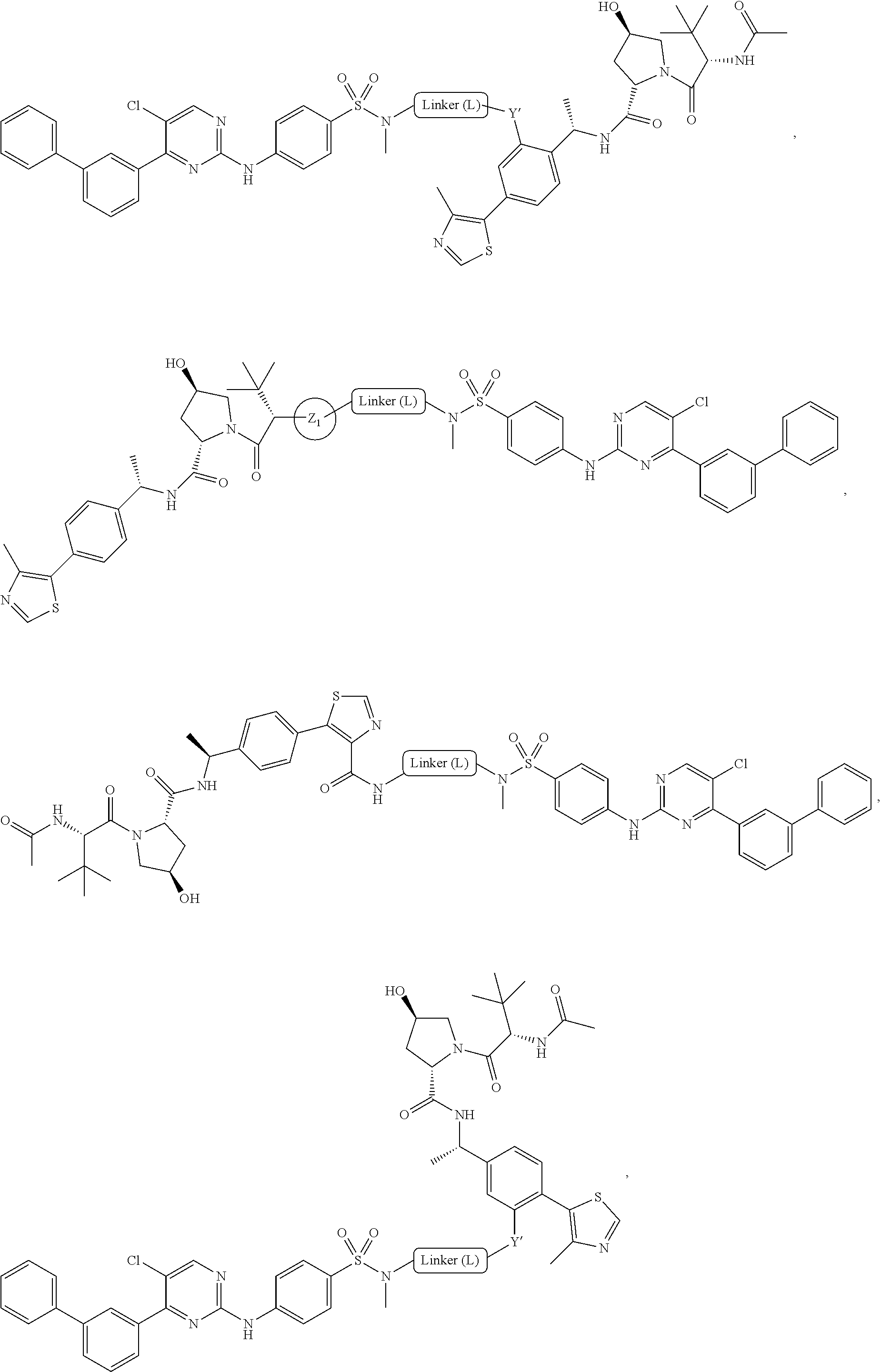

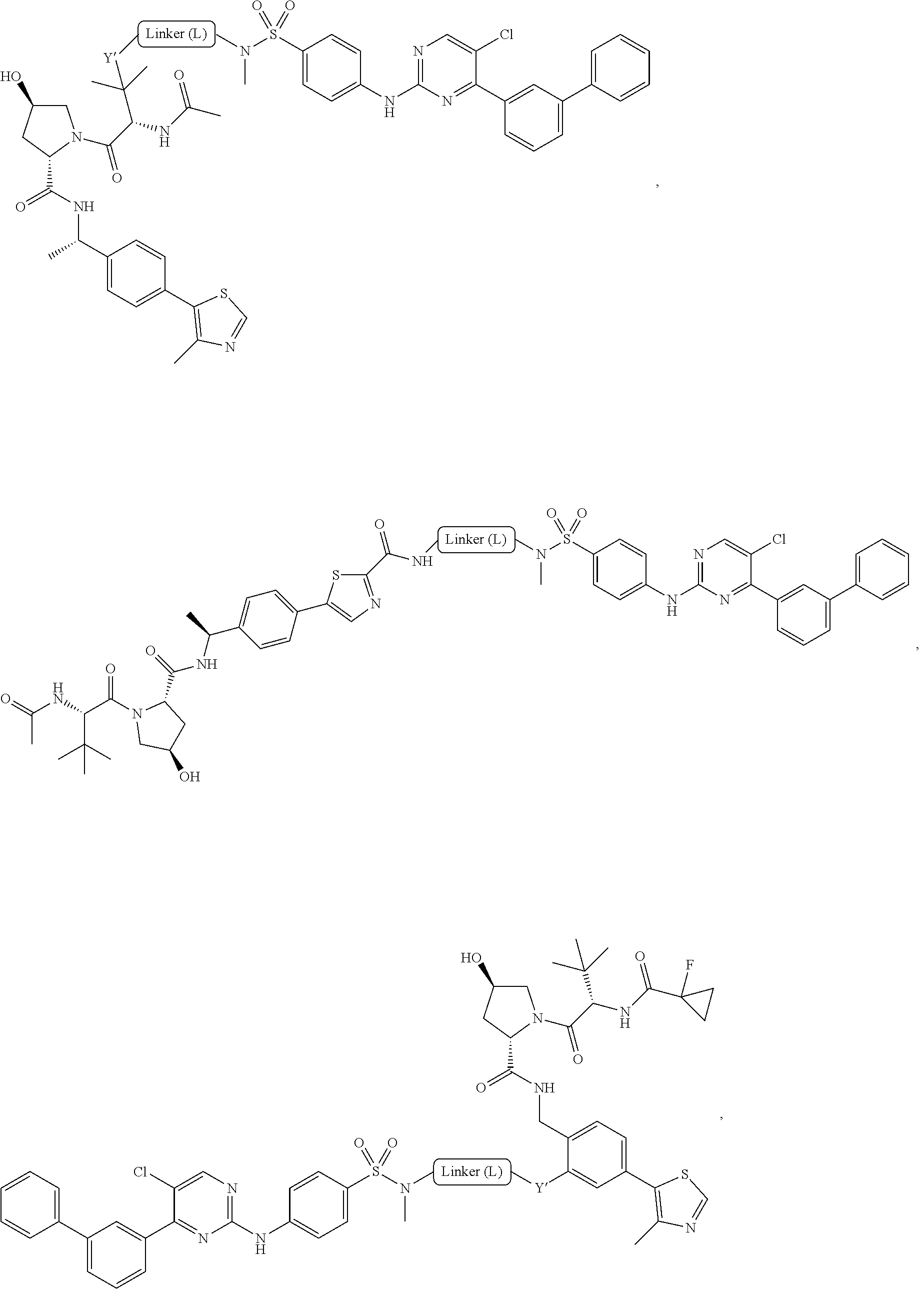

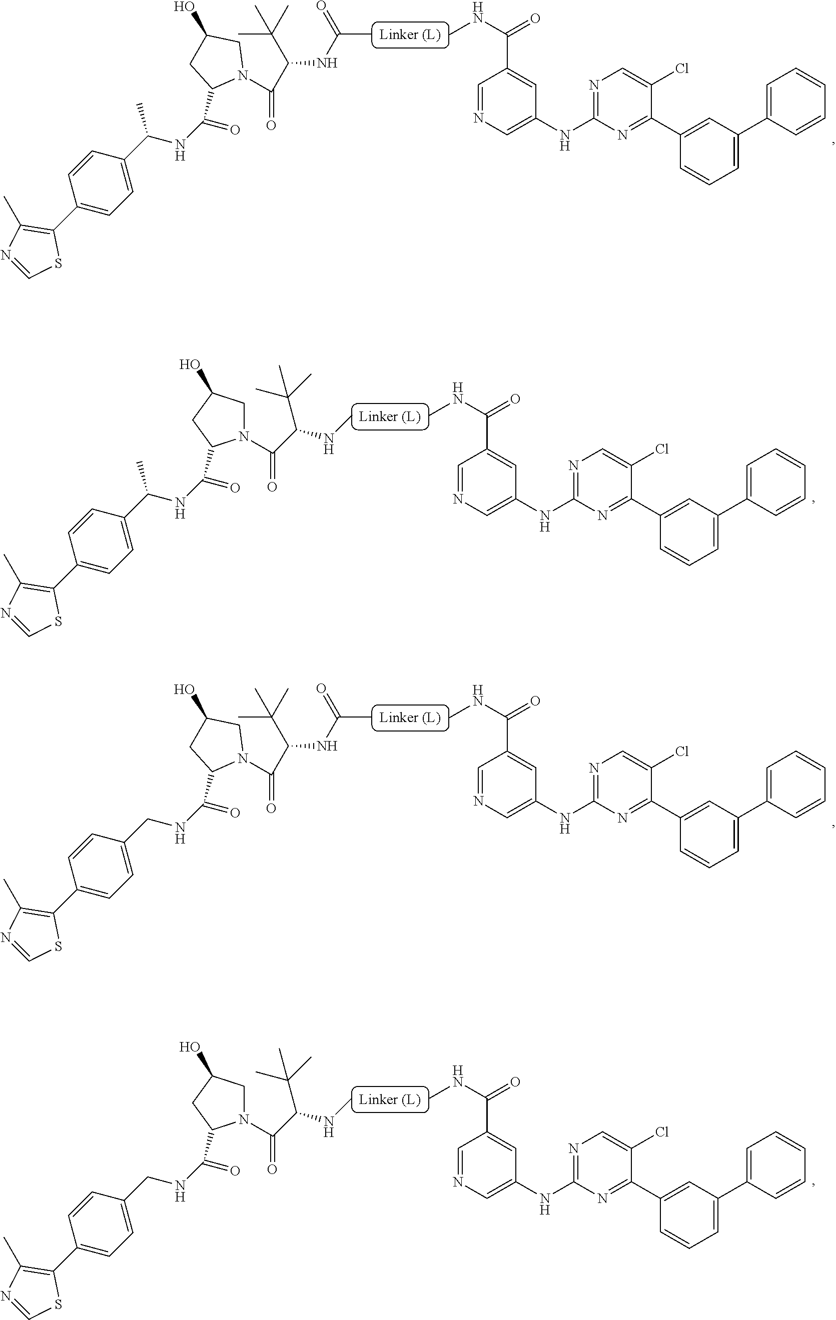

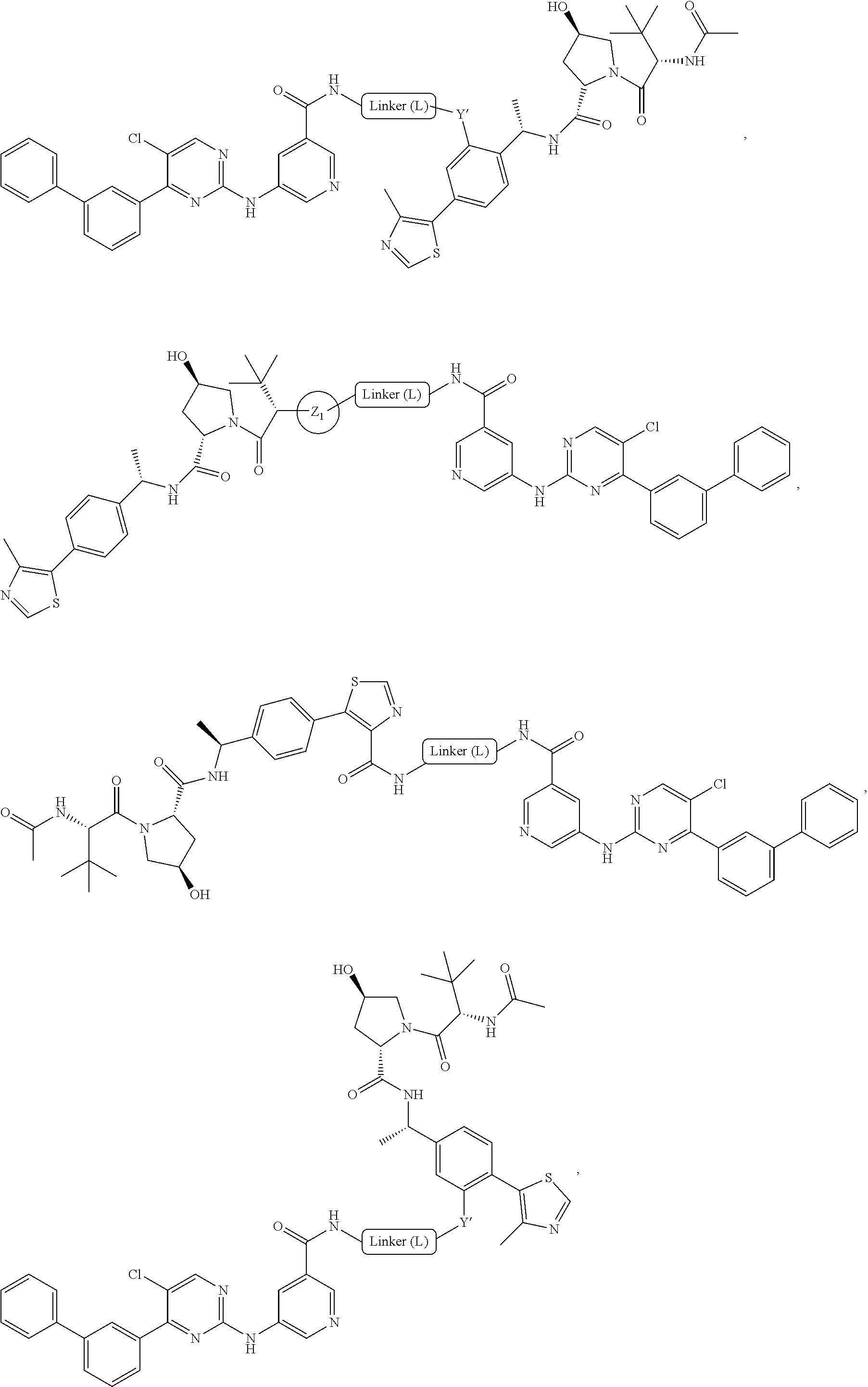

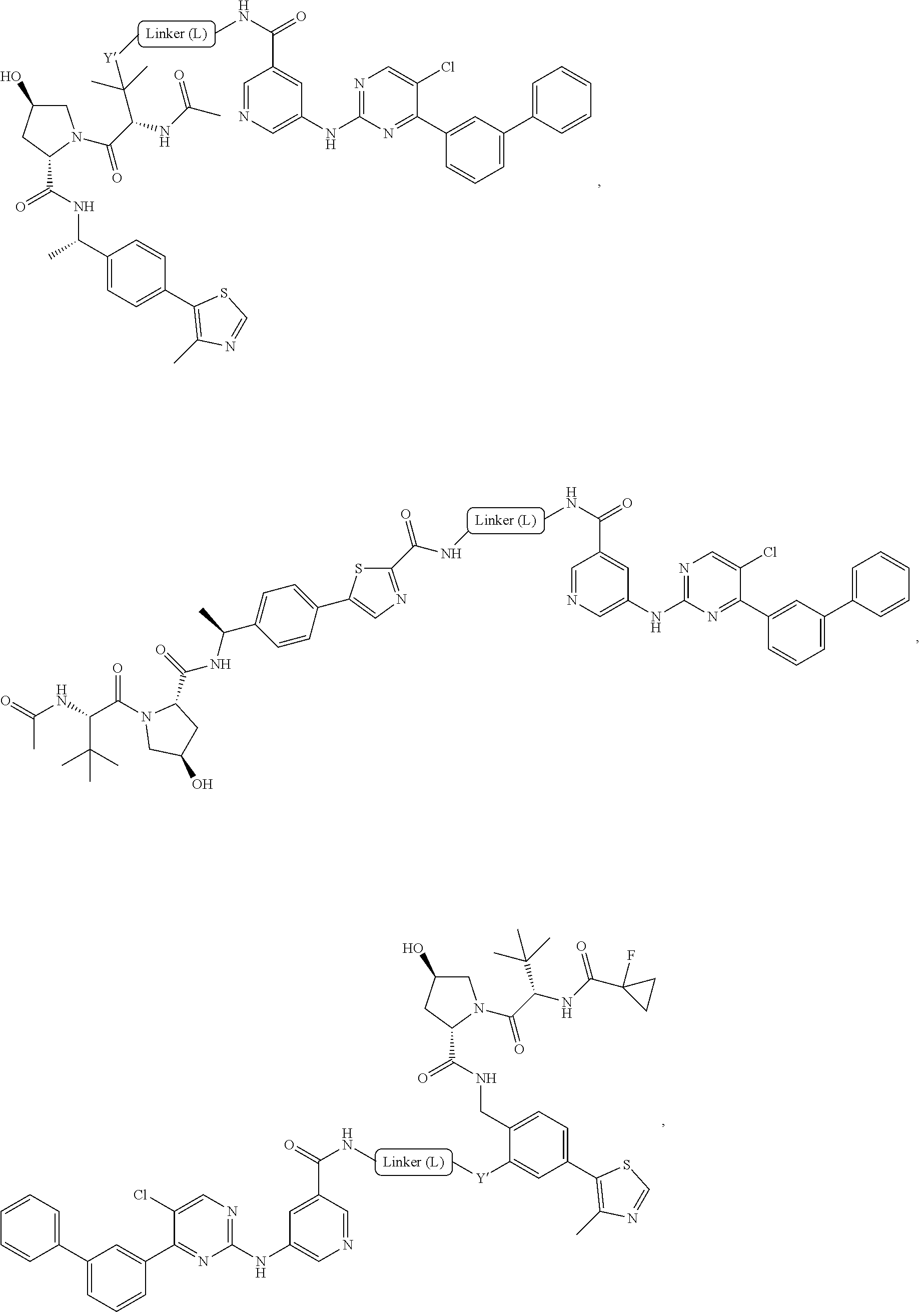

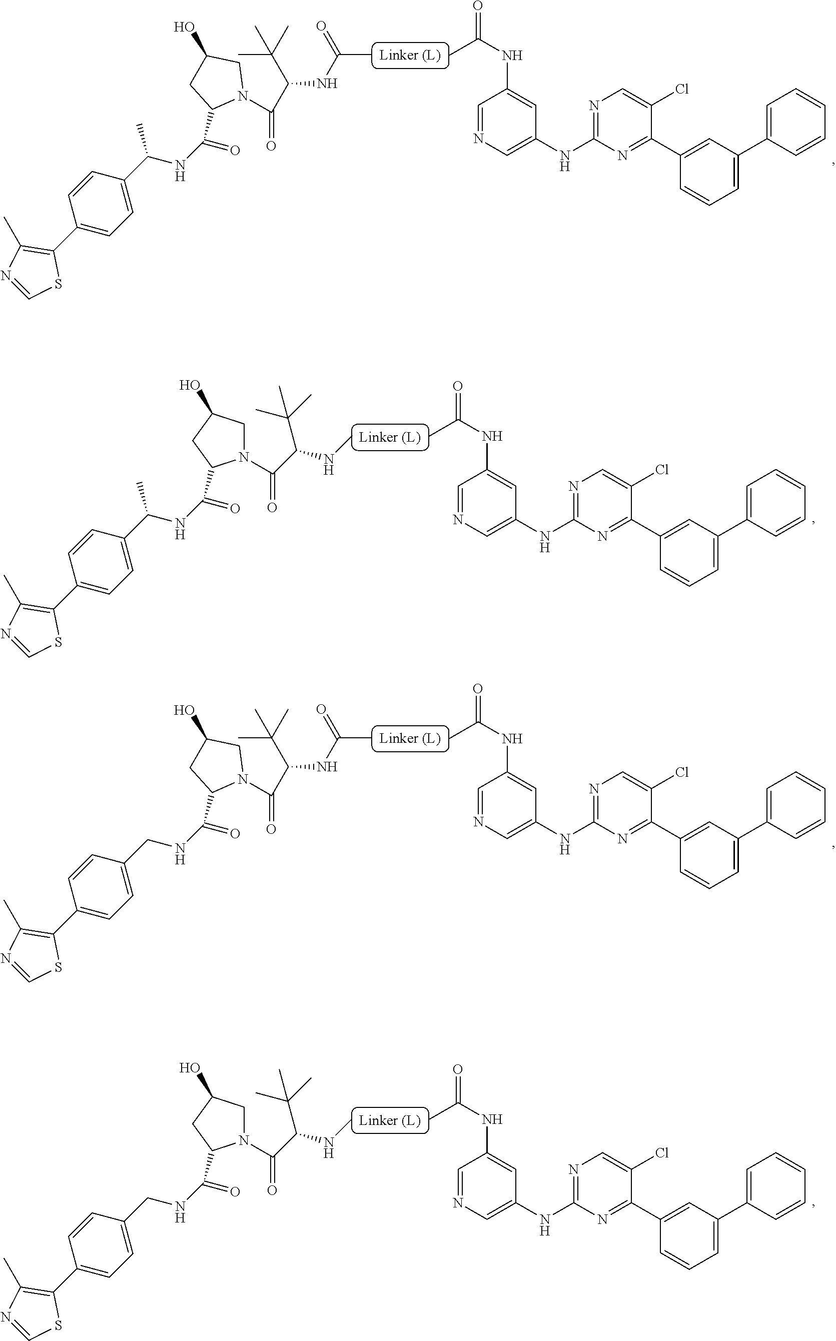

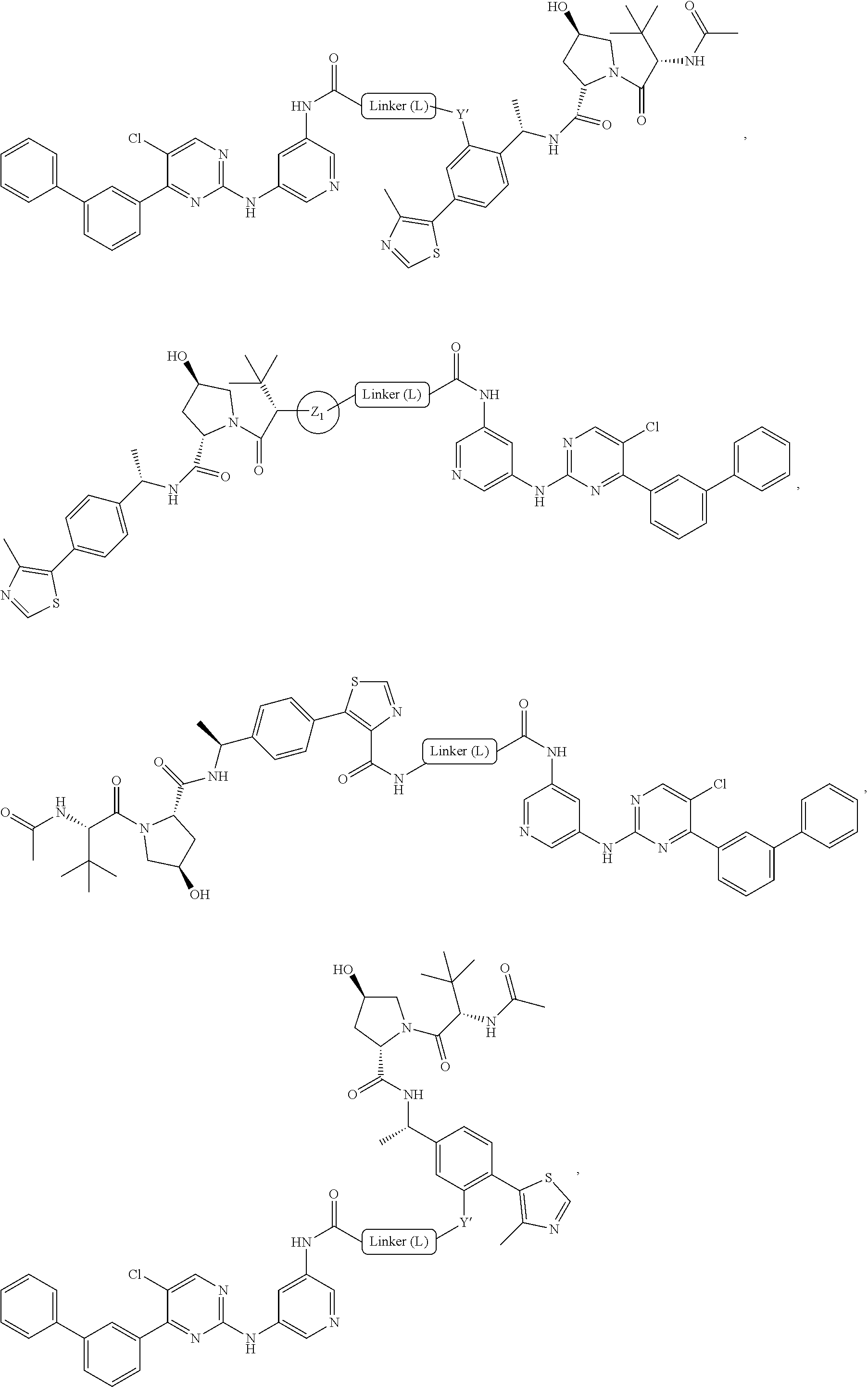

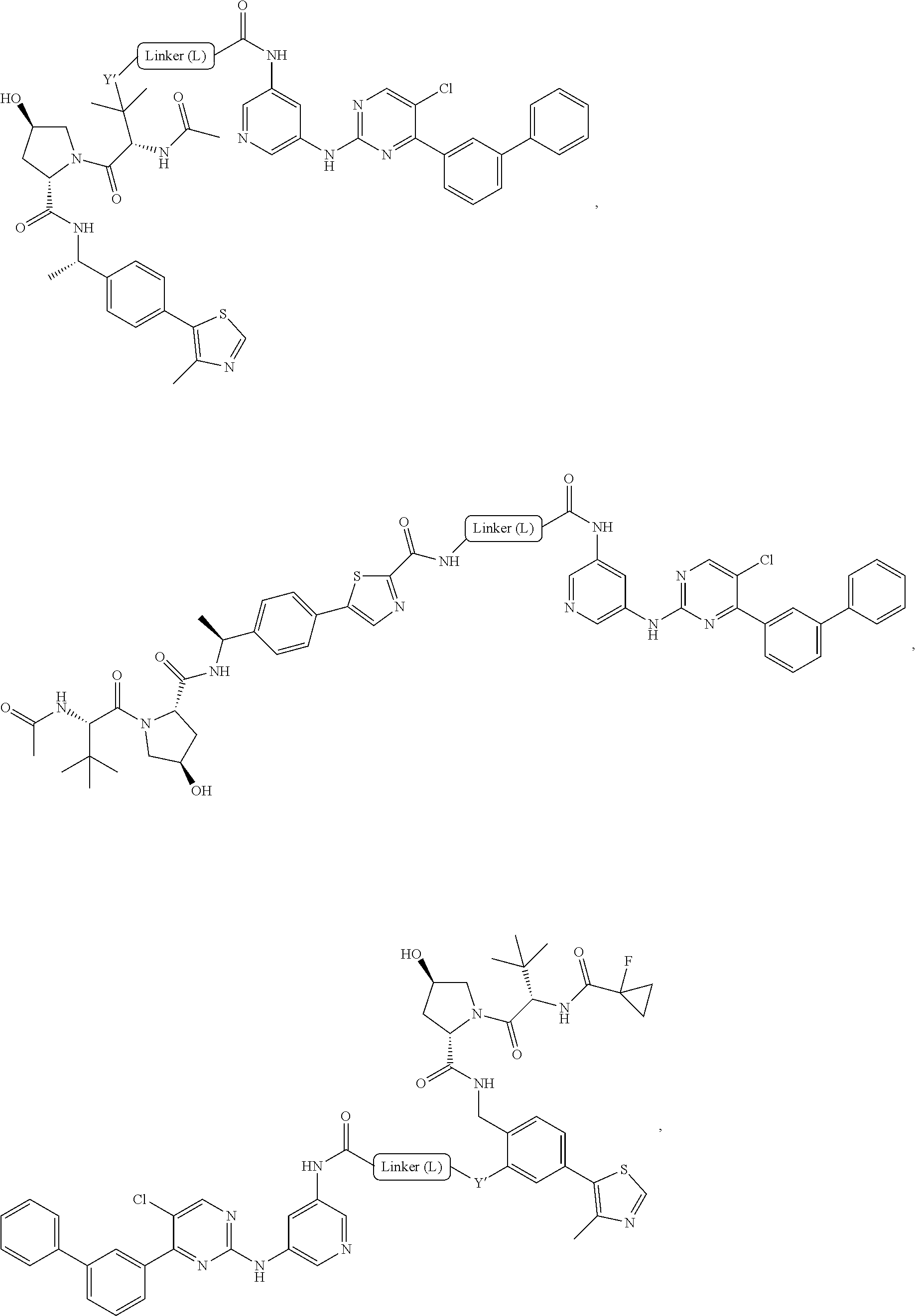

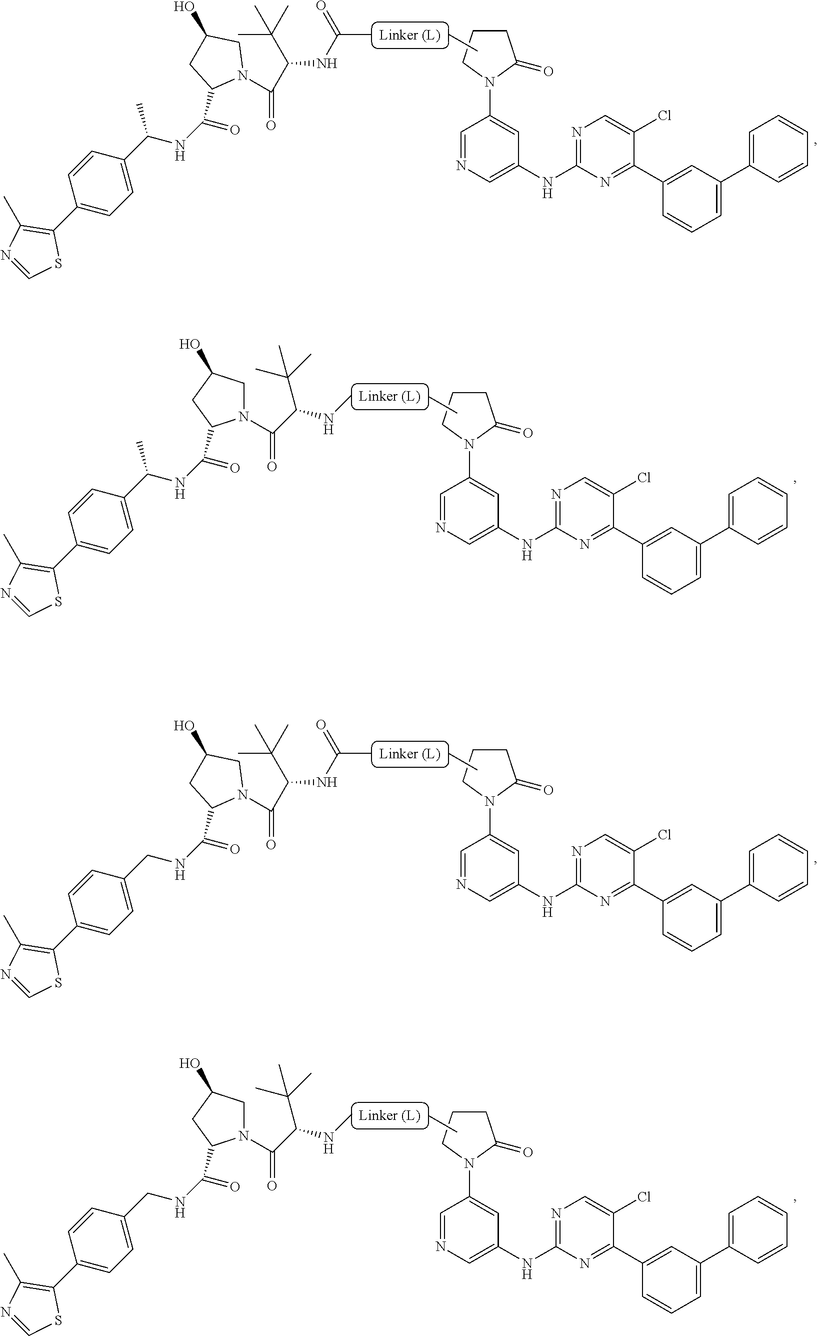

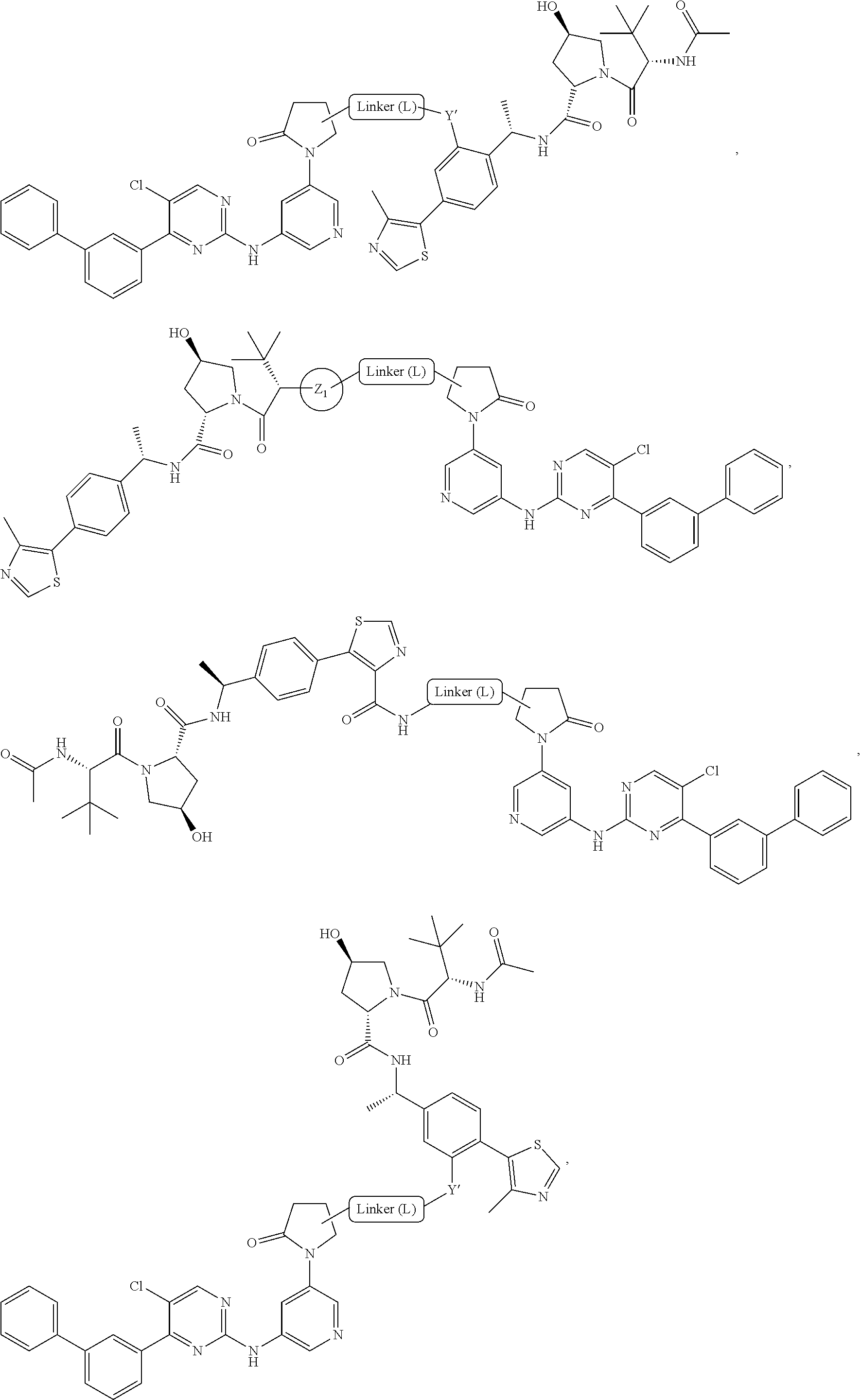

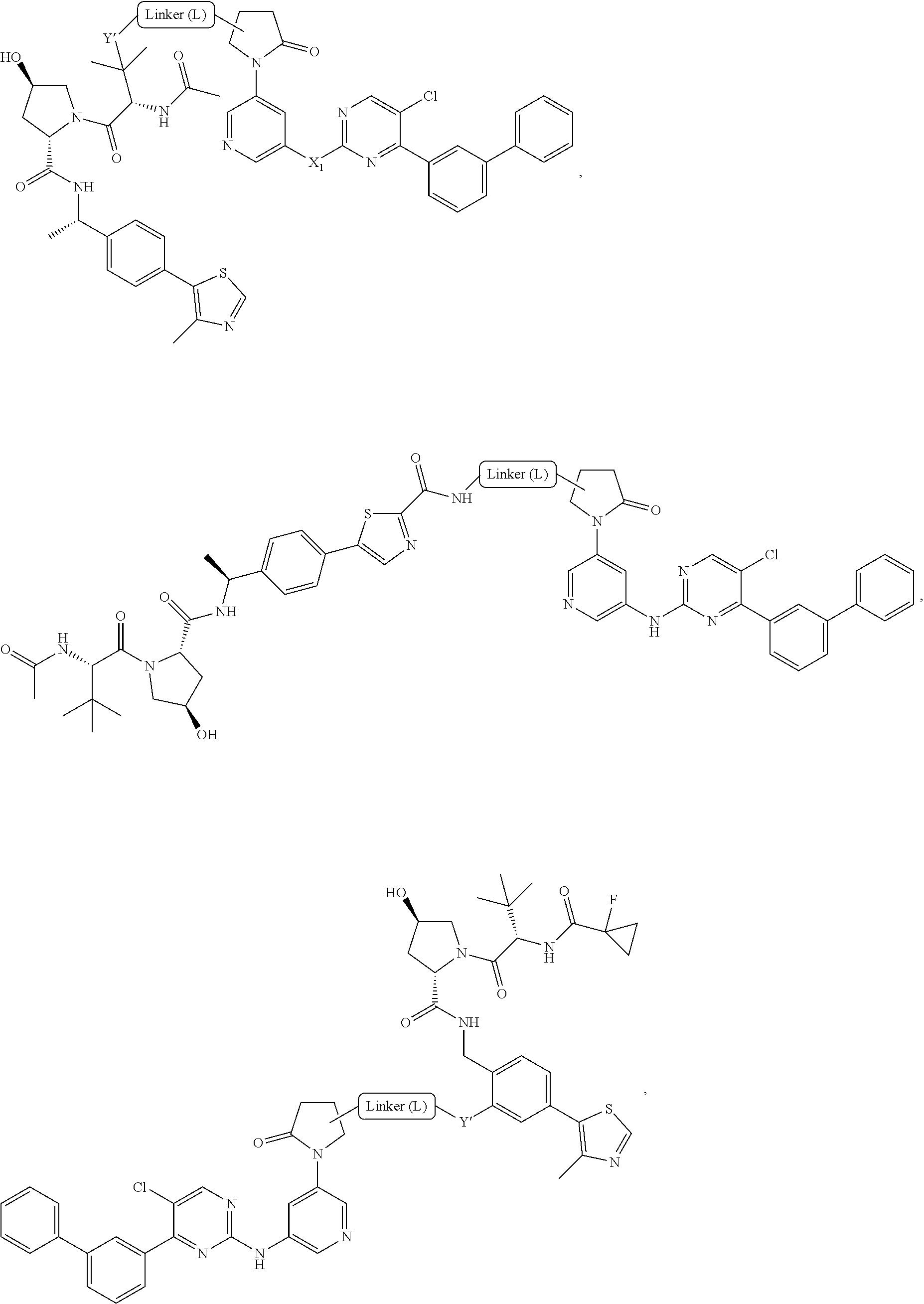

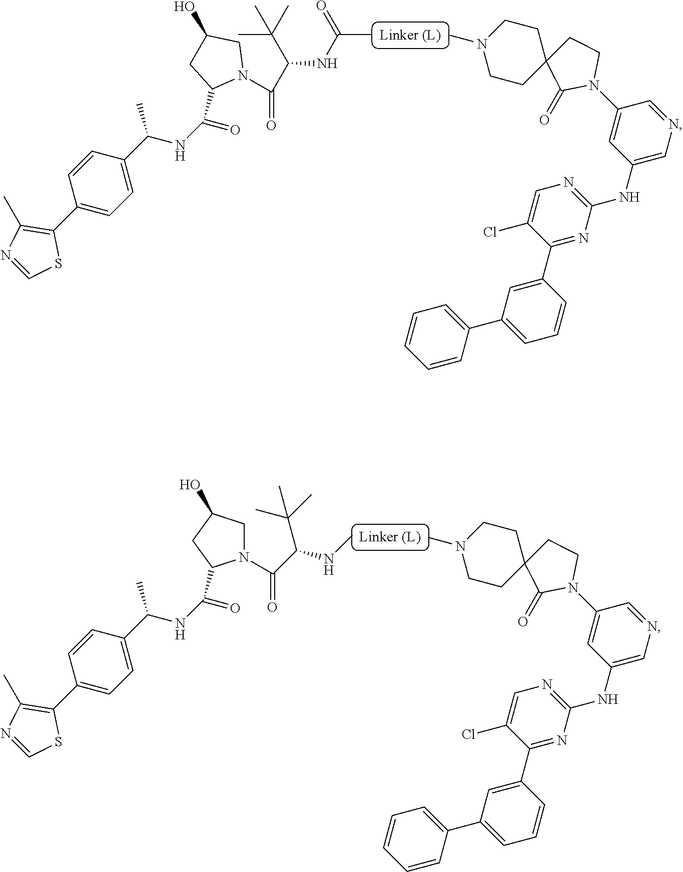

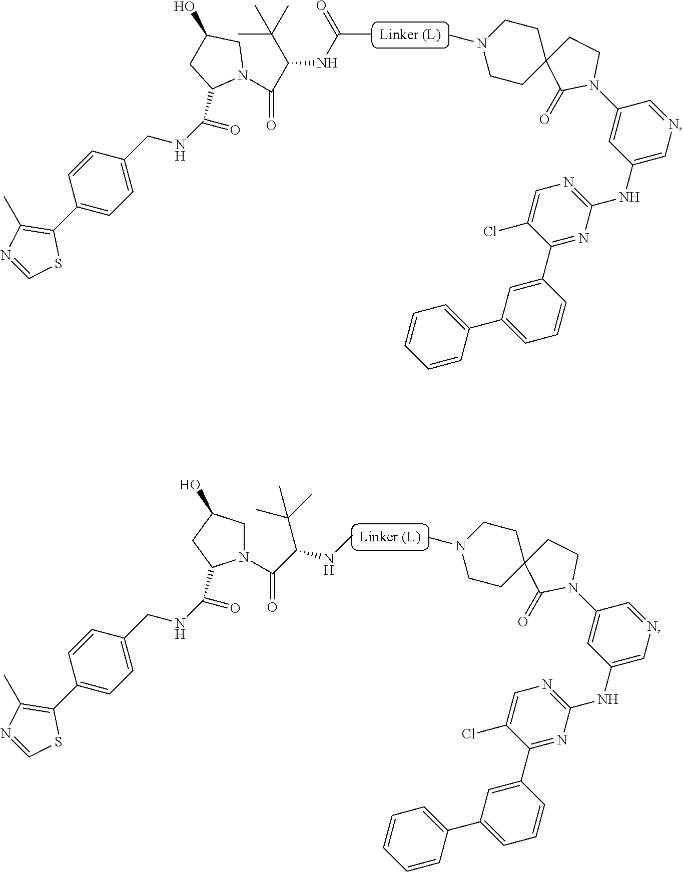

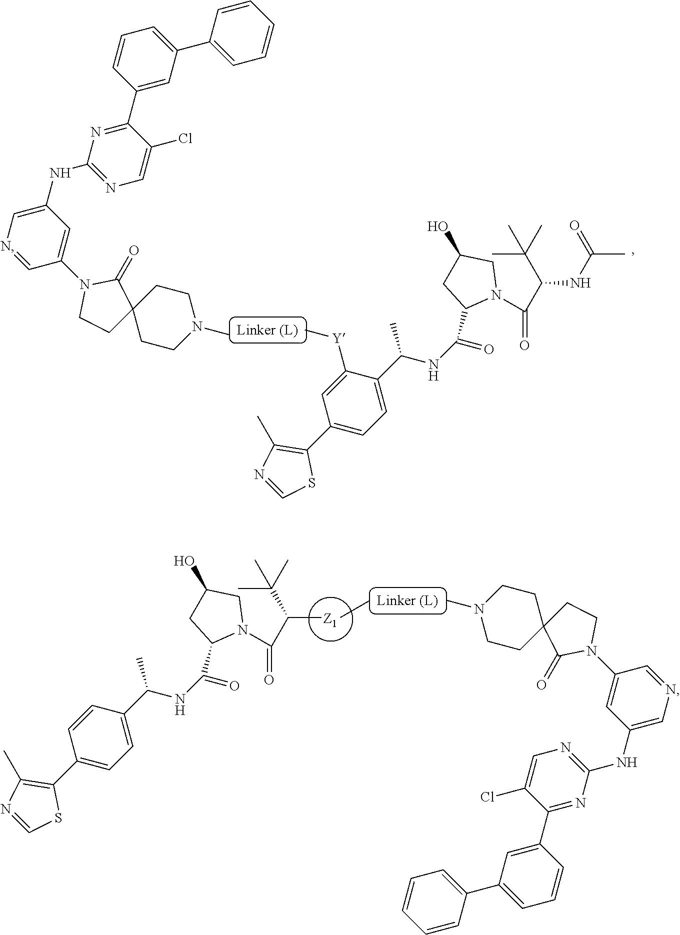

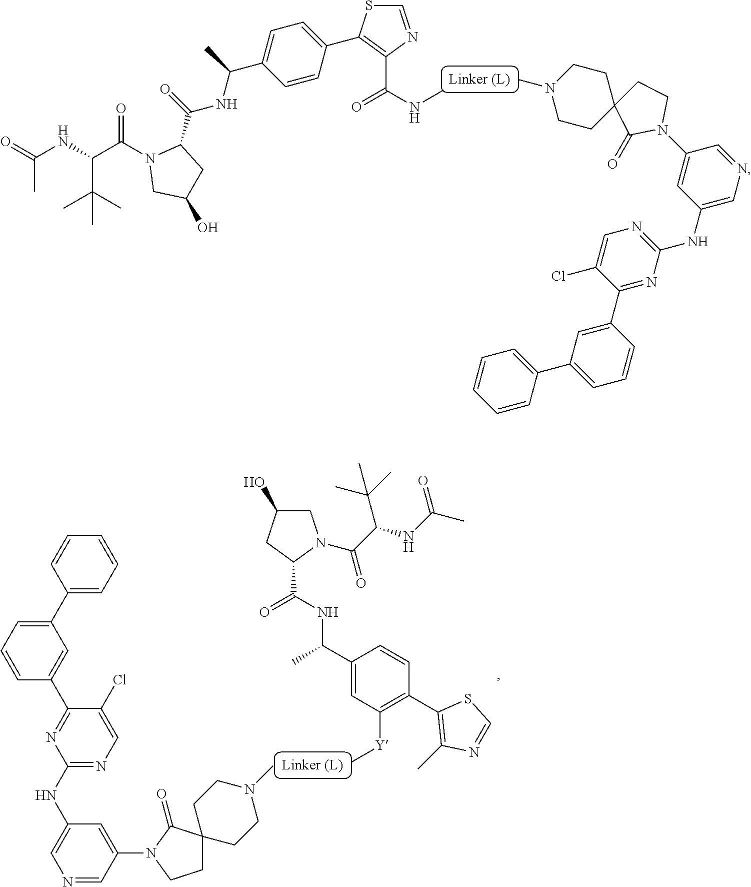

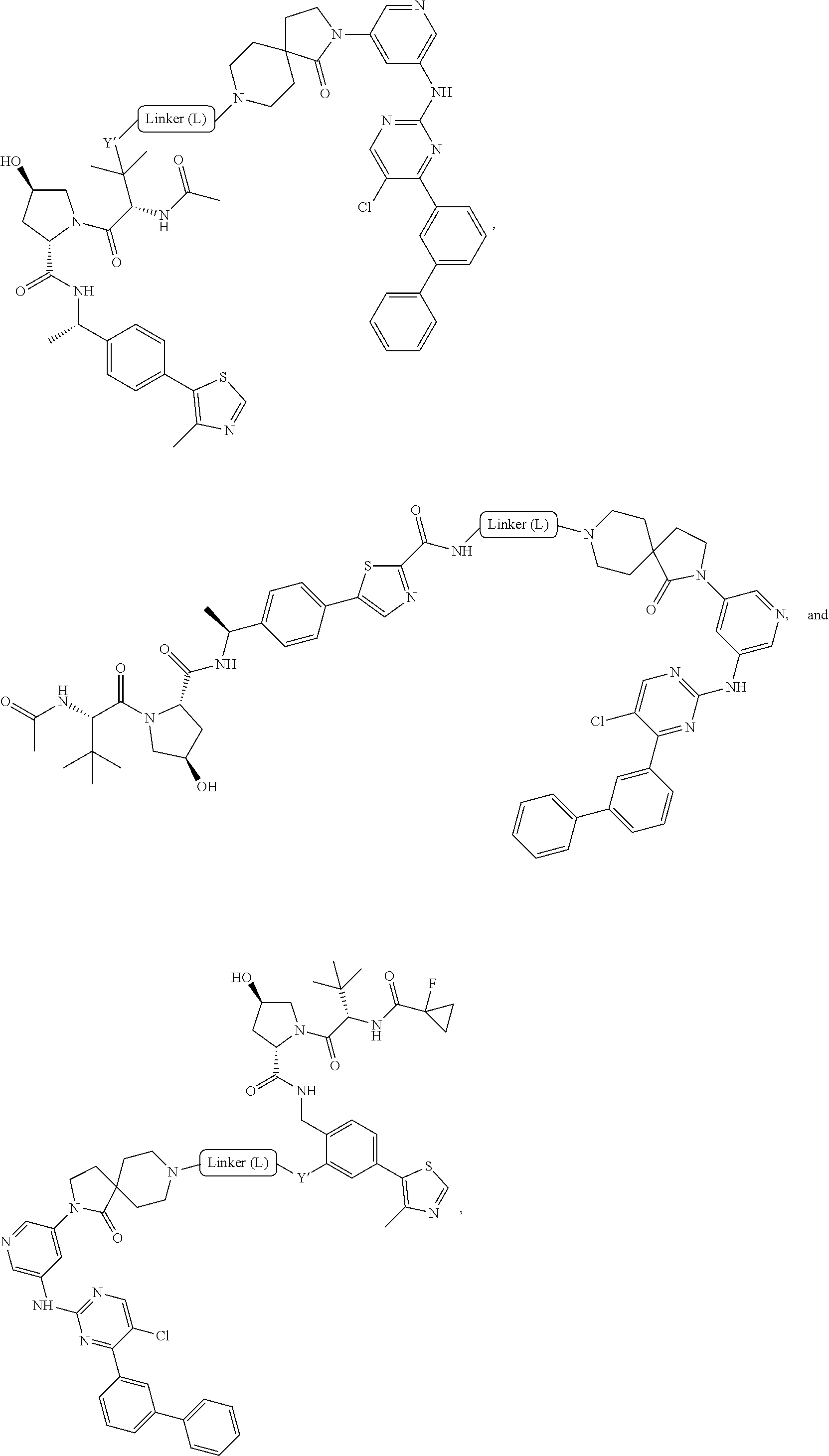

or a pharmaceutically acceptable salt or stereoisomer thereof.

Yet other degrons that bind VHL and which may be suitable for use in the present invention are disclosed in U.S. Patent Application Publication 2017/0121321 A1, which is incorporated herein by reference in its entirety.

Therefore, in some embodiments, bifunctional compounds of this invention are represented by any structures generated by the combination of structures TL1, L1 to L10, and the structures of the degrons D1 and D2, and all substructures thereof, or a pharmaceutically acceptable salt or stereoisomer thereof.

In some embodiments, bifunctional compounds of the present invention are represented by any one of the following structures:

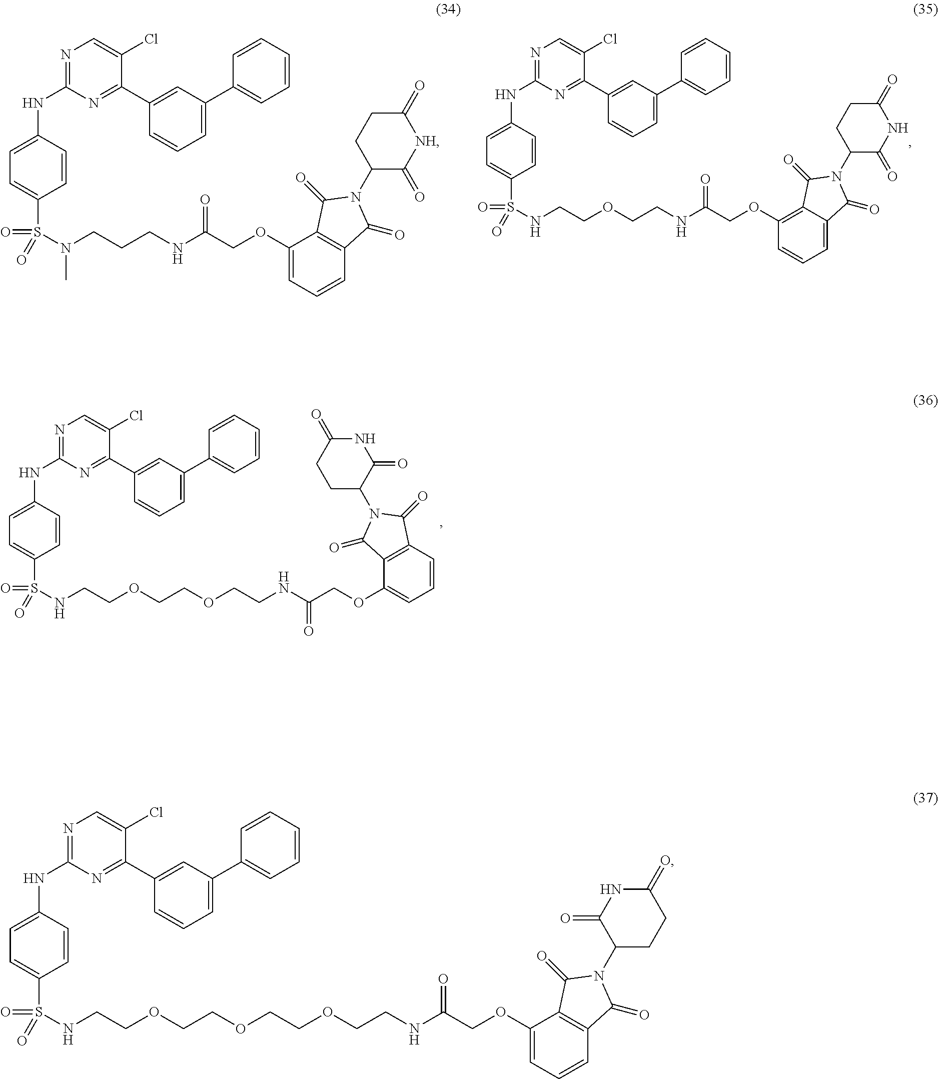 [SMILES: CN(CCCNC(=O)COc1cccc2c1C(=O)N(C1CCC(=O)NC1=O)C2=O)S(=O)(=O)c1ccc(Nc2ncc(Cl)c(-c3cccc(-c4ccccc4)c3)n2)cc1 ; O=C(COc1cccc2c1C(=O)N(C1CCC(=O)NC1=O)C2=O)NCCOCCNS(=O)(=O)c1ccc(Nc2ncc(Cl)c(-c3cccc(-c4ccccc4)c3)n2)cc1 ; O=C(COc1cccc2c1C(=O)N(C1CCC(=O)NC1=O)C2=O)NCCOCCOCCNS(=O)(=O)c1ccc(Nc2ncc(Cl)c(-c3cccc(-c4ccccc4)c3)n2)cc1 ; O=C(COc1cccc2c1C(=O)N(C1CCC(=O)NC1=O)C2=O)NCCOCCOCCOCCNS(=O)(=O)c1ccc(Nc2ncc(Cl)c(-c3cccc(-c4ccccc4)c3)n2)cc1]

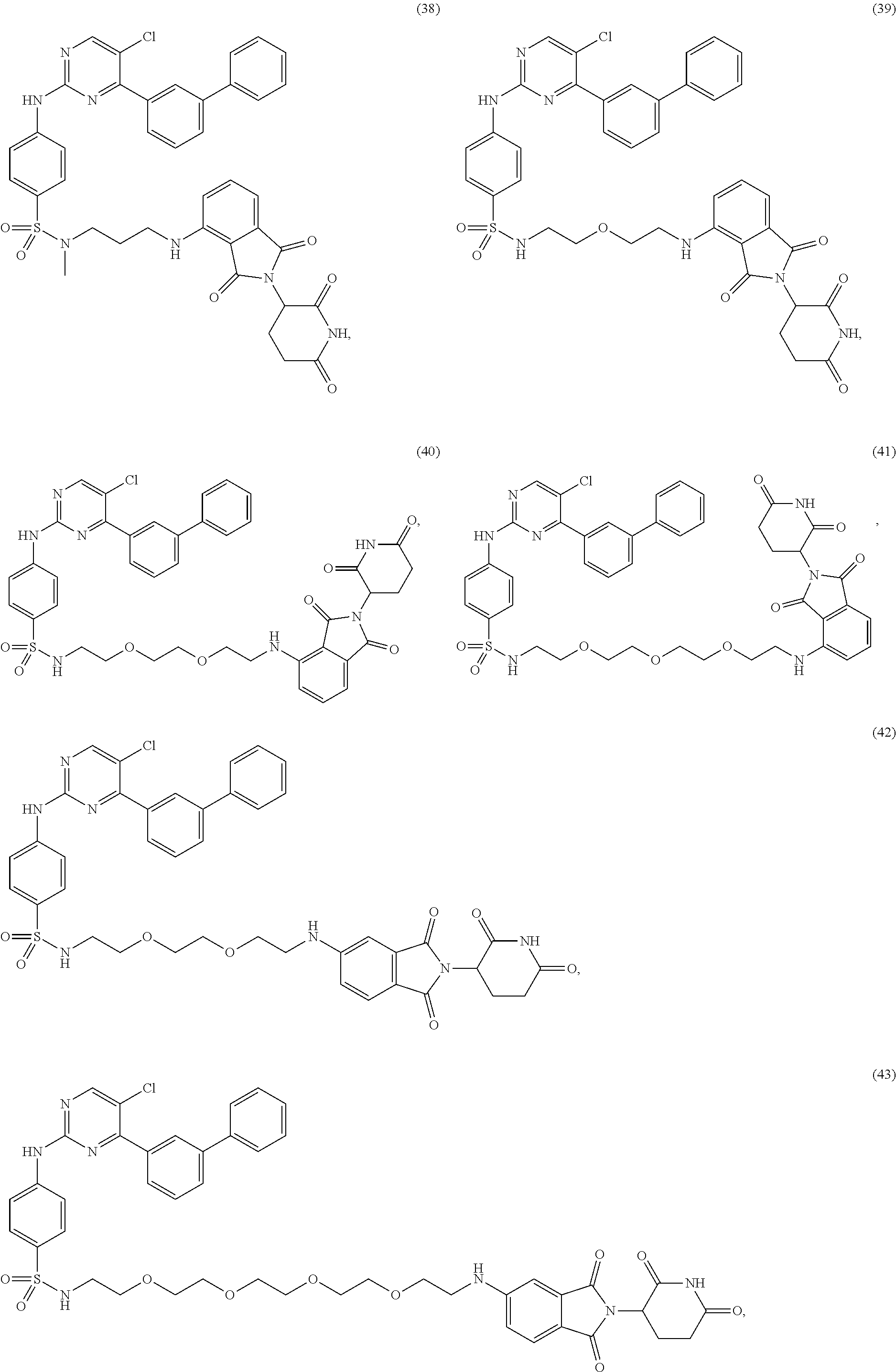

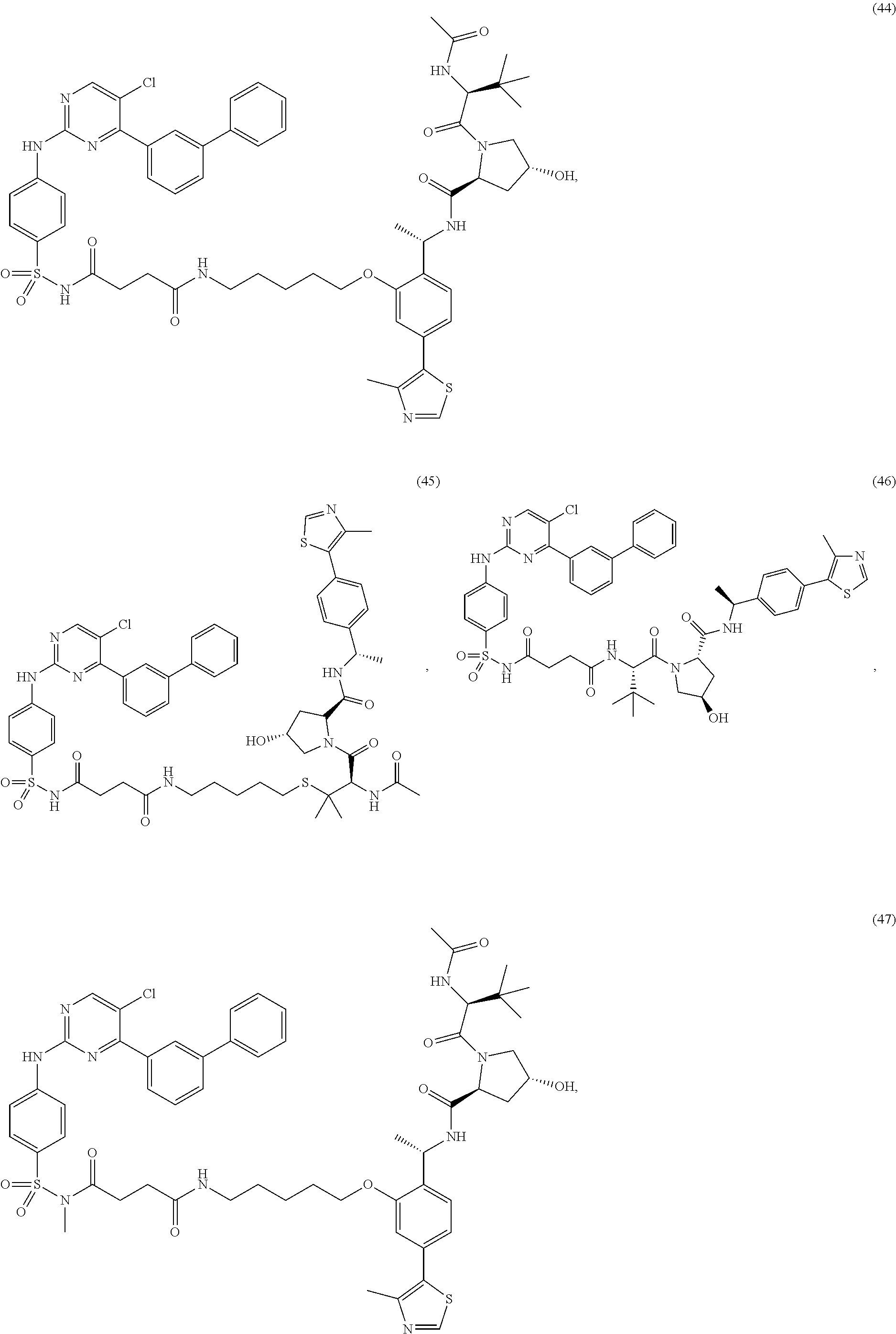

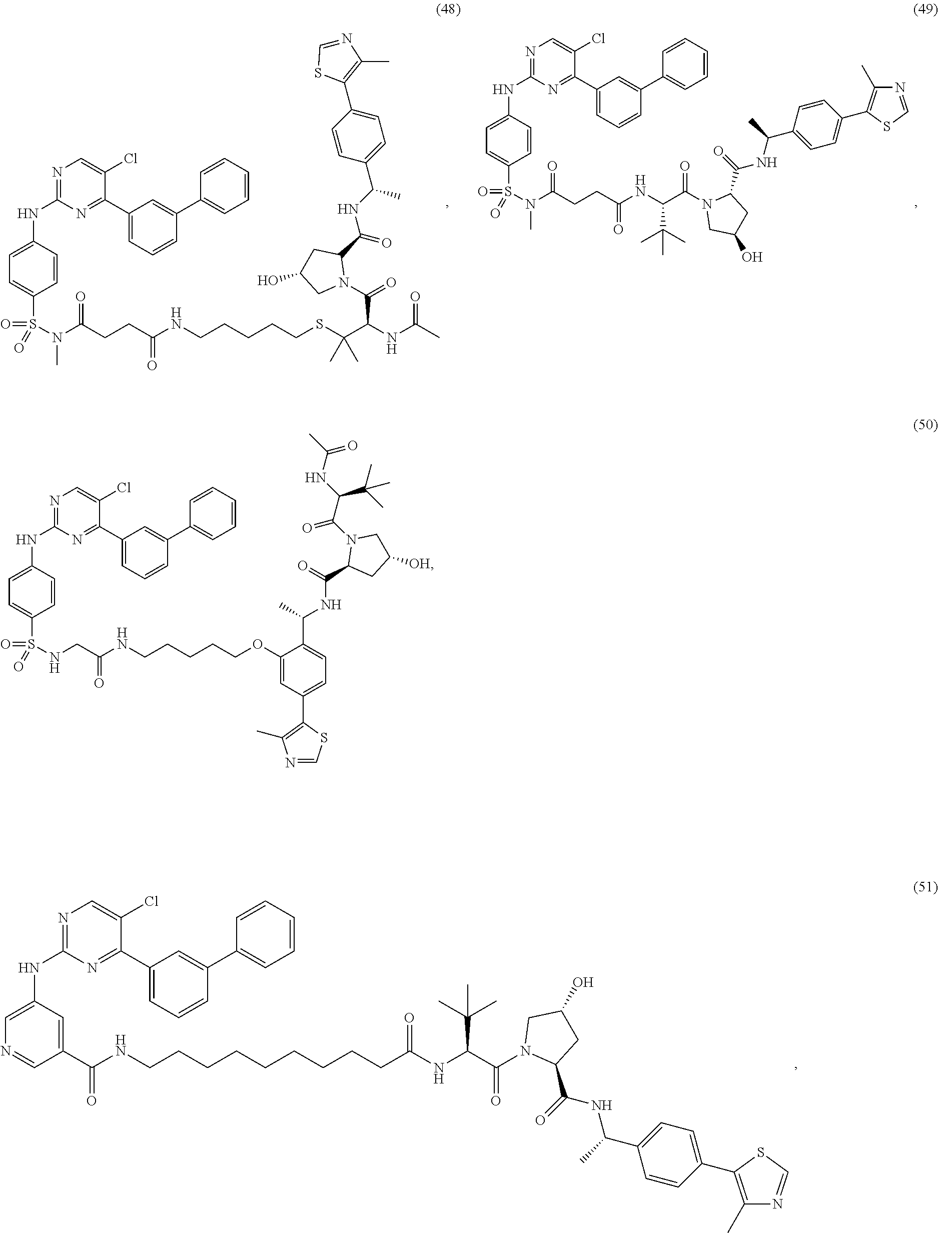

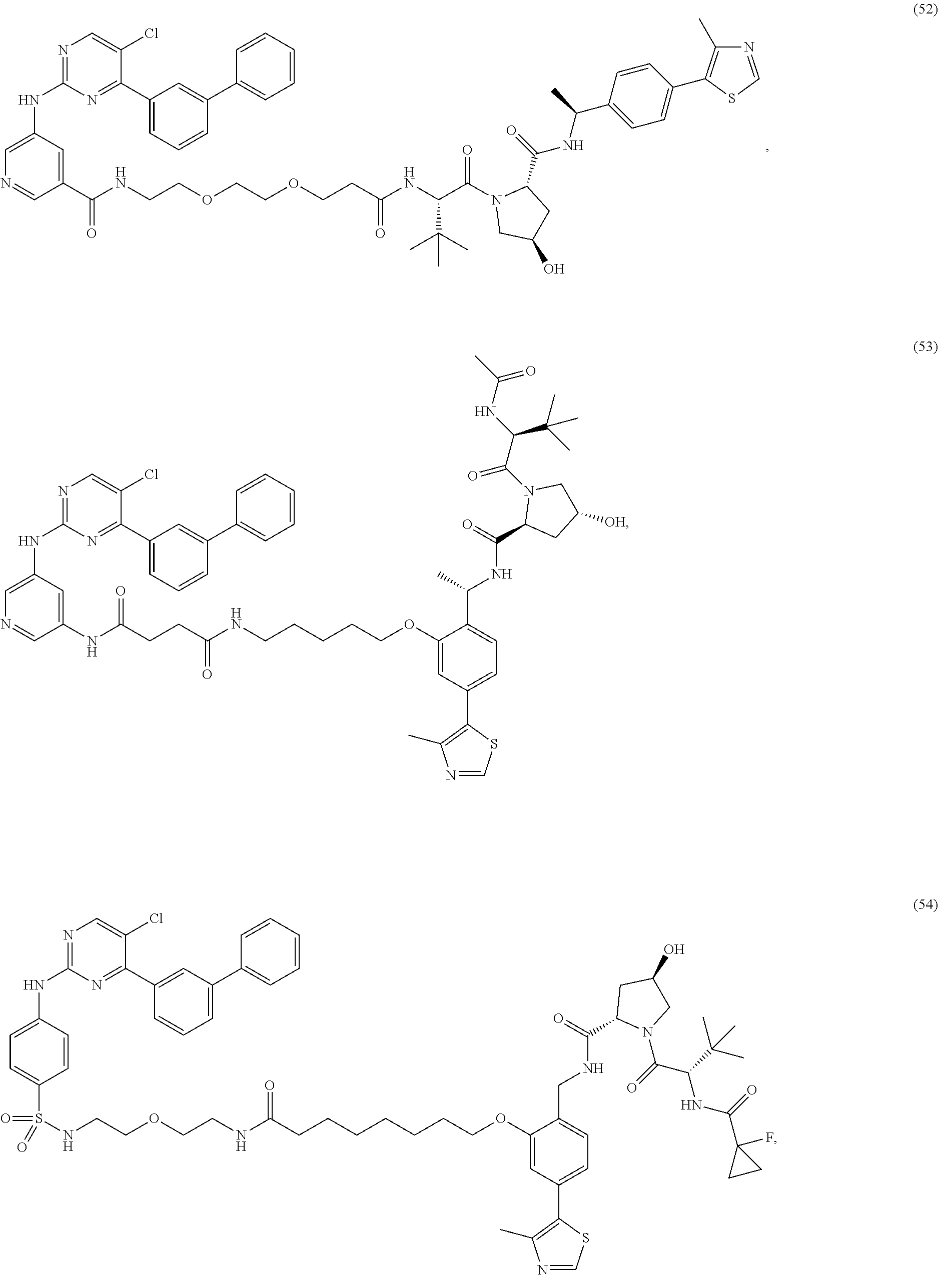 [SMILES: Cc1ncsc1-c1ccc([C@H](C)NC(=O)[C@@H]2C[C@@H](O)CN2C(=O)[C@@H](NC(=O)CCOCCOCCNC(=O)c2cncc(Nc3ncc(Cl)c(-c4cccc(-c5ccccc5)c4)n3)c2)C(C)(C)C)cc1 ; CC(=O)N[C@H](C(=O)N1C[C@H](O)C[C@H]1C(=O)N[C@@H](C)c1ccc(-c2scnc2C)cc1OCCCCCNC(=O)CCC(=O)Nc1cncc(Nc2ncc(Cl)c(-c3cccc(-c4ccccc4)c3)n2)c1)C(C)(C)C ; Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)NCCOCCNS(=O)(=O)c2ccc(Nc3ncc(Cl)c(-c4cccc(-c5ccccc5)c4)n3)cc2)c1]

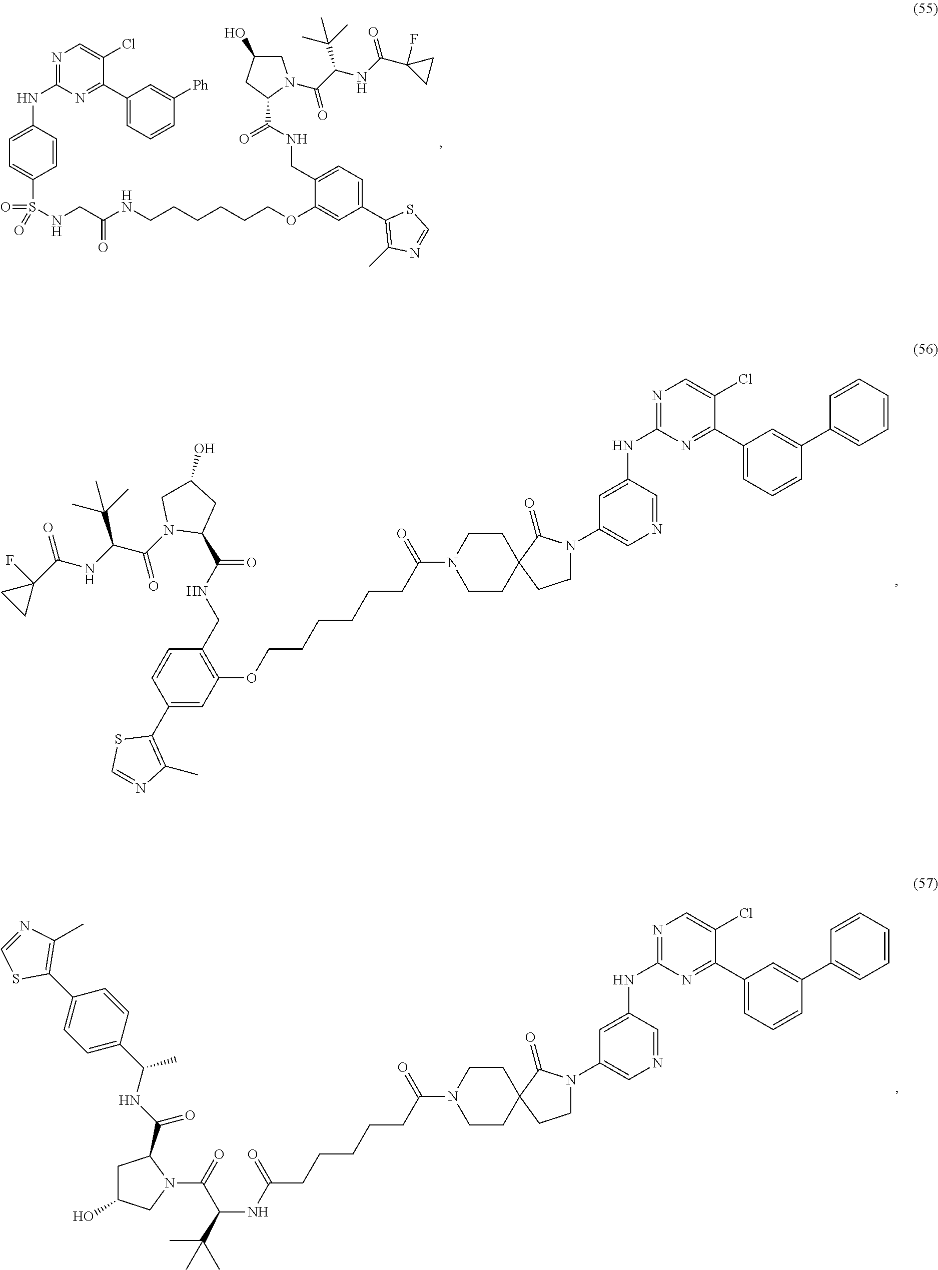 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCNC(=O)CNS(=O)(=O)c2ccc(Nc3ncc(Cl)c(-c4cccc(-c5ccccc5)c4)n3)cc2)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCC3(CC2)CCN(c2cncc(Nc4ncc(Cl)c(-c5cccc(-c6ccccc6)c5)n4)c2)C3=O)c1 ; Cc1ncsc1-c1ccc([C@H](C)NC(=O)[C@@H]2C[C@@H](O)CN2C(=O)[C@@H](NC(=O)CCCCCC(=O)N2CCC3(CC2)CCN(c2cncc(Nc4ncc(Cl)c(-c5cccc(-c6ccccc6)c5)n4)c2)C3=O)C(C)(C)C)cc1]

or a pharmaceutically acceptable salt or stereoisomer thereof.

Compounds of the present invention (i.e., compounds of formula I or I′ and bifunctional compounds of formula II or II′) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Compounds of the invention may have at least one chiral center and therefore may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; therefore, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

The compounds of the invention embrace isotopic derivatives that have at least one desired isotopic substitution of an atom, at an amount above the natural abundance of the isotope, i.e., enriched. In one embodiment, the compound includes deuterium or multiple deuterium atoms.

Substitution with heavier isotopes such as deuterium, i.e. ²H, may afford certain therapeutic advantages resulting from greater metabolic stability, for example, increased in vivo half-life or reduced dosage requirements, and therefore may be advantageous in some circumstances.

In addition to isotopic derivatives, the terms “compounds of formula (I or I′)” and “bifunctional compounds of formula (II or II′)” embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds.

### Methods of Synthesis

In another aspect, the present invention is directed to methods for making compounds of formula (I or I′) and bifunctional compounds of formula (II or II′), and their pharmaceutically acceptable salts and stereoisomers. Broadly, the inventive compounds and pharmaceutically acceptable salts or stereoisomers thereof may be prepared by any process known to be applicable to the preparation of chemically related compounds. The compounds of the present invention will be better understood in connection with the synthetic schemes and procedures that described in various working examples and which illustrate non-limiting methods by which the compounds of the invention may be prepared.

The bifunctional compounds of Formula II or II′ can be prepared by methods known by those skilled in the art. In one non-limiting example the disclosed bifunctional compounds can be made by the following schemes.

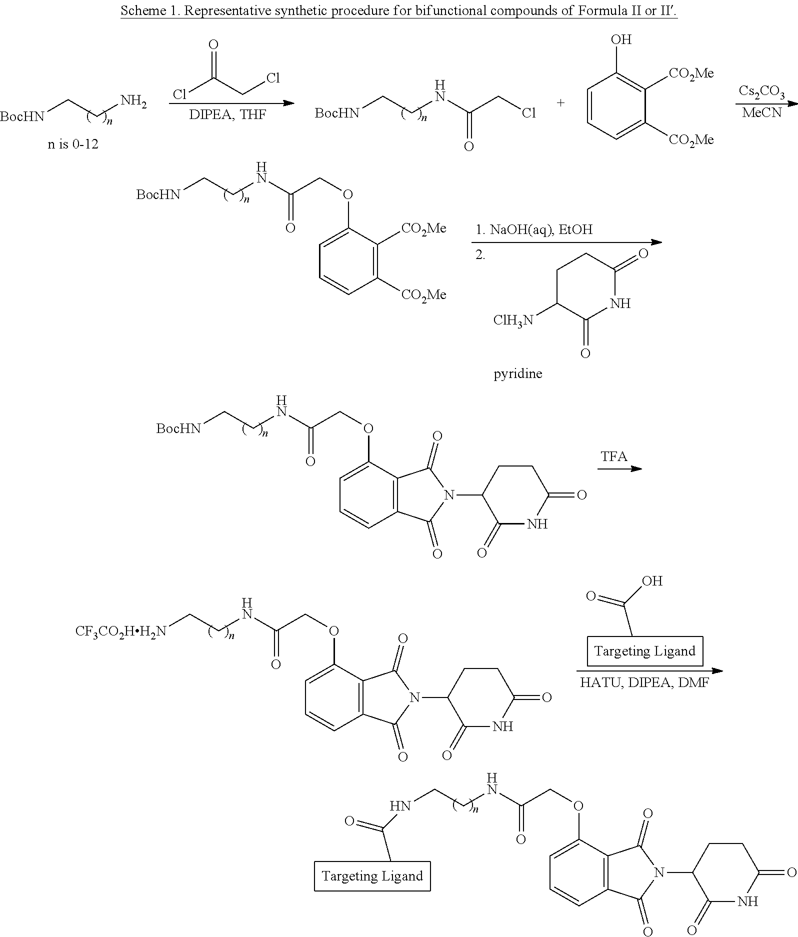

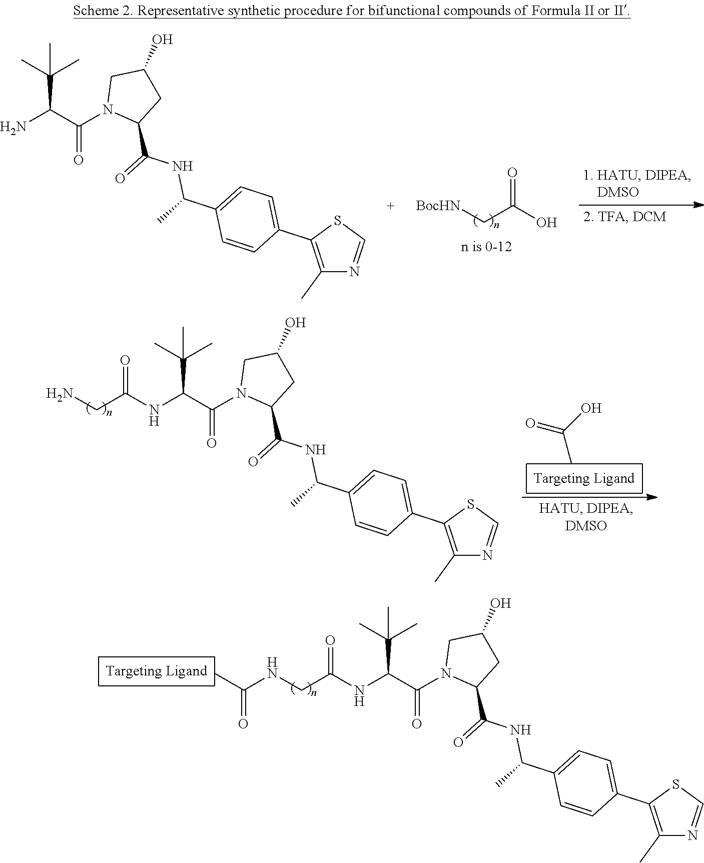

In some embodiments, the methods described above are carried out in solvent. In some embodiments, the aprotic solvent is DCM, DMSO, DMF, CHCl₃, CCl₄, DCE, toluene, MeCN, THF, MeOH, or EtOH. In some embodiments, the methods described above are carried out on a solid phase.

In some embodiments, the reaction includes the use of a base. In some embodiments, the base is used in excess at about 10 equivalents. In some embodiments, the base is used in excess at about 5 equivalents. In some embodiments, the base is used in excess at about 3 equivalents.

In some embodiments, the reaction includes the use of an acid. In some embodiments, the acid is used in excess at about 10 equivalents. In some embodiments, the acid is used in excess at about 5 equivalents. In some embodiments, the acid is used in excess at about 3 equivalents.

In some embodiments, the reaction is carried out at a temperature from about −78° C. to 100° C. In some embodiments, the reaction is carried out at a temperature from about 0° C.-60° C. In some embodiments, the reaction is carried out at a temperature is about 20° C.-25° C.

In some embodiments, the reaction involves a coupling reagent. In some embodiments, the coupling reagent is HATU (1-[Bis(dimethylamino)methylene]-1H-1,2,3-triazolo[4,5-b]pyridinium-3-oxide hexafluorophosphate), PyAOP ((7-Azabenzotriazol-1-yloxy)tripyrrolidinophosphonium hexafluorophosphate), BOP (benzotriazol-1-yloxytris(dimethylamino)phosphonium hexafluorophosphate), HBTU (2-(1H-benzotriazol-1-yl)-1,1,3,3-tetramethyluronium hexafluorophosphate), or PyBOP (benzotriazol-1-yloxytripyrrolidinophosphonium hexafluorophosphate).

Synthetic schemes for synthesizing bifunctional compounds are known in the art. See, e.g., Powell et al., J. Med. Chem., 61(9):4249-4255 (2018) and Lai et al., Anew. Chem. Int. Ed., 55:807-810 (2016), each of which is incorporated herein by reference in its entirety.

### Pharmaceutical Compositions

Another aspect of the present invention is directed to a pharmaceutical composition that includes a therapeutically effective amount of a compound of formula (I or I′) or a bifunctional compound of formula (II or II′), or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I or I′) and bifunctional compounds of formula (I1 or II′) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York), each of which is incorporated herein by reference in its entirety. The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal).

In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the inventive compound may be administered relatively quickly such as in the case of a single-dose treatment and/or an acute condition.

In some embodiments, the inventive compounds are formulated for oral or intravenous administration (e.g., systemic intravenous injection).

Accordingly, compounds of formula (I or I′) and bifunctional compounds of formula (II or II′) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I or I′) and bifunctional compounds of formula (II or II′) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I or I′) and bifunctional compounds of formula (II or II′) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I or I′) and bifunctional compounds of formula (II or II′) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount.

In some embodiments, capsules and cartridges including gelatin, for example, for use in an inhaler or insufflator, may be formulated containing a powder mix of the compound and a suitable powder base such as lactose or starch.

Compounds of formula (I or I′) and bifunctional compounds of formula (II or II′) may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating inventive compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997), each of which is incorporated herein by reference in its entirety. Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I or I′) or a bifunctional compound of formula (II or II′), or a pharmaceutically acceptable salt or a stereoisomer thereof; or a composition including a compound of formula (I or I′) or a bifunctional compound of formula (II or II′), or a pharmaceutically acceptable salt or a stereoisomer thereof, effective in producing the desired therapeutic response in a particular patient in need thereof. Therefore, the term “therapeutically effective amount” includes the amount of a compound of formula (I or I′) or a bifunctional compound of formula (II or II′), or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer, autophagy-dependent disease (e.g., neurodegenerative disorder)) cells, or reduces the amount of at least one other of PIP4K2A, PIP4K2B, and PIP4K2C in diseased cells.

The total daily dosage of the inventive compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the inventive compound; and like factors well known in the medical arts (see, for example, Goodman and Gilman's The Pharmacological Basis of Therapeutics, 10th Edition, A. Gilman, J. Hardman and L. Limbird, eds., McGraw-Hill Press, 155-173, 2001), which is incorporated herein by reference in its entirety.

Compounds of formula (I or I′) and bifunctional compounds of formula (II or II′), and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of an inventive compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders by reducing the level or activity of at least one of PIP4K2A, PIP4K2B, and PIP4K2C, that entails administration of a therapeutically effective amount of a compound formula (I or I′) or a bifunctional compound of formula (II or II′), or a pharmaceutically acceptable salt or stereoisomer thereof, to a subject in need thereof.

In some embodiments, the level or activity of at least one of PIP4K2A, PIP4K2B, and PIP4K2C in the subject is reduced by, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to an untreated control subject.

A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Degradation or depletion of PIP4K2A, PIP4K2B, and/or PIP4K2C proteins is useful for treating and/or diagnosing cancer. In some cases, the PIP4K2C protein is targeted because it is expressed at higher levels in immune cells than the PIP4K2A and PIP4K2B proteins. Degradation or depletion of the PIP4K2A protein, the PIP4K2B protein, and/or particularly the PIP4K2C protein can enhance immune responses against cancer and tumors. Depletion or degradation of PIP4Ks, particularly PIP4K2B protein, is useful for treatment of insulin resistance.

In some embodiments, the inventive compounds may be used to reduce the level of PIP4K2B in a patient suffering from or suspected of suffering from insulin resistance. In some embodiments, the inventive compounds may be used to decrease the level of PIP4K2C in a patient suffering from or suspected of suffering from cancer, immune deficiency, autoimmune disease, or infectious disease, or a combination thereof.

In some aspects, the present invention is directed to methods of enhancing immune functions in a subject in need thereof, comprising administering to a subject a therapeutically effective amount of a compound of formula (I or I′) or a bifunctional compound of formula (II or II′), or pharmaceutically acceptable salt or stereoisomer thereof.

In some embodiments, immune functions in the subject are enhanced by, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to an untreated control subject.

In some aspect, the present invention is directed to methods of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of a compound of formula (I or I′) or a bifunctional compound of formula (II or II′), or pharmaceutically acceptable salt or stereoisomer thereof. The reduction in the scaffolding or interaction may stimulate the immune system.

In some embodiments, compounds of formula (I or I′) and bifunctional compounds of formula (II or II′), and their pharmaceutically acceptable salts and stereoisomers may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, metabolic diseases, and allergic and genetic diseases.

In some embodiments, the inventive compounds and their pharmaceutically acceptable salts and stereoisomers may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the inventive compounds and their pharmaceutically acceptable salts and stereoisomers may be useful in the treatment of autoimmune diseases and disorders. As used herein, the term “autoimmune disease” refers to conditions where the immune system produces antibodies that attack normal body tissues. Representative examples of such diseases include autoimmune hematological disorders (e.g., hemolytic anemia, aplastic anemia, anhidrotic ectodermal dysplasia, pure red cell anemia and idiopathic thrombocytopenia), Sjogren's syndrome, Hashimoto thyroiditis, rheumatoid arthritis, juvenile (type 1) diabetes, polymyositis, scleroderma, Addison's disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy, Wegener granulomatosis, autoimmune oophoritis, sarcoidosis, rheumatic carditis, ankylosing spondylitis, Grave's disease, autoimmune thrombocytopenic purpura, psoriasis, psoriatic arthritis, dermatitis herpetiformis, ulcerative colitis, and temporal arteritis.

In other embodiments, the methods are directed to treating subjects having cancer. Broadly, the compounds of formula (I or I′) or bifunctional compounds of formula (II or II′), and their pharmaceutically acceptable salts and stereoisomers may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the inventive compounds include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain.

Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma.

Lung cancer also includes lung neoplasms having histologic and ultrastructural heterogeneity (e.g., mixed cell types). In some embodiments, an inventive may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is a hematological cancer e.g., leukemia, lymphoma or multiple myeloma.

The compounds of formula (I or I′) or bifunctional compounds of formula (II or II′), and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present invention may entail administration of a compound of formula (I or I′) or a bifunctional compound of formula (II or I1′), or a pharmaceutically acceptable salt or stereoisomer thereof or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the inventive compound may be dosed twice a day (BID) over the course of two and a half days (for a total of 5 doses) or once a day (QD) over the course of two days (for a total of 2 doses). In other embodiments, the inventive compound may be dosed once a day (QD) over the course of five days.

### Combination Therapy

The compounds of formula (I or I′) and bifunctional compounds of formula (II or II′) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I or I′) or a bifunctional compound of formula (II or II′), and their pharmaceutically acceptable salts and stereoisomers in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference, 60th ed., 2006, which is incorporated herein by reference in its entirety. For example, anti-cancer agents that may be suitable for use in combination with the inventive compounds are known in the art. See, e.g., U.S. Pat. No. 9,101,622 (Section 5.2 thereof) and U.S. Pat. No. 9,345,705 B2 (Columns 12-18 thereof), each of which is incorporated herein by reference in its entirety. Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, a compound of formula (I or I′) or a bifunctional compound of formula (II or II′), or a pharmaceutically acceptable salt or a stereoisomer thereof and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, a compound of formula (I or I′) or a bifunctional compound of formula (II or II′), or a pharmaceutically acceptable salt or stereoisomer thereof and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The inventive compounds and their pharmaceutically acceptable salts and stereoisomers and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of formula (I or I′) or a bifunctional compound of formula (II or II′), or a pharmaceutical composition thereof. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: General Information

Unless otherwise noted, reagents and solvents were obtained from commercial suppliers and were used without further purification. ¹H NMR spectra were recorded on 500 MHz Bruker Avance III spectrometer, and chemical shifts are reported in parts per million (ppm, d) downfield from tetramethylsilane (TMS). Coupling constants (J) are reported in Hz. Spin multiplicities are described as s (singlet), brs (broad singlet), d (doublet), t (triplet), q (quartet), and m (multiplet). Mass spectra were obtained on a Waters™ Acquity ultra performance liquid chromatography (UPLC). Preparative high performance liquid chromatography (HPLC) was performed on a Waters™ Sunfire C18 column (19 mm×50 mm, 5 μM) using a gradient of 15-95% methanol in water containing 0.05% trifluoroacetic acid (TFA) over 60 min at a flow rate of 43 mL/min. Flash column chromatography was carried out using prepacked silica cartridges (from 4 g up to 24 g) from RediSep® and eluted using an Isco Companion system. Purities of assayed compounds were in all cases greater than 95%, as determined by reverse-phase HPLC analysis.

### Abbreviations Used

dppf, 1,1′-Ferrocenediyl-bis(diphenylphosphine); DCM, dichloromethane; TFA, trifluoroacetic acid; HOAc, acetic acid; DMAP, 4-(Dimethylamino)pyridine; DIPEA, N,N-diisopropylethylamine; EDCI, N-(3-Dimethylaminopropyl)-N′-ethylcarbodiimide hydrochloride; DMSO, dimethyl sulfoxide; rt, room temperature; XPhos, 2-dicyclohexylphosphino-2′,4′,6′-triisopropylbiphenyl; HATU, 1-[bis(dimethylamino)methylene]-1H-1,2,3-triazolo[4,5-b]pyridinium 3-oxid hexafluorophosphate.

### Example 2: Synthesis of N-((4-((4-([1,1′-biphenyl]-3-yl)-5-chloropyrimidin-2-yl)amino)-3-methylphenyl)sulfonyl)-N-methylacetamide (3)

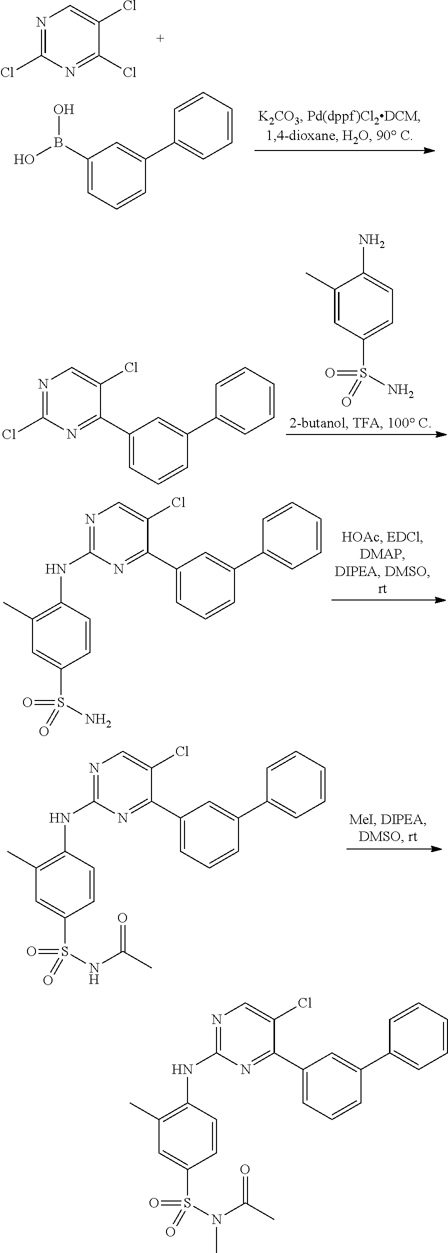

### 4-([1,1′-biphenyl]-3-yl)-2,5-dichloropyrimidine

K₂CO₃ (300.0 mg, 2.18 mmol) and Pd(dppf)Cl₂·DCM (66.0 mg, 0.081 mmol) were added to a solution of 2,4,5-trichloropyrimidine (200.0 mg, 1.09 mmol) and 3-biphenylboronic acid (216.0 mg, 1.09 mmol) in 1,4-dixoane/H₂O (v/v=2/1, 5.0 mL). The reaction mixture stirred at 90° C. for 3 hours. The reaction mixture was purified via column chromatography (silica gel, eluted with 0% to 10% hexane in ethyl acetate) to give the title compound as a white solid (300.0 mg, 91%). ¹H NMR (500 MHz, DMSO-d) S 9.04 (s, 1H), 8.08 (t, J=1.5 Hz, 1H), 7.90 (dt, J=7.5, 1.5 Hz, 1H), 7.82 (dt, J=8.0, 1.5 Hz, 1H), 7.72 (dd, J=9.0, 1.5 Hz, 2H), 7.68 (t, J=8.0 Hz, 1H), 7.51 (t, J=7.5 Hz, 2H), 7.42 (t, J=7.5 Hz, 1H). MS (ESI) for C₁₆H₁₁Cl₂N₂[M+H]⁺: m/z calcd, 301.03; found, 301.23.

### 4-((4-([1,1′-biphenyl]-3-yl)-5-chloropyrimidin-2-yl)amino)-3-methylbenzenesulfonamide

TFA (50 pL, 0.64 mmol) was added to a solution of 4-([1,1′-biphenyl]-3-yl)-2,5-dichloropyrimidine (97.0 mg, 0.32 mmol) and 4-amino-3-methylbenzenesulfonamide (60.0 mg, 0.32 mmol) in 2-butanol (2.0 mL). The reaction mixture stirred at 100° C. for 3 hours. The reaction mixture was purified directly via prep HPLC to give the title compound as a white solid (8.2 mg, 6%). ¹H NMR (500 MHz, DMSO-d₆) δ 9.38 (s, 1H), 8.61 (s, 1H), 8.06 (s, 1H), 7.85-7.77 (m, 3H), 7.73-7.66 (m, 3H), 7.65-7.60 (m, 2H), 7.51 (t, J=8.0 Hz, 2H), 7.41 (t, J=7.5 Hz, 1H), 7.23 (s, 2H), 2.34 (s, 3H). MS (ESI) for C₂₃H₂₀ClN₄O₂S [M+H]⁺: m/z calcd, 451.10; found, 451.40.

### N-((4-((4-([1,1′-biphenyl]-3-yl)-5-chloropyrimidin-2-yl)amino)-3-methylphenyl)sulfonyl)acetamide

HOAc (20.0 mg, 0.33 mmol), DMAP (8.0 mg, 0.066 mmol), DIPEA (0.11 mL, 0.66 mmol), and EDCI (19.0 mg, 0.099 mmol) were added to a solution of 4-((4-([1,1′-biphenyl]-3-yl)-5-chloropyrimidin-2-yl)amino)-3-methylbenzenesulfonamide (30.0 mg, 0.066 mmol) in DMSO (1.0 mL). The reaction mixture stirred at room temperature (rt) for 18 hours. The reaction mixture was purified directly via prep HPLC to give the title compound as a white solid (9.0 mg, 27%). MS (ESI) for C₂₅H₂₂ClN₄O₃S [M+H]⁺: m/z calcd, 493.11; found, 493.44.

### N-((4-((4-([1,1′-biphenyl]-3-yl)-5-chloropyrimidin-2-yl)amino)-3-methylphenyl)sulfonyl)-N-methylacetamide (3)

DIPEA (0.1 mL, 0.58 mmol) and Mel (5.0 mg, 0.036 mmol) were added to a solution of N-((4-((4-([1,1′-biphenyl]-3-yl)-5-chloropyrimidin-2-yl)amino)-3-methylphenyl)sulfonyl)acetamide (3.6 mg, 7.3 μmol) in DMSO (1.0 mL). The reaction mixture stirred at rt for 1 hour. The reaction mixture was purified directly via prep HPLC to give the title compound as a white solid (2.1 mg, 56%). ¹H NMR (500 MHz, DMSO-d₆) δ 9.44 (s, 1H), 8.67 (s, 1H), 8.07 (t, J=1.5 Hz, 1H), 8.01 (d, J=8.5 Hz, 1H), 7.83 (td, J=7.5, 2.0 Hz, 2H), 7.78 (d, J=1.5 Hz, 1H), 7.75 (dd, J=8.5, 2.5 Hz, 1H), 7.71 (dd, J=8.5, 1.0 Hz, 2H), 7.63 (t, J=8.0 Hz, 1H), 7.51 (t, J=7.5 Hz, 2H), 7.41 (t, J=7.5 Hz, 1H), 3.27 (s, 3H), 2.48 (s, 3H), 2.25 (s, 3H). MS (ESI) for C₂₆H₂₄ClN₄O₃S [M+H]⁺: m/z calcd, 507.13; found, 507.06.

### Example 3: Synthesis of N-(4-((4-([1,1′-biphenyl]-3-yl)-5-chloropyrimidin-2-yl)amino)-3-methylphenyl)methanesulfonamide (6)

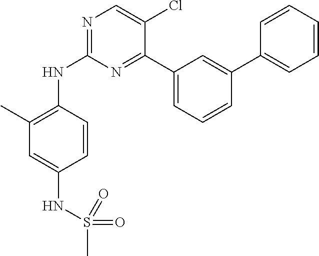 [SMILES: Cc1cc(NS(C)(=O)=O)ccc1Nc1ncc(Cl)c(-c2cccc(-c3ccccc3)c2)n1]

Following a similar procedure as in Example 2, compound 6 was isolated as a white solid (5.5 mg, 23%) using N-(4-Amino-3-methylphenyl)methanesulfonamide. ¹H NMR (500 MHz, DMSO-d₆) δ 9.58 (s, 1H), 9.14 (s, 1H), 8.51 (s, 1H), 8.02 (s, 1H), 7.81 (d, J=8.0 Hz, 1H), 7.77 (d, J=8.0 Hz, 1H), 7.69 (d, J=8.0 Hz, 2H), 7.61 (t, J=8.0 Hz, 1H), 7.51 (t, J=8.0 Hz, 2H), 7.45-7.39 (m, 2H), 7.09-7.03 (m, 2H), 2.94 (s, 3H), 2.21 (s, 3H). MS (ESI) for C₂₄H₂₂ClN₄O₂S [M+H]⁺: m/z calcd, 465.12; found, 465.09.

### Example 4: Synthesis of 4-((4-([1,1′-biphenyl]-3-yl)-5-chloropyrimidin-2-yl)amino)-3-methylbenzamide (5)

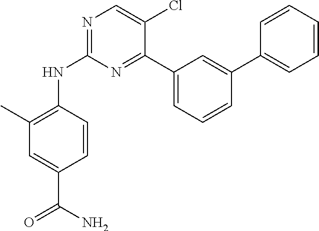 [SMILES: Cc1cc(C(N)=O)ccc1Nc1ncc(Cl)c(-c2cccc(-c3ccccc3)c2)n1]

Following a similar procedure as in Example 2, compound 5 was isolated as a white solid (4.0 mg, 14%) using 4-amino-3-methylbenzamide. ¹H NMR (500 MHz, DMSO-D₆) δ 9.27 (s, 1H), 8.59 (s, 1H), 8.05 (s, 1H), 7.85 (s, 1H), 7.83 (d, J=8.0 Hz, 1H), 7.79 (d, J=8.0 Hz, 1H), 7.76 (s, 1H), 7.72-7.68 (m, 4H), 7.62 (t, 1=8.0 Hz, 1H), 7.51 (t, J=8.0 Hz, 2H), 7.41 (t, J=7.5 Hz, 1H), 7.22 (s, 1H), 2.30 (s, 3H). MS (ESI) for C₂₄H₂₀ClN₄O [M+H]⁺: m/z calcd, 415.13; found, 415.16.

### Example 5: Synthesis of N-(4-((4-([1,1′-biphenyl]-3-yl)-5-chloropyrimidin-2-yl)amino)-3-methylphenyl)acetamide (8)

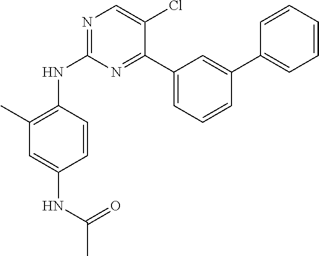 [SMILES: CC(=O)Nc1ccc(Nc2ncc(Cl)c(-c3cccc(-c4ccccc4)c3)n2)c(C)c1]

Following a similar procedure as in Example 2, compound 8 was isolated as a white solid (2.0 mg, 7%) using N-(4-amino-3-methylphenyl)acetamide. ¹H NMR (500 MHz, DMSO-d₆) δ 9.84 (s, 1H), 9.09 (s, 1H), 8.49 (s, 1H), 8.02 (s, 1H), 7.81 (d, J=8.0 Hz, 1H), 7.76 (d, J=7.5 Hz, 1H), 7.69 (d, J=7.5 Hz, 2H), 7.60 (t, J=7.5 Hz, 1H), 7.51 (t, J=8.0 Hz, 2H), 7.45-7.37 (m, 3H), 7.34 (d, J=8.5 Hz, 1H), 2.20 (s, 3H), 2.03 (s, 3H). MS (ESI) for C₂₅H₂₂ClN₄O [M+H]⁺: m/z calcd, 429.15; found, 429.21.

### Example 6: Synthesis of N-(3-((4-([1,1′-biphenyl]-3-yl)-5-chloropyrimidin-2-yl)amino)-4-methylphenyl)methanesulfonamide (7)

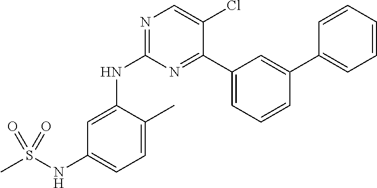 [SMILES: Cc1ccc(NS(C)(=O)=O)cc1Nc1ncc(Cl)c(-c2cccc(-c3ccccc3)c2)n1]

Following a similar procedure as in Example 2, compound 7 was isolated as a white solid (8.7 mg, 37%) using N-(3-amino-4-methylphenyl)methanesulfonamide. ¹H NMR (500 MHz, DMSO-d₆) δ 9.61 (s, 1H), 9.21 (s, 1H), 8.55 (s, 1H), 8.01 (s, 1H), 7.81 (d, J=8.0 Hz, 1H), 7.77 (d, J=7.5 Hz, 1H), 7.70 (d, J=8.0 Hz, 2H), 7.60 (t, J=8.0 Hz, 1H), 7.50 (t, J=7.5 Hz, 2H), 7.43-7.38 (m, 2H), 7.17 (d, J=8.0 Hz, 1H), 6.92 (dd, J=8.5, 2.0 Hz, 1H), 2.86 (s, 3H), 2.19 (s, 3H). MS (ESI) for C₂₄H₂₂ClN₄O₂S [M+H]⁺: m/z calcd, 465.12; found, 465.15.

### Example 7: Synthesis of 3-((4-([1,1′-biphenyl]-3-yl)-5-chloropyrimidin-2-yl)amino)-4-methylbenzamide (10)

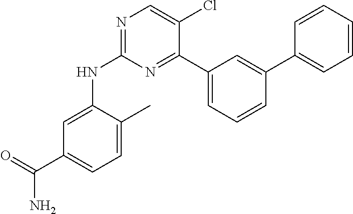 [SMILES: Cc1ccc(C(N)=O)cc1Nc1ncc(Cl)c(-c2cccc(-c3ccccc3)c2)n1]

Following a similar procedure as in Example 2, compound 10 was isolated as a light yellow solid (8.0 mg, 28%) using 3-amino-4-methylbenzamide. ¹H NMR (500 MHz, DMSO-d₆) δ 9.29 (s, 1H), 8.56 (s, 1H), 8.03 (d, J=1.5 Hz, 2H), 7.90 (s, 1H), 7.83-7.77 (m, 2H), 7.69 (d, J=7.0 Hz, 2H), 7.64-7.57 (m, 2H), 7.51 (t, J=8.0 Hz, 2H), 7.40 (t, J=7.5 Hz, 1H), 7.30 (d, J=8.0 Hz, 1H), 7.27 (s, 1H), 2.28 (s, 3H). MS (ESI) for C₂₄H₂₀ClN₄O [M+H]⁺: m/z calcd, 415.13; found, 415.16.

### Example 8: Synthesis of N-(3-((4-([1,1′-biphenyl]-3-yl)-5-chloropyrimidin-2-yl)amino)-4-methylphenyl)acetamide (9)

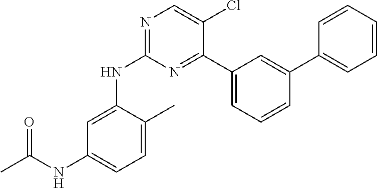 [SMILES: CC(=O)Nc1ccc(C)c(Nc2ncc(Cl)c(-c3cccc(-c4ccccc4)c3)n2)c1]

Following a similar procedure as in Example 2, compound 9 was isolated as a white solid (1.2 mg, 5%) using N-(3-amino-4-methylphenyl)acetamide. ¹H NMR (500 MHz, DMSO-d₆) δ 9.85 (s, 1H), 9.17 (s, 1H), 8.53 (s, 1H), 8.03 (s, 1H), 7.82-7.77 (m, 3H), 7.68 (d, J=8.0 Hz, 2H), 7.60 (t, J=8.0 Hz, 1H), 7.49 (t, J=8.0 Hz, 2H), 7.40 (t, J=7.5 Hz, 1H), 7.26 (dd, J=8.5, 2.0 Hz, 1H), 7.12 (d, J=8.0 Hz, 1H), 2.17 (s, 3H), 1.99 (s, 3H). MS (ESI) for C₂₅H₂₂ClN₄O [M+H]⁺: m/z calcd, 429.15; found, 429.21.

### Example 9: Synthesis of N-(5-((4-([1,1′-biphenyl]-3-yl)-5-chloropyrimidin-2-yl)amino)pyridin-3-yl)acetamide (11)

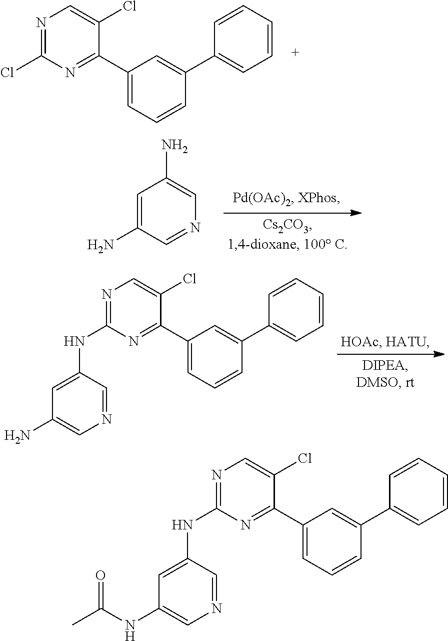

### N³-(4-([1,1′-biphenyl]-3-yl)-5-chloropyrimidin-2-yl)pyridine-3,5-diamine

Cs₂CO₃ 108.0 mg, 0.33 mmol), XPhos (15.7 mg, 0.033 mmol), and Pd(OAc)₂ (7.4 mg, 0.033 mmol) was added to a solution of 4-([1,1′-biphenyl]-3-yl)-2,5-dichloropyrimidine (100.0 mg, 0.33 mmol) and 3,5-pyridinediamine (36.0 mg, 0.33 mmol) in 1,4-dixoane (3.0 mL). The reaction mixture stirred at 100° C. for 18 hours. The reaction mixture was purified directly via prep HPLC to give the title compound as a yellow viscous oil (65.0 mg, 52%). MS (ESI) for C₂₁H₁₇ClN₅ [M+H]⁺: m/z calcd, 374.12; found, 374.15.

### N-(5-((4-([1,1′-biphenyl]-3-yl)-5-chloropyrimidin-2-yl)amino)pyridin-3-yl)acetamide

HOAc (6.0 mg, 0.095 mmol), DIPEA (33 pL, 0.19 mmol), and HATU (11.0 mg, 0.029 mmol) was added to a solution of N³-(4-([1,1′-biphenyl]-3-yl)-5-chloropyrimidin-2-yl)pyridine-3,5-diamine (7.0 mg, 0.019 mmol) in DMSO (1.0 mL). The reaction mixture stirred at rt for 3 hours. The reaction mixture was purified directly via prep HPLC to give the title compound as a yellow solid (1.0 mg, 12%). ¹H NMR (500 MHz, DMSO-d₆) δ 10.29 (s, 1H), 10.26 (s, 1H), 8.72 (s, 2H), 8.54 (s, 1H), 8.43 (s, 1H), 8.11 (t, J=2.0 Hz, 1H), 7.86 (t, J=8.0 Hz, 2H), 7.73 (dd, J=8.5, 1.5 Hz, 2H), 7.66 (t, J=8.0 Hz, 1H), 7.51 (t, J=8.0 Hz, 2H), 7.41 (t, J=7.5 Hz, 1H), 2.05 (s, 3H). MS (ESI) for C₂₃H₁₉ClN₅O [M+H]⁺: m/z calcd, 416.13; found, 416.18.

### Example 10: Synthesis of 5-((4-([1,1′-biphenyl]-3-yl)-5-chloropyrimidin-2-yl)amino)nicotinamide (16)

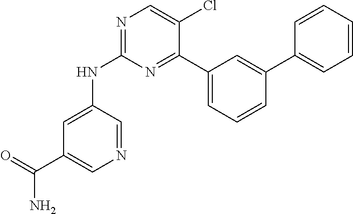 [SMILES: NC(=O)c1cncc(Nc2ncc(Cl)c(-c3cccc(-c4ccccc4)c3)n2)c1]

Following a similar procedure as in Example 9, compound 16 was isolated as a yellow solid (5.2 mg, 13%) using 5-aminonicotinamide. ¹H NMR (500 MHz, DMSO-d₆) δ 10.37 (s, 1H), 9.07 (s, 1H), 8.76 (s, 1H), 8.70-8.66 (m, 2H), 8.14 (s, 1H), 8.11 (t, J=1.5 Hz, 1H), 7.86 (dt, J=8.0, 2.0 Hz, 2H), 7.74 (d, J=7.5 Hz, 2H), 7.66 (t, J=8.0 Hz, 1H), 7.61 (s, 1H), 7.51 (t, J=8.0 Hz, 2H), 7.41 (t, J=7.5 Hz, 1H). MS (ESI) for C₂₂H₁₇ClN₅O [M+H]⁺: m/z calcd, 402.11; found, 402.30.

### Example 11: Synthesis of N-(5-((4-([1,1′-biphenyl]-3-yl)-5-chloropyrimidin-2-yl)amino)pyridin-3-yl)benzamide (13)

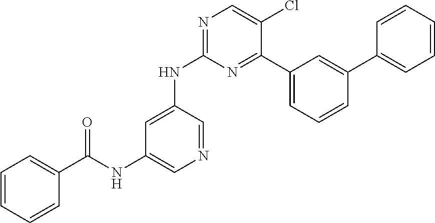 [SMILES: O=C(Nc1cncc(Nc2ncc(Cl)c(-c3cccc(-c4ccccc4)c3)n2)c1)c1ccccc1]

Following a similar procedure as in Example 9, compound 13 was isolated as a yellow solid (4.3 mg, 27%) using N-(5-aminopyridin-3-yl)benzamide. ¹H NMR (500 MHz, DMSO-d₆) δ 10.77 (s, 1H), 10.59 (s, 1H), 8.90 (s, 2H), 8.78 (s, 1H), 8.73 (s, 1H), 8.13 (s, 1H), 7.97 (d, 0.1=7.0 Hz, 2H), 7.90 (d, J=7.5 Hz, 1H), 7.86 (d, J=8.0 Hz, 1H), 7.73 (d, J=7.5 Hz, 2H), 7.68-7.62 (m, 2H), 7.56 (t, J=7.5 Hz, 2H), 7.48 (t, J=7.5 Hz, 2H), 7.39 (t, J=7.5 Hz, 1H). MS (ESI) for C₂₈H₂₁ClN₅O [M+H]⁺: m/z calcd, 478.14; found, 478.46.

### Example 12: Synthesis of N-(5-((5-chloro-4-(3-(pyridin-3-yl)phenyl)pyrimidin-2-yl)amino)pyridin-3-ylbenzamide (14)

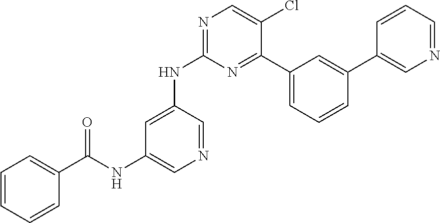 [SMILES: O=C(Nc1cncc(Nc2ncc(Cl)c(-c3cccc(-c4cccnc4)c3)n2)c1)c1ccccc1]

Following a similar procedure as in Example 9, compound 14 was isolated as a yellow solid (3.0 mg, 18%) using N-(5-aminopyridin-3-yl)benzamide and 2,5-dichloro-4-(3-(pyridin-3-yl)phenyl)pyrimidine. ¹H NMR (500 MHz, DMSO-d₆) δ 10.74 (s, 1H), 10.59 (s, 1H), 9.08 (d, J=2.0 Hz, 1H), 8.96 (s, 1H), 8.87 (d, J=2.0 Hz, 1H), 8.80 (s, 1H), 8.70-8.67 (m, 2H), 8.38 (d, J=7.5 Hz, 1H), 8.24 (s, 1H), 7.98 (dd, J=8.0, 2.0 Hz, 2H), 7.92 (d, J=7.5 Hz, 2H), 7.73 (t, J=7.5 Hz, 1H), 7.69-7.61 (m, 2H), 7.55 (t, J=7.5 Hz, 2H). MS (ESI) for C₂₇H₂₀ClN₆O [M+H]⁺: m/z calcd, 479.14; found, 479.42.

### Example 13: Synthesis of N-(3-((4-([1,1′-biphenyl]-3-yl)-5-chloropyrimidin-2-yl)amino)phenyl)benzamide (12)

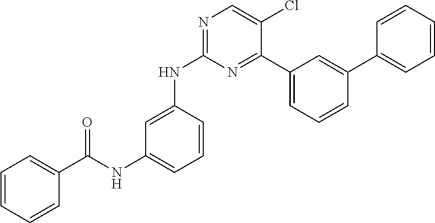 [SMILES: O=C(Nc1cccc(Nc2ncc(Cl)c(-c3cccc(-c4ccccc4)c3)n2)c1)c1ccccc1]

Following a similar procedure as in Example 9, compound 12 was isolated as a yellow solid (3.9 mg, 15%) using N-(3-aminophenyl)benzamide. ¹H NMR (500 MHz, DMSO-d₆) δ 10.24 (s, 1H), 9.97 (s, 1H), 8.65 (s, 1H), 8.24 (s, 1H), 8.12 (s, 1H), 7.94 (d, J=7.0 Hz, 2H), 7.89 (d, J=8.0 Hz, 1H), 7.83 (d, J=8.0 Hz, 1H), 7.72 (d, J=7.5 Hz, 2H), 7.63 (t, J=7.5 Hz, 1H), 7.61-7.56 (m, 1H), 7.55-7.49 (m, 3H), 7.47 (t, J=7.5 Hz, 2H), 7.38 (t, J=7.5 Hz, 1H), 7.35 (d, J=8.0 Hz, 1H), 7.26 (t, J=8.0 Hz, 1H). MS (ESI) for C₂₉H₂₂ClN₄O [M+H]⁺: m/z calcd, 477.15; found, 477.39.

### Example 14: Synthesis of 1-(54(4-([1,1′-biphenyl]-3-yl)-5-chloropyrimidin-2-yl)amino)pyridin-3-yl)pyrrolidin-2-one (17)

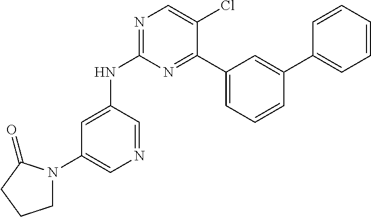 [SMILES: O=C1CCCN1c1cncc(Nc2ncc(Cl)c(-c3cccc(-c4ccccc4)c3)n2)c1]

Following a similar procedure as in Example 9, compound 17 was isolated as a yellow solid (2.9 mg, 10%) using 1-(5-aminopyridin-3-yl)pyrolidin-2-one. ¹H NMR (500 MHz, DMSO-d₆) δ 10.50 (s, 1H), 8.77 (s, 3H), 8.72 (s, 1H), 8.15 (t, J=1.5 Hz, 1H), 7.91-7.86 (m, 2H), 7.75 (dd, J=9.0, 1.5 Hz, 2H), 7.67 (t, J=8.0 Hz, 1H), 7.51 (t, J=8.0 Hz, 2H), 7.42 (t, J=7.0 Hz, 1H), 3.76 (t, J=7.0 Hz, 2H), 2.45 (t, J=8.0 Hz, 2H), 1.97-1.89 (m, 2H). MS (ESI) for C₂₅H₂₁ClN₅O [M+H]⁺: m/z calcd, 442.14; found, 442.42.

### Example 15: Synthesis of (S)-2-((4-([1,1′-biphenyl]-3-yl)-5-chloropyrimidin-2-yl)amino)-6,6a,7,8-tetrahydro-9H-pyrido[2,3-b]pyrrolo[1,2-d][1,4]oxazin-9-one (15)

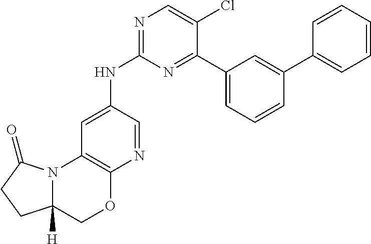 [SMILES: O=C1CC[C@H]2COc3ncc(Nc4ncc(Cl)c(-c5cccc(-c6ccccc6)c5)n4)cc3N12]

Following a similar procedure as in Example 9, compound 15 was isolated as a yellow solid (2.8 mg, 12%) using (S)-2-amino-6,6a,7,8-tetrahydro-9H-pyrido[2,3-b]pyrrolo[1,2-d][1,4]oxazin-9-one. ¹H NMR (500 MHz, DMSO-d₆) δ 9.96 (s, 1H), 9.13 (d, J=1.5 Hz, 1H), 8.63 (s, 1H), 8.25 (d, J=2.5 Hz, 1H), 8.09 (s, 1H), 7.87 (d, J=8.0 Hz, 1H), 7.83 (d, J=8.0 Hz, 1H), 7.72 (d, J=7.0 Hz, 2H), 7.63 (t, J=8.0 Hz, 1H), 7.50 (t, J=7.5 Hz, 2H), 7.41 (t, J=8.0 Hz, 1H), 4.58 (dd, J=11.5, 3.5 Hz, 1H), 3.90 (t, J=10.5 Hz, 2H), 2.65-2.55 (m, 1H), 2.39-2.31 (m, 1H), 2.24-2.16 (m, 1H), 1.73-1.63 (m, 1H). MS (ESI) for C₂₆H₂₁ClN₅O₂ [M+H]⁺: m/z calcd, 470.14; found, 470.39.

### Example 16: Synthesis of 5-((4-([1,1′-biphenyl]-3-yl)-5-chloropyrimidin-2-yl)amino)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)nicotinamide (51)

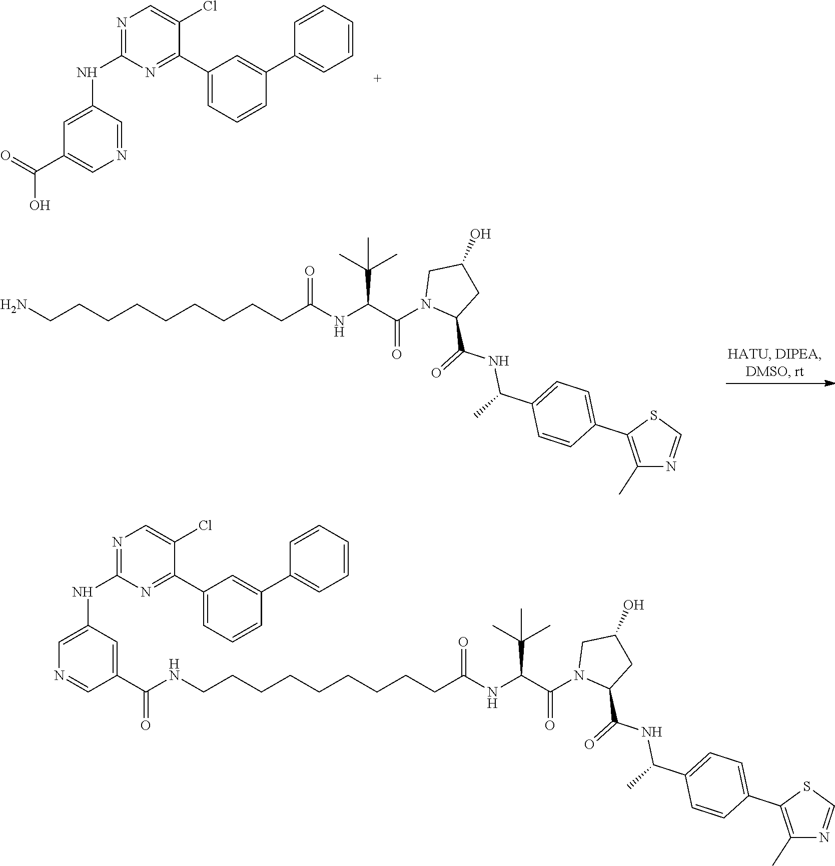 [SMILES: O=C(O)c1cncc(Nc2ncc(Cl)c(-c3cccc(-c4ccccc4)c3)n2)c1 ; Cc1ncsc1-c1ccc([C@H](C)NC(=O)[C@@H]2C[C@@H](O)CN2C(=O)[C@@H](NC(=O)CCCCCCCCCN)C(C)(C)C)cc1 ; Cc1ncsc1-c1ccc([C@H](C)NC(=O)[C@@H]2C[C@@H](O)CN2C(=O)[C@@H](NC(=O)CCCCCCCCCNC(=O)c2cncc(Nc3ncc(Cl)c(-c4cccc(-c5ccccc5)c4)n3)c2)C(C)(C)C)cc1]

### 5-((4-([1,1′-biphenyl]-3-yl)-5-chloropyrimidin-2-yl)amino)nicotinic acid

Following a similar procedure as in Example 9, the title compound was isolated as a yellow solid (40.0 mg, 7%) using 5-aminonicotinic acid. MS (ESI) for C₂₂H₁₆ClN₄O₂ [M+H]⁺: m/z calcd, 403.10; found, 403.32.

### 5-((4-([1,1′-biphenyl]-3-yl)-5-chloropyrimidin-2-yl)amino)-N-(10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)nicotinamide

DIPEA (30 pL, 0.17 mmol) and HATU (10.0 mg, 0.025 mmol) were added to a solution of 5-((4-([1,1′-biphenyl]-3-yl)-5-chloropyrimidin-2-yl)amino)nicotinic acid (7.0 mg, 0.017 mmol) and (2S,4R)-1-((S)-2-(10-aminodecanamido)-3,3-dimethylbutanoyl)-4-hydroxy-N—((S)-1-(4-(4-methylthiazol-5-yl)phenyl)ethyl)pyrrolidine-2-carboxamide (10.7 mg, 0.017 mmol) in DMSO (1.0 mL). The reaction mixture stirred at rt for 10 minutes. The reaction mixture was purified directly via prep HPLC to give the title compound as a yellow viscous oil (7.5 mg, 43%). ¹H NMR (500 MHz, DMSO-d₆) δ 10.45 (s, 1H), 9.10 (d, J=2.0 Hz, 1H), 8.99 (s, 1H), 8.77 (s, 1H), 8.72 (s, 1H), 8.65 (s, 2H), 8.36 (d, J=8.0 Hz, 1H), 8.11 (s, 1H), 7.86 (d, J=8.0 Hz, 2H), 7.79-7.73 (m, 3H), 7.66 (t, J=7.5 Hz, 1H), 7.50 (t, J=7.5 Hz, 2H), 7.45-7.40 (m, 3H), 7.38 (t, J=7.5 Hz, 2H), 4.95-4.88 (m, 1H), 4.51 (d, J=9.5 Hz, 1H), 4.42 (t, J=7.5 Hz, 1H), 4.27 (s, 1H), 3.64-3.56 (m, 2H), 3.27-3.21 (m, 2H), 2.45 (s, 3H), 2.28-2.20 (m, 1H), 2.14-2.06 (m, 1H), 2.04-1.97 (m, 1H), 1.83-1.75 (m, 1H), 1.54-1.44 (m, 4H), 1.37 (d, J=7.0 Hz, 3H), 1.31-1.20 (m, 10H), 0.93 (s, 9H). MS (ESI) for C₅₅H₆₅ClN₉O₅S [M+H]⁺: m/z calcd, 998.45; found, 998.91.

### Example 17: Synthesis of 5-((4-([1,1′-biphenyl]-3-yl)-5-chloropyrimidin-2-yl)amino)-N-(2-(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethoxy)ethyl)nicotinamide (52)

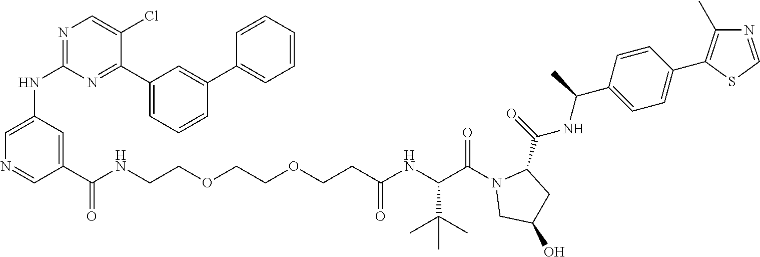 [SMILES: Cc1ncsc1-c1ccc([C@H](C)NC(=O)[C@@H]2C[C@@H](O)CN2C(=O)[C@@H](NC(=O)CCOCCOCCNC(=O)c2cncc(Nc3ncc(Cl)c(-c4cccc(-c5ccccc5)c4)n3)c2)C(C)(C)C)cc1]

Following a similar procedure as in Example 16, the title compound was isolated as a yellow viscous oil (5.8 mg, 33%) using (2S,4R)-1-((S)-2-(3-(2-(2-aminoethoxy)ethoxy)propanamido)-3,3-dimethylbutanoyl)-4-hydroxy-N—((S)-1-(4-(4-methylthiazol-5-yl)phenyl)ethyl)pyrrolidine-2-carboxamide. ¹H NMR (500 MHz, DMSO-d₆) δ 10.44 (s, 1H), 9.11 (d, J=2.5 Hz, 1H), 8.99 (s, 1H), 8.78-8.73 (m, 2H), 8.71 (s, 1H), 8.66 (s, 1H), 8.37 (d, J=8.5 Hz, 1H), 8.11 (s, 1H), 7.88-7.84 (m, 3H), 7.74 (d, J=8.0 Hz, 2H), 7.66 (t, J=8.0 Hz, 1H), 7.51 (t, J=7.5 Hz, 2H), 7.44-7.41 (m, 3H), 7.38 (t, J=8.0 Hz, 2H), 4.94-4.86 (m, 1H), 4.52 (d, J=9.5 Hz, 1H), 4.42 (t, J=8.0 Hz, 1H), 4.27 (s, 1H), 3.63-40 (m, 12H), 2.55-2.50 (m, 1H), 2.45 (s, 3H), 2.38-2.31 (m, 1H), 2.04-1.97 (m, 1H), 1.82-1.75 (m, 1H), 1.36 (d, J=6.5 Hz, 3H), 0.92 (s, 9H). MS (ESI) for C₅₂H₃₉ClN₉O₇S [M+H]⁺: m/z calcd, 988.39; found, 989.04.

### Example 18: Synthesis of N1-(5-((4-([1,1′-biphenyl]-3-yl)-5-chloropyrimidin-2-yl)amino)pyridin-3-yl)-N4-(5-(2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)succinimide (53)

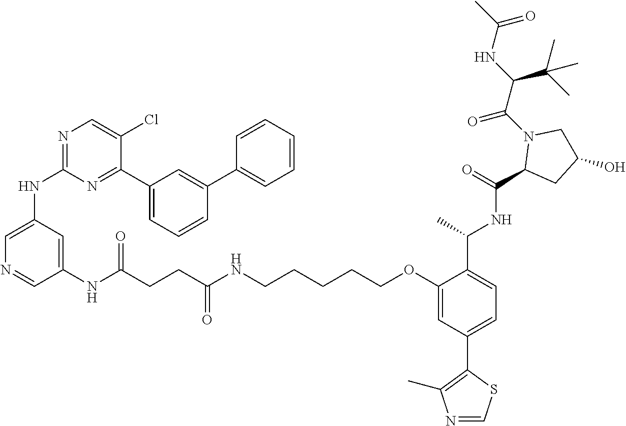 [SMILES: CC(=O)N[C@H](C(=O)N1C[C@H](O)C[C@H]1C(=O)N[C@@H](C)c1ccc(-c2scnc2C)cc1OCCCCCNC(=O)CCC(=O)Nc1cncc(Nc2ncc(Cl)c(-c3cccc(-c4ccccc4)c3)n2)c1)C(C)(C)C]

Following a similar amide coupling procedure as in Example 16, the title compound was isolated as a yellow viscous oil (3.5 mg, 31%) using 4-((5-((4-([1,1′-biphenyl]-3-yl)-5-chloropyrimidin-2-yl)amino)pyridin-3-yl)amino)-4-oxobutanoic acid and (2S,4R)-1-((S)-2-acetamido-3,3-dimethylbutanoyl)-N—((S)-1-(2-((5-aminopentyl)oxy)-4-(4-methylthiazol-5-yl)phenyl)ethyl)-4-hydroxypyrrolidine-2-carboxamide. MS (ESI) for C₅₅H₆₄ClN₁₀O₇S [M+H]⁺: m/z calcd, 1043.44; found, 1043.45.

### Example 19: Synthesis of (2S,4R)—N-(2-((8-((2-(2-((4-((4-([1,1′-biphenyl]-3-vi)-5-chloropyrimidin-2-yl)amino)phenyl)sulfonamido)ethoxy)ethyl)amino)-8-oxooctyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (54)

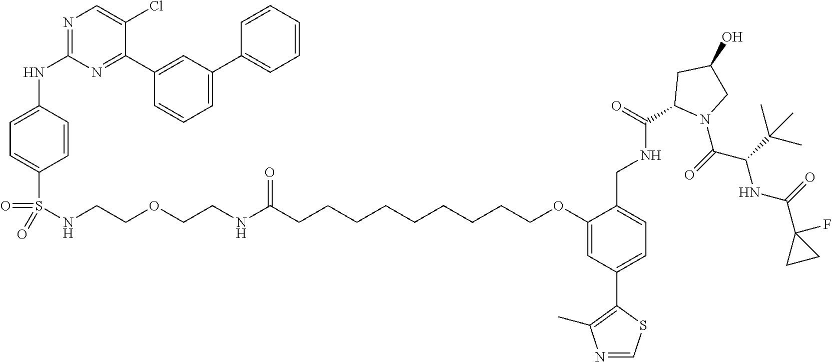 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCC(=O)NCCOCCNS(=O)(=O)c2ccc(Nc3ncc(Cl)c(-c4cccc(-c5ccccc5)c4)n3)cc2)c1]

Following a similar amide coupling procedure as in Example 16, the title compound was isolated as a yellow viscous oil (7.1 mg, 48%) using 4-((4-([1,1′-biphenyl]-3-yl)-5-chloropyrimidin-2-yl)amino)-N-(2-(2-aminoethoxy)ethyl)benzenesulfonamide and 8-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)octanoic acid. MS (ESI) for C₆₀H₇₂ClFN₉O₉S₂ [M+H]⁺: m/z calcd, 1080.46; found, 1180.45.

### Example 20: Synthesis of (2S,4R)—N-(2-((6-(2-((4-((4-([1,1′-biphenyl]-3-yl)-5-chloropyrimidin-2-yl)amino)phenyl)sulfonamido)acetamido)hexyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (55)

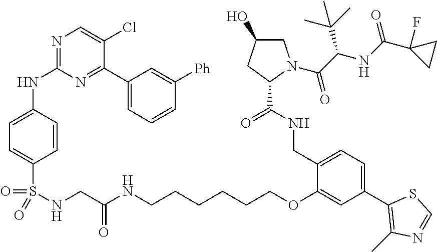 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCNC(=O)CNS(=O)(=O)c2ccc(Nc3ncc(Cl)c(-c4cccc(-c5ccccc5)c4)n3)cc2)c1]

Following a similar amide coupling procedure as in Example 16, the title compound was isolated as a yellow viscous oil (5.1 mg, 37%) using ((4-((4-([1,1′-biphenyl]-3-yl)-5-chloropyrimidin-2-yl)amino)phenyl)sulfonyl)glycine and (2S,4R)—N-(2-((6-aminohexyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide. MS (ESI) for C₅₆H₆₄ClFN₉O₈S₂ [M+H]⁺: m/z calcd, 1108.40; found, 1108.44.

### Example 21: Synthesis of (2S,4R)—N—((S)-1-(2-((5-(2-((4-((4-([1,1′-biphenyl]-3-yl-5-chloropyrimidin-2-yl)amino)phenyl)sulfonamido)acetamido)pentyl)oxy)-4-(4-methylthiazol-5-yl)phenyl)ethyl)-1-((S)-2-acetamido-3,3-dimethylbutanoyl)-4-hydroxypyrrolidine-2-carboxamide (50)

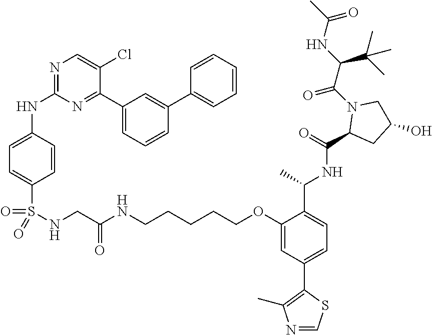 [SMILES: CC(=O)N[C@H](C(=O)N1C[C@H](O)C[C@H]1C(=O)N[C@@H](C)c1ccc(-c2scnc2C)cc1OCCCCCNC(=O)CNS(=O)(=O)c1ccc(Nc2ncc(Cl)c(-c3cccc(-c4ccccc4)c3)n2)cc1)C(C)(C)C]

Following a similar amide coupling procedure as in Example 16, the title compound was isolated as a yellow viscous oil (4.8 mg, 37%) using ((4-((4-([1,1′-biphenyl]-3-yl)-5-chloropyrimidin-2-yl)amino)phenyl)sulfonyl)glycine and (2S,4R)-1-((S)-2-acetamido-3,3-dimethylbutanoyl)-N—((S)-1-(2-((5-aminopentyl)oxy)-4-(4-methylthiazol-5-yl)phenyl)ethyl)-4-hydroxypyrrolidine-2-carboxamide. M S (ESI) for C₅₄H₆₃ClN₉O₈S₂ [M+H]⁺: m/z calcd, 1064.39; found, 1064.65.

### Example 22: Synthesis of N1-((4-((4-([1,1′-biphenyl]-3-yl)-5-chloropyrimidin-2-yl)amino)phenyl)sulfonyl)-N4-(5-2-((S)-1-((2S,4R)-1-((S)-2-acetamido-3,3-dimethylbutanoyl)-4-hydroxypyrrolidine-2-carboxamido)ethyl)-5-(4-methylthiazol-5-yl)phenoxy)pentyl)-N1-methylsuccinamide (47)

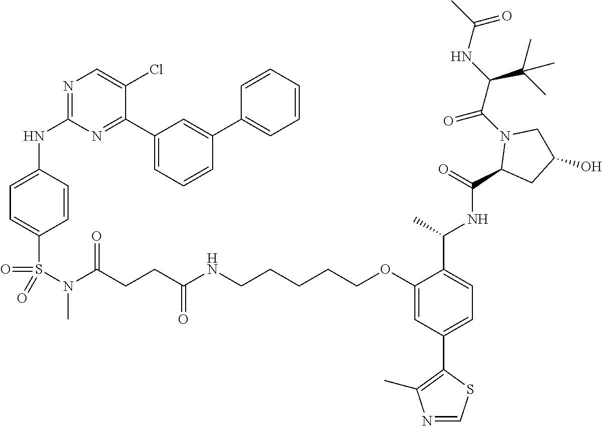 [SMILES: CC(=O)N[C@H](C(=O)N1C[C@H](O)C[C@H]1C(=O)N[C@@H](C)c1ccc(-c2scnc2C)cc1OCCCCCNC(=O)CCC(=O)N(C)S(=O)(=O)c1ccc(Nc2ncc(Cl)c(-c3cccc(-c4ccccc4)c3)n2)cc1)C(C)(C)C]

Following a similar amide coupling procedure as in Example 16, the title compound was isolated as a yellow viscous oil (4.6 mg, 64%) using 4-((4-((4-([1,1′-biphenyl]-3-yl)-5-chloropyrimidin-2-yl)amino)-N-methylphenyl)sulfonamido)-4-oxobutanoic acid and (2S,4R)-1-((S)-2-acetamido-3,3-dimethylbutanoyl)-N—((S)-1-(2-((5-aminopentyl)oxy)-4-(4-methylthiazol-5-yl)phenyl)ethyl)-4-hydroxypyrrolidine-2-carboxamide. MS (ESI) for C₅₇H₆₇ClN₉O₉S₂ [M+H]⁺: m/z calcd, 1120.42; found, 965.40 (−155).

### Example 23: Synthesis of N-(2-(2-(2-((4-((4-([1,1′-biphenyl]-3-yl)-5-chloro-1|4-pyrimidin-2-yl)amino)phenyl)sulfonamido)ethoxy)ethoxy)ethyl)-2-((2-(2,6-dioxopiperidin-3-yl)-1,3-dioxoisoindolin-4-yl)oxy)acetamide (36)

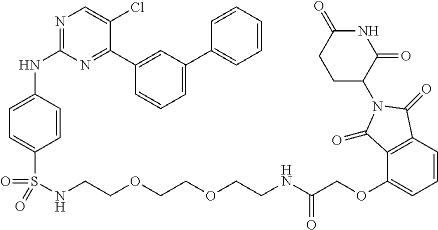 [SMILES: O=C(COc1cccc2c1C(=O)N(C1CCC(=O)NC1=O)C2=O)NCCOCCOCCNS(=O)(=O)c1ccc(Nc2ncc(Cl)c(-c3cccc(-c4ccccc4)c3)n2)cc1]

Following a similar amide coupling procedure as in Example 16, the title compound was isolated as a yellow viscous oil (2.1 mg, 13%) using 4-((4-([1,1′-biphenyl]-3-yl)-5-chloropyrimidin-2-yl)amino)-N-(2-(2-(2-aminoethoxy)ethoxy)ethyl)benzenesulfonamide and 2-((2-(2,6-dioxopiperidin-3-yl)-1,3-dioxoisoindolin-4-yl)oxy)acetic acid. MS (ESI) for C₄₃H₄₁ClN₇O₁₀S [M+H]⁺: m/z calcd, 882.23; found, 882.18.

### Example 24: Synthesis of N-(2-(2-(2-(2-((4-((4([1,1′-biphenyl]-3-yl)-5-chloropyrimidin-2-yl)amino)phenyl)sulfonamido)ethoxy)ethoxy)ethoxy)ethyl)-2-((2-(2,6-dioxopiperidin-3-yl)-1,3-dioxoisoindolin-4-yl oxy)acetamide (37)

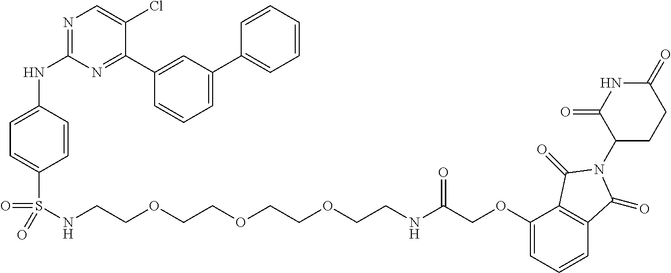 [SMILES: O=C(COc1cccc2c1C(=O)N(C1CCC(=O)NC1=O)C2=O)NCCOCCOCCOCCNS(=O)(=O)c1ccc(Nc2ncc(Cl)c(-c3cccc(-c4ccccc4)c3)n2)cc1]

Following a similar amide coupling procedure as in Example 16, the title compound was isolated as a yellow viscous oil (3.3 mg, 16%) using 4-((4-([1,1′-biphenyl]-3-yl)-5-chloropyrimidin-2-yl)amino)-N-(2-(2-(2-(2-aminoethoxy)ethoxy)ethoxy)ethyl)benzenesulfonamide and 2-((2-(2,6-dioxopiperidin-3-yl)-1,3-dioxoisoindolin-4-yl)oxy)acetic acid. MS (ESI) for C₄₅H₄₅ClN₇O₁₁S [M+H]⁺: m/z calcd, 926.26; found, 926.30.

### Example 25: Synthesis of (2S,4R)—N-(2-((7-(2-(5-((4-([1,1′-biphenyl]-3-yl)-5-chloropyrimidin-2-yl)amino)pyridin-3-yl)-1-oxo-2,8-diazaspiro[4,5]decan-8-yl)-7-oxoheptyl)oxy)-4-(4-methylthiazol-5-yl)benzyl)-1-((S)-2(1-fluorocyclopropane-1-carboxamido)-3,3-dimethylbutanoyl)-4-hydroxypyrrolidine-2-carboxamide (56)

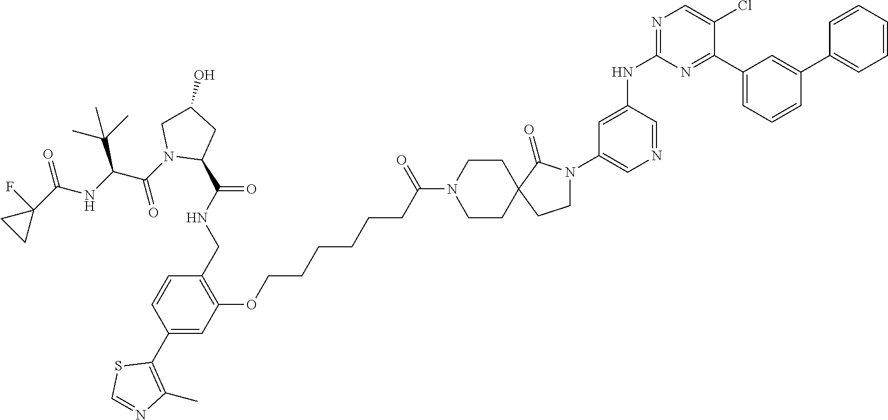 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCC3(CC2)CCN(c2cncc(Nc4ncc(Cl)c(-c5cccc(-c6ccccc6)c5)n4)c2)C3=O)c1]

Following a similar amide coupling procedure as in Example 16, the title compound was isolated as a white solid (7.6 mg, 54%). ¹H NMR (500 MHz, DMSO-d₆) δ 10.59 (s, 1H), 8.98 (s, 1H), 8.90 (s, 1H), 8.86-8.78 (m, 3H), 8.49 (t, J=6.0 Hz, 1H), 8.16 (s, 1H), 7.88 (t, J=7.5 Hz, 2H), 7.76 (d, J=7.5 Hz, 2H), 7.66 (t, J=8.0 Hz, 1H), 7.51 (t, J=7.5 Hz, 2H), 7.45-7.39 (m, 2H), 7.28 (d, J=8.5 Hz, 1H), 7.00 (s, 1H), 6.95 (d, J=7.5 Hz, 1H), 4.59 (d, J=9.5 Hz, 1H), 4.52 (t, J=8.0 Hz, 1H), 4.37-4.27 (m, 2H), 4.24-4.17 (m, 2H), 4.05 (t, J=6.0 Hz, 2H), 3.85-3.70 (m, 3H), 3.67-3.57 (m, 2H), 3.18-3.10 (m, 1H), 2.85-2.75 (m, 1H), 2.45 (s, 3H), 2.34 (t, J=7.0 Hz, 2H), 2.11-2.05 (m, 1H), 1.98-1.88 (m, 3H), 1.80-1.72 (m, 2H), 1.68-1.60 (m, 1H), 1.58-1.16 (m, 13H), 0.95 (s, 9H). MS (ESI) for C₆₂H₇₁ClFN₁₀O₇S [M+H]⁺: m/z calcd, 1153.49; found, 1153.50.

### Example 26: Synthesis of ((2S,4R)-1-((S)-2-(7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptanamido)-3,3-dimethylbutanoyl)-4-hydroxy-N—((S)-1-(4-(4-methylthiazol-5-yl)phenylethyl)pyrrolidine-2-carboxamide (57)

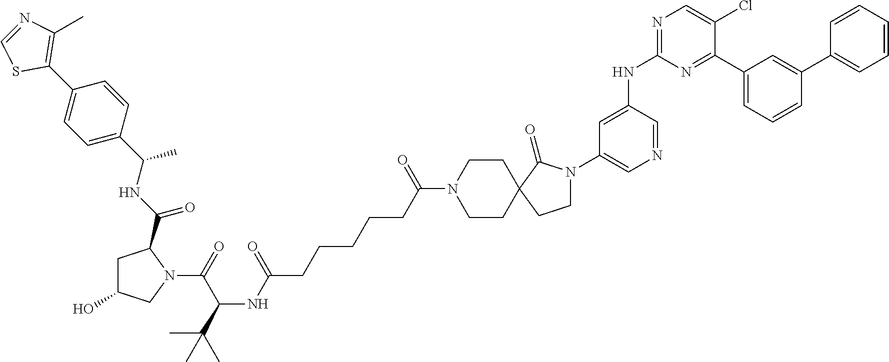 [SMILES: Cc1ncsc1-c1ccc([C@H](C)NC(=O)[C@@H]2C[C@@H](O)CN2C(=O)[C@@H](NC(=O)CCCCCC(=O)N2CCC3(CC2)CCN(c2cncc(Nc4ncc(Cl)c(-c5cccc(-c6ccccc6)c5)n4)c2)C3=O)C(C)(C)C)cc1]

Following a similar amide coupling procedure as in Example 16, the title compound was isolated as a white solid (3.0 mg, 27%). ¹H NMR (500 MHz, DMSO-d₆) δ 10.65 (s, 1H), 8.99 (s, 1H), 8.92 (s, 1H), 8.88-8.78 (m, 3H), 8.36 (d, J=7.5 Hz, 1H), 8.16 (s, 1H), 7.89 (t, J=7.5 Hz, 2H), 7.79 (d, J=9.0 Hz, 1H), 7.76 (d, J=8.0 Hz, 2H), 7.66 (t, J=8.0 Hz, 1H), 7.52 (t, J=8.0 Hz, 2H), 7.45-7.40 (m, 3H), 7.38 (d, J=8.0 Hz, 2H), 4.95-4.86 (m, 1H), 4.52 (d, J=9.0 Hz, 1H), 4.42 (t, J=8.0 Hz, 1H), 4.28 (s, 1H), 4.24-4.15 (m, 1H), 3.84-3.70 (m, 3H), 3.64-3.57 (m, 2H), 3.18-3.10 (m, 1H), 2.86-2.76 (m, 1H), 2.45 (s, 3H), 2.34-2.22 (m, 3H), 2.16-2.09 (m, 1H), 2.04-1.92 (m, 3H), 1.83-1.76 (m, 1H), 1.69-1.60 (m, 1H), 1.56-1.21 (m, 9H), 1.37 (d, J=7.0 Hz, 3H), 0.94 (s, 9H). MS (ESI) for C₅₉H₆₈ClN₁₀O₆S [M+H]⁺: m/z calcd, 1079.47; found, 1079.55.

### Example 27; Inhibition Activity

TABLE 1
Inhibitory activity of inventive compounds

| Compound | PIP4K2C | PIP4K2B | PIP4K2A |
|---|---|---|---|
| # | K_{d} (nM) | K_{d} (nM) | IC₅₀ (nM) |
| 1 | 36 | >10,000 | >10,000 |
| 2 | 56 | >10,000 | >10,000 |
| 3 | 29 | >10,000 | 8,760 |
| 4 | 64 | >10,000 | >10,000 |
| 5 | 46 | — | — |
| 6 | 43 | — | — |
| 7 | 150 | — | — |
| 8 | 270 | — | — |
| 9 | 1,400 | >10,000 | — |
| 10 | 65 | — | — |
| 11 | 7.2 | >10,000 | — |
| 12 | 140 | >10,000 | — |
| 13 | 21 | >10,000 | — |
| 14 | 38 | >10,000 | — |
| 15 | 0.45 | >10,000 | — |
| 36 | 460 | — | — |
| 53 | 110 | — | — |
| 56 | 620 | >10,000 | — |
| 57 | 42 | >10,000 | — |

The inhibitory activity of the compounds were evaluated using KINOMEscan® PIP5K2B (also known as PIP4K2B) and KINOMEscan® PIP5K2C (also known as PIP4k2C) Assays (Eurofins Discovery) following the manufacturer's standards. The data shown in Table 1 demonstrate that the inventive compounds tested selectively inhibited PIP4K2C activity.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound represented by formula (I): 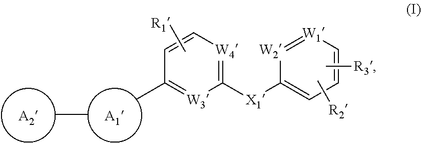
or a pharmaceutically acceptable salt or stereoisomer thereof, wherein:
X₁′ is a bond, CH₂, CHMe, NH, NMe, or O;
W₁′ is CR₄′ or N;
W₂′ is CR₄′ or N;
each R₄′ is independently hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, C(O)NH₂, C(O)NH(C₁-C₆) alkyl, NHC(O)(C₁-C₆) alkyl, optionally substituted C₅-C₆ carbocyclyl or optionally substituted 5- or 6-membered heterocyclyl;
W₃′ is CH or N;
W₄′ is CH or N;
R₁′ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;
R₂′ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl or optionally substituted 5- or 6-membered heterocyclyl, or
R₂′ and R₃′, together with the carbon atoms to which they are attached, form an optionally substituted 5- to 10-membered heterocyclyl;
R₃′ is hydrogen, (C₁-C₆) alkoxy, NH(C₁-C₆) alkyl, SO₂NH₂, SO₂NH(C₁-C₆) alkyl, SO₂N((C₁-C₆) alkyl)₂, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, NHC(O)aryl, or NHC(O)NH(C₁-C₆) alkyl, wherein said (C₁-C₆) alkyl group of R₃′ is optionally substituted; and
A₁′ and A₂′ are each independently optionally substituted aryl or optionally substituted 5- to 6-membered heteroaryl.

2. The compound of claim 1, wherein X₁′ is NH.

3. The compound of claim 1, wherein W₄′ is N.

4. The compound of claim 1, wherein W₃′ is N.

5. The compound of claim 1, wherein R₁′ is halogen.

6. The compound of claim 5, wherein R₁′ is chloro.

7. The compound of claim 1, wherein R₁′ is CN.

8. The compound of claim 1, wherein W₁′ is CR₄′.

9. The compound of claim 8, wherein R₄′ is hydrogen or methyl.

10. The compound of claim 1, wherein W₁′ is N.

11. The compound of claim 1, wherein W₂′ is CR₄′.

12. The compound of claim 11, wherein R₄′ is C(O)NH₂, or NHC(O)Me.

13. The compound of claim 1, wherein W₂′ is N.

14. The compound of claim 1, wherein R₂′ is hydrogen, methyl, halogen, OMe, OCF₃, NH₂, NHMe, NMe₂, or an optionally substituted 5- or 6-membered heterocyclyl, containing 1 or 2 heteroatoms selected from N, O, and S.

15. The compound of claim 1, wherein R₃′ SO₂NH₂, SO₂NH(C₁-C₆) alkyl, SO₂N((C₁-C₆) alkyl)C(O)(C₁-C₆) alkyl, NHSO₂(C₁-C₆) alkyl, C(O)NH₂, C(O)NH(C₁-C₆) alkyl, NHC(O)(C₁-C₆) alkyl, or NHC(O)aryl, wherein said alkyl group is optionally substituted.

16. The compound of claim 1, wherein A₁′ is phenyl.

17. The compound of claim 1, wherein A₁′ is 5- to 6-membered heteroaryl, containing 1 to 3 heteroatoms selected from N, O, and S.

18. The compound of claim 17, wherein A₁′ is optionally substituted pyrrole, imidazole, pyrazole, thiazole, triazole, or pyridine.

19. The compound of claim 1, wherein A₂′ is optionally substituted phenyl.

20. The compound of claim 1, wherein A₂′ is optionally substituted 5- to 6-membered heteroaryl, containing 1 to 3 heteroatoms selected from N, O, and S.

21. The compound of claim 20, wherein A₂′ is optionally substituted thiophene, pyrrole, pyrimidine, or pyridazine.

22. The compound of claim 1, wherein the compound of formula (I) is represented by any one of Formulas Ia-Ii:

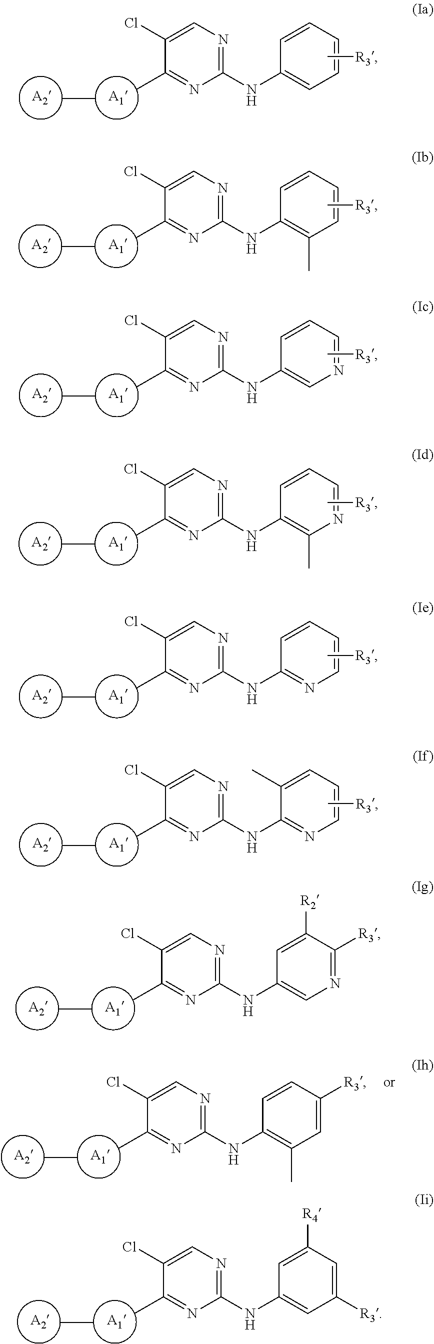

23. The compound of claim 1, which is: 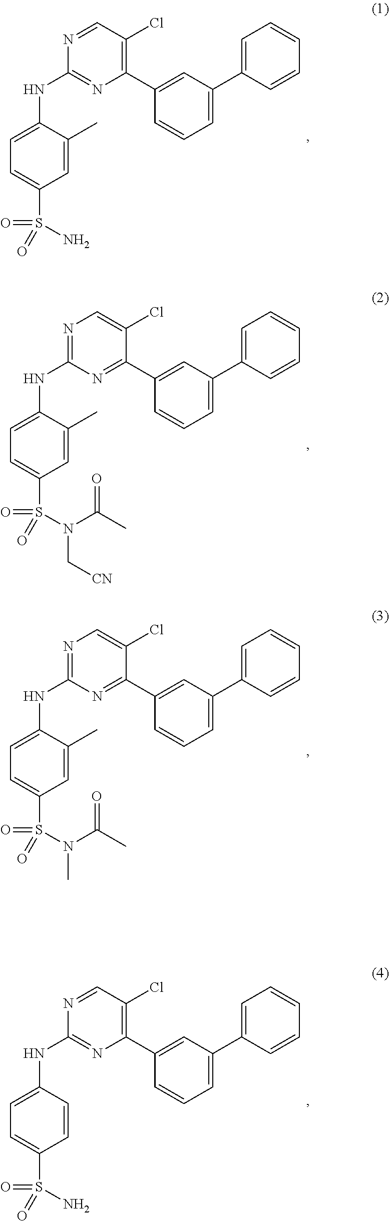 [SMILES: Cc1cc(S(N)(=O)=O)ccc1Nc1ncc(Cl)c(-c2cccc(-c3ccccc3)c2)n1 ; CC(=O)N(CC#N)S(=O)(=O)c1ccc(Nc2ncc(Cl)c(-c3cccc(-c4ccccc4)c3)n2)c(C)c1 ; CC(=O)N(C)S(=O)(=O)c1ccc(Nc2ncc(Cl)c(-c3cccc(-c4ccccc4)c3)n2)c(C)c1 ; NS(=O)(=O)c1ccc(Nc2ncc(Cl)c(-c3cccc(-c4ccccc4)c3)n2)cc1] 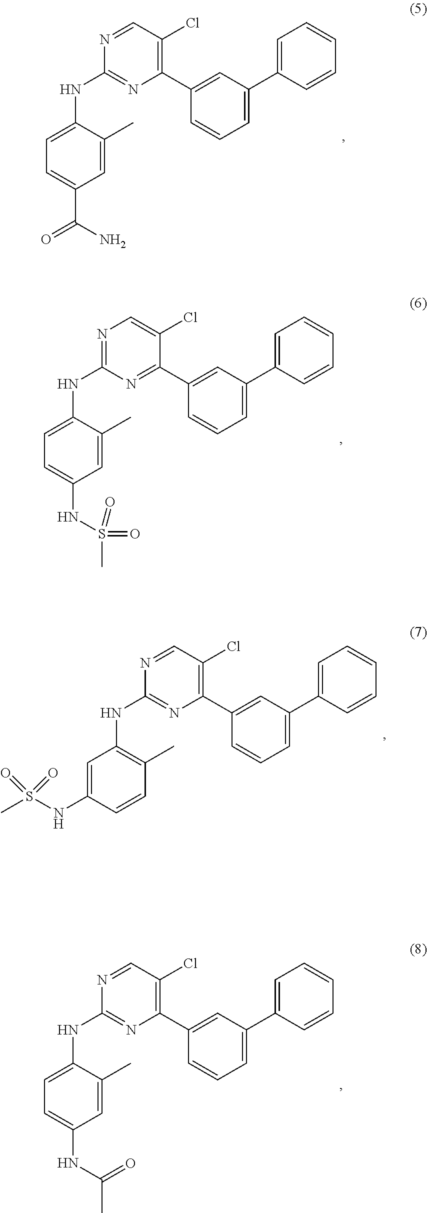 [SMILES: Cc1cc(C(N)=O)ccc1Nc1ncc(Cl)c(-c2cccc(-c3ccccc3)c2)n1 ; Cc1cc(NS(C)(=O)=O)ccc1Nc1ncc(Cl)c(-c2cccc(-c3ccccc3)c2)n1 ; Cc1ccc(NS(C)(=O)=O)cc1Nc1ncc(Cl)c(-c2cccc(-c3ccccc3)c2)n1 ; CC(=O)Nc1ccc(Nc2ncc(Cl)c(-c3cccc(-c4ccccc4)c3)n2)c(C)c1] 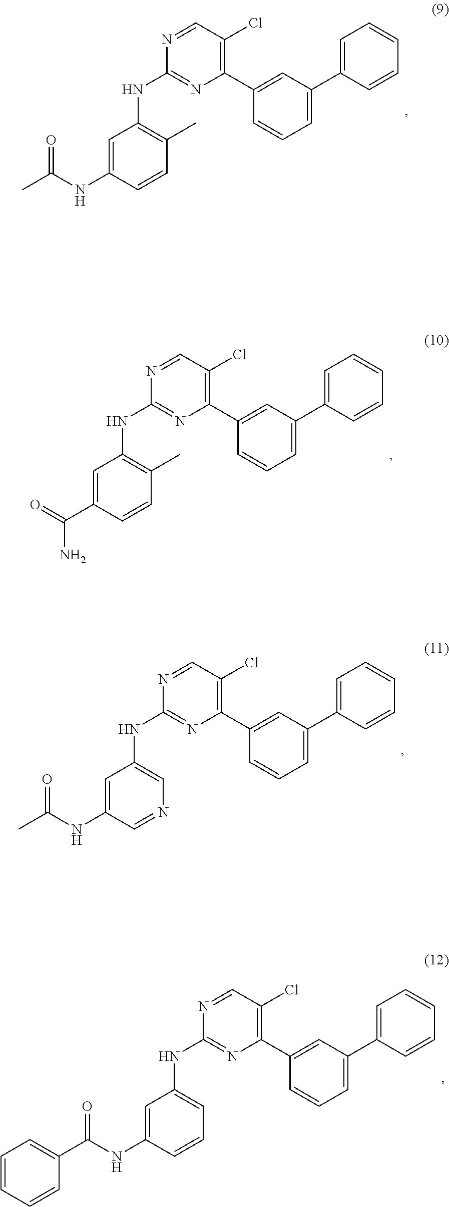 [SMILES: CC(=O)Nc1ccc(C)c(Nc2ncc(Cl)c(-c3cccc(-c4ccccc4)c3)n2)c1 ; Cc1ccc(C(N)=O)cc1Nc1ncc(Cl)c(-c2cccc(-c3ccccc3)c2)n1 ; CC(=O)Nc1cncc(Nc2ncc(Cl)c(-c3cccc(-c4ccccc4)c3)n2)c1 ; O=C(Nc1cccc(Nc2ncc(Cl)c(-c3cccc(-c4ccccc4)c3)n2)c1)c1ccccc1] 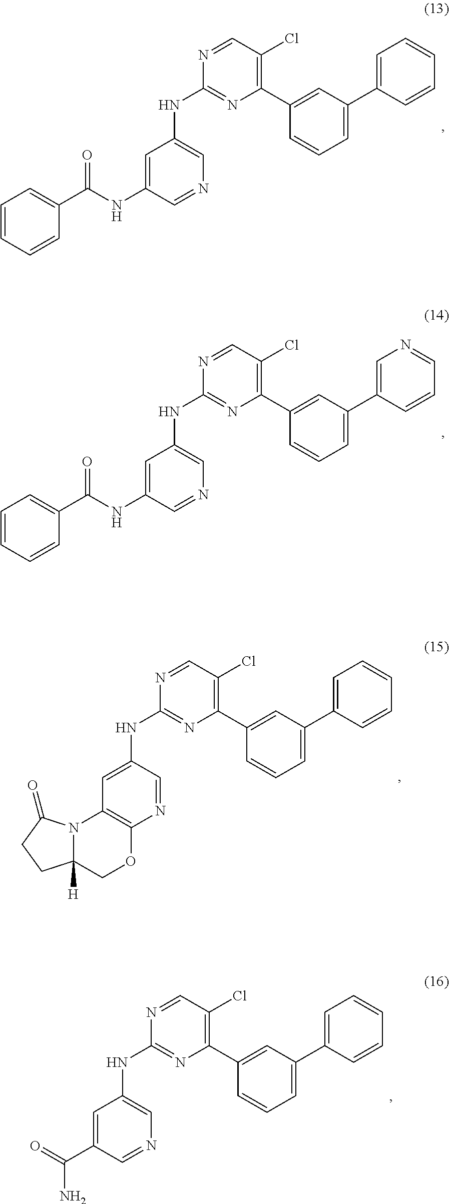 [SMILES: O=C(Nc1cncc(Nc2ncc(Cl)c(-c3cccc(-c4ccccc4)c3)n2)c1)c1ccccc1 ; O=C(Nc1cncc(Nc2ncc(Cl)c(-c3cccc(-c4cccnc4)c3)n2)c1)c1ccccc1 ; O=C1CC[C@H]2COc3ncc(Nc4ncc(Cl)c(-c5cccc(-c6ccccc6)c5)n4)cc3N12 ; NC(=O)c1cncc(Nc2ncc(Cl)c(-c3cccc(-c4ccccc4)c3)n2)c1] 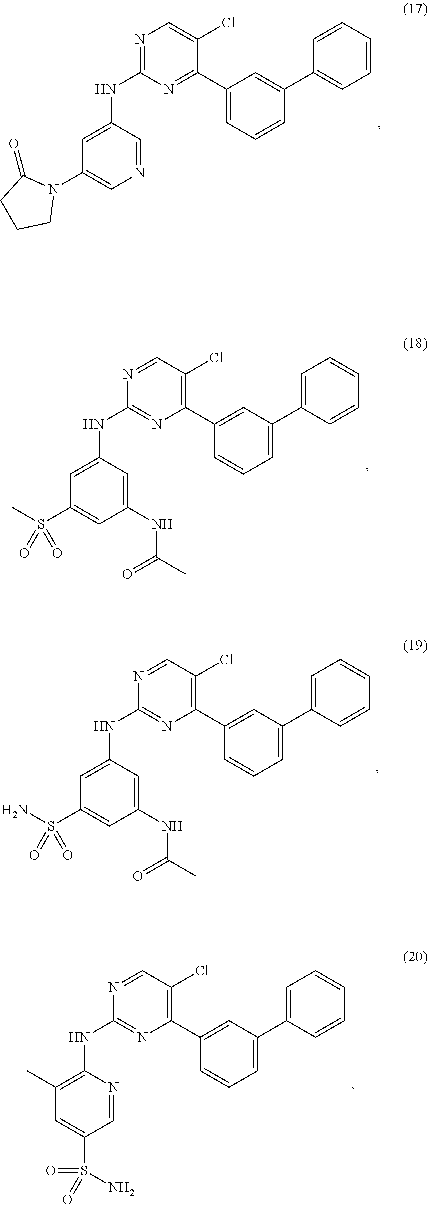 [SMILES: O=C1CCCN1c1cncc(Nc2ncc(Cl)c(-c3cccc(-c4ccccc4)c3)n2)c1 ; CC(=O)Nc1cc(Nc2ncc(Cl)c(-c3cccc(-c4ccccc4)c3)n2)cc(S(C)(=O)=O)c1 ; CC(=O)Nc1cc(Nc2ncc(Cl)c(-c3cccc(-c4ccccc4)c3)n2)cc(S(N)(=O)=O)c1 ; Cc1cc(S(N)(=O)=O)cnc1Nc1ncc(Cl)c(-c2cccc(-c3ccccc3)c2)n1] 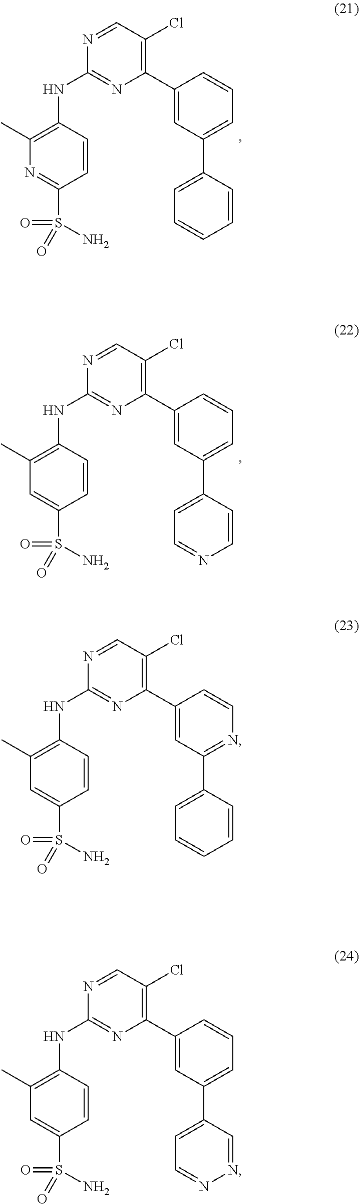 [SMILES: Cc1nc(S(N)(=O)=O)ccc1Nc1ncc(Cl)c(-c2cccc(-c3ccccc3)c2)n1 ; Cc1cc(S(N)(=O)=O)ccc1Nc1ncc(Cl)c(-c2cccc(-c3ccncc3)c2)n1 ; Cc1cc(S(N)(=O)=O)ccc1Nc1ncc(Cl)c(-c2ccnc(-c3ccccc3)c2)n1 ; Cc1cc(S(N)(=O)=O)ccc1Nc1ncc(Cl)c(-c2cccc(-c3ccnnc3)c2)n1] 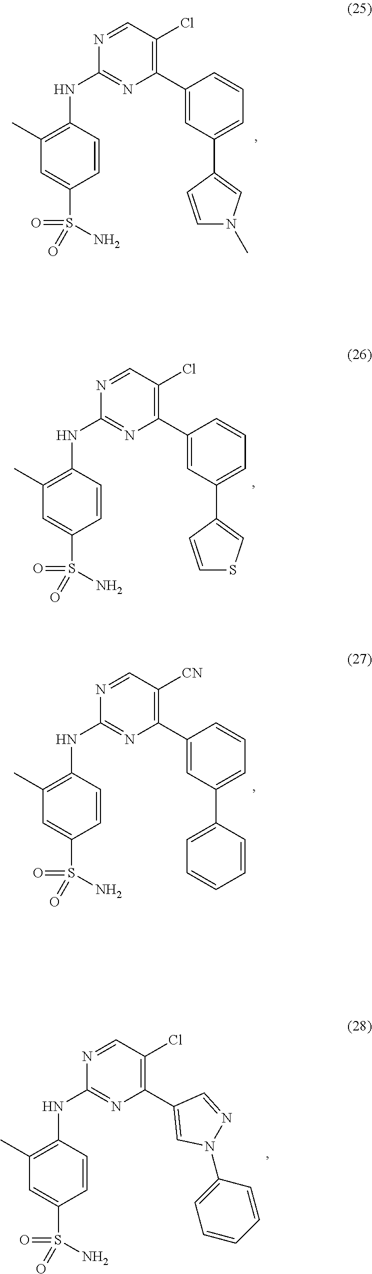 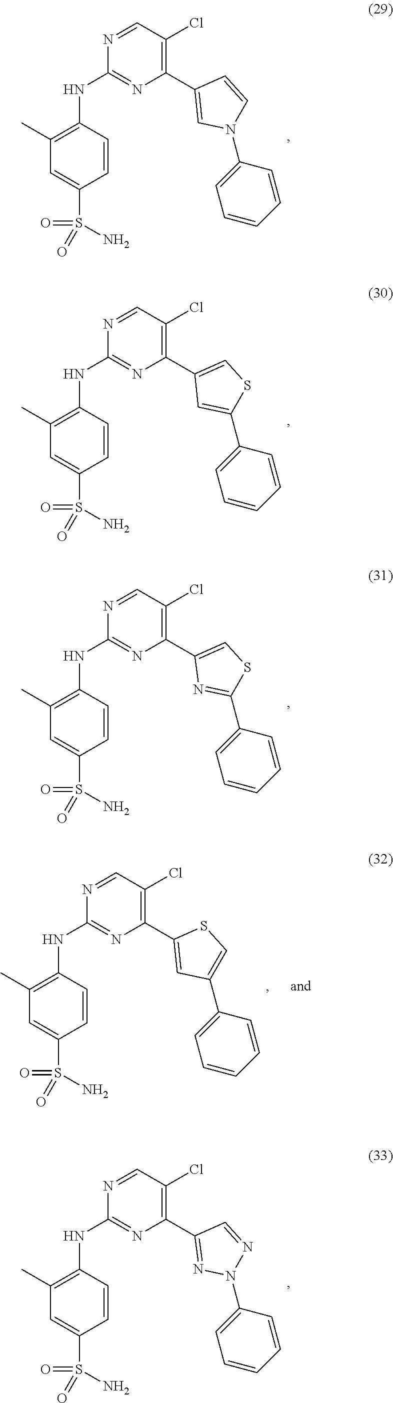 [SMILES: Cc1cc(S(N)(=O)=O)ccc1Nc1ncc(Cl)c(-c2ccn(-c3ccccc3)c2)n1 ; Cc1cc(S(N)(=O)=O)ccc1Nc1ncc(Cl)c(-c2csc(-c3ccccc3)c2)n1 ; Cc1cc(S(N)(=O)=O)ccc1Nc1ncc(Cl)c(-c2csc(-c3ccccc3)n2)n1 ; Cc1cc(S(N)(=O)=O)ccc1Nc1ncc(Cl)c(-c2cc(-c3ccccc3)cs2)n1 ; Cc1cc(S(N)(=O)=O)ccc1Nc1ncc(Cl)c(-c2cnn(-c3ccccc3)n2)n1]
or a pharmaceutically acceptable salt or stereoisomer thereof.

24. A pharmaceutical composition, comprising a therapeutically effective amount of the compound or pharmaceutically acceptable salt or stereoisomer thereof of claim 1, and a pharmaceutically acceptable carrier.

25. A method of treating a disease or disorder by modulating the level or activity of at least one of PIP4K2A, PIP4K2B, and PIP4K2C, comprising administering to a subject in need thereof a therapeutically effective amount of the compound or pharmaceutically acceptable salt or stereoisomer thereof of claim 1, or
a method of modulating immune functions in a subject in need thereof, comprising administering to a subject a therapeutically effective amount of the compound or pharmaceutically acceptable salt or stereoisomer thereof of claim 1, or
a method of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of the compound or pharmaceutically acceptable salt or stereoisomer thereof of claim 1.

26.-34. (canceled)

35. A bifunctional compound, comprising a targeting ligand that binds phosphatidylinositol-5-phosphate 4-kinase type 2 gamma (PIP4K2C) and a degron covalently attached to the targeting ligand by a linker, wherein the bifunctional compound has a structure represented by formula (II): 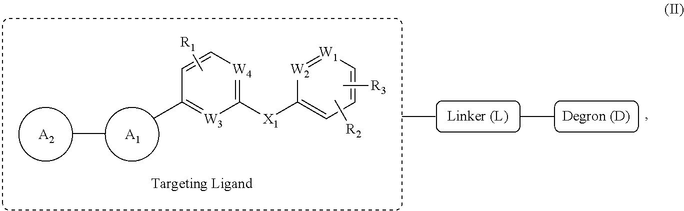
or a pharmaceutically acceptable salt or stereoisomer thereof, wherein:
X₁ is a bond, CH₂, CHMe, NH, NMe, or 0;
W₁ is CR₄ or N;
W₂ is CR₄ or N;
each R₄ is independently hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl or optionally substituted 5- or 6-membered heterocyclyl;
W₃ is CH or N;
W₄ is CH or N;
R₁ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, NH₂, OH, or CN;
R₂ is hydrogen, optionally substituted (C₁-C₆) alkyl, optionally substituted (C₁-C₆) haloalkyl, optionally substituted (C₁-C₆) alkoxy, optionally substituted (C₁-C₆) haloalkoxy, halogen, NO₂, OH, CN, optionally substituted amino, optionally substituted C₅-C₆ carbocyclyl, optionally substituted aryl, optionally substituted 5- or 6-membered heterocyclyl, or optionally substituted heteroaryl;
R₃ is (C₁-C₆) alkoxy, NH(C₁-C₆) alkyl, NHaryl, NH-5- or 6-membered heteroaryl, NHC(O)aryl, NHC(O)-5- or 6-membered heteroaryl, NHC(O)(C₁-C₆) alkyl, NHSO₂(C₁-C₆) alkyl, NHSO₂aryl, SO₂NH₂, SO₂NH(C₁-C₆) alkyl, SO₂NHaryl, C(O)NH₂, C(O)NH(C₁-C₆) alkyl, C(O)NHaryl, C(O)NH-5- or 6-membered heteroaryl, or optionally substituted 5- to 10-membered heterocyclyl, or R₃ is O, NH, N(C₁-C₆) alkyl, SO₂NH, SO₂N(C₁-C₆) alkyl, C(O)NH, or optionally substituted 5- to 10-membered heterocyclyl, when R₃ is bound to the Linker; and
A₁ and A₂ are each independently optionally substituted aryl or optionally substituted 5- to 6-membered heteroaryl; and
the degron is a moiety that binds an E3 ubiquitin ligase.

36. The bifunctional compound of claim 35, wherein formula (II) is of formula IIa: 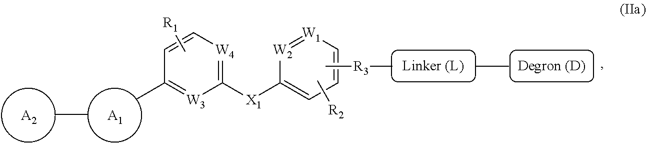
or a pharmaceutically acceptable salt or stereoisomer thereof.

37. The bifunctional compound of claim 35, wherein the targeting ligand is of Formula TL-1a, TL-1b, TL-1c, TL-1d, TL-1e, or TL-1f:

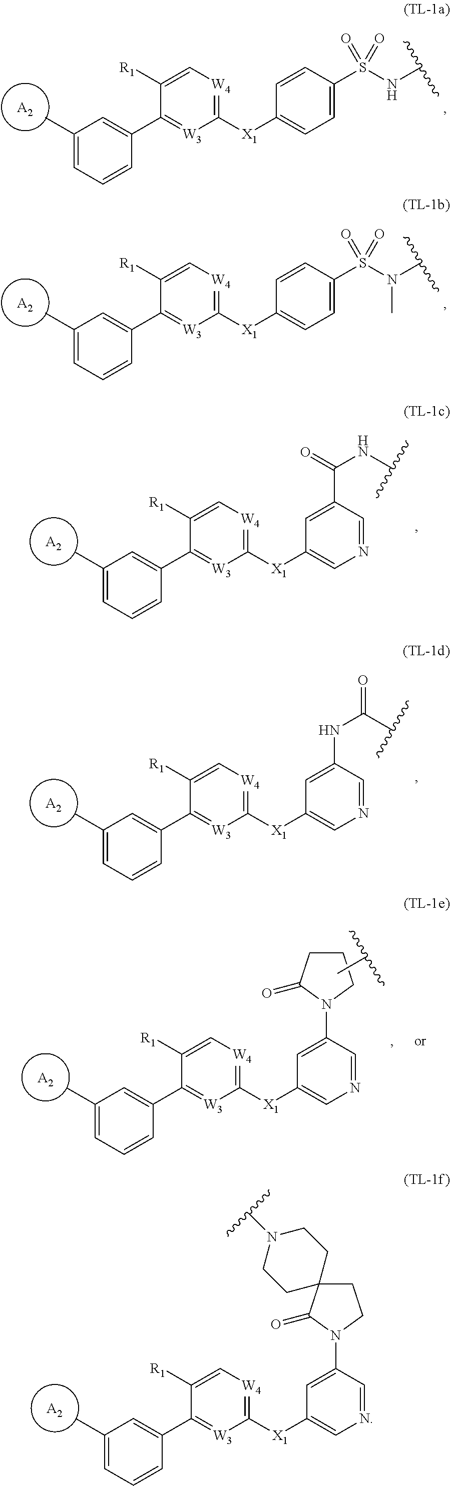

38. The bifunctional compound of claim 35, wherein X₁ is NH.

39. The bifunctional compound of claim 35, wherein W₄ is N.

40. The bifunctional compound of claim 35, wherein W₃ is N.

41. The bifunctional compound of claim 35, wherein R₁ is halogen.

42. The bifunctional compound of claim 41, wherein R₁ is chloro.

43. The bifunctional compound of claim 35, wherein A₂ is phenyl.

44. The bifunctional compound of claim 35, wherein the targeting ligand is of Formula TL-1a1, TL-1b1, TL-1c1, TL-1d1, TL-1e1, or TL-1f1:

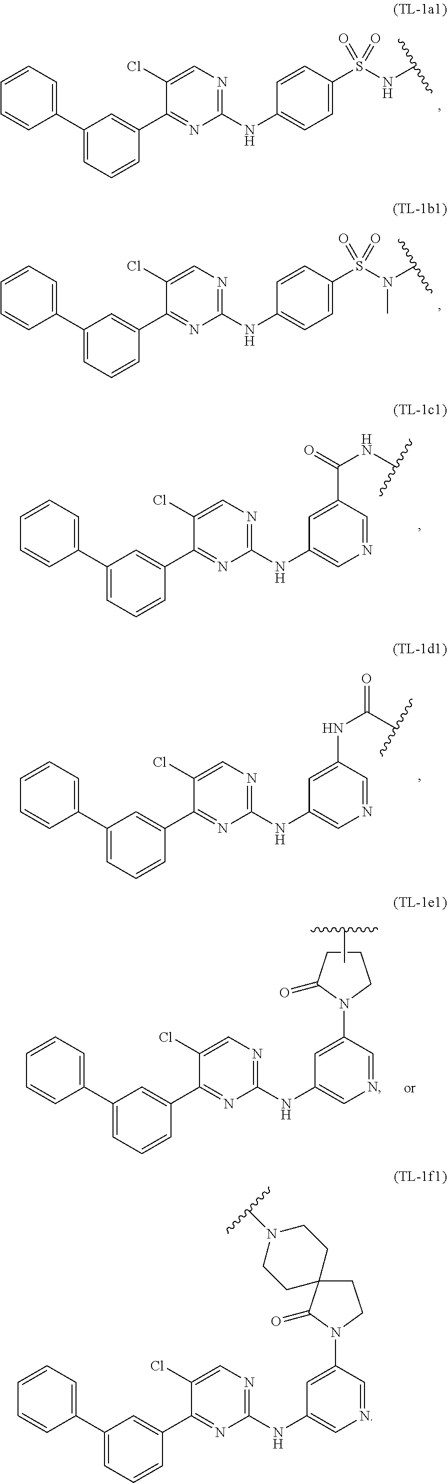

45. The bifunctional compound of claim 35, wherein the linker comprises a single alkylene chain or a bivalent alkylene chain, either of which may be interrupted by, and/or terminate at either or both termini in a —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂₀—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, or 5- to 12-membered heteroarylene, or any combination thereof, wherein R′ is H or C₁-C₆ alkyl, wherein the interrupting and the one or both terminating groups may be the same or different.

46. The bifunctional compound of claim 45, wherein the alkylene chain is interrupted by, and/or terminates (at either or both termini) with —O—, 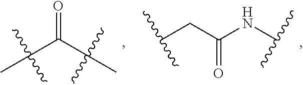
or a combination thereof.

47. The bifunctional compound of claim 46, wherein the alkylene chain has 3-12 alkylene units.

48. The bifunctional compound of claim 35, wherein the linker comprises a single polyethylene glycol chain which may be interrupted by, and/or terminates at either or both termini in a —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂₀—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

49. The bifunctional compound of claim 48, wherein the linker terminates at either or both termini with 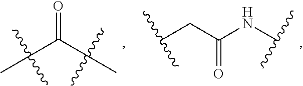
or a combination thereof.

50. The bifunctional compound of claim 48, wherein the polyethylene glycol chain has 1-6 PEG units.

51. The bifunctional compound of claim 35, wherein the linker is represented by any one of structures L10a-L10-v:

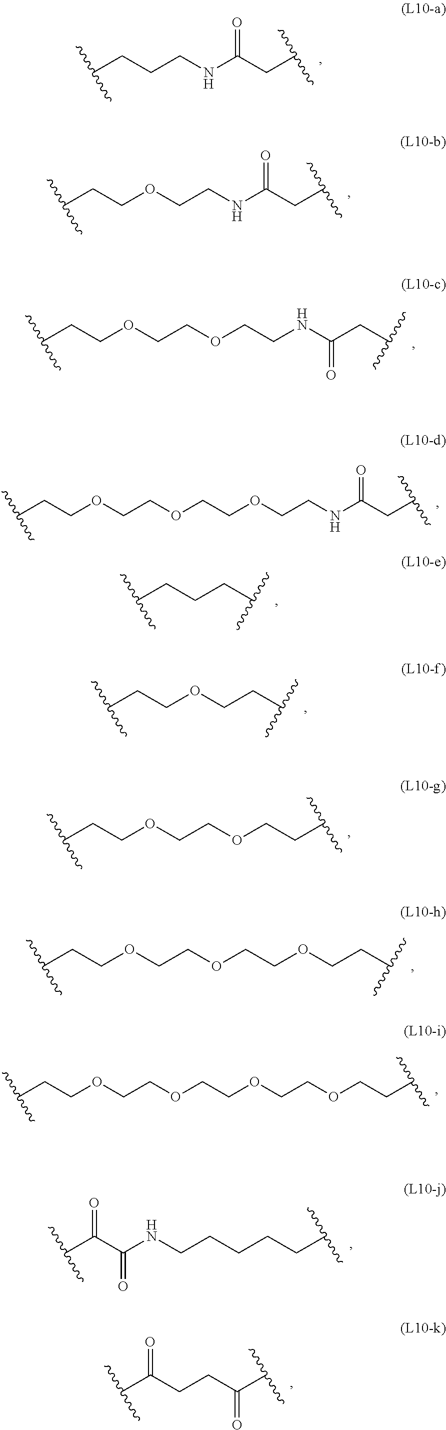

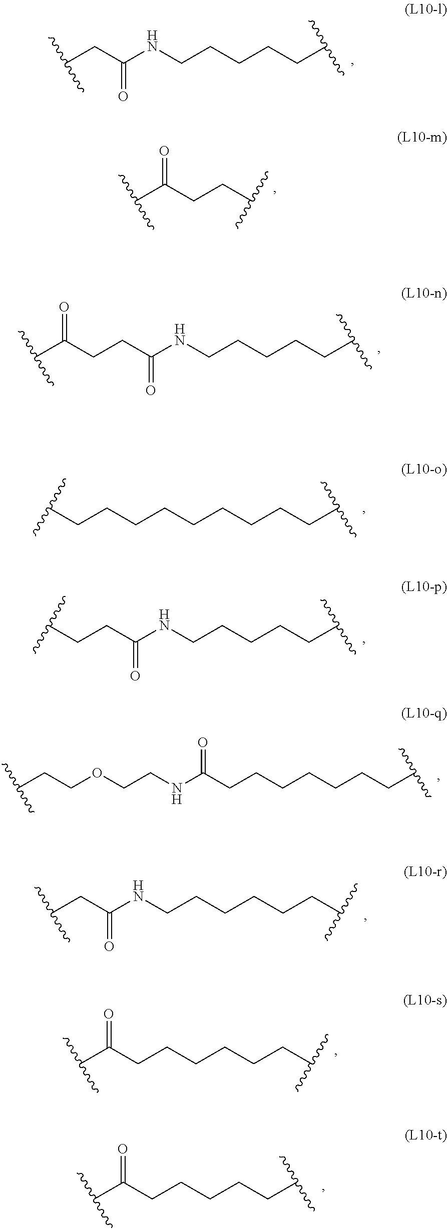

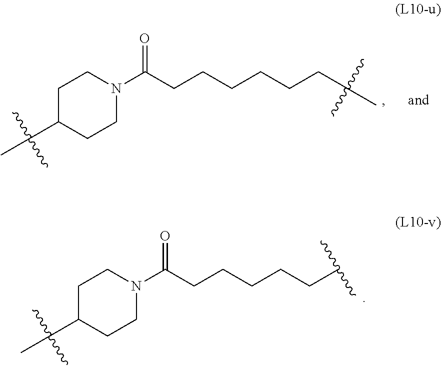 [SMILES: *CCCCCCC(=O)N1CCC(*)CC1 ; *CCCCCC(=O)N1CCC(*)CC1]

52. The bifunctional compound of claim 35, wherein the degron binds cereblon (CRBN).

53. The bifunctional compound of claim 52, wherein the degron is represented b an one of structures (D1-a) to D1-d): 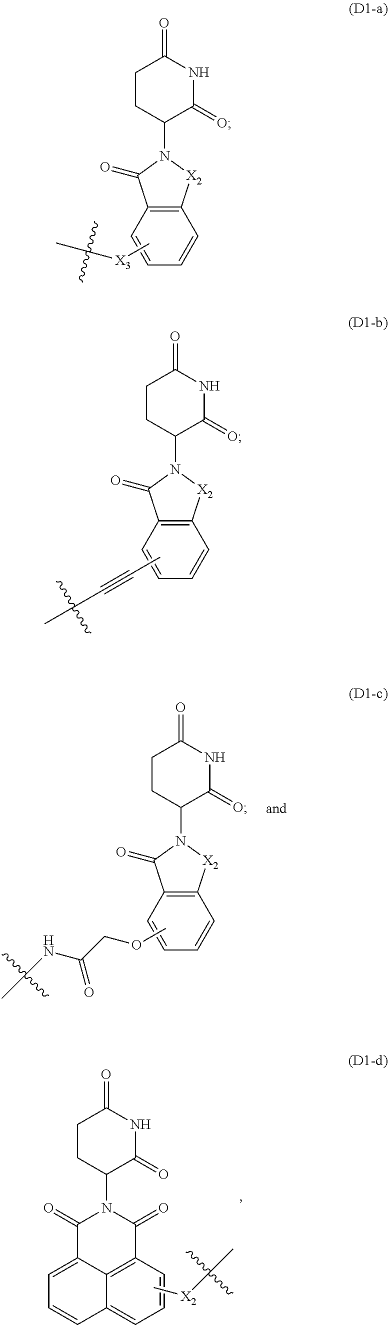
wherein X₂is CH₂ or C(O) and X₃ is CR″₁R″₂, NR″₁R″₂, NH, O, or S, wherein R″₁ and R″₂ are independently hydrogen, halogen, OH, NH₂, C₁-C₃ alkyl, C₁-C₃ alkoxyl, or C₁-C₃ alkylamine, or R″₁ and R″₂, together with the atoms to which they are bound, form a C₃-C₇ carbocyclic or 3- to 7-membered heterocyclic ring.

54. The bifunctional compound of claim 35, wherein the degron binds von Hippel Landau tumor suppressor (VHL).

55. The bifunctional compound of claim 54, wherein the degron is represented by any one of structures (D2-a) to (D2-j): 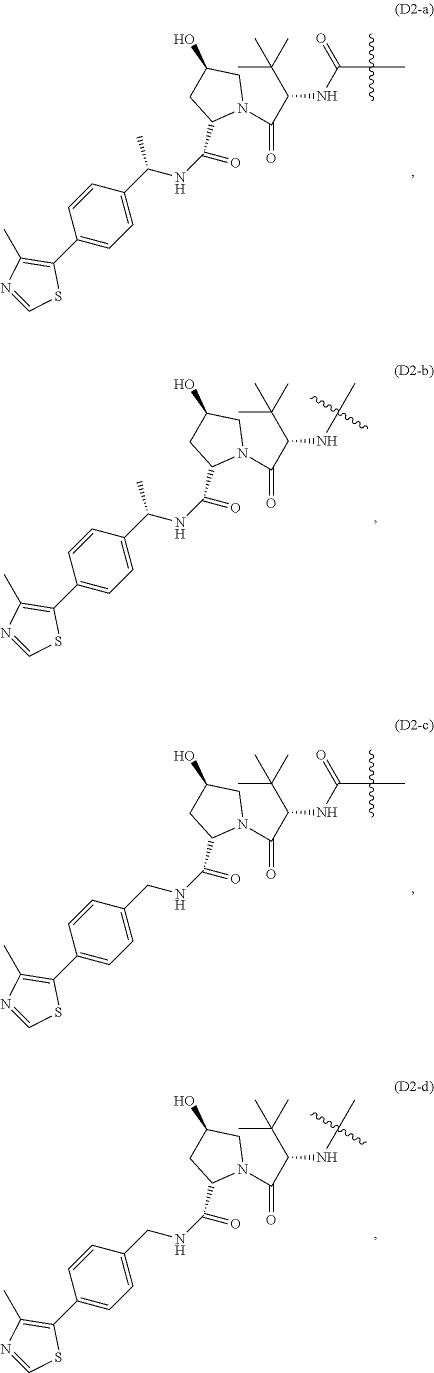 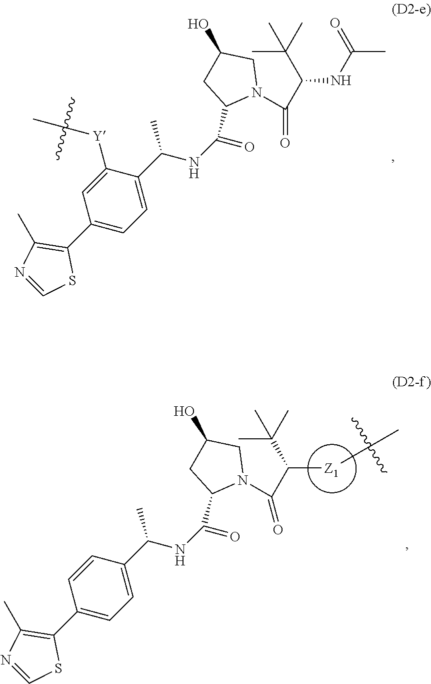
wherein Z₁ is a C₅-C₆ carbocyclic or 5- or 6-membered heterocyclic group, containing 1 to 3 heteroatoms selected from N, O, and S, 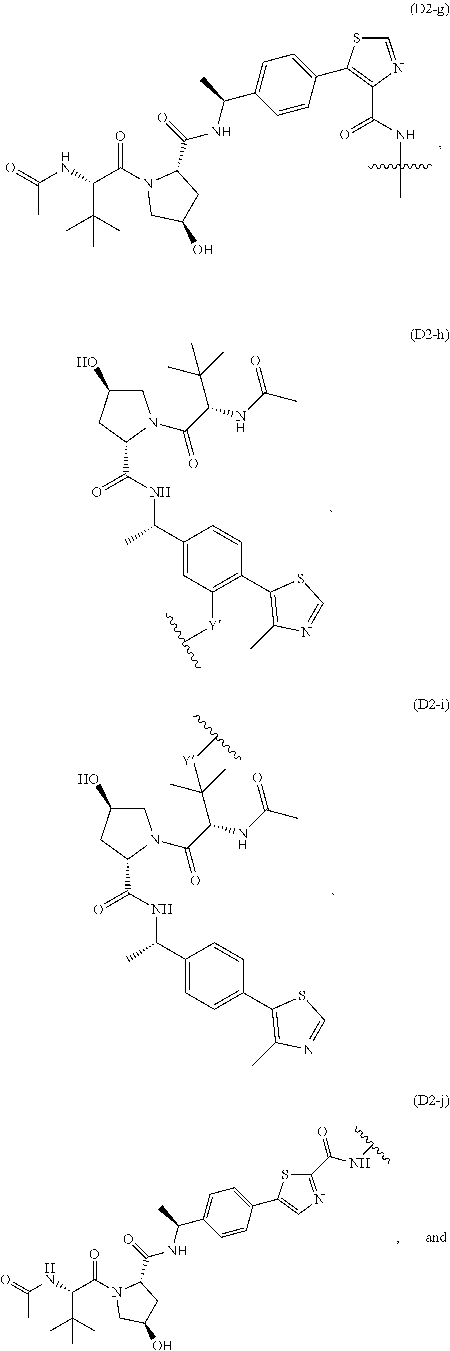 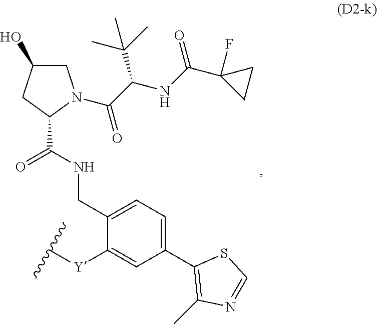
wherein Y′ is a bond, CH₂, NH, NMe, O, or S, or a stereoisomer thereof.

56. The bifunctional compound of claim 55, wherein Z is

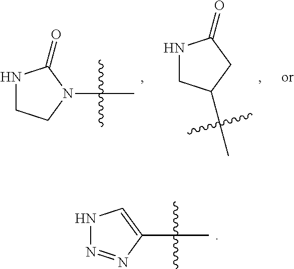 [SMILES: *N1CCNC1=O ; *C1CNC(=O)C1 ; *c1c[nH]nn1]

57. The bifunctional compound of claim 55, wherein Y′ is O.

58. The bifunctional compound of claim 35, which is: 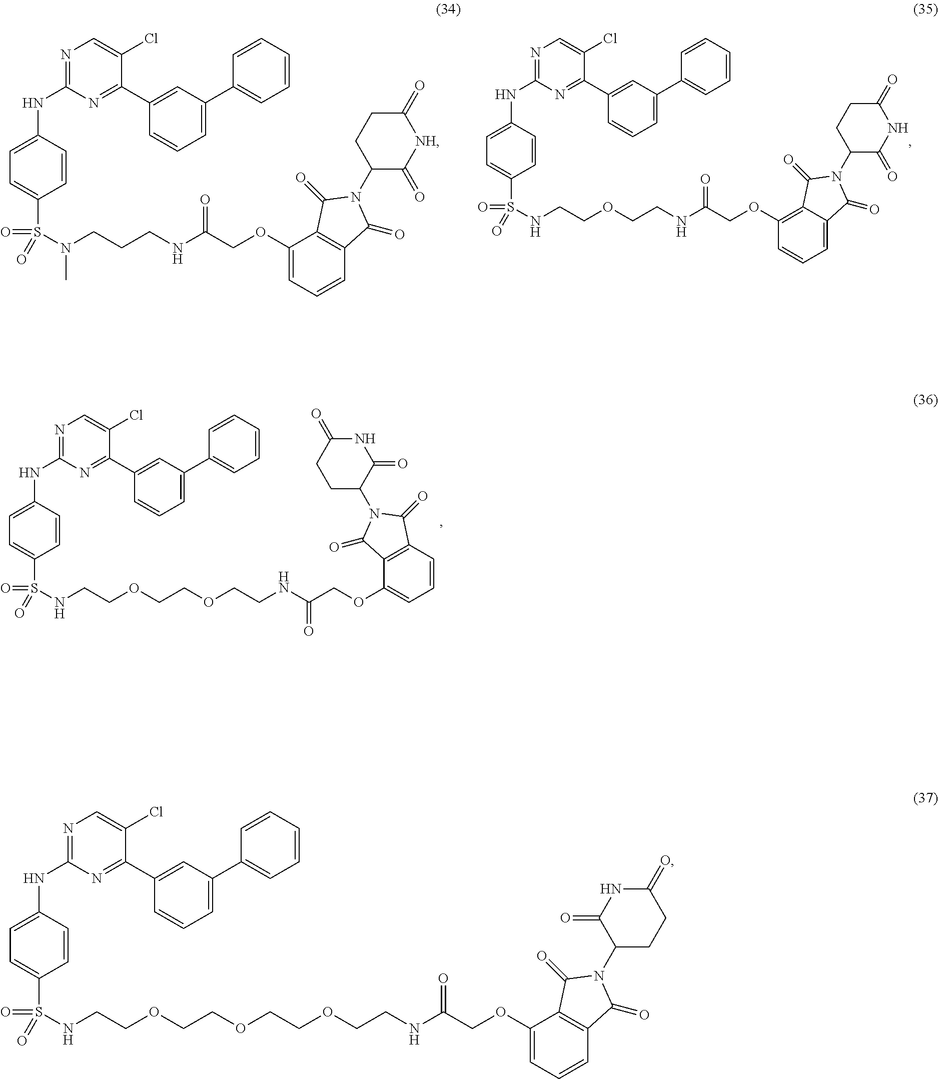 [SMILES: CN(CCCNC(=O)COc1cccc2c1C(=O)N(C1CCC(=O)NC1=O)C2=O)S(=O)(=O)c1ccc(Nc2ncc(Cl)c(-c3cccc(-c4ccccc4)c3)n2)cc1 ; O=C(COc1cccc2c1C(=O)N(C1CCC(=O)NC1=O)C2=O)NCCOCCNS(=O)(=O)c1ccc(Nc2ncc(Cl)c(-c3cccc(-c4ccccc4)c3)n2)cc1 ; O=C(COc1cccc2c1C(=O)N(C1CCC(=O)NC1=O)C2=O)NCCOCCOCCNS(=O)(=O)c1ccc(Nc2ncc(Cl)c(-c3cccc(-c4ccccc4)c3)n2)cc1 ; O=C(COc1cccc2c1C(=O)N(C1CCC(=O)NC1=O)C2=O)NCCOCCOCCOCCNS(=O)(=O)c1ccc(Nc2ncc(Cl)c(-c3cccc(-c4ccccc4)c3)n2)cc1] 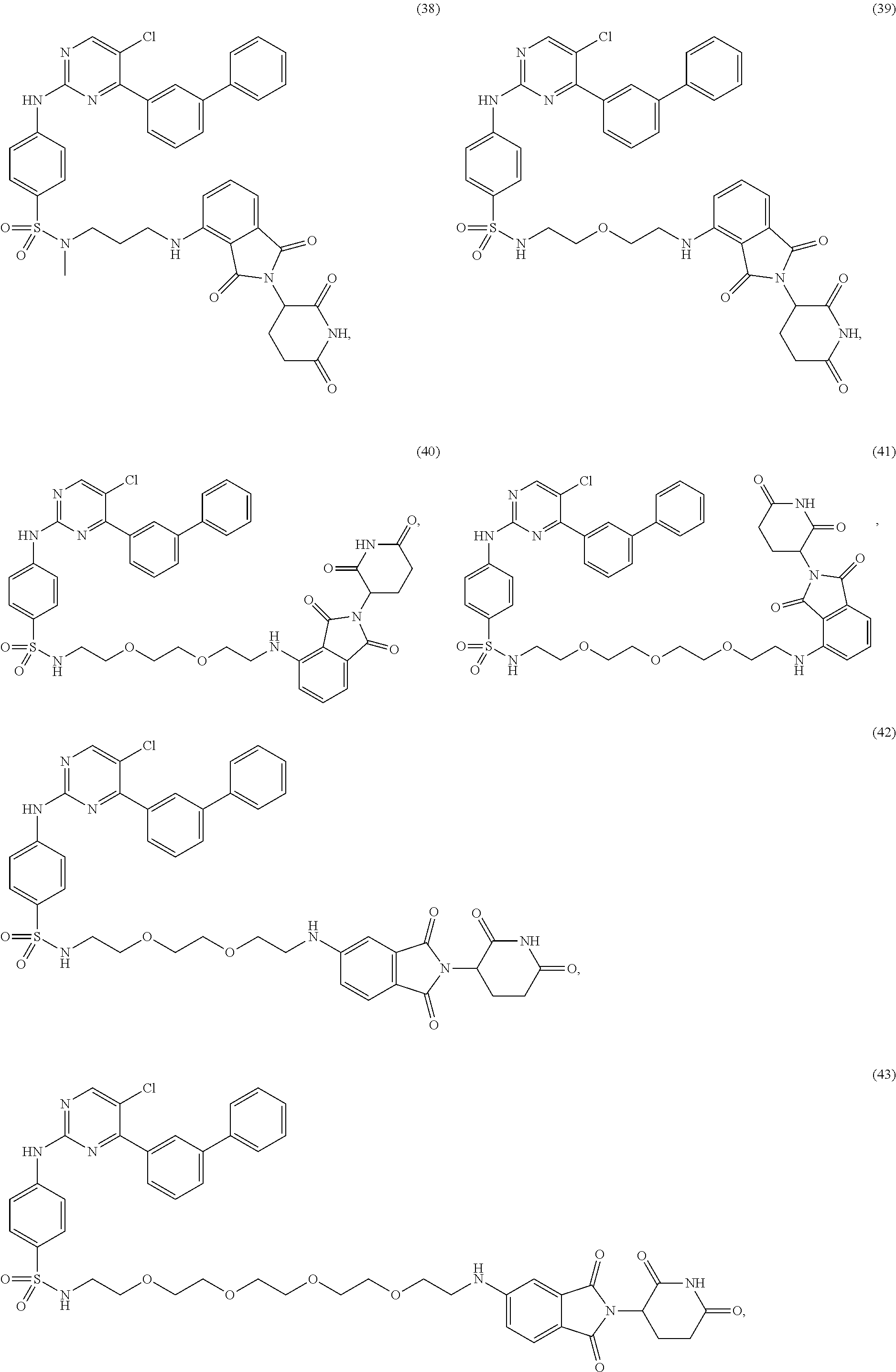 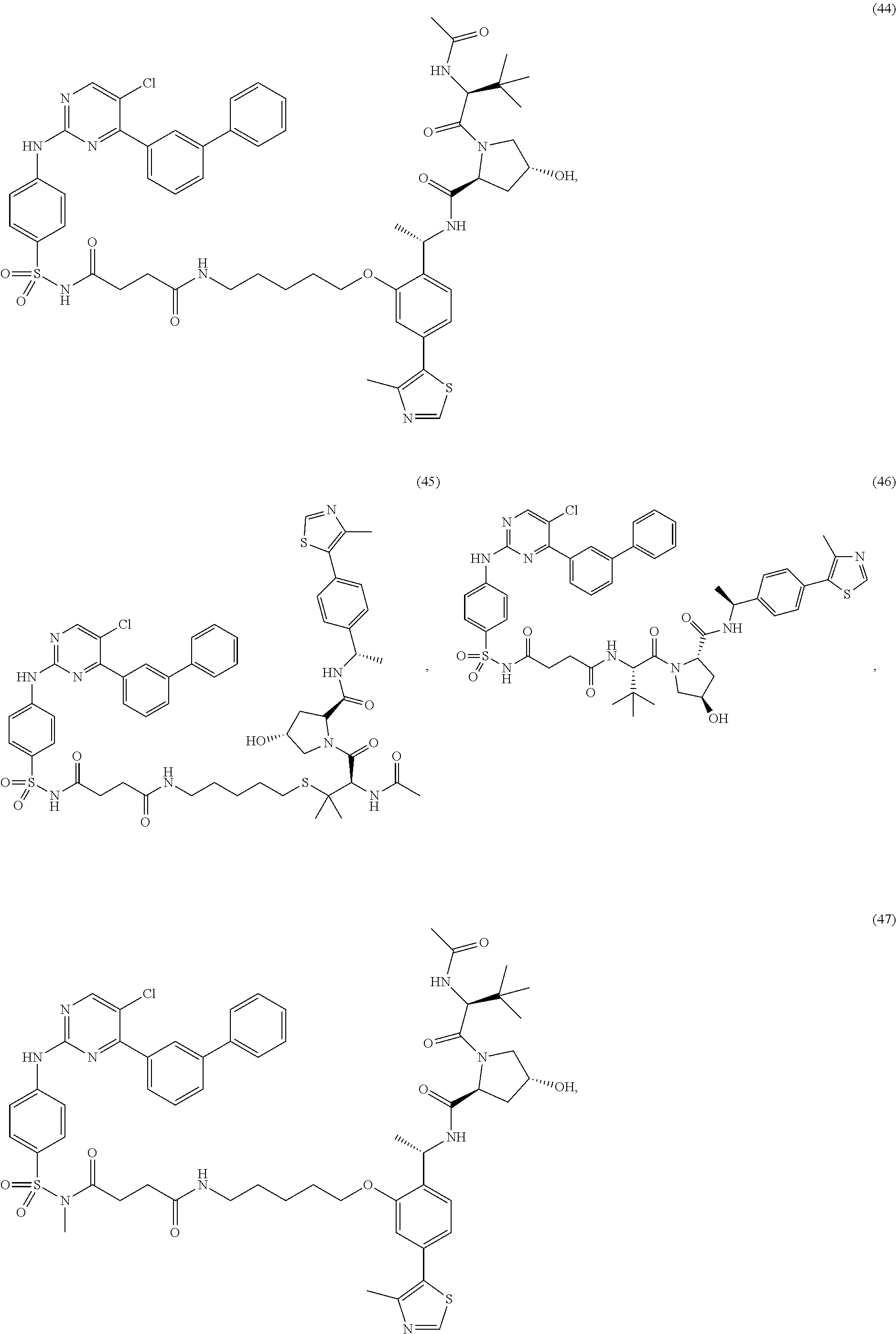 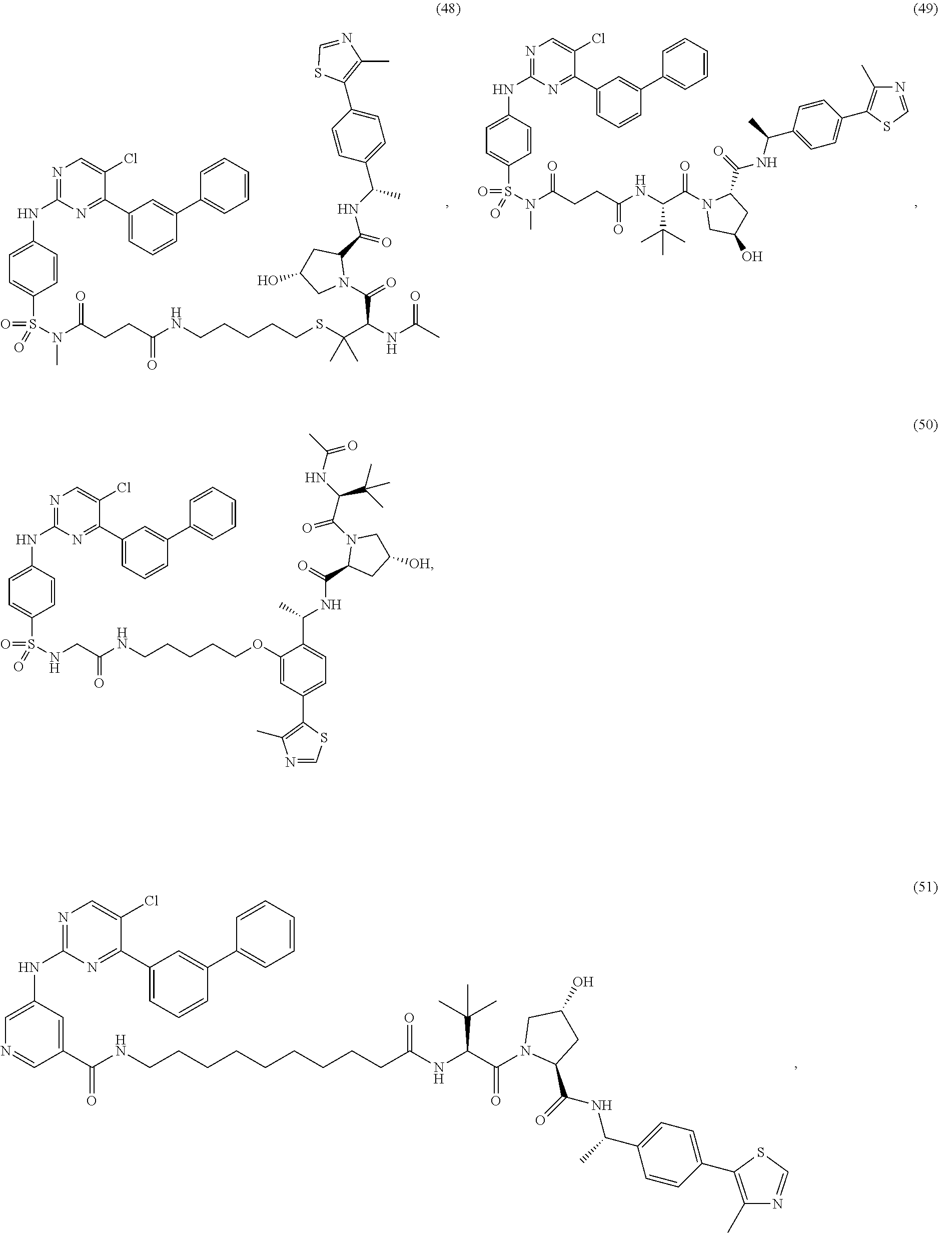 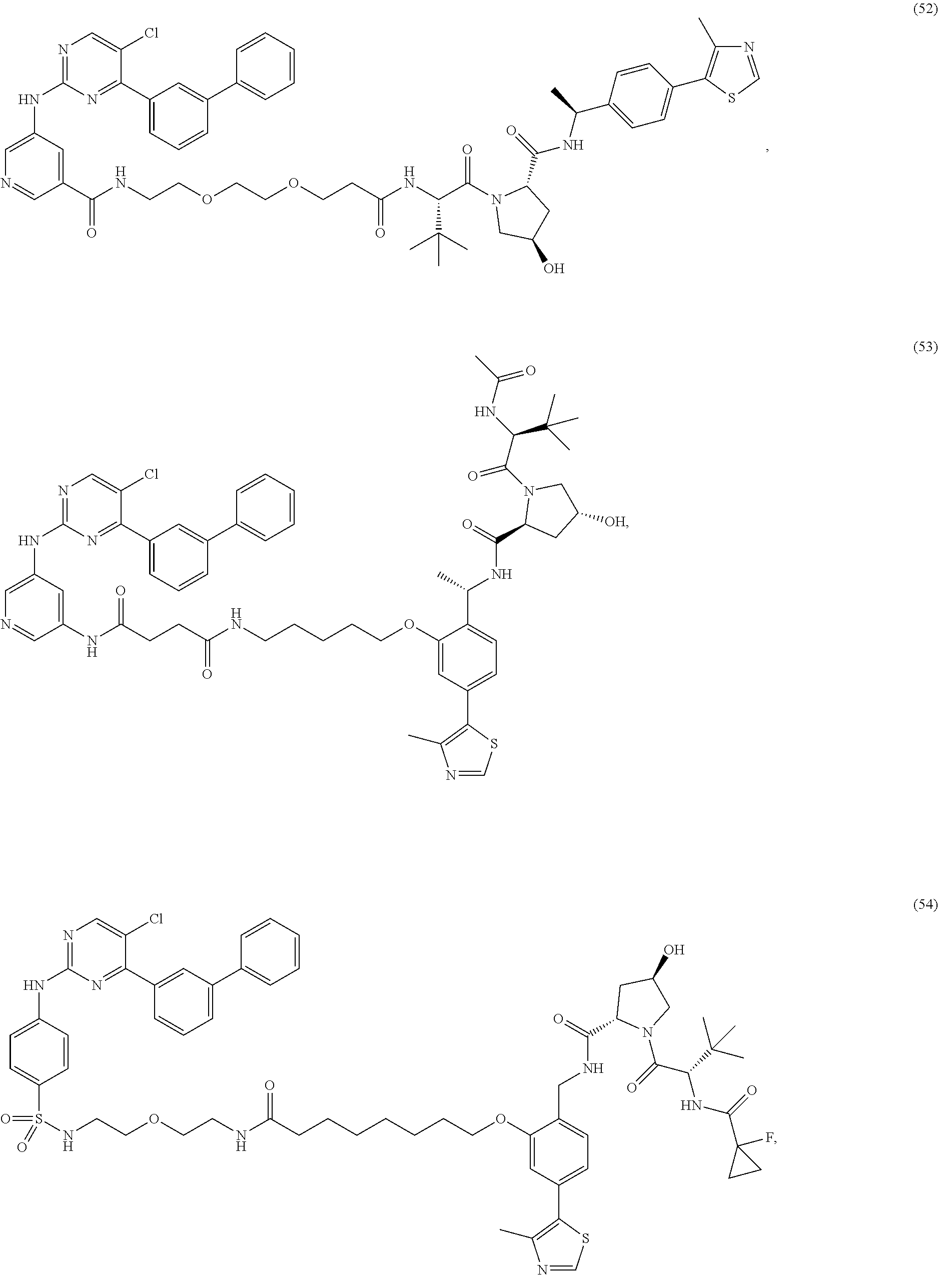 [SMILES: Cc1ncsc1-c1ccc([C@H](C)NC(=O)[C@@H]2C[C@@H](O)CN2C(=O)[C@@H](NC(=O)CCOCCOCCNC(=O)c2cncc(Nc3ncc(Cl)c(-c4cccc(-c5ccccc5)c4)n3)c2)C(C)(C)C)cc1 ; CC(=O)N[C@H](C(=O)N1C[C@H](O)C[C@H]1C(=O)N[C@@H](C)c1ccc(-c2scnc2C)cc1OCCCCCNC(=O)CCC(=O)Nc1cncc(Nc2ncc(Cl)c(-c3cccc(-c4ccccc4)c3)n2)c1)C(C)(C)C ; Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)NCCOCCNS(=O)(=O)c2ccc(Nc3ncc(Cl)c(-c4cccc(-c5ccccc5)c4)n3)cc2)c1] 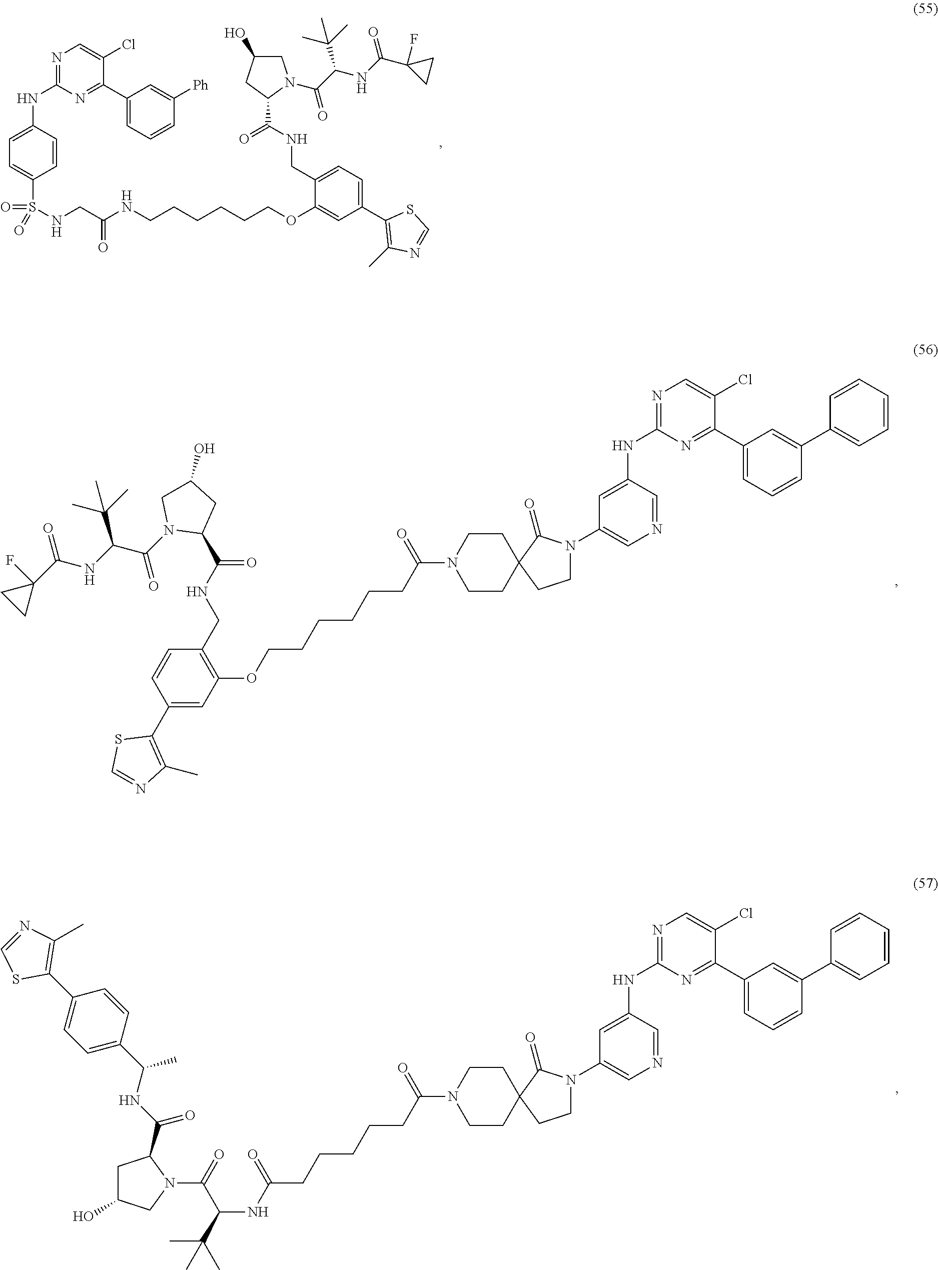 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCNC(=O)CNS(=O)(=O)c2ccc(Nc3ncc(Cl)c(-c4cccc(-c5ccccc5)c4)n3)cc2)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCC3(CC2)CCN(c2cncc(Nc4ncc(Cl)c(-c5cccc(-c6ccccc6)c5)n4)c2)C3=O)c1 ; Cc1ncsc1-c1ccc([C@H](C)NC(=O)[C@@H]2C[C@@H](O)CN2C(=O)[C@@H](NC(=O)CCCCCC(=O)N2CCC3(CC2)CCN(c2cncc(Nc4ncc(Cl)c(-c5cccc(-c6ccccc6)c5)n4)c2)C3=O)C(C)(C)C)cc1]
or a pharmaceutically acceptable salt or stereoisomer thereof.

59. A pharmaceutical composition, comprising a therapeutically effective amount of the bifunctional compound or pharmaceutically acceptable salt or stereoisomer thereof of claim 35, and a pharmaceutically acceptable carrier.

60. A method of treating a disease or disorder by modulating the level or activity of at least one of PIP4K2A, PIP4K2B, and PIP4K2C, comprising administering to a subject in need thereof a therapeutically effective amount of the bifunctional compound or pharmaceutically acceptable salt or stereoisomer thereof of claim 35, or
a method of modulating immune functions in a subject in need thereof, comprising administering to a subject a therapeutically effective amount of the bifunctional compound or pharmaceutically acceptable salt or stereoisomer thereof of claim 35, or a method of stimulating/activating the immune system by reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of the bifunctional compound or pharmaceutically acceptable salt or stereoisomer thereof of claim 35.

61.-69. (canceled)
